(19) United States
(12) Patent Application Publication
(10) Pub. No.: US 2012/0202690 A1
(43) Pub. Date: Aug. 9, 2012

(54) HERBICIDAL COMPOUNDS

(75) Inventors: William Guy Whittingham, Bracknell (GB); Caroline Louise Winn, Bracknell (GB); Julie Evelyne Blanc, Bracknell (FR); Shuji Hachisu, Bracknell (GB); Matthew Brian Hotson, Bracknell (GB); Harry Glithro, Bracknell (GB)

(73) Assignee: SYNGENTA LIMITED, Greensboro, NC (US)

(21) Appl. No.: 13/502,054

(22) PCT Filed: Oct. 8, 2010
(86) PCT No.: PCT/GB2010/001890

§ 371 Date: Apr. 13, 2012

(30) Foreign Application Priority Data
Oct. 13, 2009 (GB) 0917934.2

(51) Int. Cl. A01N 43/90; A01N 25/32; C07D 495/04; A01P 13/00; C07D 475/00; C07D 475/14; C07D 498/04; C07D 487/04; C07D 471/04

(52) U.S. Cl. 504/103; 544/280; 504/241; 504/136; 544/278; 544/279; 546/113; 504/246; 504/130; 544/257; 544/251; 544/105; 504/225; 544/264

(57) ABSTRACT

The present invention relates to substituted heterobicyclic carboxylic acid derivatives, as well as N-oxides and agriculturally acceptable salts thereof, and their use in controlling plant growth, particularly undesirable plant growth, in crops of useful plants. The invention extends to herbicidal compositions comprising such compounds, N-oxides and/or salts as well as mixtures of the same with one or more further active ingredients and/or a safener.

The present invention relates to certain substituted heterobicyclic carboxylic acid derivatives, to processes for their preparation, herbicidal compositions comprising them, and their use in controlling plants or inhibiting plant growth.

Herbicidal 4-aminopicolinates are disclosed in WO01/51468, WO03/011853, WO2004/089906, WO2005/016887, WO2005/063721 and WO2006/062979.

WO2009/029735 discloses intermediates of the formula

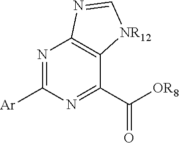

which are stated to be useful in the preparation of herbicidal compounds.

This invention seeks to provide alternative herbicidal compounds.

In a first aspect, the invention provides compound having the formula (I):

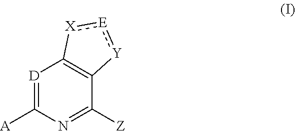

or a salt or N-oxide thereof,

wherein:

A is halogen, C2-C6 alkenyl optionally substituted by 1 to 3 groups R¹, C3-C8 cycloalkyl optionally substituted by 1 to 3 groups R¹, C1-C6 alkylthio optionally substituted by 1 to 3 groups R¹, C6-C10 aryl optionally substituted by 1 to 3 groups R² or a mono- or bicyclic heteroaryl group having 5 to 10 ring atoms and at least one ring atom which is nitrogen, oxygen or sulfur optionally substituted by 1 to 3 groups R²;

D is N or CR³;

X is O, S, N or NR⁴;

Y is CR⁵, CR⁵R⁶, N, NR⁵, O or S;

E is —(CR⁷R⁸)ₙ—;

n is 1, 2 or 3;

[symbol] is a bond that is optionally single or double

Z is C(O)R⁹, C(S)R¹⁰, or C(═NR¹¹)R¹²;

each R¹ is independently halogen, hydroxyl, nitro, amino, C1-C3 alkylamino, di(C1-C3)alkylamino, cyano, C1-C3 alkyl, C1-C3 haloalkyl, C2-C3 alkenyl, C1-C3 alkoxy, C1-C3 haloalkoxy, C1-C3 alkylthio, C1-C3 alkylsulphonyl, C2-C6 carboxyalkyl, carboxyl, C2-C6 alkoxycarbonyl, C2-C7 alkylcarbonyloxy or C6-C10 aryl optionally substituted by 1 to 3 groups R²;

each R² is independently halogen, hydroxyl, nitro, amino, cyano, C1-C3 alkyl, C1-C3 haloalkyl, C1-C3 alkoxy, C1-C3 haloalkoxy, C1-C3 alkylthio, C1-C3 haloalkylthio, C1-C3 alkylsulphonyl, C1-C3 alkylsulphonyloxy, C2-C6 carboxyalkyl, C2-C6 alkoxycarbonyl, C2-C7 alkylcarbonyloxy, C1-C3 alkylamino, or di(C1-C3 alkyl)amino;

R³ is hydrogen, halogen, C1-C3 alkyl, C1-C3 haloalkyl, C2-C4 alkoxyalkyl, C2-C4 alkenyl, C2-C4 haloalkenyl, or cyclopropyl optionally substituted by 1 to 3 groups R¹;

R⁴ is hydrogen, C1-C6 alkyl optionally substituted by 1 to 3 groups R¹³, C2-C6 alkenyl optionally substituted by 1 to 3 groups R¹³, C2-C6 alkynyl optionally substituted by 1 to 3 groups R¹³, C3-C8 cycloalkyl optionally substituted by 1 to 3 groups R¹³, C1-C6 acyl optionally substituted by 1 to 3 groups R¹, C1-C6 alkoxycarbonyl optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R², C1-C6 alkylsulphonyl optionally substituted by 1 to 3 groups R¹ or C6-C10 arylsulphonyl optionally substituted by 1 to 3 groups R²;

each of R⁵ and R⁶ is independently hydrogen, halogen, C1-C6 alkyl optionally substituted by 1 to 3 groups R¹, C1-C6 alkoxy, C6-C10 aryl optionally substituted by 1 to 3 groups R², carboxyl, C1-C7 acyl, C2-C7 alkoxycarbonyl, or, taken together with the carbon atom to which they are attached, R⁵ and R⁶ form a C1-C6 alkenyl group optionally substituted by 1 to 3 groups R¹, a carbonyl group, or a C3-C6 cycloalkyl group optionally substituted by 1 to 3 groups R¹;

each of R⁷ and R⁸ is independently hydrogen, halogen, C1-C6 alkyl optionally substituted by 1 to 3 groups R¹, C1-C6 alkoxy, C6-C10 aryl optionally substituted by 1 to 3 groups R², carboxyl, C1-C7 acyl, C2-C7 alkoxycarbonyl, or R⁷ represents an additional bond between the carbon atom to which it is attached and the adjacent ring atom or, taken together with the carbon atom to which they are attached, R⁷ and R⁸ form a C1-C6 alkenyl group optionally substituted by 1 to 3 groups R¹, a carbonyl group, or a C3-C6 cycloalkyl group optionally substituted by 1 to 3 groups R¹ or, when n is 2 or 3, taken together with the carbon atoms to which they are attached, any two R⁷ and R⁸ form a 5- or 6-membered saturated, unsaturated or aromatic ring, the ring optionally including 1 to 3 ring atoms which are independently selected from nitrogen, oxygen or sulphur and optionally substituted by 1 to 3 groups R¹;

R⁹ is hydrogen, hydroxyl, C1-C10 alkoxy optionally substituted by C1-C6 alkoxy, C1-C6 alkoxy-C1-C6alkoxy, phenyl, C5-C10 heteroaryl or C3-C10 heterocyclyl, C2-C10 alkenyloxy, C3-C8 cycloalkoxy optionally substituted by C1-C6 alkoxy or phenyl, C1-C6 alkylthio, amino, C1-C6 alkylamino, di(C1-C6 alkyl)amino, or (C1-C6 alkyl)(C1-C6 alkoxy)amino;

R¹⁰ is C1-C10 alkoxy optionally substituted by C1-C6 alkoxy or phenyl, C2-C10 alkenyloxy, C3-C8 cycloalkoxy optionally substituted by C1-C6 alkoxy or phenyl, C1-C6 alkylthio, amino, C1-C6 alkylamino, or di(C1-C6 alkyl)amino;

R¹¹ is hydrogen, C1-C6 alkyl, C1-C6 alkoxy, C3-C8 cycloalkoxy, amino, C1-C6 alkylamino, or di(C1-C6 alkyl)amino;

R¹² is hydrogen, C1-C6 alkoxy, C3-C8 cycloalkoxy, C1-C6 alkylthio, amino, C1-C6 alkylamino, or di(C1-C6 alkyl)amino;

each R¹³ is independently cyano, hydroxyl, carboxyl, C3-C6 cycloalkyl, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R², C1-C4 alkoxy; C1-C4 alkoxy(C1-C4)alkoxy; C1-C4 alkoxycarbonyl; or tri(C1-C4)alkylsilyl;

provided that
(i) when Y is NR⁵, X is N, Z is C(O)R⁹, D is N, E is —(CR⁷R⁸)ₙ—, R⁵ is alkyl or haloalkyl, R⁷ represents an additional bond to X, and R⁹ is alkoxy, then R⁸ is other than H;
(ii) when XEY is —N(R⁴)C(O)NH—, Z is not C(O)NH₂, C(O)NHCH₃ or C(O)N(CH₃)₂;
(iii) the compound of formula (I) is not:
   9-benzyl-9H-purine-2,6-dicarboxamide;
   9-(2-hydroxyethyl)-2-(prop-1-enyl)-9H-purine-6-carboxamide;
   9-(2-hydroxyethyl)-2-phenyl-9H-purine-6-carboxamide;
   9-phenyl-2-(pyridin-3-yl)-9H-purine-6-carboxamide;
   2-(3-hydroxyphenyl)-9-(2-methoxyphenyl)-9H-purine-6-carboxamide;
   2-(2-hydroxyphenyl)-9-(2-methoxyphenyl)-purine-6-carboxamide;
   6-oxo-8-phenyl-2-(pyridin-3-yl)-5,6,7,8-tetrahydropteridine-4-carboxamide;
   6-oxo-8-phenyl-2-(pyridin-4-yl)-5,6,7,8-tetrahydropteridine-4-carboxamide;
   2-(3-hydroxyphenyl)-8-(2-methoxyphenyl)-6-oxo-5,6,7,8-tetrahydropteridine-4-carboxamide;
   2-chloro-9-phenyl-9H-purine-6-carboxylic acid;
   2-chloro-9-methyl-9H-purine-6-carboxylic acid;
   2-chloro-9-methyl-9H-purine-6-carboxylic acid ethyl ester;
   2-chloro-9-ethoxycarbonylmethyl-9H-purine-6-carboxylic acid ethyl ester.

In a second aspect, the invention relates to a herbicidal composition comprising a compound of formula (I), wherein A is halogen, C1-C6 alkyl optionally substituted by 1 to 3 groups R¹, C1-6 haloalkyl optionally substituted by 1 to 3 groups R¹, C2-C6 alkenyl optionally substituted by 1 to 3 groups R¹, C3-C8 cycloalkyl optionally substituted by 1 to 3 groups R¹, C1-C6 alkylthio optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R² and D, X, E, Y and Z are as defined above without the provisos (i), (ii) and (iii) together with at least one agriculturally acceptable adjuvant or diluent.

In a third aspect, the invention relates to the use of a compound of formula (I), wherein A is halogen, C1-C6 alkyl optionally substituted by 1 to 3 groups R¹, C1-6 haloalkyl optionally substituted by 1 to 3 groups R¹, C2-C6 alkenyl optionally substituted by 1 to 3 groups R¹, C3-C8 cycloalkyl optionally substituted by 1 to 3 groups R¹, C1-C6 alkylthio optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R² and D, X, E, Y and Z are as defined above without the provisos (i), (ii) and (iii) or composition as defined above as a herbicide.

In a fourth aspect, the invention relates to a method of controlling weeds in crops of useful plants, comprising applying to said weeds or to the locus of said weeds, or to said useful crop plants, a compound of formula (I), wherein A is halogen, C1-C6 alkyl optionally substituted by 1 to 3 groups R¹, C1-6 haloalkyl optionally substituted by 1 to 3 groups R¹, C2-C6 alkenyl optionally substituted by 1 to 3 groups R¹, C3-C8 cycloalkyl optionally substituted by 1 to 3 groups R¹, C1-C6 alkylthio optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R² and D, X, E, Y and Z are as defined above without the provisos (i), (ii) and (iii) or composition as defined above.

In a fifth aspect, the invention relates to a process for the preparation of compounds of formula (I).

In a sixth aspect, the invention relates to intermediates useful in the preparation of compounds of formula (I).

Tautomers

The compounds of formula (I) may exist as different geometric isomers, or in different tautomeric forms. This invention covers all such isomers and tautomers, and mixtures thereof in all proportions, as well as isotopic forms such as deuterated compounds. Zwitterionic forms are also covered. For example, compounds of formula (II) may exist in equilibrium with the zwitterionic forms (III) and (IV).

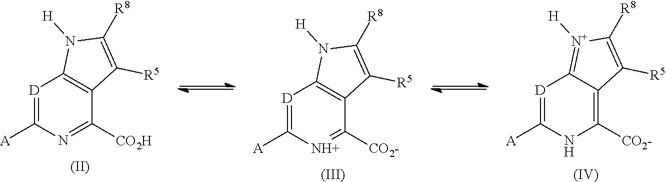

Asymmetry

The compounds of this invention may contain an asymmetric carbon atom and some of the compounds of this invention may contain one or more asymmetric centers and may thus give rise to optical isomers and diastereomers. While shown without respect to stereochemistry, the present invention includes such optical isomers and diastereomers; as well as the racemic and resolved, enantiomerically pure R and S stereoisomers; as well as other mixtures of the R and S stereoisomers and agrochemically acceptable salts thereof. It is recognized that one optical isomer, including diastereomer and enantiomer, or stereoisomer may have favorable properties over the other. Thus when disclosing and claiming the invention, when one racemic mixture is disclosed, it is clearly contemplated that both optical isomers, including diastereomers and enantiomers, or stereoisomers substantially free of the other are disclosed and claimed as well.

“Alkyl”, as used herein refers to an aliphatic hydrocarbon chain and includes straight and branched chains e. g. of 1 to 6 carbon atoms such as methyl, ethyl, n-propyl, isopropyl, n-butyl, isobutyl, sec-butyl, t-butyl, n-pentyl, isopentyl, neo-pentyl, n-hexyl, and isohexyl.

“Alkenyl”, as used herein, refers to an aliphatic hydrocarbon chain having at least one double bond, and preferably one double bond, and includes straight and branched chains e. g. of 2 to 6 carbon atoms such as ethenyl, propenyl, isopropenyl, but-1-enyl, but-2-enyl, but-3-enyl, 2-methypropenyl.

“Alkynyl”, as used herein, refers to an aliphatic hydrocarbon chain having at least one triple bond, and preferably one triple bond, and includes straight and branched chains e. g. of 2 to 6 carbon atoms such as ethynyl, propynyl, but-1-ynyl, but-2-ynyl and but-3-ynyl.

“Cycloalkyl”, as used herein, refers to a cyclic, saturated hydrocarbon group having from 3 to 8 ring carbon atoms. Examples of cycloalkyl groups are cyclopropyl, cyclobutyl, cyclopentyl, cyclohexyl, cycloheptyl and cyclooctyl.

“Alkoxy” as used herein refers to the group —O-alkyl, wherein alkyl is as defined above. Examples of alkoxy groups include methoxy, ethoxy, n-propoxy, isopropoxy, n-butoxy, isobutoxy, sec-butoxy, t-butoxy, n-pentoxy, isopentoxy, neo-pentoxy, n-hexyloxy, and isohexyloxy.

“Alkenyloxy” as used herein refers to the group —O-alkenyl, wherein alkenyl is as defined above.

“Cycloalkoxy” as used herein refers to the group —O-cycloalkyl, wherein cycloalkyl is as defined above. Examples of cycloalkoxy groups are cyclopropoxy, cyclobutoxy, cyclopentoxy, cyclohexyloxy, cycloheptyloxy and cyclooctyloxy.

“Alkoxyalkyl” as used herein refers to the group I -alkyl-O-alkyl, where each alkyl is, independently, as defined above.

“Alkoxyalkoxy” means a radical —Oalkyl-O-alkyl, wherein each alkyl is, independently, as defined above.

“Alkylthio” as used herein refers to the group —S-alkyl, wherein alkyl is as defined above. Examples of Alkylthio groups are methylthio, ethylthio, n-propylthio, isopropylthio, n-butylthio, isobutylthio, sec-butylthio, t-butylthio, n-pentylthio, isopentylthio, neo-pentylthio, n-hexylthio, and isohexylthio.

“Haloalkylthio” means a radical —S-haloalkyl, where haloalkyl is as defined below.

“Alkyl sulphinyl” refers to the group —S(O)-alkyl, wherein alkyl is as defined above.

“Alkylsulphonyl” refers to the group —S(O)₂-alkyl, wherein alkyl is as defined above.

“Alkylsulphonyloxy” refers to the group —O—S(O₂)-alkyl, wherein alkyl is as defined above.

“Halogen”, “halide” and “halo” refer to iodine, bromine, chlorine and fluorine.

“Haloalkyl” as used herein refers to an alkyl group as defined above wherein at least one hydrogen atom has been replaced with a halogen atom as defined above. Examples of haloalkyl groups include chloromethyl, dichloromethyl, trichloromethyl, fluoromethyl, difluoromethyl and trifluoromethyl. Preferred haloalkyl groups are fluoroalkyl groups (i.e. haloalkyl groups, containing fluorine as the only halogen). More highly preferred haloalkyl groups are perfluoroalkyl groups, i.e. alkyl groups wherein all the hydrogen atoms are replaced with fluorine atoms.

“Haloalkenyl” as used herein refers to an alkenyl group as defined above wherein at least one hydrogen atom has been replaced with a halogen atom as defined above.

“Haloalkoxy” refers to an alkoxy group as defined above wherein at least one of the hydrogen atoms on the alkyl moiety has been replaced with a halogen atom as defined above.

“Acyl” as used herein refers to the group —C(O)-alkyl or —C(O)H, wherein the alkyl group is as defined above. Examples of acyl groups are formyl, acetyl, pivaloyl etc.

“Alkoxycarbonyl” refers to the group —C(O)—O-alkyl, wherein the alkyl group is as defined above. Examples of alkoxycarbonyl groups include methoxycarbonyl, ethoxycarbonyl, i-propoxycarbonyl, n-propoxycarbonyl, n-butoxycarbonyl and s-butoxycarbonyl etc.

“Alkylcarbonyloxy” refers to the group —OC(O)-alkyl, wherein alkyl is as defined above.

“Carboxyalkyl” refers to the group -alkyl-COOH, wherein alkyl is as defined above.

“Alkylamino” refers to the group —NH-alkyl, wherein alkyl is as defined above. Examples of alkylamino groups are methylamino, ethylamino, n-propylamino, i-propylamino etc.

“Dialkylamino” refers to the group —N(alkyl)alkyl′, wherein alkyl and alkyl′ are both alkyl groups as defined above which may be the same or different. Examples of dialkylamino groups are dimethylamino, diethylamino, di-n-propylamino, methylethylamino, methyisopropylamino, etc.

“Dialkylphosphonyl” refers to the group —P(O)(O-alkyl)(O-alkyl′), wherein alkyl and alkyl′ are both alkyl groups as defined above which may be the same or different. Examples of dialkylphosphonyl groups are dimethylphosphonyl, diethylphosphonyl, ethyl methyl phosphonyl etc.

“Alkylene” refers to a branched or linear divalent hydrocarbon radical. Examples of alkylene are methylene, 1,1-ethylene, 1,2-ethylene, 1,1-propylene, 1,2-propylene, 1,3-propylene and 2,2-propylene etc.

“Trialkylsilyl” refers to the group —Si(alkyl)₃, wherein each alkyl is, independently, as defined above.

“Aryl” as used herein refers to an unsaturated aromatic carbocyclic group of from 6 to 10 carbon atoms having a single ring (e. g., phenyl) or multiple condensed (fused) rings, at least one of which is aromatic (e.g., indanyl, naphthyl). Preferred aryl groups include phenyl, naphthyl and the like.

“Aryloxy” refers to the group —O-aryl, wherein aryl is as defined above. Preferred aryloxy groups include phenoxy, naphthyloxy and the like.

“Arylalkyl” refers to the group -alkyl-aryl, wherein aryl and alkyl are as defined above.

“Arylsulphonyl” refers to the group —S(O)₂-aryl, wherein aryl is as defined above.

“Heteroaryl” refers to a ring system containing 5 to 10 ring atoms, at least one ring heteroatom and consisting either of a single aromatic ring or of two or more fused rings, at least one of which is aromatic. Preferably, single rings will contain up to three and bicyclic systems up to four heteroatoms which will preferably be chosen from nitrogen, oxygen and sulfur. Examples of such groups include pyridyl, pyridazinyl, pyrimidinyl, pyrazinyl, triazinyl, furanyl, thiophenyl, oxazolyl, isoxazolyl, oxadiazolyl, thiazolyl, isothiazolyl, thiadiazolyl, pyrrolyl, pyrazolyl, imidazolyl, triazolyl and tetrazolyl. Examples of bicyclic groups are benzothiophenyl, benzimidazolyl, benzothiadiazolyl, methylenedioxyphenyl, quinolinyl, cinnolinyl, quinoxalinyl and pyrazolo[1,5-a]pyrimidinyl.

“Heteroaryloxy” refers to the group —O-heteroaryl, wherein heteroaryl is as defined above.

“Heterocyclyl” refers to a non-aromatic ring system containing 3 to 10 ring atoms, at least one ring heteroatom and consisting either of a single ring or of two or more fused rings. Preferably, single rings will contain up to three and bicyclic systems up to four heteroatoms which will preferably be chosen from nitrogen, oxygen and sulfur. Examples of such groups include pyrrolidinyl, imidazolinyl, pyrazolidinyl, piperidyl, piperazinyl, quinuclidinyl, morpholinyl, together with unsaturated or partially unsaturated analogues such as 4,5,6,7-tetrahydro-benzothiophenyl, chromen-4-onyl, 9H-fluorenyl, 3,4-dihydro-2H-benzo-1,4-dioxepinyl, 2,3-dihydro-benzofuranyl, piperidinyl, 1,3-dioxolanyl, 1,3-dioxanyl, 4,5-dihydro-isoxazolyl, tetrahydrofuranyl and morpholinyl.

“Optionally substituted” as used herein means the group referred to can be substituted at one or more positions by any one or any combination of the radicals listed thereafter. For most groups, one or more hydrogen atoms are replaced by the radicals listed thereafter. For halogenated groups, for example, haloalkyl groups, one or more halogen atoms are replaced by the radicals listed thereafter.

Salts

Suitable salts include those derived from alkali or alkaline earth metals and those derived from ammonia and amines. Preferred cations include sodium, potassium, magnesium, and ammonium cations of the formula N⁺(R¹⁹R²⁰R²¹R²²)wherein R¹⁹, R²⁰, R²¹ and R²² are independently selected from hydrogen, C1-C6 alkyl and C1-C6 hydroxyalkyl. Salts of the compounds of Formula I can be prepared by treatment of compounds of Formula I with a metal hydroxide, such as sodium hydroxide, or an amine, such as ammonia, trimethylamine, diethanolamine, 2-methylthiopropylamine, bisallylamine, 2-butoxyethylamine, morpholine, cyclododecylamine, benzylamine, or triisopropanolamine. Amine salts are often preferred forms of the compounds of Formula I because they are water-soluble and lend themselves to the preparation of desirable aqueous based herbicidal compositions.

Acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic moiety.

Preferred values of A, D, E, X, Y, Z and R¹ to R¹³ are set out below.

In one embodiment, A is halogen, C2-C6 alkenyl optionally substituted by 1 to 3 groups R¹, C3-C8 cycloalkyl optionally substituted by 1 to 3 groups R¹, C1-C6 alkylthio optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R²;

D is N or CR³;

X is O, S, N or NR⁴;

Y is CR⁵, CR⁵R⁶, N, NR⁵, O or S;

E is —(CR⁷R⁸)ₙ—;

n is 1, 2 or 3;

[symbol] is a bond that is optionally single or double

Z is C(O)R⁹, C(S)R¹⁰, or C(═NR¹¹)R¹²;

each R¹ is independently halogen, hydroxyl, nitro, amino, C1-C3 alkylamino, di(C1-C3)alkylamino, cyano, C1-C3 alkyl, C1-C3 haloalkyl, C2-C3 alkenyl, C1-C3 alkoxy, C1-C3 haloalkoxy, C1-C3 alkylthio, C1-C3 alkylsulphonyl, C2-C6 carboxyalkyl, carboxy, C2-C6 alkoxycarbonyl or C2-C7 alkylcarbonyloxy;

each R² is independently halogen, hydroxyl, nitro, amino, cyano, C1-C3 alkyl, C1-C3 haloalkyl, C1-C3 alkoxy, C1-C3 haloalkoxy, C1-C3 alkylthio, C1-C3 haloalkylthio, C1-C3 alkylsulphonyl, C1-C3 alkylsulphonyloxy, C2-C6 carboxyalkyl, C2-C6 alkoxycarbonyl, C2-C7 alkylcarbonyloxy, C1-C3 alkylamino, or di(C1-C3 alkyl)amino;

R³ is hydrogen, halogen, C1-C3 alkyl, C1-C3 haloalkyl, C2-C4 alkoxyalkyl, C2-C4 alkenyl, C2-C4 haloalkenyl, or cyclopropyl optionally substituted by 1 to 3 groups R¹;

R⁴ is hydrogen, C1-C6 alkyl optionally substituted by 1 to 3 groups R¹³, C2-C6 alkenyl optionally substituted by 1 to 3 groups R¹³, C2-C6 alkynyl optionally substituted by 1 to 3 groups R¹³, C3-C8 cycloalkyl optionally substituted by 1 to 3 groups R¹³, C1-C6 acyl optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R² or C1-C6 alkylsulphonyl optionally substituted by 1 to 3 groups R¹;

each of R⁵ and R⁶ is independently hydrogen, halogen, C1-C6 alkyl optionally substituted by 1 to 3 groups R¹, C1-C6 alkoxy, C6-C10 aryl optionally substituted by 1 to 3 groups R², carboxyl, C1-C7 acyl, C2-C7 alkoxycarbonyl, or, taken together with the carbon atom to which they are attached, R⁵ and R⁶ form a C1-C6 alkenyl group optionally substituted by 1 to 3 groups R¹, a carbonyl group, or a C3-C6 cycloalkyl group optionally substituted by 1 to 3 groups R¹;

each of R⁷ and R⁸ is independently hydrogen, halogen, C1-C6 alkyl optionally substituted by 1 to 3 groups R¹, C1-C6 alkoxy, C6-C10 aryl optionally substituted by 1 to 3 groups R², carboxyl, C1-C7 acyl, C2-C7 alkoxycarbonyl, or R⁷ represents an additional bond between the carbon atom to which it is attached and the adjacent ring atom or, taken together with the carbon atom to which they are attached, R⁷ and R⁸ form a C1-C6 alkenyl group optionally substituted by 1 to 3 groups R¹, a carbonyl group, or a C3-C6 cycloalkyl group optionally substituted by 1 to 3 groups R¹;

R⁹ is hydrogen, hydroxyl, C1-C10 alkoxy optionally substituted by C1-C6 alkoxy or phenyl, C2-C10 alkenyloxy, C3-C8 cycloalkoxy optionally substituted by C1-C6 alkoxy or phenyl, C1-C6 alkylthio, amino, C1-C6 alkylamino, or di(C1-C6 alkyl)amino;

R¹⁰ is C1-C10 alkoxy optionally substituted by C1-C6 alkoxy or phenyl, C2-C10 alkenyloxy, C3-C8 cycloalkoxy optionally substituted by C1-C6 alkoxy or phenyl, C1-C6 alkylthio, amino, C1-C6 alkylamino, or di(C1-C6 alkyl)amino;

R¹¹ is hydrogen, C1-C6 alkyl, C1-C6 alkoxy, C3-C8 cycloalkoxy, amino, C1-C6 alkylamino, or di(C1-C6 alkyl)amino;

R¹² is hydrogen, C1-C6 alkoxy, C3-C8 cycloalkoxy, C1-C6 alkylthio, amino, C1-C6 alkylamino, or di(C1-C6 alkyl)amino;

each R¹³ is independently cyano, hydroxyl, C3-C6 cycloalkyl, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R², C1-C4 alkoxy; C1-C4 alkoxy(C1-C4)alkoxy; C1-C4 alkoxycarbonyl; or tri(C1-C4)alkylsilyl;

provided that
(i) when Y is NR⁵, X is N, Z is C(O)R⁹, D is N, E is —(CR⁷R⁸)ₙ—, R⁵ is alkyl or haloalkyl, R⁷ represents an additional bond to X, and R⁹ is alkoxy, then R⁸ is other than H;
(ii) when XEY is —N(R⁴)C(O)NH—, Z is not C(O)NH₂, C(O)NHCH₃ or C(O)N(CH₃)₂;
(iii) the compound of formula (I) is not:
   9-benzyl-9H-purine-2,6-dicarboxamide;
   9-(2-hydroxyethyl)-2-(prop-1-enyl)-9H-purine-6-carboxamide;
   9-(2-hydroxyethyl)-2-phenyl-9H-purine-6-carboxamide;
   9-phenyl-2-(pyridin-3-yl)-9H-purine-6-carboxamide;
   2-(3-hydroxyphenyl)-9-(2-methoxyphenyl)-9H-purine-6-carboxamide;
   2-(2-hydroxyphenyl)-9-(2-methoxyphenyl)-purine-6-carboxamide;
   6-oxo-8-phenyl-2-(pyridin-3-yl)-5,6,7,8-tetrahydropteridine-4-carboxamide;
   6-oxo-8-phenyl-2-(pyridin-4-yl)-5,6,7,8-tetrahydropteridine-4-carboxamide;
   2-(3-hydroxyphenyl)-8-(2-methoxyphenyl)-6-oxo-5,6,7,8-tetrahydropteridine-4-carboxamide;
   2-chloro-9-phenyl-9H-purine-6-carboxylic acid;
   2-chloro-9-methyl-9H-purine-6-carboxylic acid;
   2-chloro-9-methyl-9H-purine-6-carboxylic acid ethyl ester;
   2-chloro-9-ethoxycarbonylmethyl-9H-purine-6-carboxylic acid ethyl ester.

A is preferably halogen, C2-C6 alkenyl, C3-C8 cycloalkyl optionally substituted by 1 to 3 groups R¹, C6-C10 aryl optionally substituted by 1 to 3 groups R², or a mono- or bicyclic heteroaryl group having 5 to 10 ring atoms and at least one ring atom which is nitrogen, oxygen or sulfur optionally substituted by 1 to 3 groups R².

Examples of group A include 4-chloro-2-fluoro-3-methoxyphenyl, 4-chloro-3-dimethylamino-2-fluorophenyl, 4-chloro-3-fluorophenyl, 4-chlorophenyl, cyclopropyl and chloro.

More preferably, A is phenyl optionally substituted by 1 to 3 groups R², or C3-C6 cycloalkyl (preferably cyclopropyl) optionally substituted by 1 to 3 groups R¹.

In an very preferred embodiment, A is cyclopropyl or trisubstituted phenyl, wherein the substituents are independently R². More preferably, A is 2,3,4-trisubstituted phenyl, wherein the substituents are independently R². More preferably, A is 4-chloro-2-fluoro-3-methoxyphenyl, or 4-chloro-3-dimethylamino-2-fluorophenyl. Most preferably, A is 4-chloro-2-fluoro-3-methoxyphenyl.

Preferably, D is N, CH, CF, CCl, or CMe. More preferably, D is N or CH.

Preferably, n is 1 or 2. More preferably, n is 1.

Preferably X is O, S, or NR⁴. More preferably, X is S or NR⁴. Still more preferably, X is NR⁴.

Examples of X are S, and NR⁴.

Preferably, Y is CR⁵, CR⁵R⁶, N, or NR⁵. More preferably, Y is CR⁵ or CR⁵R⁶. Most preferably, Y is CR⁵.

Preferably, Z is C(O)R⁹ and R⁹ is as defined above. More preferably, Z is C(O)R⁹ and R⁹ is hydroxyl, C1-C6 alkoxy optionally substituted by C1-C6 alkoxy-C1-C6 alkoxy, C5-C10 heteroaryl or C3-C10 heterocyclyl, phenyl(C1-C2)alkoxy, (C1-C3)alkoxy(C1-C6)alkoxy or C3-C6 alkenyloxy.

Examples of group Z are CO₂CH₂CH═CH₂, CO₂CH₂CH₂OEt, CO₂CH₂CH₂On-Bu, CO₂CH(CH₃)CH₂On-Bu, CO₂CH₂CH₂OCH₂CH₂OCH₃, CO₂i-Pr, CO₂n-Pr, CO₂n-octyl, CO₂CH(CH₃)-n-pentyl, CO₂CH₂(2-furanyl), CO₂CH₂(2-tetrahydrofuranyl), CO₂CH₂Ph, CO₂Et, CO₂Me and CO₂H.

More preferably, Z is C(O)R⁹, wherein R⁹ is hydroxyl, C1-C6 alkoxy, phenyl(C1-C2)alkoxy, or (C1-C3)alkoxy(C1-C6)alkoxy.

More preferably, Z is C(O)R⁹ wherein R⁹ is hydroxyl, C1-C6 alkoxy or phenyl(C1-C2)alkoxy. More preferably, Z is CO₂H or CO₂Me. Most preferably, Z is CO₂Me.

Preferably, each R¹ is independently halogen, cyano, C1-C2 alkyl, C1-C2 haloalkyl, C1-C2 alkoxy, C1-C2 haloalkoxy, or C1-C3 alkoxycarbonyl;

Preferably, each R² is independently halogen, C1-C2 alkyl, C1-C2 haloalkyl, C1-C2 alkoxy, C1-C2 haloalkoxy, C1-C3 alkylamino, or di(C1-C3 alkyl)amino. More preferably, each R² is independently halogen, methoxy, or dimethylamino.

Preferably, R³ is hydrogen or halogen. More preferably, R³ is hydrogen, fluorine or chlorine. Most preferably, R³ is hydrogen.

Preferably, R⁴ is hydrogen, C1-C2alkyl, C1-C2alkoxyC1-C2alkyl, carboxyC1-C2alkyl, C3-C6cycloalkylC1-C2alkyl, C1-C5acyl, C1-C3alkoxycarbonyl, phenylC1-C2alkyl, wherein the phenyl is optionally substituted by one to three groups R², furanylC1-C2alkyl, wherein the furanyl is optionally substituted by one to three groups R², pyridylC1-2alkyl, wherein the pyridyl is optionally substituted by one to three groups R², C1-2alkylsulphonyl or phenylsulphonyl wherein the phenyl is optionally substituted by one to three groups R².

More preferably, R⁴ is hydrogen, C1-C2alkyl, phenylC1-C2alkyl, furanylC1-C2alkyl or pyridylC1-2alkyl. More preferably, R⁴ is hydrogen, methyl or benzyl. Most preferably, R⁴ is hydrogen.

Examples of R⁴ are hydrogen, methyl and benzyl.

Preferably, each of R⁵ and R⁶ is independently selected from hydrogen, halogen, C1-4 alkyl optionally substituted by phenyl, C1-C4 haloalkyl, or phenyl optionally substituted by 1-3 groups R², or, taken together, represent a C1-C4 alkylene group.

More preferably, each of R⁵ and R⁶ is independently selected from hydrogen, halogen, C1-4 alkyl, C1-C4 haloalkyl, or phenyl optionally substituted by 1-3 groups R², or, taken together, represent a C1-C4 alkylene group. More preferably, each of R⁵ and R⁶ is independently selected from hydrogen and C1-C4 alkyl, more preferably methyl.

Examples of R⁵ and R⁶ are hydrogen, methyl, or taken together are methylene.

Preferably, each of R⁷ is H or Me or represents an additional bond between the carbon atom to which it is attached and the adjacent ring atom or together with R⁸ represents ═O.

More preferably, each of R⁷ represents an additional bond between the carbon atom to which it is attached and the adjacent ring atom. More preferably, R⁷ represents an additional bond to Y.

Preferably, R⁸ is hydrogen, halogen, C1-6 alkyl, C1-6 haloalkyl, or phenyl optionally substituted by 1-3 groups R² or together with R⁷ represents ═O.

More preferably, R⁸ is hydrogen, halogen, C1-6 alkyl, C1-6 haloalkyl, or phenyl optionally substituted by 1-3 groups R². More preferably, R⁸ is selected from H and C1-C6 alkyl. More preferably, R⁸ is hydrogen or methyl.

Examples of R⁸ include hydrogen, methyl and phenyl.

In a particularly preferred embodiment, the compound of the invention has the formula (II)

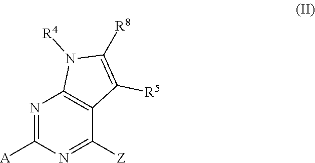

wherein A, Z, R⁴, R⁵ and R⁸ have the values ascribed above. In this embodiment, it is preferred that A is phenyl optionally substituted by 1 to 3 groups R² or cyclopropyl optionally substituted by 1 to 3 groups R¹. In this embodiment, it is more preferred that A is phenyl optionally substituted by 1 to 3 groups R². It is furthermore preferred that Z is C(O)R⁹, wherein R⁹ is selected from hydroxyl and C1-C6 alkoxy; that R⁴ is H; that R⁵ is selected from H and C1-C6 alkyl, and that R⁸ is selected from H and C1-C6 alkyl.

In an alternative, particularly preferred embodiment, the compound of the invention has the formula (III)

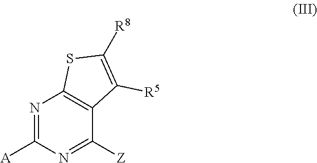

wherein A, Z, R⁵ and R⁸ have the values ascribed above. In this embodiment, it is preferred that A is phenyl optionally substituted by 1 to 3 groups R². It is furthermore preferred that Z is C(O)R⁹, wherein R⁹ is selected from hydroxyl and C1-C6 alkoxy; that R⁵ is selected from H and C1-C6 alkyl, and that R⁸ is selected from H and C1-C6 alkyl.

In an alternative, particularly preferred embodiment, the compound of the invention has the formula (IV)

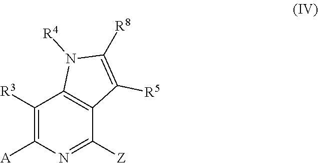

wherein A, Z, R³, R⁴, R⁵ and R⁸ have the values ascribed above. In this embodiment, it is preferred that A is phenyl optionally substituted by 1 to 3 groups R² or halogen. In this embodiment, it is more preferred that A is phenyl optionally substituted by 1 to 3 groups R². It is furthermore preferred that Z is C(O)R⁹, wherein R⁹ is selected from hydroxyl and C1-C6 alkoxy; that R³ is H, fluor or chloro; that R⁴ is H; that R⁵ is selected from H and C1-C6 alkyl; and that R⁸ is selected from H and C1-C6 alkyl.

The compounds described below are illustrative of novel compounds of the invention.

Table 1 below provides 136 compounds designated compounds 1-1 to 1-136 respectively, of formula (1A) wherein D is N and X is NH.

TABLE 1

| | |
|---|---|
| | (1A) |
| 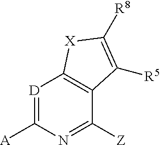 | |

| Com- | | | | |
|---|---|---|---|---|
| pound | | | | |
| Num- | Substituent Values | | | |
| ber | A | R⁵ | R⁸ | Z |
| 1-1 | cyclopropyl | H | H | CO₂H |
| 1-2 | cyclopropyl | H | H | CO₂Me |
| 1-3 | cyclopropyl | Me | H | CO₂H |
| 1-4 | cyclopropyl | Me | H | CO₂Me |
| 1-5 | cyclopropyl | H | Me | CO₂H |
| 1-6 | cyclopropyl | H | Me | CO₂Me |
| 1-7 | cyclopropyl | Me | Me | CO₂H |
| 1-8 | cyclopropyl | Me | Me | CO₂Me |
| 1-9 | 4-chlorophenyl | H | H | CO₂H |
| 1-10 | 4-chlorophenyl | H | H | CO₂Me |
| 1-11 | 4-chlorophenyl | Me | H | CO₂H |
| 1-12 | 4-chlorophenyl | Me | H | CO₂Me |
| 1-13 | 4-chlorophenyl | H | Me | CO₂H |
| 1-14 | 4-chlorophenyl | H | Me | CO₂Me |
| 1-15 | 4-chlorophenyl | Me | Me | CO₂H |
| 1-16 | 4-chlorophenyl | Me | Me | CO₂Me |
| 1-17 | 4-chloro-3-fluorophenyl | H | H | CO₂H |
| 1-18 | 4-chloro-3-fluorophenyl | H | H | CO₂Me |
| 1-19 | 4-chloro-3-fluorophenyl | Me | H | CO₂H |
| 1-20 | 4-chloro-3-fluorophenyl | Me | H | CO₂Me |
| 1-21 | 4-chloro-3-fluorophenyl | H | Me | CO₂H |
| 1-22 | 4-chloro-3-fluorophenyl | H | Me | CO₂Me |
| 1-23 | 4-chloro-3-fluorophenyl | Me | Me | CO₂H |
| 1-24 | 4-chloro-3-fluorophenyl | Me | Me | CO₂Me |
| 1-25 | 4-chloro-3-fluorophenyl | H | H | CO₂Et |
| 1-26 | 4-chloro-3-fluorophenyl | H | H | CO₂n-Pr |
| 1-27 | 4-chloro-3-fluorophenyl | H | H | CO₂i-Pr |
| 1-28 | 4-chloro-3-fluorophenyl | H | H | CO₂CH₂CH═CH₂ |
| 1-29 | 4-chloro-3-fluorophenyl | H | H | CO₂CH₂CH₂OMe |
| 1-30 | 4-chloro-3-fluorophenyl | H | H | CO₂CH₂Ph |
| 1-31 | 4-chloro-3-fluorophenyl | Me | H | CO₂Et |
| 1-32 | 4-chloro-3-fluorophenyl | Me | H | CO₂n-Pr |
| 1-33 | 4-chloro-3-fluorophenyl | Me | H | CO₂i-Pr |
| 1-34 | 4-chloro-3-fluorophenyl | Me | H | CO₂CH₂CH═CH₂ |
| 1-35 | 4-chloro-3-fluorophenyl | Me | H | CO₂CH₂CH₂OMe |
| 1-36 | 4-chloro-3-fluorophenyl | Me | H | CO₂CH₂Ph |
| 1-37 | 4-chloro-3-fluorophenyl | H | Me | CO₂Et |
| 1-38 | 4-chloro-3-fluorophenyl | H | Me | CO₂n-Pr |
| 1-39 | 4-chloro-3-fluorophenyl | H | Me | CO₂i-Pr |
| 1-40 | 4-chloro-3-fluorophenyl | H | Me | CO₂CH₂CH═CH₂ |
| 1-41 | 4-chloro-3-fluorophenyl | H | Me | CO₂CH₂CH₂OMe |
| 1-42 | 4-chloro-3-fluorophenyl | H | Me | CO₂CH₂Ph |
| 1-43 | 4-chloro-3-fluorophenyl | Me | Me | CO₂Et |
| 1-44 | 4-chloro-3-fluorophenyl | Me | Me | CO₂n-Pr |
| 1-45 | 4-chloro-3-fluorophenyl | Me | Me | CO₂i-Pr |
| 1-46 | 4-chloro-3-fluorophenyl | Me | Me | CO₂CH₂CH═CH₂ |
| 1-47 | 4-chloro-3-fluorophenyl | Me | Me | CO₂CH₂CH₂OMe |
| 1-48 | 4-chloro-3-fluorophenyl | Me | Me | CO₂CH₂Ph |
| 1-49 | 4-chloro-3-fluorophenyl | Cl | H | CO₂H |
| 1-50 | 4-chloro-3-fluorophenyl | Cl | H | CO₂Me |
| 1-51 | 4-chloro-3-fluorophenyl | Cl | Me | CO₂H |
| 1-52 | 4-chloro-3-fluorophenyl | Cl | Me | CO₂Me |
| 1-53 | 4-chloro-3-fluorophenyl | H | Ph | CO₂H |
| 1-54 | 4-chloro-3-fluorophenyl | H | Ph | CO₂Me |
| 1-55 | 4-chloro-3-fluorophenyl | Me | Ph | CO₂H |
| 1-56 | 4-chloro-3-fluorophenyl | Me | Ph | CO₂Me |
| 1-57 | 4-chloro-3-fluorophenyl | H | Cl | CO₂H |
| 1-58 | 4-chloro-3-fluorophenyl | H | Cl | CO₂Me |
| 1-59 | 4-chloro-3-fluorophenyl | Me | Cl | CO₂H |
| 1-60 | 4-chloro-3-fluorophenyl | Me | Cl | CO₂Me |
| 1-61 | 4-chloro-2-fluoro-3- | H | H | CO₂H |
| | methoxyphenyl | | | |
| 1-62 | 4-chloro-2-fluoro-3- | H | H | CO₂Me |
| | methoxyphenyl | | | |
| 1-63 | 4-chloro-2-fluoro-3- | Me | H | CO₂H |
| | methoxyphenyl | | | |
| 1-64 | 4-chloro-2-fluoro-3- | Me | H | CO₂Me |
| | methoxyphenyl | | | |
| 1-65 | 4-chloro-2-fluoro-3- | H | Me | CO₂H |
| | methoxyphenyl | | | |
| 1-66 | 4-chloro-2-fluoro-3- | H | Me | CO₂Me |
| | methoxyphenyl | | | |
| 1-67 | 4-chloro-2-fluoro-3- | Me | Me | CO₂H |
| | methoxyphenyl | | | |
| 1-68 | 4-chloro-2-fluoro-3- | Me | Me | CO₂Me |
| | methoxyphenyl | | | |
| 1-69 | 4-chloro-2-fluoro-3- | H | H | CO₂Et |
| | methoxyphenyl | | | |
| 1-70 | 4-chloro-2-fluoro-3- | H | H | CO₂n-Pr |
| | methoxyphenyl | | | |
| 1-71 | 4-chloro-2-fluoro-3- | H | H | CO₂i-Pr |
| | methoxyphenyl | | | |
| 1-72 | 4-chloro-2-fluoro-3- | H | H | CO₂CH₂CH═CH₂ |
| | methoxyphenyl | | | |
| 1-73 | 4-chloro-2-fluoro-3- | H | H | CO₂CH₂CH₂OMe |
| | methoxyphenyl | | | |
| 1-74 | 4-chloro-2-fluoro-3- | H | H | CO₂CH₂Ph |
| | methoxyphenyl | | | |
| 1-75 | 4-chloro-2-fluoro-3- | Me | H | CO₂Et |
| | methoxyphenyl | | | |
| 1-76 | 4-chloro-2-fluoro-3- | Me | H | CO₂n-Pr |
| | methoxyphenyl | | | |
| 1-77 | 4-chloro-2-fluoro-3- | Me | H | CO₂n-octyl |
| | methoxyphenyl | | | |
| 1-78 | 4-chloro-2-fluoro-3- | Me | H | CO₂i-Pr |
| | methoxyphenyl | | | |
| 1-79 | 4-chloro-2-fluoro-3- | Me | H | CO₂CH(Me)n-pentyl |
| | methoxyphenyl | | | |
| 1-80 | 4-chloro-2-fluoro-3- | Me | H | CO₂CH₂CH═CH₂ |
| | methoxyphenyl | | | |
| 1-81 | 4-chloro-2-fluoro-3- | Me | H | CO₂CH₂CH₂OMe |
| | methoxyphenyl | | | |
| 1-82 | 4-chloro-2-fluoro-3- | Me | H | CO₂CH₂CH₂OEt |
| | methoxyphenyl | | | |
| 1-83 | 4-chloro-2-fluoro-3- | Me | H | CO₂CH₂CH₂On-Bu |
| | methoxyphenyl | | | |
| 1-84 | 4-chloro-2-fluoro-3- | Me | H | CO₂CH(Me)CH₂On-Bu |
| | methoxyphenyl | | | |
| 1-85 | 4-chloro-2-fluoro-3- | Me | H | CO₂CH₂CH₂OCH₂CH₂OMe |
| | methoxyphenyl | | | |
| 1-86 | 4-chloro-2-fluoro-3- | Me | H | CO₂CH₂Ph |
| | methoxyphenyl | | | |
| 1-87 | 4-chloro-2-fluoro-3- | Me | H | CO₂CH₂(2-furanyl) |
| | methoxyphenyl | | | |
| 1-88 | 4-chloro-2-fluoro-3- | Me | H | CO₂CH₂(2- |
| | methoxyphenyl | | | tetrahydrofuranyl) |
| 1-89 | 4-chloro-2-fluoro-3- | H | Me | CO₂Et |
| | methoxyphenyl | | | |
| 1-90 | 4-chloro-2-fluoro-3- | H | Me | CO₂n-Pr |
| | methoxyphenyl | | | |
| 1-91 | 4-chloro-2-fluoro-3- | H | Me | CO₂i-Pr |
| | methoxyphenyl | | | |
| 1-92 | 4-chloro-2-fluoro-3- | H | Me | CO₂CH₂CH═CH₂ |
| | methoxyphenyl | | | |
| 1-93 | 4-chloro-2-fluoro-3- | H | Me | CO₂CH₂CH₂OMe |
| | methoxyphenyl | | | |
| 1-94 | 4-chloro-2-fluoro-3- | H | Me | CO₂CH₂Ph |
| | methoxyphenyl | | | |
| 1-95 | 4-chloro-2-fluoro-3- | Me | Me | CO₂Et |
| | methoxyphenyl | | | |
| 1-96 | 4-chloro-2-fluoro-3- | Me | Me | CO₂n-Pr |
| | methoxyphenyl | | | |
| 1-97 | 4-chloro-2-fluoro-3- | Me | Me | CO₂i-Pr |
| | methoxyphenyl | | | |
| 1-98 | 4-chloro-2-fluoro-3- | Me | Me | CO₂CH₂CH═CH₂ |
| | methoxyphenyl | | | |
| 1-99 | 4-chloro-2-fluoro-3- | Me | Me | CO₂CH₂CH₂OMe |
| | methoxyphenyl | | | |
| 1-100 | 4-chloro-2-fluoro-3- | Me | Me | CO₂CH₂Ph |
| | methoxyphenyl | | | |
| 1-101 | 4-chloro-2-fluoro-3- | Cl | H | CO₂H |
| | methoxyphenyl | | | |
| 1-102 | 4-chloro-2-fluoro-3- | Cl | H | CO₂Me |
| | methoxyphenyl | | | |
| 1-103 | 4-chloro-2-fluoro-3- | Cl | Me | CO₂H |
| | methoxyphenyl | | | |
| 1-104 | 4-chloro-2-fluoro-3- | Cl | Me | CO₂Me |
| | methoxyphenyl | | | |
| 1-105 | 4-chloro-2-fluoro-3- | H | Ph | CO₂H |
| | methoxyphenyl | | | |
| 1-106 | 4-chloro-2-fluoro-3- | H | Ph | CO₂Me |
| | methoxyphenyl | | | |
| 1-107 | 4-chloro-2-fluoro-3- | Me | Ph | CO₂H |
| | methoxyphenyl | | | |
| 1-108 | 4-chloro-2-fluoro-3- | Me | Ph | CO₂Me |
| | methoxyphenyl | | | |
| 1-109 | 4-chloro-2-fluoro-3- | H | Cl | CO₂H |
| | methoxyphenyl | | | |
| 1-110 | 4-chloro-2-fluoro-3- | H | Cl | CO₂Me |
| | methoxyphenyl | | | |
| 1-111 | 4-chloro-2-fluoro-3- | Me | Cl | CO₂H |
| | methoxyphenyl | | | |
| 1-112 | 4-chloro-2-fluoro-3- | Me | Cl | CO₂Me |
| | methoxyphenyl | | | |
| 1-113 | 4-chloro-3- | H | H | CO₂H |
| | dimethylamino-2- | | | |
| | fluorophenyl | | | |
| 1-114 | 4-chloro-3- | H | H | CO₂Me |
| | dimethylamino-2- | | | |
| | fluorophenyl | | | |
| 1-115 | 4-chloro-3- | Me | H | CO₂H |
| | dimethylamino-2- | | | |
| | fluorophenyl | | | |
| 1-116 | 4-chloro-3- | Me | H | CO₂Me |
| | dimethylamino-2- | | | |
| | fluorophenyl | | | |
| 1-117 | 4-chloro-3- | H | Me | CO₂H |
| | dimethylamino-2- | | | |
| | fluorophenyl | | | |
| 1-118 | 4-chloro-3- | H | Me | CO₂Me |
| | dimethylamino-2- | | | |
| | fluorophenyl | | | |
| 1-119 | 4-chloro-3- | Me | Me | CO₂H |
| | dimethylamino-2- | | | |
| | fluorophenyl | | | |
| 1-120 | 4-chloro-3- | Me | Me | CO₂Me |
| | dimethylamino-2- | | | |
| | fluorophenyl | | | |
| 1-121 | Cl | H | H | CO₂H |
| 1-122 | Cl | H | H | CO₂Me |
| 1-123 | Cl | Me | H | CO₂H |
| 1-124 | Cl | Me | H | CO₂Me |
| 1-125 | Cl | H | Me | CO₂H |
| 1-126 | Cl | H | Me | CO₂Me |
| 1-127 | Cl | Me | Me | CO₂H |
| 1-128 | Cl | Me | Me | CO₂Me |
| 1-129 | Cl | H | Cl | CO₂H |
| 1-130 | Cl | H | Cl | CO₂Me |
| 1-131 | Cl | Cl | H | CO₂H |
| 1-132 | Cl | Cl | H | CO₂Me |
| 1-133 | Cl | Me | Cl | CO₂H |
| 1-134 | Cl | Me | Cl | CO₂Me |
| 1-135 | Cl | Cl | Me | CO₂H |
| 1-136 | Cl | Cl | Me | CO₂Me |

136 compounds are described, designated compounds 2-1 to 2-136 respectively, of formula (1A) wherein D is N and X is NMe, and the values of A, R⁵, R⁸ and Z are as defined in Table 1.

136 compounds are described, designated compounds 3-1 to 3-136 respectively, of formula (1A) wherein D is N and X is NCH₂OEt, and the values of A, R⁵, R⁸ and Z are as defined in Table 1.

136 compounds are described, designated compounds 4-1 to 4-136 respectively, of formula (1A) wherein D is N and X is NCH₂CO₂H, and the values of A, R⁵, R⁸ and Z are as defined in Table 1.

136 compounds are described, designated compounds 5-1 to 5-136 respectively, of formula (1A) wherein D is N and X is NCH₂(cyclopropyl), and the values of A, R⁵, R⁸ and Z are as defined in Table 1.

136 compounds are described, designated compounds 6-1 to 6-136 respectively, of formula (1A) wherein D is N and X is NCH₂Ph, and the values of A, R⁵, R⁸ and Z are as defined in Table 1.

136 compounds are described, designated compounds 7-1 to 7-136 respectively, of formula (1A) wherein D is N and X is NCH(Me)Ph, and the values of A, R⁵, R⁸ and Z are as defined in Table 1.

136 compounds are described, designated compounds 8-1 to 8-136 respectively, of formula (1A) wherein D is N and X is NCH₂(2-nitrophenyl), and the values of A, R⁵, R⁸ and Z are as defined in Table 1.

136 compounds are described, designated compounds 9-1 to 9-136 respectively, of formula (1A) wherein D is N and X is NCH₂(4-fluorophenyl), and the values of A, R⁵, R⁸ and Z are as defined in Table 1.

136 compounds are described, designated compounds 10-1 to 10-136 respectively, of formula (1A) wherein D is N and X is NCH₂(4-methoxyphenyl), and the values of A, R⁵, R⁸ and Z are as defined in Table 1.

136 compounds are described, designated compounds 11-1 to 11-136 respectively, of formula (1A) wherein D is N and X is NCH₂(2-furanyl), and the values of A, R⁵, R⁸ and Z are as defined in Table 1.

136 compounds are described, designated compounds 12-1 to 12-136 respectively, of formula (1A) wherein D is N and X is NCH₂(5-trifluoromethylfuran-2-yl), and the values of A, R⁵, R⁸ and Z are as defined in Table 1.

136 compounds are described, designated compounds 13-1 to 13-136 respectively, of formula (1A) wherein D is N and X is NCHMe(4-pyridyl), and the values of A, R⁵, R⁸ and Z are as defined in Table 1.

136 compounds are described, designated compounds 14-1 to 14-136 respectively, of formula (1A) wherein D is N and X is NCH₂(3-chloropyrid-2-yl), and the values of A, R⁵, R⁸ and Z are as defined in Table 1.

136 compounds are described, designated compounds 15-1 to 15-136 respectively, of formula (1A) wherein D is N and X is NCOMe, and the values of A, R⁵, R⁸ and Z are as defined in Table 1.

136 compounds are described, designated compounds 16-1 to 16-136 respectively, of formula (1A) wherein D is N and X is NCOCMe₃, and the values of A, R⁵, R⁸ and Z are as defined in Table 1.

136 compounds are described, designated compounds 17-1 to 17-136 respectively, of formula (1A) wherein D is N and X is NCO₂Me, and the values of A, R⁵, R⁸ and Z are as defined in Table 1.

136 compounds are described, designated compounds 18-1 to 18-136 respectively, of formula (1A) wherein D is N and X is NSO₂Me, and the values of A, R⁵, R⁸ and Z are as defined in Table 1.

136 compounds are described, designated compounds 19-1 to 19-136 respectively, of formula (1A) wherein D is N and X is NSO₂(4-methylphenyl), and the values of A, R⁵, R⁸ and Z are as defined in Table 1.

136 compounds are described, designated compounds 20-1 to 20-136 respectively, of formula (1A) wherein D is N and X is O, and the values of A, R⁵, R⁸ and Z are as defined in Table 1.

136 compounds are described, designated compounds 21-1 to 21-136 respectively, of formula (1A) wherein D is N and X is S, and the values of A, R⁵, R⁸ and Z are as defined in Table 1.

136 compounds are described, designated compounds 22-1 to 22-136 respectively, of formula (1A) wherein D is CH and X is NH, and the values of A, R⁵, R⁸ and Z are as defined in Table 1.

136 compounds are described, designated compounds 23-1 to 23-136 respectively, of formula (1A) wherein D is CH and X is NMe, and the values of A, R⁵, R⁸ and Z are as defined in Table 1.

136 compounds are described, designated compounds 24-1 to 24-136 respectively, of formula (1A) wherein D is CH and X is NCH₂OEt, and the values of A, R⁵, R⁸ and Z are as defined in Table 1.

136 compounds are described, designated compounds 25-1 to 25-136 respectively, of formula (1A) wherein D is CH and X is NCH₂CO₂H, and the values of A, R⁵, R⁸ and Z are as defined in Table 1.

136 compounds are described, designated compounds 26-1 to 26-136 respectively, of formula (1A) wherein D is CH and X is NCH₂(cyclopropyl), and the values of A, R⁵, R⁸ and Z are as defined in Table 1.

136 compounds are described, designated compounds 27-1 to 27-136 respectively, of formula (1A) wherein D is CH and X is NCH₂Ph, and the values of A, R⁵, R⁸ and Z are as defined in Table 1.

136 compounds are described, designated compounds 28-1 to 28-136 respectively, of formula (1A) wherein D is CH and X is NCH(Me)Ph, and the values of A, R⁵, R⁸ and Z are as defined in Table 1.

136 compounds are described, designated compounds 29-1 to 29-136 respectively, of formula (1A) wherein D is CH and X is NCH₂(2-nitrophenyl), and the values of A, R⁵, R⁸ and Z are as defined in Table 1.

136 compounds are described, designated compounds 30-1 to 30-136 respectively, of formula (1A) wherein D is CH and X is NCH₂(4-fluorophenyl), and the values of A, R⁵, R⁸ and Z are as defined in Table 1.

136 compounds are described, designated compounds 31-1 to 31-136 respectively, of formula (1A) wherein D is CH and X is NCH₂(4-methoxyphenyl), and the values of A, R⁵, R⁸ and Z are as defined in Table 1.

136 compounds are described, designated compounds 32-1 to 32-136 respectively, of formula (1A) wherein D is CH and X is NCH₂(2-furanyl), and the values of A, R⁵, R⁸ and Z are as defined in Table 1.

136 compounds are described, designated compounds 33-1 to 33-136 respectively, of formula (1A) wherein D is CH and X is NCH₂(5-trifluoromethylfuran-2-yl), and the values of A, R⁵, R⁸ and Z are as defined in Table 1.

136 compounds are described, designated compounds 34-1 to 34-136 respectively, of formula (1A) wherein D is CH and X is NCHMe(4-pyridyl), and the values of A, R⁵, R⁸ and Z are as defined in Table 1.

136 compounds are described, designated compounds 35-1 to 35-136 respectively, of formula (1A) wherein D is CH and X is NCH₂(3-chloropyrid-2-yl), and the values of A, R⁵, R⁸ and Z are as defined in Table 1.

136 compounds are described, designated compounds 36-1 to 36-136 respectively, of formula (1A) wherein D is CH and X is NCOMe, and the values of A, R⁵, R⁸ and Z are as defined in Table 1.

136 compounds are described, designated compounds 37-1 to 37-136 respectively, of formula (1A) wherein D is CH and X is NCOCMe₃, and the values of A, R⁵, R⁸ and Z are as defined in Table 1.

136 compounds are described, designated compounds 38-1 to 38-136 respectively, of formula (1A) wherein D is CH and X is NCO₂Me, and the values of A, R⁵, R⁸ and Z are as defined in Table 1.

136 compounds are described, designated compounds 39-1 to 39-136 respectively, of formula (1A) wherein D is CH and X is NSO₂Me, and the values of A, R⁵, R⁸ and Z are as defined in Table 1.

136 compounds are described, designated compounds 40-1 to 40-136 respectively, of formula (1A) wherein D is CH and X is NSO₂(4-methylphenyl), and the values of A, R⁵, R⁸ and Z are as defined in Table 1.

136 compounds are described, designated compounds 41-1 to 41-136 respectively, of formula (1A) wherein D is CH and X is O, and the values of A, R⁵, R⁸ and Z are as defined in Table 1.

136 compounds are described, designated compounds 42-1 to 42-136 respectively, of formula (1A) wherein D is CH and X is S, and the values of A, R⁵, R⁸ and Z are as defined in Table 1.

136 compounds are described, designated compounds 43-1 to 43-136 respectively, of formula (1A) wherein D is CF and X is NH, and the values of A, R⁵, R⁸ and Z are as defined in Table 1.

136 compounds are described, designated compounds 44-1 to 44-136 respectively, of formula (1A) wherein D is CF and X is NMe, and the values of A, R⁵, R⁸ and Z are as defined in Table 1.

136 compounds are described, designated compounds 45-1 to 45-136 respectively, of formula (1A) wherein D is CF and X is O, and the values of A, R⁵, R⁸ and Z are as defined in Table 1.

136 compounds are described, designated compounds 46-1 to 46-136 respectively, of formula (1A) wherein D is CF and X is S, and the values of A, R⁵, R⁸ and Z are as defined in Table 1.

136 compounds are described, designated compounds 47-1 to 47-136 respectively, of formula (1A) wherein D is CCl and X is NH, and the values of A, R⁵, R⁸ and Z are as defined in Table 1.

136 compounds are described, designated compounds 48-1 to 48-136 respectively, of formula (1A) wherein D is CCl and X is NMe, and the values of A, R⁵, R⁸ and Z are as defined in Table 1.

136 compounds are described, designated compounds 49-1 to 49-136 respectively, of formula (1A) wherein D is CCl and X is O, and the values of A, R⁵, R⁸ and Z are as defined in Table 1.

136 compounds are described, designated compounds 50-1 to 50-136 respectively, of formula (1A) wherein D is CCl and X is S, and the values of A, R⁵, R⁸ and Z are as defined in Table 1.

136 compounds are described, designated compounds 51-1 to 51-136 respectively, of formula (1A) wherein D is CMe and X is NH, and the values of A, R⁵, R⁸ and Z are as defined in Table 1.

136 compounds are described, designated compounds 52-1 to 52-136 respectively, of formula (1A) wherein D is CMe and X is NMe, and the values of A, R⁵, R⁸ and Z are as defined in Table 1.

136 compounds are described, designated compounds 53-1 to 53-136 respectively, of formula (1A) wherein D is CMe and X is O, and the values of A, R⁵, R⁸ and Z are as defined in Table 1.

136 compounds are described, designated compounds 54-1 to 54-136 respectively, of formula (1A) wherein D is CMe and X is S, and the values of A, R⁵, R⁸ and Z are as defined in Table 1.

Table 2 below provides 252 compounds designated compounds 55-1 to 55-252 respectively, of formula (1B) wherein D is N and X is NH.

TABLE 2

| | |
|---|---|
| | (1B) |
| 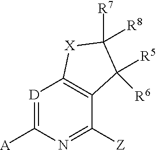 | |

| Compound | Substituent Values | | | | | |
|---|---|---|---|---|---|---|
| Number | A | R⁷ | R⁸ | R⁵ | R⁶ | Z |
| 55-1 | cyclopropyl | H | H | H | H | CO₂H |
| 55-2 | cyclopropyl | H | H | H | H | CO₂Me |
| 55-3 | cyclopropyl | H | H | Me | H | CO₂H |
| 55-4 | cyclopropyl | H | H | Me | H | CO₂Me |
| 55-5 | cyclopropyl | Me | H | H | H | CO₂H |
| 55-6 | cyclopropyl | Me | H | H | H | CO₂Me |
| 55-7 | cyclopropyl | Me | H | Me | H | CO₂H |
| 55-8 | cyclopropyl | Me | H | Me | H | CO₂Me |
| 55-9 | 4-chlorophenyl | H | H | H | H | CO₂H |
| 55-10 | 4-chlorophenyl | H | H | H | H | CO₂Me |
| 55-11 | 4-chlorophenyl | H | H | Me | H | CO₂H |
| 55-12 | 4-chlorophenyl | H | H | Me | H | CO₂Me |
| 55-13 | 4-chlorophenyl | Me | H | H | H | CO₂H |
| 55-14 | 4-chlorophenyl | Me | H | H | H | CO₂Me |
| 55-15 | 4-chlorophenyl | Me | H | Me | H | CO₂H |
| 55-16 | 4-chlorophenyl | Me | H | Me | H | CO₂Me |
| 55-17 | 4-chloro-3- | H | H | H | H | CO₂H |
| | fluorophenyl | | | | | |
| 55-18 | 4-chloro-3- | H | H | H | H | CO₂Me |
| | fluorophenyl | | | | | |
| 55-19 | 4-chloro-3- | H | H | Me | H | CO₂H |
| | fluorophenyl | | | | | |
| 55-20 | 4-chloro-3- | H | H | Me | H | CO₂Me |
| | fluorophenyl | | | | | |
| 55-21 | 4-chloro-3- | Me | H | H | H | CO₂H |
| | fluorophenyl | | | | | |
| 55-22 | 4-chloro-3- | Me | H | H | H | CO₂Me |
| | fluorophenyl | | | | | |
| 55-23 | 4-chloro-3- | Me | H | Me | H | CO₂H |
| | fluorophenyl | | | | | |
| 55-24 | 4-chloro-3- | Me | H | Me | H | CO₂Me |
| | fluorophenyl | | | | | |
| 55-25 | 4-chloro-3- | H | H | H | H | CO₂Et |
| | fluorophenyl | | | | | |
| 55-26 | 4-chloro-3- | H | H | H | H | CO₂n-Pr |
| | fluorophenyl | | | | | |
| 55-27 | 4-chloro-3- | H | H | H | H | CO₂i-Pr |
| | fluorophenyl | | | | | |
| 55-28 | 4-chloro-3- | H | H | H | H | CO₂CH₂CH═CH₂ |
| | fluorophenyl | | | | | |
| 55-29 | 4-chloro-3- | H | H | H | H | CO₂CH₂CH₂OMe |
| | fluorophenyl | | | | | |
| 55-30 | 4-chloro-3- | H | H | H | H | CO₂CH₂Ph |
| | fluorophenyl | | | | | |
| 55-31 | 4-chloro-3- | H | H | Me | H | CO₂Et |
| | fluorophenyl | | | | | |
| 55-32 | 4-chloro-3- | H | H | Me | H | CO₂n-Pr |
| | fluorophenyl | | | | | |
| 55-33 | 4-chloro-3- | H | H | Me | H | CO₂i-Pr |
| | fluorophenyl | | | | | |
| 55-34 | 4-chloro-3- | H | H | Me | H | CO₂CH₂CH═CH₂ |
| | fluorophenyl | | | | | |
| 55-35 | 4-chloro-3- | H | H | Me | H | CO₂CH₂CH₂OMe |
| | fluorophenyl | | | | | |
| 55-36 | 4-chloro-3- | H | H | Me | H | CO₂CH₂Ph |
| | fluorophenyl | | | | | |
| 55-37 | 4-chloro-3- | Me | H | H | H | CO₂Et |
| | fluorophenyl | | | | | |
| 55-38 | 4-chloro-3- | Me | H | H | H | CO₂n-Pr |
| | fluorophenyl | | | | | |
| 55-39 | 4-chloro-3- | Me | H | H | H | CO₂i-Pr |
| | fluorophenyl | | | | | |
| 55-40 | 4-chloro-3- | Me | H | H | H | CO₂CH₂CH═CH₂ |
| | fluorophenyl | | | | | |
| 55-41 | 4-chloro-3- | Me | H | H | H | CO₂CH₂CH₂OMe |
| | fluorophenyl | | | | | |
| 55-42 | 4-chloro-3- | Me | H | H | H | CO₂CH₂Ph |
| | fluorophenyl | | | | | |
| 55-43 | 4-chloro-3- | Me | H | Me | H | CO₂Et |
| | fluorophenyl | | | | | |
| 55-44 | 4-chloro-3- | Me | H | Me | H | CO₂n-Pr |
| | fluorophenyl | | | | | |
| 55-45 | 4-chloro-3- | Me | H | Me | H | CO₂i-Pr |
| | fluorophenyl | | | | | |
| 55-46 | 4-chloro-3- | Me | H | Me | H | CO₂CH₂CH═CH₂ |
| | fluorophenyl | | | | | |
| 55-47 | 4-chloro-3- | Me | H | Me | H | CO₂CH₂CH₂OMe |
| | fluorophenyl | | | | | |
| 55-48 | 4-chloro-3- | Me | H | Me | H | CO₂CH₂Ph |
| | fluorophenyl | | | | | |
| 55-49 | 4-chloro-3- | ═O | | H | H | CO₂H |
| | fluorophenyl | | | | | |
| 55-50 | 4-chloro-3- | ═O | | H | H | CO₂Me |
| | fluorophenyl | | | | | |
| 55-51 | 4-chloro-3- | ═O | | Me | H | CO₂H |
| | fluorophenyl | | | | | |
| 55-52 | 4-chloro-3- | ═O | | Me | H | CO₂Me |
| | fluorophenyl | | | | | |
| 55-53 | 4-chloro-3- | CH₂CH₂ | | H | H | CO₂H |
| | fluorophenyl | | | | | |
| 55-54 | 4-chloro-3- | CH₂CH₂ | | H | H | CO₂Me |
| | fluorophenyl | | | | | |
| 55-55 | 4-chloro-3- | CH₂CH₂ | | Me | H | CO₂H |
| | fluorophenyl | | | | | |
| 55-56 | 4-chloro-3- | CH₂CH₂ | | Me | H | CO₂Me |
| | fluorophenyl | | | | | |
| 55-57 | 4-chloro-2-fluoro-3- | H | H | H | H | CO₂H |
| | methoxyphenyl | | | | | |
| 55-58 | 4-chloro-2-fluoro-3- | H | H | H | H | CO₂Me |
| | methoxyphenyl | | | | | |
| 55-59 | 4-chloro-2-fluoro-3- | H | H | Me | H | CO₂H |
| | methoxyphenyl | | | | | |
| 55-60 | 4-chloro-2-fluoro-3- | H | H | Me | H | CO₂Me |
| | methoxyphenyl | | | | | |
| 55-61 | 4-chloro-2-fluoro-3- | Me | H | H | H | CO₂H |
| | methoxyphenyl | | | | | |
| 55-62 | 4-chloro-2-fluoro-3- | Me | H | H | H | CO₂Me |
| | methoxyphenyl | | | | | |
| 55-63 | 4-chloro-2-fluoro-3- | Me | H | Me | H | CO₂H |
| | methoxyphenyl | | | | | |
| 55-64 | 4-chloro-2-fluoro-3- | Me | H | Me | H | CO₂Me |
| | methoxyphenyl | | | | | |
| 55-65 | 4-chloro-2-fluoro-3- | H | H | H | H | CO₂Et |
| | methoxyphenyl | | | | | |
| 55-66 | 4-chloro-2-fluoro-3- | H | H | H | H | CO₂n-Pr |
| | methoxyphenyl | | | | | |
| 55-67 | 4-chloro-2-fluoro-3- | H | H | H | H | CO₂i-Pr |
| | methoxyphenyl | | | | | |
| 55-68 | 4-chloro-2-fluoro-3- | H | H | H | H | CO₂CH₂CH═CH₂ |
| | methoxyphenyl | | | | | |
| 55-69 | 4-chloro-2-fluoro-3- | H | H | H | H | CO₂CH₂CH₂OMe |
| | methoxyphenyl | | | | | |
| 55-70 | 4-chloro-2-fluoro-3- | H | H | H | H | CO₂CH₂Ph |
| | methoxyphenyl | | | | | |
| 55-71 | 4-chloro-2-fluoro-3- | H | H | Me | H | CO₂Et |
| | methoxyphenyl | | | | | |
| 55-72 | 4-chloro-2-fluoro-3- | H | H | Me | H | CO₂n-Pr |
| | methoxyphenyl | | | | | |
| 55-73 | 4-chloro-2-fluoro-3- | H | H | Me | H | CO₂i-Pr |
| | methoxyphenyl | | | | | |
| 55-74 | 4-chloro-2-fluoro-3- | H | H | Me | H | CO₂CH₂CH═CH₂ |
| | methoxyphenyl | | | | | |
| 55-75 | 4-chloro-2-fluoro-3- | H | H | Me | H | CO₂CH₂CH₂OMe |
| | methoxyphenyl | | | | | |
| 55-76 | 4-chloro-2-fluoro-3- | H | H | Me | H | CO₂CH₂Ph |
| | methoxyphenyl | | | | | |
| 55-77 | 4-chloro-2-fluoro-3- | Me | H | H | H | CO₂Et |
| | methoxyphenyl | | | | | |
| 55-78 | 4-chloro-2-fluoro-3- | Me | H | H | H | CO₂n-Pr |
| | methoxyphenyl | | | | | |
| 55-79 | 4-chloro-2-fluoro-3- | Me | H | H | H | CO₂i-Pr |
| | methoxyphenyl | | | | | |
| 55-80 | 4-chloro-2-fluoro-3- | Me | H | H | H | CO₂CH₂CH═CH₂ |
| | methoxyphenyl | | | | | |
| 55-81 | 4-chloro-2-fluoro-3- | Me | H | H | H | CO₂CH₂CH₂OMe |
| | methoxyphenyl | | | | | |
| 55-82 | 4-chloro-2-fluoro-3- | Me | H | H | H | CO₂CH₂Ph |
| | methoxyphenyl | | | | | |
| 55-83 | 4-chloro-2-fluoro-3- | Me | H | Me | H | CO₂Et |
| | methoxyphenyl | | | | | |
| 55-84 | 4-chloro-2-fluoro-3- | Me | H | Me | H | CO₂n-Pr |
| | methoxyphenyl | | | | | |
| 55-85 | 4-chloro-2-fluoro-3- | Me | H | Me | H | CO₂i-Pr |
| | methoxyphenyl | | | | | |
| 55-86 | 4-chloro-2-fluoro-3- | Me | H | Me | H | CO₂CH₂CH═CH₂ |
| | methoxyphenyl | | | | | |
| 55-87 | 4-chloro-2-fluoro-3- | Me | H | Me | H | CO₂CH₂CH₂OMe |
| | methoxyphenyl | | | | | |
| 55-88 | 4-chloro-2-fluoro-3- | Me | H | Me | H | CO₂CH₂Ph |
| | methoxyphenyl | | | | | |
| 55-89 | 4-chloro-2-fluoro-3- | ═O | | H | H | CO₂H |
| | methoxyphenyl | | | | | |
| 55-90 | 4-chloro-2-fluoro-3- | ═O | | H | H | CO₂Me |
| | methoxyphenyl | | | | | |
| 55-91 | 4-chloro-2-fluoro-3- | ═O | | Me | H | CO₂H |
| | methoxyphenyl | | | | | |
| 55-92 | 4-chloro-2-fluoro-3- | ═O | | Me | H | CO₂Me |
| | methoxyphenyl | | | | | |
| 55-93 | 4-chloro-2-fluoro-3- | CH₂CH₂ | | H | H | CO₂H |
| | methoxyphenyl | | | | | |
| 55-94 | 4-chloro-2-fluoro-3- | CH₂CH₂ | | H | H | CO₂Me |
| | methoxyphenyl | | | | | |
| 55-95 | 4-chloro-2-fluoro-3- | CH₂CH₂ | | Me | H | CO₂H |
| | methoxyphenyl | | | | | |
| 55-96 | 4-chloro-2-fluoro-3- | CH₂CH₂ | | Me | H | CO₂Me |
| | methoxyphenyl | | | | | |
| 55-97 | 4-chloro-3- | H | H | H | H | CO₂H |
| | dimethylamino-2- | | | | | |
| | fluorophenyl | | | | | |
| 55-98 | 4-chloro-3- | H | H | H | H | CO₂Me |
| | dimethylamino-2- | | | | | |
| | fluorophenyl | | | | | |
| 55-99 | 4-chloro-3- | H | H | Me | H | CO₂H |
| | dimethylamino-2- | | | | | |
| | fluorophenyl | | | | | |
| 55-100 | 4-chloro-3- | H | H | Me | H | CO₂Me |
| | dimethylamino-2- | | | | | |
| | fluorophenyl | | | | | |
| 55-101 | 4-chloro-3- | Me | H | H | H | CO₂H |
| | dimethylamino-2- | | | | | |
| | fluorophenyl | | | | | |
| 55-102 | 4-chloro-3- | Me | H | H | H | CO₂Me |
| | dimethylamino-2- | | | | | |
| | fluorophenyl | | | | | |
| 55-103 | 4-chloro-3- | Me | H | Me | H | CO₂H |
| | dimethylamino-2- | | | | | |
| | fluorophenyl | | | | | |
| 55-104 | 4-chloro-3- | Me | H | Me | H | CO₂Me |
| | dimethylamino-2- | | | | | |
| | fluorophenyl | | | | | |
| 55-105 | cyclopropyl | Me | Me | H | H | CO₂H |
| 55-106 | cyclopropyl | Me | Me | H | H | CO₂Me |
| 55-107 | cyclopropyl | Me | Me | Me | H | CO₂H |
| 55-108 | cyclopropyl | Me | Me | Me | H | CO₂Me |
| 55-109 | 4-chlorophenyl | Me | Me | H | H | CO₂H |
| 55-110 | 4-chlorophenyl | Me | Me | H | H | CO₂Me |
| 55-111 | 4-chlorophenyl | Me | Me | Me | H | CO₂H |
| 55-112 | 4-chlorophenyl | Me | Me | Me | H | CO₂Me |
| 55-113 | 4-chloro-3- | Me | Me | H | H | CO₂H |
| | fluorophenyl | | | | | |
| 55-114 | 4-chloro-3- | Me | Me | H | H | CO₂Me |
| | fluorophenyl | | | | | |
| 55-115 | 4-chloro-3- | Me | Me | Me | H | CO₂H |
| | fluorophenyl | | | | | |
| 55-116 | 4-chloro-3- | Me | Me | Me | H | CO₂Me |
| | fluorophenyl | | | | | |
| 55-117 | 4-chloro-3- | Me | Me | H | H | CO₂Et |
| | fluorophenyl | | | | | |
| 55-118 | 4-chloro-3- | Me | Me | H | H | CO₂n-Pr |
| | fluorophenyl | | | | | |
| 55-119 | 4-chloro-3- | Me | Me | H | H | CO₂i-Pr |
| | fluorophenyl | | | | | |
| 55-120 | 4-chloro-3- | Me | Me | H | H | CO₂CH₂CH═CH₂ |
| | fluorophenyl | | | | | |
| 55-121 | 4-chloro-3- | Me | Me | H | H | CO₂CH₂CH₂OMe |
| | fluorophenyl | | | | | |
| 55-122 | 4-chloro-3- | Me | Me | H | H | CO₂CH₂Ph |
| | fluorophenyl | | | | | |
| 55-123 | 4-chloro-3- | Me | Me | Me | H | CO₂Et |
| | fluorophenyl | | | | | |
| 55-124 | 4-chloro-3- | Me | Me | Me | H | CO₂n-Pr |
| | fluorophenyl | | | | | |
| 55-125 | 4-chloro-3- | Me | Me | Me | H | CO₂i-Pr |
| | fluorophenyl | | | | | |
| 55-126 | 4-chloro-3- | Me | Me | Me | H | CO₂CH₂CH═CH₂ |
| | fluorophenyl | | | | | |
| 55-127 | 4-chloro-3- | Me | Me | Me | H | CO₂CH₂CH₂OMe |
| | fluorophenyl | | | | | |
| 55-128 | 4-chloro-3- | Me | Me | Me | H | CO₂CH₂Ph |
| | fluorophenyl | | | | | |
| 55-129 | 4-chloro-2-fluoro-3- | Me | Me | H | H | CO₂H |
| | methoxyphenyl | | | | | |
| 55-130 | 4-chloro-2-fluoro-3- | Me | Me | H | H | CO₂Me |
| | methoxyphenyl | | | | | |
| 55-131 | 4-chloro-2-fluoro-3- | Me | Me | Me | H | CO₂H |
| | methoxyphenyl | | | | | |
| 55-132 | 4-chloro-2-fluoro-3- | Me | Me | Me | H | CO₂Me |
| | methoxyphenyl | | | | | |
| 55-133 | 4-chloro-2-fluoro-3- | Me | Me | H | H | CO₂Et |
| | methoxyphenyl | | | | | |
| 55-134 | 4-chloro-2-fluoro-3- | Me | Me | H | H | CO₂n-Pr |
| | methoxyphenyl | | | | | |
| 55-135 | 4-chloro-2-fluoro-3- | Me | Me | H | H | CO₂i-Pr |
| | methoxyphenyl | | | | | |
| 55-136 | 4-chloro-2-fluoro-3- | Me | Me | H | H | CO₂CH₂CH═CH₂ |
| | methoxyphenyl | | | | | |
| 55-137 | 4-chloro-2-fluoro-3- | Me | Me | H | H | CO₂CH₂CH₂OMe |
| | methoxyphenyl | | | | | |
| 55-138 | 4-chloro-2-fluoro-3- | Me | Me | H | H | CO₂CH₂Ph |
| | methoxyphenyl | | | | | |
| 55-139 | 4-chloro-2-fluoro-3- | Me | Me | Me | H | CO₂Et |
| | methoxyphenyl | | | | | |
| 55-140 | 4-chloro-2-fluoro-3- | Me | Me | Me | H | CO₂n-Pr |
| | methoxyphenyl | | | | | |
| 55-141 | 4-chloro-2-fluoro-3- | Me | Me | Me | H | CO₂i-Pr |
| | methoxyphenyl | | | | | |
| 55-142 | 4-chloro-2-fluoro-3- | Me | Me | Me | H | CO₂CH₂CH═CH₂ |
| | methoxyphenyl | | | | | |
| 55-143 | 4-chloro-2-fluoro-3- | Me | Me | Me | H | CO₂CH₂CH₂OMe |
| | methoxyphenyl | | | | | |
| 55-144 | 4-chloro-2-fluoro-3- | Me | Me | Me | H | CO₂CH₂Ph |
| | methoxyphenyl | | | | | |
| 55-145 | 4-chloro-3- | Me | Me | H | H | CO₂H |
| | dimethylamino-2- | | | | | |
| | fluorophenyl | | | | | |
| 55-146 | 4-chloro-3- | Me | Me | H | H | CO₂Me |
| | dimethylamino-2- | | | | | |
| | fluorophenyl | | | | | |
| 55-147 | 4-chloro-3- | Me | Me | Me | H | CO₂H |
| | dimethylamino-2- | | | | | |
| | fluorophenyl | | | | | |
| 55-148 | 4-chloro-3- | Me | Me | Me | H | CO₂Me |
| | dimethylamino-2- | | | | | |
| | fluorophenyl | | | | | |
| 55-149 | cyclopropyl | H | H | Me | Me | CO₂H |
| 55-150 | cyclopropyl | H | H | Me | Me | CO₂Me |
| 55-151 | cyclopropyl | Me | H | Me | Me | CO₂H |
| 55-152 | cyclopropyl | Me | H | Me | Me | CO₂Me |
| 55-153 | 4-chlorophenyl | H | H | Me | Me | CO₂H |
| 55-154 | 4-chlorophenyl | H | H | Me | Me | CO₂Me |
| 55-155 | 4-chlorophenyl | Me | H | Me | Me | CO₂H |
| 55-156 | 4-chlorophenyl | Me | H | Me | Me | CO₂Me |
| 55-157 | 4-chloro-3- | H | H | Me | Me | CO₂H |
| | fluorophenyl | | | | | |
| 55-158 | 4-chloro-3- | H | H | Me | Me | CO₂Me |
| | fluorophenyl | | | | | |
| 55-159 | 4-chloro-3- | Me | H | Me | Me | CO₂H |
| | fluorophenyl | | | | | |
| 55-160 | 4-chloro-3- | Me | H | Me | Me | CO₂Me |
| | fluorophenyl | | | | | |
| 55-161 | 4-chloro-3- | H | H | Me | Me | CO₂Et |
| | fluorophenyl | | | | | |
| 55-162 | 4-chloro-3- | H | H | Me | Me | CO₂n-Pr |
| | fluorophenyl | | | | | |
| 55-163 | 4-chloro-3- | H | H | Me | Me | CO₂i-Pr |
| | fluorophenyl | | | | | |
| 55-164 | 4-chloro-3- | H | H | Me | Me | CO₂CH₂CH═CH₂ |
| | fluorophenyl | | | | | |
| 55-165 | 4-chloro-3- | H | H | Me | Me | CO₂CH₂CH₂OMe |
| | fluorophenyl | | | | | |
| 55-166 | 4-chloro-3- | H | H | Me | Me | CO₂CH₂Ph |
| | fluorophenyl | | | | | |
| 55-167 | 4-chloro-3- | Me | H | Me | Me | CO₂Et |
| | fluorophenyl | | | | | |
| 55-168 | 4-chloro-3- | Me | H | Me | Me | CO₂n-Pr |
| | fluorophenyl | | | | | |
| 55-169 | 4-chloro-3- | Me | H | Me | Me | CO₂i-Pr |
| | fluorophenyl | | | | | |
| 55-170 | 4-chloro-3- | Me | H | Me | Me | CO₂CH₂CH═CH₂ |
| | fluorophenyl | | | | | |
| 55-171 | 4-chloro-3- | Me | H | Me | Me | CO₂CH₂CH₂OMe |
| | fluorophenyl | | | | | |
| 55-172 | 4-chloro-3- | Me | H | Me | Me | CO₂CH₂Ph |
| | fluorophenyl | | | | | |
| 55-173 | 4-chloro-3- | ═O | | Me | Me | CO₂H |
| | fluorophenyl | | | | | |
| 55-174 | 4-chloro-3- | ═O | | Me | Me | CO₂Me |
| | fluorophenyl | | | | | |
| 55-175 | 4-chloro-3- | CH₂CH₂ | | Me | Me | CO₂H |
| | fluorophenyl | | | | | |
| 55-176 | 4-chloro-3- | CH₂CH₂ | | Me | Me | CO₂Me |
| | fluorophenyl | | | | | |
| 55-177 | 4-chloro-2-fluoro-3- | H | H | Me | Me | CO₂H |
| | methoxyphenyl | | | | | |
| 55-178 | 4-chloro-2-fluoro-3- | H | H | Me | Me | CO₂Me |
| | methoxyphenyl | | | | | |
| 55-179 | 4-chloro-2-fluoro-3- | Me | H | Me | Me | CO₂H |
| | methoxyphenyl | | | | | |
| 55-180 | 4-chloro-2-fluoro-3- | Me | H | Me | Me | CO₂Me |
| | methoxyphenyl | | | | | |
| 55-181 | 4-chloro-2-fluoro-3- | H | H | Me | Me | CO₂Et |
| | methoxyphenyl | | | | | |
| 55-182 | 4-chloro-2-fluoro-3- | H | H | Me | Me | CO₂n-Pr |
| | methoxyphenyl | | | | | |
| 55-183 | 4-chloro-2-fluoro-3- | H | H | Me | Me | CO₂i-Pr |
| | methoxyphenyl | | | | | |
| 55-184 | 4-chloro-2-fluoro-3- | H | H | Me | Me | CO₂CH₂CH═CH₂ |
| | methoxyphenyl | | | | | |
| 55-185 | 4-chloro-2-fluoro-3- | H | H | Me | Me | CO₂CH₂CH₂OMe |
| | methoxyphenyl | | | | | |
| 55-186 | 4-chloro-2-fluoro-3- | H | H | Me | Me | CO₂CH₂Ph |
| | methoxyphenyl | | | | | |
| 55-187 | 4-chloro-2-fluoro-3- | Me | H | Me | Me | CO₂Et |
| | methoxyphenyl | | | | | |
| 55-188 | 4-chloro-2-fluoro-3- | Me | H | Me | Me | CO₂n-Pr |
| | methoxyphenyl | | | | | |
| 55-189 | 4-chloro-2-fluoro-3- | Me | H | Me | Me | CO₂i-Pr |
| | methoxyphenyl | | | | | |
| 55-190 | 4-chloro-2-fluoro-3- | Me | H | Me | Me | CO₂CH₂CH═CH₂ |
| | methoxyphenyl | | | | | |
| 55-191 | 4-chloro-2-fluoro-3- | Me | H | Me | Me | CO₂CH₂CH₂OMe |
| | methoxyphenyl | | | | | |
| 55-192 | 4-chloro-2-fluoro-3- | Me | H | Me | Me | CO₂CH₂Ph |
| | methoxyphenyl | | | | | |
| 55-193 | 4-chloro-2-fluoro-3- | ═O | | Me | Me | CO₂H |
| | methoxyphenyl | | | | | |
| 55-194 | 4-chloro-2-fluoro-3- | ═O | | Me | Me | CO₂Me |
| | methoxyphenyl | | | | | |
| 55-195 | 4-chloro-2-fluoro-3- | CH₂CH₂ | | Me | Me | CO₂H |
| | methoxyphenyl | | | | | |
| 55-196 | 4-chloro-2-fluoro-3- | CH₂CH₂ | | Me | Me | CO₂Me |
| | methoxyphenyl | | | | | |
| 55-197 | 4-chloro-3- | H | H | Me | Me | CO₂H |
| | dimethylamino-2- | | | | | |
| | fluorophenyl | | | | | |
| 55-198 | 4-chloro-3- | H | H | Me | Me | CO₂Me |
| | dimethylamino-2- | | | | | |
| | fluorophenyl | | | | | |
| 55-199 | 4-chloro-3- | Me | H | Me | Me | CO₂H |
| | dimethylamino-2- | | | | | |
| | fluorophenyl | | | | | |
| 55-200 | 4-chloro-3- | Me | H | Me | Me | CO₂Me |
| | dimethylamino-2- | | | | | |
| | fluorophenyl | | | | | |
| 55-201 | cyclopropyl | Me | Me | Me | Me | CO₂H |
| 55-202 | cyclopropyl | Me | Me | Me | Me | CO₂Me |
| 55-203 | 4-chlorophenyl | Me | Me | Me | Me | CO₂H |
| 55-204 | 4-chlorophenyl | Me | Me | Me | Me | CO₂Me |
| 55-205 | 4-chloro-3- | Me | Me | Me | Me | CO₂H |
| | fluorophenyl | | | | | |
| 55-206 | 4-chloro-3- | Me | Me | Me | Me | CO₂Me |
| | fluorophenyl | | | | | |
| 55-207 | 4-chloro-3- | Me | Me | Me | Me | CO₂Et |
| | fluorophenyl | | | | | |
| 55-208 | 4-chloro-3- | Me | Me | Me | Me | CO₂n-Pr |
| | fluorophenyl | | | | | |
| 55-209 | 4-chloro-3- | Me | Me | Me | Me | CO₂i-Pr |
| | fluorophenyl | | | | | |
| 55-210 | 4-chloro-3- | Me | Me | Me | Me | CO₂CH₂CH═CH₂ |
| | fluorophenyl | | | | | |
| 55-211 | 4-chloro-3- | Me | Me | Me | Me | CO₂CH₂CH₂OMe |
| | fluorophenyl | | | | | |
| 55-212 | 4-chloro-3- | Me | Me | Me | Me | CO₂CH₂Ph |
| | fluorophenyl | | | | | |
| 55-213 | 4-chloro-2-fluoro-3- | Me | Me | Me | Me | CO₂H |
| | methoxyphenyl | | | | | |
| 55-214 | 4-chloro-2-fluoro-3- | Me | Me | Me | Me | CO₂Me |
| | methoxyphenyl | | | | | |
| 55-215 | 4-chloro-2-fluoro-3- | Me | Me | Me | Me | CO₂Et |
| | methoxyphenyl | | | | | |
| 55-216 | 4-chloro-2-fluoro-3- | Me | Me | Me | Me | CO₂n-Pr |
| | methoxyphenyl | | | | | |
| 55-217 | 4-chloro-2-fluoro-3- | Me | Me | Me | Me | CO₂i-Pr |
| | methoxyphenyl | | | | | |
| 55-218 | 4-chloro-2-fluoro-3- | Me | Me | Me | Me | CO₂CH₂CH═CH₂ |
| | methoxyphenyl | | | | | |
| 55-219 | 4-chloro-2-fluoro-3- | Me | Me | Me | Me | CO₂CH₂CH₂OMe |
| | methoxyphenyl | | | | | |
| 55-220 | 4-chloro-2-fluoro-3- | Me | Me | Me | Me | CO₂CH₂Ph |
| | methoxyphenyl | | | | | |
| 55-221 | 4-chloro-3- | Me | Me | Me | Me | CO₂H |
| | dimethylamino-2- | | | | | |
| | fluorophenyl | | | | | |
| 55-222 | 4-chloro-3- | Me | Me | Me | Me | CO₂Me |
| | dimethylamino-2- | | | | | |
| | fluorophenyl | | | | | |

| 55-223 | 4-chloro-3- | ═O | | ═CH₂ | CO₂H |
|---|---|---|---|---|---|
| | fluorophenyl | | | | |
| 55-224 | 4-chloro-3- | ═O | | ═CH₂ | CO₂Me |
| | fluorophenyl | | | | |
| 55-225 | 4-chloro-3- | CH₂CH₂ | | ═CH₂ | CO₂H |
| | fluorophenyl | | | | |
| 55-226 | 4-chloro-3- | CH₂CH₂ | | ═CH₂ | CO₂Me |
| | fluorophenyl | | | | |
| 55-227 | 4-chloro-2-fluoro-3- | ═O | | ═CH₂ | CO₂H |
| | methoxyphenyl | | | | |
| 55-228 | 4-chloro-2-fluoro-3- | ═O | | ═CH₂ | CO₂Me |
| | methoxyphenyl | | | | |
| 55-229 | 4-chloro-2-fluoro-3- | CH₂CH₂ | | ═CH₂ | CO₂H |
| | methoxyphenyl | | | | |
| 55-230 | 4-chloro-2-fluoro-3- | CH₂CH₂ | | ═CH₂ | CO₂Me |
| | methoxyphenyl | | | | |
| 55-231 | cyclopropyl | Me | Me | ═CH₂ | CO₂H |
| 55-232 | cyclopropyl | Me | Me | ═CH₂ | CO₂Me |
| 55-233 | 4-chlorophenyl | Me | Me | ═CH₂ | CO₂H |
| 55-234 | 4-chlorophenyl | Me | Me | ═CH₂ | CO₂Me |
| 55-235 | 4-chloro-3- | Me | Me | ═CH₂ | CO₂H |
| | fluorophenyl | | | | |
| 55-236 | 4-chloro-3- | Me | Me | ═CH₂ | CO₂Me |
| | fluorophenyl | | | | |
| 55-237 | 4-chloro-3- | Me | Me | ═CH₂ | CO₂Et |
| | fluorophenyl | | | | |
| 55-238 | 4-chloro-3- | Me | Me | ═CH₂ | CO₂n-Pr |
| | fluorophenyl | | | | |
| 55-239 | 4-chloro-3- | Me | Me | ═CH₂ | CO₂i-Pr |
| | fluorophenyl | | | | |
| 55-240 | 4-chloro-3- | Me | Me | ═CH₂ | CO₂CH₂CH═CH₂ |
| | fluorophenyl | | | | |
| 55-241 | 4-chloro-3- | Me | Me | ═CH₂ | CO₂CH₂CH₂OMe |
| | fluorophenyl | | | | |
| 55-242 | 4-chloro-3- | Me | Me | ═CH₂ | CO₂CH₂Ph |
| | fluorophenyl | | | | |
| 55-243 | 4-chloro-2-fluoro-3- | Me | Me | ═CH₂ | CO₂H |
| | methoxyphenyl | | | | |
| 55-244 | 4-chloro-2-fluoro-3- | Me | Me | ═CH₂ | CO₂Me |
| | methoxyphenyl | | | | |
| 55-245 | 4-chloro-2-fluoro-3- | Me | Me | ═CH₂ | CO₂Et |
| | methoxyphenyl | | | | |
| 55-246 | 4-chloro-2-fluoro-3- | Me | Me | ═CH₂ | CO₂n-Pr |
| | methoxyphenyl | | | | |
| 55-247 | 4-chloro-2-fluoro-3- | Me | Me | ═CH₂ | CO₂i-Pr |
| | methoxyphenyl | | | | |
| 55-248 | 4-chloro-2-fluoro-3- | Me | Me | ═CH₂ | CO₂CH₂CH═CH₂ |
| | methoxyphenyl | | | | |
| 55-249 | 4-chloro-2-fluoro-3- | Me | Me | ═CH₂ | CO₂CH₂CH₂OMe |
| | methoxyphenyl | | | | |
| 55-250 | 4-chloro-2-fluoro-3- | Me | Me | ═CH₂ | CO₂CH₂Ph |
| | methoxyphenyl | | | | |
| 55-251 | 4-chloro-3- | Me | Me | ═CH₂ | CO₂H |
| | dimethylamino-2- | | | | |
| | fluorophenyl | | | | |
| 55-252 | 4-chloro-3- | Me | Me | ═CH₂ | CO₂Me |
| | dimethylamino-2- | | | | |
| | fluorophenyl | | | | |

252 compounds are described, designated compounds 56-1 to 56-252 respectively, of formula (1B) wherein D is N and X is NMe, and the values of A, R⁵, R⁶, R⁷, R⁸ and Z are as defined in Table 2.

252 compounds are described, designated compounds 57-1 to 57-252 respectively, of formula (1B) wherein D is N and X is O, and the values of A, R⁵, R⁶, R⁷, R⁸ and Z are as defined in Table 2.

252 compounds are described, designated compounds 58-1 to 58-252 respectively, of formula (1B) wherein D is N and X is S, and the values of A, R⁵, R⁶, R⁷, R⁸ and Z are as defined in Table 2.

252 compounds are described, designated compounds 59-1 to 59-252 respectively, of formula (1B) wherein D is CH and X is NH, and the values of A, R⁵, R⁶, R⁷, R⁸ and Z are as defined in Table 2.

252 compounds are described, designated compounds 60-1 to 60-252 respectively, of formula (1B) wherein D is CH and X is NMe, and the values of A, R⁵, R⁶, R⁷, R⁸ and Z are as defined in Table 2.

252 compounds are described, designated compounds 61-1 to 61-252 respectively, of formula (1B) wherein D is CH and X is O, and the values of A, R⁵, R⁶, R⁷, R⁸ and Z are as defined in Table 2.

252 compounds are described, designated compounds 62-1 to 62-252 respectively, of formula (1B) wherein D is CH and X is S, and the values of A, R⁵, R⁶, R⁷, R⁸ and Z are as defined in Table 2.

Table 3 below provides 172 compounds designated compounds 63-1 to 63-172 respectively, of formula (1C) wherein D is N and X is NH.

TABLE 3

| | |
|---|---|
| | (1C) |
| 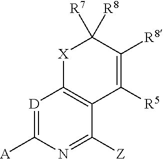 | |

| Com- | | | | | | |
|---|---|---|---|---|---|---|
| pound | | | | | | |
| Num- | Substituent Values | | | | | |
| ber | A | R⁷ | R⁸ | R^{8′} | R⁵ | Z |
| 63-1 | cyclopropyl | H | H | H | Me | CO₂H |
| 63-2 | cyclopropyl | H | H | H | Me | CO₂Me |
| 63-3 | cyclopropyl | H | H | Me | Me | CO₂H |
| 63-4 | cyclopropyl | H | H | Me | Me | CO₂Me |
| 63-5 | cyclopropyl | Me | H | H | Me | CO₂H |
| 63-6 | cyclopropyl | Me | H | H | Me | CO₂Me |
| 63-7 | cyclopropyl | Me | H | Me | Me | CO₂H |
| 63-8 | cyclopropyl | Me | H | Me | Me | CO₂Me |
| 63-9 | 4-chlorophenyl | H | H | H | Me | CO₂H |
| 63-10 | 4-chlorophenyl | H | H | H | Me | CO₂Me |
| 63-11 | 4-chlorophenyl | H | H | Me | Me | CO₂H |
| 63-12 | 4-chlorophenyl | H | H | Me | Me | CO₂Me |
| 63-13 | 4-chlorophenyl | Me | H | H | Me | CO₂H |
| 63-14 | 4-chlorophenyl | Me | H | H | Me | CO₂Me |
| 63-15 | 4-chlorophenyl | Me | H | Me | Me | CO₂H |
| 63-16 | 4-chlorophenyl | Me | H | Me | Me | CO₂Me |
| 63-17 | 4-chloro-3- | H | H | H | H | CO₂H |
| | fluorophenyl | | | | | |
| 63-18 | 4-chloro-3- | H | H | H | H | CO₂Me |
| | fluorophenyl | | | | | |
| 63-19 | 4-chloro-3- | H | H | Me | H | CO₂H |
| | fluorophenyl | | | | | |
| 63-20 | 4-chloro-3- | H | H | Me | H | CO₂Me |
| | fluorophenyl | | | | | |
| 63-21 | 4-chloro-3- | Me | H | H | H | CO₂H |
| | fluorophenyl | | | | | |
| 63-22 | 4-chloro-3- | Me | H | H | H | CO₂Me |
| | fluorophenyl | | | | | |
| 63-23 | 4-chloro-3- | Me | H | Me | H | CO₂H |
| | fluorophenyl | | | | | |
| 63-24 | 4-chloro-3- | Me | H | Me | H | CO₂Me |
| | fluorophenyl | | | | | |
| 63-25 | 4-chloro-3- | H | H | H | Me | CO₂H |
| | fluorophenyl | | | | | |
| 63-26 | 4-chloro-3- | H | H | H | Me | CO₂Me |
| | fluorophenyl | | | | | |
| 63-27 | 4-chloro-3- | H | H | Me | Me | CO₂H |
| | fluorophenyl | | | | | |
| 63-28 | 4-chloro-3- | H | H | Me | Me | CO₂Me |
| | fluorophenyl | | | | | |
| 63-29 | 4-chloro-3- | Me | H | H | Me | CO₂H |
| | fluorophenyl | | | | | |
| 63-30 | 4-chloro-3- | Me | H | H | Me | CO₂Me |
| | fluorophenyl | | | | | |
| 63-31 | 4-chloro-3- | Me | H | Me | Me | CO₂H |
| | fluorophenyl | | | | | |
| 63-32 | 4-chloro-3- | Me | H | Me | Me | CO₂Me |
| | fluorophenyl | | | | | |
| 63-33 | 4-chloro-3- | H | H | H | Me | CO₂Et |
| | fluorophenyl | | | | | |
| 63-34 | 4-chloro-3- | H | H | H | Me | CO₂n-Pr |
| | fluorophenyl | | | | | |
| 63-35 | 4-chloro-3- | H | H | H | Me | CO₂i-Pr |
| | fluorophenyl | | | | | |
| 63-36 | 4-chloro-3- | H | H | H | Me | CO₂CH₂CH═CH₂ |
| | fluorophenyl | | | | | |
| 63-37 | 4-chloro-3- | H | H | H | Me | CO₂CH₂CH₂OMe |
| | fluorophenyl | | | | | |
| 63-38 | 4-chloro-3- | H | H | H | Me | CO₂CH₂Ph |
| | fluorophenyl | | | | | |
| 63-39 | 4-chloro-3- | H | H | Me | Me | CO₂Et |
| | fluorophenyl | | | | | |
| 63-40 | 4-chloro-3- | H | H | Me | Me | CO₂n-Pr |
| | fluorophenyl | | | | | |
| 63-41 | 4-chloro-3- | H | H | Me | Me | CO₂i-Pr |
| | fluorophenyl | | | | | |
| 63-42 | 4-chloro-3- | H | H | Me | Me | CO₂CH₂CH═CH₂ |
| | fluorophenyl | | | | | |
| 63-43 | 4-chloro-3- | H | H | Me | Me | CO₂CH₂CH₂OMe |
| | fluorophenyl | | | | | |
| 63-44 | 4-chloro-3- | H | H | Me | Me | CO₂CH₂Ph |
| | fluorophenyl | | | | | |
| 63-45 | 4-chloro-3- | Me | H | H | Me | CO₂Et |
| | fluorophenyl | | | | | |
| 63-46 | 4-chloro-3- | Me | H | H | Me | CO₂n-Pr |
| | fluorophenyl | | | | | |
| 63-47 | 4-chloro-3- | Me | H | H | Me | CO₂i-Pr |
| | fluorophenyl | | | | | |
| 63-48 | 4-chloro-3- | Me | H | H | Me | CO₂CH₂CH═CH₂ |
| | fluorophenyl | | | | | |
| 63-49 | 4-chloro-3- | Me | H | H | Me | CO₂CH₂CH₂OMe |
| | fluorophenyl | | | | | |
| 63-50 | 4-chloro-3- | Me | H | H | Me | CO₂CH₂Ph |
| | fluorophenyl | | | | | |
| 63-51 | 4-chloro-3- | Me | H | Me | Me | CO₂Et |
| | fluorophenyl | | | | | |
| 63-52 | 4-chloro-3- | Me | H | Me | Me | CO₂n-Pr |
| | fluorophenyl | | | | | |
| 63-53 | 4-chloro-3- | Me | H | Me | Me | CO₂i-Pr |
| | fluorophenyl | | | | | |
| 63-54 | 4-chloro-3- | Me | H | Me | Me | CO₂CH₂CH═CH₂ |
| | fluorophenyl | | | | | |
| 63-55 | 4-chloro-3- | Me | H | Me | Me | CO₂CH₂CH₂OMe |
| | fluorophenyl | | | | | |
| 63-56 | 4-chloro-3- | Me | H | Me | Me | CO₂CH₂Ph |
| | fluorophenyl | | | | | |
| 63-57 | 4-chloro-3- | ═O | | H | Me | CO₂H |
| | fluorophenyl | | | | | |
| 63-58 | 4-chloro-3- | ═O | | H | Me | CO₂Me |
| | fluorophenyl | | | | | |
| 63-59 | 4-chloro-3- | ═O | | Me | Me | CO₂H |
| | fluorophenyl | | | | | |
| 63-60 | 4-chloro-3- | ═O | | Me | Me | CO₂Me |
| | fluorophenyl | | | | | |
| 63-61 | 4-chloro-3- | CH₂CH₂ | | H | Me | CO₂H |
| | fluorophenyl | | | | | |
| 63-62 | 4-chloro-3- | CH₂CH₂ | | H | Me | CO₂Me |
| | fluorophenyl | | | | | |
| 63-63 | 4-chloro-3- | CH₂CH₂ | | Me | Me | CO₂H |
| | fluorophenyl | | | | | |
| 63-64 | 4-chloro-3- | CH₂CH₂ | | Me | Me | CO₂Me |
| | fluorophenyl | | | | | |
| 63-65 | 4-chloro-2- | H | H | H | H | CO₂H |
| | fluoro-3- | | | | | |
| | methoxyphenyl | | | | | |
| 63-66 | 4-chloro-2- | H | H | H | H | CO₂Me |
| | fluoro-3- | | | | | |
| | methoxyphenyl | | | | | |
| 63-67 | 4-chloro-2- | H | H | Me | H | CO₂H |
| | fluoro-3- | | | | | |
| | methoxyphenyl | | | | | |
| 63-68 | 4-chloro-2- | H | H | Me | H | CO₂Me |
| | fluoro-3- | | | | | |
| | methoxyphenyl | | | | | |
| 63-69 | 4-chloro-2- | Me | H | H | H | CO₂H |
| | fluoro-3- | | | | | |
| | methoxyphenyl | | | | | |
| 63-70 | 4-chloro-2- | Me | H | H | H | CO₂Me |
| | fluoro-3- | | | | | |
| | methoxyphenyl | | | | | |
| 63-71 | 4-chloro-2- | Me | H | Me | H | CO₂H |
| | fluoro-3- | | | | | |
| | methoxyphenyl | | | | | |
| 63-72 | 4-chloro-2- | Me | H | Me | H | CO₂Me |
| | fluoro-3- | | | | | |
| | methoxyphenyl | | | | | |
| 63-73 | 4-chloro-2- | H | H | H | Me | CO₂H |
| | fluoro-3- | | | | | |
| | methoxyphenyl | | | | | |
| 63-74 | 4-chloro-2- | H | H | H | Me | CO₂Me |
| | fluoro-3- | | | | | |
| | methoxyphenyl | | | | | |
| 63-75 | 4-chloro-2- | H | H | Me | Me | CO₂H |
| | fluoro-3- | | | | | |
| | methoxyphenyl | | | | | |
| 63-76 | 4-chloro-2- | H | H | Me | Me | CO₂Me |
| | fluoro-3- | | | | | |
| | methoxyphenyl | | | | | |
| 63-77 | 4-chloro-2- | Me | H | H | Me | CO₂H |
| | fluoro-3- | | | | | |
| | methoxyphenyl | | | | | |
| 63-78 | 4-chloro-2- | Me | H | H | Me | CO₂Me |
| | fluoro-3- | | | | | |
| | methoxyphenyl | | | | | |
| 63-79 | 4-chloro-2- | Me | H | Me | Me | CO₂H |
| | fluoro-3- | | | | | |
| | methoxyphenyl | | | | | |
| 63-80 | 4-chloro-2- | Me | H | Me | Me | CO₂Me |
| | fluoro-3- | | | | | |
| | methoxyphenyl | | | | | |
| 63-81 | 4-chloro-2- | H | H | H | Me | CO₂Et |
| | fluoro-3- | | | | | |
| | methoxyphenyl | | | | | |
| 63-82 | 4-chloro-2- | H | H | H | Me | CO₂n-Pr |
| | fluoro-3- | | | | | |
| | methoxyphenyl | | | | | |
| 63-83 | 4-chloro-2- | H | H | H | Me | CO₂i-Pr |
| | fluoro-3- | | | | | |
| | methoxyphenyl | | | | | |
| 63-84 | 4-chloro-2- | H | H | H | Me | CO₂CH₂CH═CH₂ |
| | fluoro-3- | | | | | |
| | methoxyphenyl | | | | | |
| 63-85 | 4-chloro-2- | H | H | H | Me | CO₂CH₂CH₂OMe |
| | fluoro-3- | | | | | |
| | methoxyphenyl | | | | | |
| 63-86 | 4-chloro-2- | H | H | H | Me | CO₂CH₂Ph |
| | fluoro-3- | | | | | |
| | methoxyphenyl | | | | | |
| 63-87 | 4-chloro-2- | H | H | Me | Me | CO₂Et |
| | fluoro-3- | | | | | |
| | methoxyphenyl | | | | | |
| 63-88 | 4-chloro-2- | H | H | Me | Me | CO₂n-Pr |
| | fluoro-3- | | | | | |
| | methoxyphenyl | | | | | |
| 63-89 | 4-chloro-2- | H | H | Me | Me | CO₂i-Pr |
| | fluoro-3- | | | | | |
| | methoxyphenyl | | | | | |
| 63-90 | 4-chloro-2- | H | H | Me | Me | CO₂CH₂CH═CH₂ |
| | fluoro-3- | | | | | |
| | methoxyphenyl | | | | | |
| 63-91 | 4-chloro-2- | H | H | Me | Me | CO₂CH₂CH₂OMe |
| | fluoro-3- | | | | | |
| | methoxyphenyl | | | | | |
| 63-92 | 4-chloro-2- | H | H | Me | Me | CO₂CH₂Ph |
| | fluoro-3- | | | | | |
| | methoxyphenyl | | | | | |
| 63-93 | 4-chloro-2- | Me | H | H | Me | CO₂Et |
| | fluoro-3- | | | | | |
| | methoxyphenyl | | | | | |
| 63-94 | 4-chloro-2- | Me | H | H | Me | CO₂n-Pr |
| | fluoro-3- | | | | | |
| | methoxyphenyl | | | | | |
| 63-95 | 4-chloro-2- | Me | H | H | Me | CO₂i-Pr |
| | fluoro-3- | | | | | |
| | methoxyphenyl | | | | | |
| 63-96 | 4-chloro-2- | Me | H | H | Me | CO₂CH₂CH═CH₂ |
| | fluoro-3- | | | | | |
| | methoxyphenyl | | | | | |
| 63-97 | 4-chloro-2- | Me | H | H | Me | CO₂CH₂CH₂OMe |
| | fluoro-3- | | | | | |
| | methoxyphenyl | | | | | |
| 63-98 | 4-chloro-2- | Me | H | H | Me | CO₂CH₂Ph |
| | fluoro-3- | | | | | |
| | methoxyphenyl | | | | | |
| 63-99 | 4-chloro-2- | Me | H | Me | Me | CO₂Et |
| | fluoro-3- | | | | | |
| | methoxyphenyl | | | | | |
| 63-100 | 4-chloro-2- | Me | H | Me | Me | CO₂n-Pr |
| | fluoro-3- | | | | | |
| | methoxyphenyl | | | | | |
| 63-101 | 4-chloro-2- | Me | H | Me | Me | CO₂i-Pr |
| | fluoro-3- | | | | | |
| | methoxyphenyl | | | | | |
| 63-102 | 4-chloro-2- | Me | H | Me | Me | CO₂CH₂CH═CH₂ |
| | fluoro-3- | | | | | |
| | methoxyphenyl | | | | | |
| 63-103 | 4-chloro-2- | Me | H | Me | Me | CO₂CH₂CH₂OMe |
| | fluoro-3- | | | | | |
| | methoxyphenyl | | | | | |
| 63-104 | 4-chloro-2- | Me | H | Me | Me | CO₂CH₂Ph |
| | fluoro-3- | | | | | |
| | methoxyphenyl | | | | | |
| 63-105 | 4-chloro-2- | ═O | | H | Me | CO₂H |
| | fluoro-3- | | | | | |
| | methoxyphenyl | | | | | |
| 63-106 | 4-chloro-2- | ═O | | H | Me | CO₂Me |
| | fluoro-3- | | | | | |
| | methoxyphenyl | | | | | |
| 63-107 | 4-chloro-2- | ═O | | Me | Me | CO₂H |
| | fluoro-3- | | | | | |
| | methoxyphenyl | | | | | |
| 63-108 | 4-chloro-2- | ═O | | Me | Me | CO₂Me |
| | fluoro-3- | | | | | |
| | methoxyphenyl | | | | | |
| 63-109 | 4-chloro-2- | CH₂CH₂ | | H | Me | CO₂H |
| | fluoro-3- | | | | | |
| | methoxyphenyl | | | | | |
| 63-110 | 4-chloro-2- | CH₂CH₂ | | H | Me | CO₂Me |
| | fluoro-3- | | | | | |
| | methoxyphenyl | | | | | |
| 63-111 | 4-chloro-2- | CH₂CH₂ | | Me | Me | CO₂H |
| | fluoro-3- | | | | | |
| | methoxyphenyl | | | | | |
| 63-112 | 4-chloro-2- | CH₂CH₂ | | Me | Me | CO₂Me |
| | fluoro-3- | | | | | |
| | methoxyphenyl | | | | | |
| 63-113 | 4-chloro-3- | H | H | H | Me | CO₂H |
| | dimethylamino- | | | | | |
| | 2-fluorophenyl | | | | | |
| 63-114 | 4-chloro-3- | H | H | H | Me | CO₂Me |
| | dimethylamino- | | | | | |
| | 2-fluorophenyl | | | | | |
| 63-115 | 4-chloro-3- | H | H | Me | Me | CO₂H |
| | dimethylamino- | | | | | |
| | 2-fluorophenyl | | | | | |
| 63-116 | 4-chloro-3- | H | H | Me | Me | CO₂Me |
| | dimethylamino- | | | | | |
| | 2-fluorophenyl | | | | | |
| 63-117 | 4-chloro-3- | Me | H | H | Me | CO₂H |
| | dimethylamino- | | | | | |
| | 2-fluorophenyl | | | | | |
| 63-118 | 4-chloro-3- | Me | H | H | Me | CO₂Me |
| | dimethylamino- | | | | | |
| | 2-fluorophenyl | | | | | |
| 63-119 | 4-chloro-3- | Me | H | Me | Me | CO₂H |
| | dimethylamino- | | | | | |
| | 2-fluorophenyl | | | | | |
| 63-120 | 4-chloro-3- | Me | H | Me | Me | CO₂Me |
| | dimethylamino- | | | | | |
| | 2-fluorophenyl | | | | | |
| 63-121 | cyclopropyl | Me | Me | H | Me | CO₂H |
| 63-122 | cyclopropyl | Me | Me | H | Me | CO₂Me |
| 63-123 | cyclopropyl | Me | Me | Me | Me | CO₂H |
| 63-124 | cyclopropyl | Me | Me | Me | Me | CO₂Me |
| 63-125 | 4-chlorophenyl | Me | Me | H | Me | CO₂H |
| 63-126 | 4-chlorophenyl | Me | Me | H | Me | CO₂Me |
| 63-127 | 4-chlorophenyl | Me | Me | Me | Me | CO₂H |
| 63-128 | 4-chlorophenyl | Me | Me | Me | Me | CO₂Me |
| 63-129 | 4-chloro-3- | Me | Me | H | H | CO₂H |
| | fluorophenyl | | | | | |
| 63-130 | 4-chloro-3- | Me | Me | H | H | CO₂Me |
| | fluorophenyl | | | | | |
| 63-131 | 4-chloro-3- | Me | Me | Me | H | CO₂H |
| | fluorophenyl | | | | | |
| 63-132 | 4-chloro-3- | Me | Me | Me | H | CO₂Me |
| | fluorophenyl | | | | | |
| 63-133 | 4-chloro-3- | Me | Me | H | Me | CO₂H |
| | fluorophenyl | | | | | |
| 63-134 | 4-chloro-3- | Me | Me | H | Me | CO₂Me |
| | fluorophenyl | | | | | |
| 63-135 | 4-chloro-3- | Me | Me | Me | Me | CO₂H |
| | fluorophenyl | | | | | |
| 63-136 | 4-chloro-3- | Me | Me | Me | Me | CO₂Me |
| | fluorophenyl | | | | | |
| 63-137 | 4-chloro-3- | Me | Me | H | Me | CO₂Et |
| | fluorophenyl | | | | | |
| 63-138 | 4-chloro-3- | Me | Me | H | Me | CO₂n-Pr |
| | fluorophenyl | | | | | |
| 63-139 | 4-chloro-3- | Me | Me | H | Me | CO₂i-Pr |
| | fluorophenyl | | | | | |
| 63-140 | 4-chloro-3- | Me | Me | H | Me | CO₂CH₂CH═CH₂ |
| | fluorophenyl | | | | | |
| 63-141 | 4-chloro-3- | Me | Me | H | Me | CO₂CH₂CH₂OMe |
| | fluorophenyl | | | | | |
| 63-142 | 4-chloro-3- | Me | Me | H | Me | CO₂CH₂Ph |
| | fluorophenyl | | | | | |
| 63-143 | 4-chloro-3- | Me | Me | Me | Me | CO₂Et |
| | fluorophenyl | | | | | |
| 63-144 | 4-chloro-3- | Me | Me | Me | Me | CO₂n-Pr |
| | fluorophenyl | | | | | |
| 63-145 | 4-chloro-3- | Me | Me | Me | Me | CO₂i-Pr |
| | fluorophenyl | | | | | |
| 63-146 | 4-chloro-3- | Me | Me | Me | Me | CO₂CH₂CH═CH₂ |
| | fluorophenyl | | | | | |
| 63-147 | 4-chloro-3- | Me | Me | Me | Me | CO₂CH₂CH₂OMe |
| | fluorophenyl | | | | | |
| 63-148 | 4-chloro-3- | Me | Me | Me | Me | CO₂CH₂Ph |
| | fluorophenyl | | | | | |
| 63-149 | 4-chloro-2- | Me | Me | H | H | CO₂H |
| | fluoro-3- | | | | | |
| | methoxyphenyl | | | | | |
| 63-150 | 4-chloro-2- | Me | Me | H | H | CO₂Me |
| | fluoro-3- | | | | | |
| | methoxyphenyl | | | | | |
| 63-151 | 4-chloro-2- | Me | Me | Me | H | CO₂H |
| | fluoro-3- | | | | | |
| | methoxyphenyl | | | | | |
| 63-152 | 4-chloro-2- | Me | Me | Me | H | CO₂Me |
| | fluoro-3- | | | | | |
| | methoxyphenyl | | | | | |
| 63-153 | 4-chloro-2- | Me | Me | H | Me | CO₂H |
| | fluoro-3- | | | | | |
| | methoxyphenyl | | | | | |
| 63-154 | 4-chloro-2- | Me | Me | H | Me | CO₂Me |
| | fluoro-3- | | | | | |
| | methoxyphenyl | | | | | |
| 63-155 | 4-chloro-2- | Me | Me | Me | Me | CO₂H |
| | fluoro-3- | | | | | |
| | methoxyphenyl | | | | | |
| 63-156 | 4-chloro-2- | Me | Me | Me | Me | CO₂Me |
| | fluoro-3- | | | | | |
| | methoxyphenyl | | | | | |
| 63-157 | 4-chloro-2- | Me | Me | H | Me | CO₂Et |
| | fluoro-3- | | | | | |
| | methoxyphenyl | | | | | |
| 63-158 | 4-chloro-2- | Me | Me | H | Me | CO₂n-Pr |
| | fluoro-3- | | | | | |
| | methoxyphenyl | | | | | |
| 63-159 | 4-chloro-2- | Me | Me | H | Me | CO₂i-Pr |
| | fluoro-3- | | | | | |
| | methoxyphenyl | | | | | |
| 63-160 | 4-chloro-2- | Me | Me | H | Me | CO₂CH₂CH═CH₂ |
| | fluoro-3- | | | | | |
| | methoxyphenyl | | | | | |
| 63-161 | 4-chloro-2- | Me | Me | H | Me | CO₂CH₂CH₂OMe |
| | fluoro-3- | | | | | |
| | methoxyphenyl | | | | | |
| 63-162 | 4-chloro-2- | Me | Me | H | Me | CO₂CH₂Ph |
| | fluoro-3- | | | | | |
| | methoxyphenyl | | | | | |
| 63-163 | 4-chloro-2- | Me | Me | Me | Me | CO₂Et |
| | fluoro-3- | | | | | |
| | methoxyphenyl | | | | | |
| 63-164 | 4-chloro-2- | Me | Me | Me | Me | CO₂n-Pr |
| | fluoro-3- | | | | | |
| | methoxyphenyl | | | | | |
| 63-165 | 4-chloro-2- | Me | Me | Me | Me | CO₂i-Pr |
| | fluoro-3- | | | | | |
| | methoxyphenyl | | | | | |
| 63-166 | 4-chloro-2- | Me | Me | Me | Me | CO₂CH₂CH═CH₂ |
| | fluoro-3- | | | | | |
| | methoxyphenyl | | | | | |
| 63-167 | 4-chloro-2- | Me | Me | Me | Me | CO₂CH₂CH₂OMe |
| | fluoro-3- | | | | | |
| | methoxyphenyl | | | | | |
| 63-168 | 4-chloro-2- | Me | Me | Me | Me | CO₂CH₂Ph |
| | fluoro-3- | | | | | |
| | methoxyphenyl | | | | | |
| 63-169 | 4-chloro-3- | Me | Me | H | Me | CO₂H |
| | dimethylamino- | | | | | |
| | 2-fluorophenyl | | | | | |
| 63-170 | 4-chloro-3- | Me | Me | H | Me | CO₂Me |
| | dimethylamino- | | | | | |
| | 2-fluorophenyl | | | | | |
| 63-171 | 4-chloro-3- | Me | Me | Me | Me | CO₂H |
| | dimethylamino- | | | | | |
| | 2-fluorophenyl | | | | | |
| 63-172 | 4-chloro-3- | Me | Me | Me | Me | CO₂Me |
| | dimethylamino- | | | | | |
| | 2-fluorophenyl | | | | | |

172 compounds are described, designated compounds 64-1 to 64-172 respectively, of formula (1C) wherein D is N and X is NMe, and the values of A, R⁵, R⁷, R⁸, R⁸′ and Z are as defined in Table 3.

172 compounds are described, designated compounds 65-1 to 65-172 respectively, of formula (1C) wherein D is N and X is O, and the values of A, R⁵, R⁷, R⁸, R⁸′ and Z are as defined in Table 3.

172 compounds are described, designated compounds 66-1 to 66-172 respectively, of formula (1C) wherein D is N and X is S, and the values of A, R⁵, R⁷, R⁸, R⁸′ and Z are as defined in Table 3.

172 compounds are described, designated compounds 67-1 to 67-172 respectively, of formula (1C) wherein D is CH and X is NH, and the values of A, R⁵, R⁷, R⁸, R⁸′ and Z are as defined in Table 3.

172 compounds are described, designated compounds 68-1 to 68-172 respectively, of formula (1C) wherein D is CH and X is NMe, and the values of A, R⁵, R⁷, R⁸, R⁸′ and Z are as defined in Table 3.

172 compounds are described, designated compounds 69-1 to 69-172 respectively, of formula (1C) wherein D is CH and X is O, and the values of A, R⁵, R⁷, R⁸, R⁸′ and Z are as defined in Table 3.

172 compounds are described, designated compounds 70-1 to 70-172 respectively, of formula (1C) wherein D is CH and X is S, and the values of A, R⁵, R⁷, R⁸, R⁸′ and Z are as defined in Table 3.

Table 4 below provides 240 compounds designated compounds 71-1 to 71-240 respectively, of formula (1D) wherein D is N and X is NH.

TABLE 4

| | |
|---|---|
| | (1D) |
| 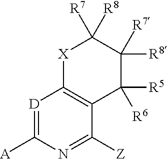 | |

| Compound | Substituent Values | | | | | | | |
|---|---|---|---|---|---|---|---|---|
| Number | A | R⁷ | R⁸ | R^{7′} | R^{8′} | R⁵ | R⁶ | Z |
| 71-1 | cyclopropyl | H | H | H | H | H | H | CO₂H |
| 71-2 | cyclopropyl | H | H | H | H | H | H | CO₂Me |
| 71-3 | cyclopropyl | H | H | H | H | H | Me | CO₂H |
| 71-4 | cyclopropyl | H | H | H | H | H | Me | CO₂Me |
| 71-5 | cyclopropyl | H | H | H | H | Me | Me | CO₂H |
| 71-6 | cyclopropyl | H | H | H | H | Me | Me | CO₂Me |
| 71-7 | cyclopropyl | H | H | H | Me | H | H | CO₂H |
| 71-8 | cyclopropyl | H | H | H | Me | H | H | CO₂Me |
| 71-9 | cyclopropyl | H | H | H | Me | H | Me | CO₂H |
| 71-10 | cyclopropyl | H | H | H | Me | H | Me | CO₂Me |
| 71-11 | cyclopropyl | H | H | H | Me | Me | Me | CO₂H |
| 71-12 | cyclopropyl | H | H | H | Me | Me | Me | CO₂Me |
| 71-13 | cyclopropyl | H | H | Me | Me | H | H | CO₂H |
| 71-14 | cyclopropyl | H | H | Me | Me | H | H | CO₂Me |
| 71-15 | cyclopropyl | H | H | Me | Me | H | Me | CO₂H |
| 71-16 | cyclopropyl | H | H | Me | Me | H | Me | CO₂Me |
| 71-17 | cyclopropyl | H | H | Me | Me | Me | Me | CO₂H |
| 71-18 | cyclopropyl | H | H | Me | Me | Me | Me | CO₂Me |
| 71-19 | cyclopropyl | H | H | Me | Me | ═CH₂ | | CO₂H |
| 71-20 | cyclopropyl | H | H | Me | Me | ═CH₂ | | CO₂Me |
| 71-21 | cyclopropyl | H | Me | H | H | H | H | CO₂H |
| 71-22 | cyclopropyl | H | Me | H | H | H | H | CO₂Me |
| 71-23 | cyclopropyl | H | Me | H | H | H | Me | CO₂H |
| 71-24 | cyclopropyl | H | Me | H | H | H | Me | CO₂Me |
| 71-25 | cyclopropyl | H | Me | H | H | Me | Me | CO₂H |
| 71-26 | cyclopropyl | H | Me | H | H | Me | Me | CO₂Me |
| 71-27 | cyclopropyl | H | Me | H | Me | H | H | CO₂H |
| 71-28 | cyclopropyl | H | Me | H | Me | H | H | CO₂Me |
| 71-29 | cyclopropyl | H | Me | H | Me | H | Me | CO₂H |
| 71-30 | cyclopropyl | H | Me | H | Me | H | Me | CO₂Me |
| 71-31 | cyclopropyl | H | Me | H | Me | Me | Me | CO₂H |
| 71-32 | cyclopropyl | H | Me | H | Me | Me | Me | CO₂Me |
| 71-33 | cyclopropyl | H | Me | Me | Me | H | H | CO₂H |
| 71-34 | cyclopropyl | H | Me | Me | Me | H | H | CO₂Me |
| 71-35 | cyclopropyl | H | Me | Me | Me | H | Me | CO₂H |
| 71-36 | cyclopropyl | H | Me | Me | Me | H | Me | CO₂Me |
| 71-37 | cyclopropyl | H | Me | Me | Me | Me | Me | CO₂H |
| 71-38 | cyclopropyl | H | Me | Me | Me | Me | Me | CO₂Me |
| 71-39 | cyclopropyl | H | Me | Me | Me | ═CH₂ | | CO₂H |
| 71-40 | cyclopropyl | H | Me | Me | Me | ═CH₂ | | CO₂Me |
| 71-41 | cyclopropyl | Me | Me | H | H | H | H | CO₂H |
| 71-42 | cyclopropyl | Me | Me | H | H | H | H | CO₂Me |
| 71-43 | cyclopropyl | Me | Me | H | H | H | Me | CO₂H |
| 71-44 | cyclopropyl | Me | Me | H | H | H | Me | CO₂Me |
| 71-45 | cyclopropyl | Me | Me | H | H | Me | Me | CO₂H |
| 71-46 | cyclopropyl | Me | Me | H | H | Me | Me | CO₂Me |
| 71-47 | cyclopropyl | Me | Me | H | Me | H | H | CO₂H |
| 71-48 | cyclopropyl | Me | Me | H | Me | H | H | CO₂Me |
| 71-49 | cyclopropyl | Me | Me | H | Me | H | Me | CO₂H |
| 71-50 | cyclopropyl | Me | Me | H | Me | H | Me | CO₂Me |
| 71-51 | cyclopropyl | Me | Me | H | Me | Me | Me | CO₂H |
| 71-52 | cyclopropyl | Me | Me | H | Me | Me | Me | CO₂Me |
| 71-53 | cyclopropyl | Me | Me | Me | Me | H | H | CO₂H |
| 71-54 | cyclopropyl | Me | Me | Me | Me | H | H | CO₂Me |
| 71-55 | cyclopropyl | Me | Me | Me | Me | H | Me | CO₂H |
| 71-56 | cyclopropyl | Me | Me | Me | Me | H | Me | CO₂Me |
| 71-57 | cyclopropyl | Me | Me | Me | Me | Me | Me | CO₂H |
| 71-58 | cyclopropyl | Me | Me | Me | Me | Me | Me | CO₂Me |
| 71-59 | cyclopropyl | Me | Me | Me | Me | ═CH₂ | | CO₂H |
| 71-60 | cyclopropyl | Me | Me | Me | Me | ═CH₂ | | CO₂Me |
| 71-61 | cyclopropyl | ═O | | H | H | H | H | CO₂H |
| 71-62 | cyclopropyl | ═O | | H | H | H | H | CO₂Me |
| 71-63 | cyclopropyl | ═O | | H | H | H | Me | CO₂H |
| 71-64 | cyclopropyl | ═O | | H | H | H | Me | CO₂Me |
| 71-65 | cyclopropyl | ═O | | H | H | Me | Me | CO₂H |
| 71-66 | cyclopropyl | ═O | | H | H | Me | Me | CO₂Me |
| 71-67 | cyclopropyl | ═O | | H | Me | H | H | CO₂H |
| 71-68 | cyclopropyl | ═O | | H | Me | H | H | CO₂Me |
| 71-69 | cyclopropyl | ═O | | H | Me | H | Me | CO₂H |
| 71-70 | cyclopropyl | ═O | | H | Me | H | Me | CO₂Me |
| 71-71 | cyclopropyl | ═O | | H | Me | Me | Me | CO₂H |
| 71-72 | cyclopropyl | ═O | | H | Me | Me | Me | CO₂Me |
| 71-73 | cyclopropyl | ═O | | Me | Me | H | H | CO₂H |
| 71-74 | cyclopropyl | ═O | | Me | Me | H | H | CO₂Me |
| 71-75 | cyclopropyl | ═O | | Me | Me | H | Me | CO₂H |
| 71-76 | cyclopropyl | ═O | | Me | Me | H | Me | CO₂Me |
| 71-77 | cyclopropyl | ═O | | Me | Me | Me | Me | CO₂H |
| 71-78 | cyclopropyl | ═O | | Me | Me | Me | Me | CO₂Me |
| 71-79 | cyclopropyl | ═O | | Me | Me | ═CH₂ | | CO₂H |
| 71-80 | cyclopropyl | ═O | | Me | Me | ═CH₂ | | CO₂Me |
| 71-81 | 4-chloro-3- | H | H | H | H | H | H | CO₂H |
| | fluorophenyl | | | | | | | |
| 71-82 | 4-chloro-3- | H | H | H | H | H | H | CO₂Me |
| | fluorophenyl | | | | | | | |
| 71-83 | 4-chloro-3- | H | H | H | H | H | Me | CO₂H |
| | fluorophenyl | | | | | | | |
| 71-84 | 4-chloro-3- | H | H | H | H | H | Me | CO₂Me |
| | fluorophenyl | | | | | | | |
| 71-85 | 4-chloro-3- | H | H | H | H | Me | Me | CO₂H |
| | fluorophenyl | | | | | | | |
| 71-86 | 4-chloro-3- | H | H | H | H | Me | Me | CO₂Me |
| | fluorophenyl | | | | | | | |
| 71-87 | 4-chloro-3- | H | H | H | Me | H | H | CO₂H |
| | fluorophenyl | | | | | | | |
| 71-88 | 4-chloro-3- | H | H | H | Me | H | H | CO₂Me |
| | fluorophenyl | | | | | | | |
| 71-89 | 4-chloro-3- | H | H | H | Me | H | Me | CO₂H |
| | fluorophenyl | | | | | | | |
| 71-90 | 4-chloro-3- | H | H | H | Me | H | Me | CO₂Me |
| | fluorophenyl | | | | | | | |
| 71-91 | 4-chloro-3- | H | H | H | Me | Me | Me | CO₂H |
| | fluorophenyl | | | | | | | |
| 71-92 | 4-chloro-3- | H | H | H | Me | Me | Me | CO₂Me |
| | fluorophenyl | | | | | | | |
| 71-93 | 4-chloro-3- | H | H | Me | Me | H | H | CO₂H |
| | fluorophenyl | | | | | | | |
| 71-94 | 4-chloro-3- | H | H | Me | Me | H | H | CO₂Me |
| | fluorophenyl | | | | | | | |
| 71-95 | 4-chloro-3- | H | H | Me | Me | H | Me | CO₂H |
| | fluorophenyl | | | | | | | |
| 71-96 | 4-chloro-3- | H | H | Me | Me | H | Me | CO₂Me |
| | fluorophenyl | | | | | | | |
| 71-97 | 4-chloro-3- | H | H | Me | Me | Me | Me | CO₂H |
| | fluorophenyl | | | | | | | |
| 71-98 | 4-chloro-3- | H | H | Me | Me | Me | Me | CO₂Me |
| | fluorophenyl | | | | | | | |
| 71-99 | 4-chloro-3- | H | H | Me | Me | ═CH₂ | | CO₂H |
| | fluorophenyl | | | | | | | |
| 71-100 | 4-chloro-3- | H | H | Me | Me | ═CH₂ | | CO₂Me |
| | fluorophenyl | | | | | | | |
| 71-101 | 4-chloro-3- | H | Me | H | H | H | H | CO₂H |
| | fluorophenyl | | | | | | | |
| 71-102 | 4-chloro-3- | H | Me | H | H | H | H | CO₂Me |
| | fluorophenyl | | | | | | | |
| 71-103 | 4-chloro-3- | H | Me | H | H | H | Me | CO₂H |
| | fluorophenyl | | | | | | | |
| 71-104 | 4-chloro-3- | H | Me | H | H | H | Me | CO₂Me |
| | fluorophenyl | | | | | | | |
| 71-105 | 4-chloro-3- | H | Me | H | H | Me | Me | CO₂H |
| | fluorophenyl | | | | | | | |
| 71-106 | 4-chloro-3- | H | Me | H | H | Me | Me | CO₂Me |
| | fluorophenyl | | | | | | | |
| 71-107 | 4-chloro-3- | H | Me | H | Me | H | H | CO₂H |
| | fluorophenyl | | | | | | | |
| 71-108 | 4-chloro-3- | H | Me | H | Me | H | H | CO₂Me |
| | fluorophenyl | | | | | | | |
| 71-109 | 4-chloro-3- | H | Me | H | Me | H | Me | CO₂H |
| | fluorophenyl | | | | | | | |
| 71-110 | 4-chloro-3- | H | Me | H | Me | H | Me | CO₂Me |
| | fluorophenyl | | | | | | | |
| 71-111 | 4-chloro-3- | H | Me | H | Me | Me | Me | CO₂H |
| | fluorophenyl | | | | | | | |
| 71-112 | 4-chloro-3- | H | Me | H | Me | Me | Me | CO₂Me |
| | fluorophenyl | | | | | | | |
| 71-113 | 4-chloro-3- | H | Me | Me | Me | H | H | CO₂H |
| | fluorophenyl | | | | | | | |
| 71-114 | 4-chloro-3- | H | Me | Me | Me | H | H | CO₂Me |
| | fluorophenyl | | | | | | | |
| 71-115 | 4-chloro-3- | H | Me | Me | Me | H | Me | CO₂H |
| | fluorophenyl | | | | | | | |
| 71-116 | 4-chloro-3- | H | Me | Me | Me | H | Me | CO₂Me |
| | fluorophenyl | | | | | | | |
| 71-117 | 4-chloro-3- | H | Me | Me | Me | Me | Me | CO₂H |
| | fluorophenyl | | | | | | | |
| 71-118 | 4-chloro-3- | H | Me | Me | Me | Me | Me | CO₂Me |
| | fluorophenyl | | | | | | | |
| 71-119 | 4-chloro-3- | H | Me | Me | Me | ═CH₂ | | CO₂H |
| | fluorophenyl | | | | | | | |
| 71-120 | 4-chloro-3- | H | Me | Me | Me | ═CH₂ | | CO₂Me |
| | fluorophenyl | | | | | | | |
| 71-121 | 4-chloro-3- | Me | Me | H | H | H | H | CO₂H |
| | fluorophenyl | | | | | | | |
| 71-122 | 4-chloro-3- | Me | Me | H | H | H | H | CO₂Me |
| | fluorophenyl | | | | | | | |
| 71-123 | 4-chloro-3- | Me | Me | H | H | H | Me | CO₂H |
| | fluorophenyl | | | | | | | |
| 71-124 | 4-chloro-3- | Me | Me | H | H | H | Me | CO₂Me |
| | fluorophenyl | | | | | | | |
| 71-125 | 4-chloro-3- | Me | Me | H | H | Me | Me | CO₂H |
| | fluorophenyl | | | | | | | |
| 71-126 | 4-chloro-3- | Me | Me | H | H | Me | Me | CO₂Me |
| | fluorophenyl | | | | | | | |
| 71-127 | 4-chloro-3- | Me | Me | H | Me | H | H | CO₂H |
| | fluorophenyl | | | | | | | |
| 71-128 | 4-chloro-3- | Me | Me | H | Me | H | H | CO₂Me |
| | fluorophenyl | | | | | | | |
| 71-129 | 4-chloro-3- | Me | Me | H | Me | H | Me | CO₂H |
| | fluorophenyl | | | | | | | |
| 71-130 | 4-chloro-3- | Me | Me | H | Me | H | Me | CO₂Me |
| | fluorophenyl | | | | | | | |
| 71-131 | 4-chloro-3- | Me | Me | H | Me | Me | Me | CO₂H |
| | fluorophenyl | | | | | | | |
| 71-132 | 4-chloro-3- | Me | Me | H | Me | Me | Me | CO₂Me |
| | fluorophenyl | | | | | | | |
| 71-133 | 4-chloro-3- | Me | Me | Me | Me | H | H | CO₂H |
| | fluorophenyl | | | | | | | |
| 71-134 | 4-chloro-3- | Me | Me | Me | Me | H | H | CO₂Me |
| | fluorophenyl | | | | | | | |
| 71-135 | 4-chloro-3- | Me | Me | Me | Me | H | Me | CO₂H |
| | fluorophenyl | | | | | | | |
| 71-136 | 4-chloro-3- | Me | Me | Me | Me | H | Me | CO₂Me |
| | fluorophenyl | | | | | | | |
| 71-137 | 4-chloro-3- | Me | Me | Me | Me | Me | Me | CO₂H |
| | fluorophenyl | | | | | | | |
| 71-138 | 4-chloro-3- | Me | Me | Me | Me | Me | Me | CO₂Me |
| | fluorophenyl | | | | | | | |
| 71-139 | 4-chloro-3- | Me | Me | Me | Me | ═CH₂ | | CO₂H |
| | fluorophenyl | | | | | | | |
| 71-140 | 4-chloro-3- | Me | Me | Me | Me | ═CH₂ | | CO₂Me |
| | fluorophenyl | | | | | | | |
| 71-141 | 4-chloro-3- | ═O | | H | H | H | H | CO₂H |
| | fluorophenyl | | | | | | | |
| 71-142 | 4-chloro-3- | ═O | | H | H | H | H | CO₂Me |
| | fluorophenyl | | | | | | | |
| 71-143 | 4-chloro-3- | ═O | | H | H | H | Me | CO₂H |
| | fluorophenyl | | | | | | | |
| 71-144 | 4-chloro-3- | ═O | | H | H | H | Me | CO₂Me |
| | fluorophenyl | | | | | | | |
| 71-145 | 4-chloro-3- | ═O | | H | H | Me | Me | CO₂H |
| | fluorophenyl | | | | | | | |
| 71-146 | 4-chloro-3- | ═O | | H | H | Me | Me | CO₂Me |
| | fluorophenyl | | | | | | | |
| 71-147 | 4-chloro-3- | ═O | | H | Me | H | H | CO₂H |
| | fluorophenyl | | | | | | | |
| 71-148 | 4-chloro-3- | ═O | | H | Me | H | H | CO₂Me |
| | fluorophenyl | | | | | | | |
| 71-149 | 4-chloro-3- | ═O | | H | Me | H | Me | CO₂H |
| | fluorophenyl | | | | | | | |
| 71-150 | 4-chloro-3- | ═O | | H | Me | H | Me | CO₂Me |
| | fluorophenyl | | | | | | | |
| 71-151 | 4-chloro-3- | ═O | | H | Me | Me | Me | CO₂H |
| | fluorophenyl | | | | | | | |
| 71-152 | 4-chloro-3- | ═O | | H | Me | Me | Me | CO₂Me |
| | fluorophenyl | | | | | | | |
| 71-153 | 4-chloro-3- | ═O | | Me | Me | H | H | CO₂H |
| | fluorophenyl | | | | | | | |
| 71-154 | 4-chloro-3- | ═O | | Me | Me | H | H | CO₂Me |
| | fluorophenyl | | | | | | | |
| 71-155 | 4-chloro-3- | ═O | | Me | Me | H | Me | CO₂H |
| | fluorophenyl | | | | | | | |
| 71-156 | 4-chloro-3- | ═O | | Me | Me | H | Me | CO₂Me |
| | fluorophenyl | | | | | | | |
| 71-157 | 4-chloro-3- | ═O | | Me | Me | Me | Me | CO₂H |
| | fluorophenyl | | | | | | | |
| 71-158 | 4-chloro-3- | ═O | | Me | Me | Me | Me | CO₂Me |
| | fluorophenyl | | | | | | | |
| 71-159 | 4-chloro-3- | ═O | | Me | Me | ═CH₂ | | CO₂H |
| | fluorophenyl | | | | | | | |
| 71-160 | 4-chloro-3- | ═O | | Me | Me | ═CH₂ | | CO₂Me |
| | fluorophenyl | | | | | | | |
| 71-161 | 4-chloro-2-fluoro-3- | H | H | H | H | H | H | CO₂H |
| | methoxyphenyl | | | | | | | |
| 71-162 | 4-chloro-2-fluoro-3- | H | H | H | H | H | H | CO₂Me |
| | methoxyphenyl | | | | | | | |
| 71-163 | 4-chloro-2-fluoro-3- | H | H | H | H | H | Me | CO₂H |
| | methoxyphenyl | | | | | | | |
| 71-164 | 4-chloro-2-fluoro-3- | H | H | H | H | H | Me | CO₂Me |
| | methoxyphenyl | | | | | | | |
| 71-165 | 4-chloro-2-fluoro-3- | H | H | H | H | Me | Me | CO₂H |
| | methoxyphenyl | | | | | | | |
| 71-166 | 4-chloro-2-fluoro-3- | H | H | H | H | Me | Me | CO₂Me |
| | methoxyphenyl | | | | | | | |
| 71-167 | 4-chloro-2-fluoro-3- | H | H | H | Me | H | H | CO₂H |
| | methoxyphenyl | | | | | | | |
| 71-168 | 4-chloro-2-fluoro-3- | H | H | H | Me | H | H | CO₂Me |
| | methoxyphenyl | | | | | | | |
| 71-169 | 4-chloro-2-fluoro-3- | H | H | H | Me | H | Me | CO₂H |
| | methoxyphenyl | | | | | | | |
| 71-170 | 4-chloro-2-fluoro-3- | H | H | H | Me | H | Me | CO₂Me |
| | methoxyphenyl | | | | | | | |
| 71-171 | 4-chloro-2-fluoro-3- | H | H | H | Me | Me | Me | CO₂H |
| | methoxyphenyl | | | | | | | |
| 71-172 | 4-chloro-2-fluoro-3- | H | H | H | Me | Me | Me | CO₂Me |
| | methoxyphenyl | | | | | | | |
| 71-173 | 4-chloro-2-fluoro-3- | H | H | Me | Me | H | H | CO₂H |
| | methoxyphenyl | | | | | | | |
| 71-174 | 4-chloro-2-fluoro-3- | H | H | Me | Me | H | H | CO₂Me |
| | methoxyphenyl | | | | | | | |
| 71-175 | 4-chloro-2-fluoro-3- | H | H | Me | Me | H | Me | CO₂H |
| | methoxyphenyl | | | | | | | |
| 71-176 | 4-chloro-2-fluoro-3- | H | H | Me | Me | H | Me | CO₂Me |
| | methoxyphenyl | | | | | | | |
| 71-177 | 4-chloro-2-fluoro-3- | H | H | Me | Me | Me | Me | CO₂H |
| | methoxyphenyl | | | | | | | |
| 71-178 | 4-chloro-2-fluoro-3- | H | H | Me | Me | Me | Me | CO₂Me |
| | methoxyphenyl | | | | | | | |
| 71-179 | 4-chloro-2-fluoro-3- | H | H | Me | Me | ═CH₂ | | CO₂H |
| | methoxyphenyl | | | | | | | |
| 71-180 | 4-chloro-2-fluoro-3- | H | H | Me | Me | ═CH₂ | | CO₂Me |
| | methoxyphenyl | | | | | | | |
| 71-181 | 4-chloro-2-fluoro-3- | H | Me | H | H | H | H | CO₂H |
| | methoxyphenyl | | | | | | | |
| 71-182 | 4-chloro-2-fluoro-3- | H | Me | H | H | H | H | CO₂Me |
| | methoxyphenyl | | | | | | | |
| 71-183 | 4-chloro-2-fluoro-3- | H | Me | H | H | H | Me | CO₂H |
| | methoxyphenyl | | | | | | | |
| 71-184 | 4-chloro-2-fluoro-3- | H | Me | H | H | H | Me | CO₂Me |
| | methoxyphenyl | | | | | | | |
| 71-185 | 4-chloro-2-fluoro-3- | H | Me | H | H | Me | Me | CO₂H |
| | methoxyphenyl | | | | | | | |
| 71-186 | 4-chloro-2-fluoro-3- | H | Me | H | H | Me | Me | CO₂Me |
| | methoxyphenyl | | | | | | | |
| 71-187 | 4-chloro-2-fluoro-3- | H | Me | H | Me | H | H | CO₂H |
| | methoxyphenyl | | | | | | | |
| 71-188 | 4-chloro-2-fluoro-3- | H | Me | H | Me | H | H | CO₂Me |
| | methoxyphenyl | | | | | | | |
| 71-189 | 4-chloro-2-fluoro-3- | H | Me | H | Me | H | Me | CO₂H |
| | methoxyphenyl | | | | | | | |
| 71-190 | 4-chloro-2-fluoro-3- | H | Me | H | Me | H | Me | CO₂Me |
| | methoxyphenyl | | | | | | | |
| 71-191 | 4-chloro-2-fluoro-3- | H | Me | H | Me | Me | Me | CO₂H |
| | methoxyphenyl | | | | | | | |
| 71-192 | 4-chloro-2-fluoro-3- | H | Me | H | Me | Me | Me | CO₂Me |
| | methoxyphenyl | | | | | | | |
| 71-193 | 4-chloro-2-fluoro-3- | H | Me | Me | Me | H | H | CO₂H |
| | methoxyphenyl | | | | | | | |
| 71-194 | 4-chloro-2-fluoro-3- | H | Me | Me | Me | H | H | CO₂Me |
| | methoxyphenyl | | | | | | | |
| 71-195 | 4-chloro-2-fluoro-3- | H | Me | Me | Me | H | Me | CO₂H |
| | methoxyphenyl | | | | | | | |
| 71-196 | 4-chloro-2-fluoro-3- | H | Me | Me | Me | H | Me | CO₂Me |
| | methoxyphenyl | | | | | | | |
| 71-197 | 4-chloro-2-fluoro-3- | H | Me | Me | Me | Me | Me | CO₂H |
| | methoxyphenyl | | | | | | | |
| 71-198 | 4-chloro-2-fluoro-3- | H | Me | Me | Me | Me | Me | CO₂Me |
| | methoxyphenyl | | | | | | | |
| 71-199 | 4-chloro-2-fluoro-3- | H | Me | Me | Me | ═CH₂ | | CO₂H |
| | methoxyphenyl | | | | | | | |
| 71-200 | 4-chloro-2-fluoro-3- | H | Me | Me | Me | ═CH₂ | | CO₂Me |
| | methoxyphenyl | | | | | | | |
| 71-201 | 4-chloro-2-fluoro-3- | Me | Me | H | H | H | H | CO₂H |
| | methoxyphenyl | | | | | | | |
| 71-202 | 4-chloro-2-fluoro-3- | Me | Me | H | H | H | H | CO₂Me |
| | methoxyphenyl | | | | | | | |
| 71-203 | 4-chloro-2-fluoro-3- | Me | Me | H | H | H | Me | CO₂H |
| | methoxyphenyl | | | | | | | |
| 71-204 | 4-chloro-2-fluoro-3- | Me | Me | H | H | H | Me | CO₂Me |
| | methoxyphenyl | | | | | | | |
| 71-205 | 4-chloro-2-fluoro-3- | Me | Me | H | H | Me | Me | CO₂H |
| | methoxyphenyl | | | | | | | |
| 71-206 | 4-chloro-2-fluoro-3- | Me | Me | H | H | Me | Me | CO₂Me |
| | methoxyphenyl | | | | | | | |
| 71-207 | 4-chloro-2-fluoro-3- | Me | Me | H | Me | H | H | CO₂H |
| | methoxyphenyl | | | | | | | |
| 71-208 | 4-chloro-2-fluoro-3- | Me | Me | H | Me | H | H | CO₂Me |
| | methoxyphenyl | | | | | | | |
| 71-209 | 4-chloro-2-fluoro-3- | Me | Me | H | Me | H | Me | CO₂H |
| | methoxyphenyl | | | | | | | |
| 71-210 | 4-chloro-2-fluoro-3- | Me | Me | H | Me | H | Me | CO₂Me |
| | methoxyphenyl | | | | | | | |
| 71-211 | 4-chloro-2-fluoro-3- | Me | Me | H | Me | Me | Me | CO₂H |
| | methoxyphenyl | | | | | | | |
| 71-212 | 4-chloro-2-fluoro-3- | Me | Me | H | Me | Me | Me | CO₂Me |
| | methoxyphenyl | | | | | | | |
| 71-213 | 4-chloro-2-fluoro-3- | Me | Me | Me | Me | H | H | CO₂H |
| | methoxyphenyl | | | | | | | |
| 71-214 | 4-chloro-2-fluoro-3- | Me | Me | Me | Me | H | H | CO₂Me |
| | methoxyphenyl | | | | | | | |
| 71-215 | 4-chloro-2-fluoro-3- | Me | Me | Me | Me | H | Me | CO₂H |
| | methoxyphenyl | | | | | | | |
| 71-216 | 4-chloro-2-fluoro-3- | Me | Me | Me | Me | H | Me | CO₂Me |
| | methoxyphenyl | | | | | | | |
| 71-217 | 4-chloro-2-fluoro-3- | Me | Me | Me | Me | Me | Me | CO₂H |
| | methoxyphenyl | | | | | | | |
| 71-218 | 4-chloro-2-fluoro-3- | Me | Me | Me | Me | Me | Me | CO₂Me |
| | methoxyphenyl | | | | | | | |

| | | | | | | | |
|---|---|---|---|---|---|---|---|
| 71-219 | 4-chloro-2-fluoro-3- | Me | Me | Me | Me | ═CH₂ | CO₂H |
| | methoxyphenyl | | | | | | |
| 71-220 | 4-chloro-2-fluoro-3- | Me | Me | Me | Me | ═CH₂ | CO₂Me |
| | methoxyphenyl | | | | | | |

| | | | | | | | |
|---|---|---|---|---|---|---|---|
| 71-221 | 4-chloro-2-fluoro-3- | ═O | H | H | H | H | CO₂H |
| | methoxyphenyl | | | | | | |
| 71-222 | 4-chloro-2-fluoro-3- | ═O | H | H | H | H | CO₂Me |
| | methoxyphenyl | | | | | | |
| 71-223 | 4-chloro-2-fluoro-3- | ═O | H | H | H | Me | CO₂H |
| | methoxyphenyl | | | | | | |
| 71-224 | 4-chloro-2-fluoro-3- | ═O | H | H | H | Me | CO₂Me |
| | methoxyphenyl | | | | | | |
| 71-225 | 4-chloro-2-fluoro-3- | ═O | H | H | Me | Me | CO₂H |
| | methoxyphenyl | | | | | | |
| 71-226 | 4-chloro-2-fluoro-3- | ═O | H | H | Me | Me | CO₂Me |
| | methoxyphenyl | | | | | | |
| 71-227 | 4-chloro-2-fluoro-3- | ═O | H | Me | H | H | CO₂H |
| | methoxyphenyl | | | | | | |
| 71-228 | 4-chloro-2-fluoro-3- | ═O | H | Me | H | H | CO₂Me |
| | methoxyphenyl | | | | | | |
| 71-229 | 4-chloro-2-fluoro-3- | ═O | H | Me | H | Me | CO₂H |
| | methoxyphenyl | | | | | | |
| 71-230 | 4-chloro-2-fluoro-3- | ═O | H | Me | H | Me | CO₂Me |
| | methoxyphenyl | | | | | | |
| 71-231 | 4-chloro-2-fluoro-3- | ═O | H | Me | Me | Me | CO₂H |
| | methoxyphenyl | | | | | | |
| 71-232 | 4-chloro-2-fluoro-3- | ═O | H | Me | Me | Me | CO₂Me |
| | methoxyphenyl | | | | | | |
| 71-233 | 4-chloro-2-fluoro-3- | ═O | Me | Me | H | H | CO₂H |
| | methoxyphenyl | | | | | | |
| 71-234 | 4-chloro-2-fluoro-3- | ═O | Me | Me | H | H | CO₂Me |
| | methoxyphenyl | | | | | | |
| 71-235 | 4-chloro-2-fluoro-3- | ═O | Me | Me | H | Me | CO₂H |
| | methoxyphenyl | | | | | | |
| 71-236 | 4-chloro-2-fluoro-3- | ═O | Me | Me | H | Me | CO₂Me |
| | methoxyphenyl | | | | | | |
| 71-237 | 4-chloro-2-fluoro-3- | ═O | Me | Me | Me | Me | CO₂H |
| | methoxyphenyl | | | | | | |
| 71-238 | 4-chloro-2-fluoro-3- | ═O | Me | Me | Me | Me | CO₂Me |
| | methoxyphenyl | | | | | | |

| | | | | | | |
|---|---|---|---|---|---|---|
| 71-239 | 4-chloro-2-fluoro-3- | ═O | Me | Me | ═CH₂ | CO₂H |
| | methoxyphenyl | | | | | |
| 71-240 | 4-chloro-2-fluoro-3- | ═O | Me | Me | ═CH₂ | CO₂Me |
| | methoxyphenyl | | | | | |

240 compounds are described, designated compounds 72-1 to 72-240 respectively, of formula (1D) wherein D is N and X is NMe, and the values of A, R⁵, R⁶, R⁷, R⁷′, R⁸, R⁸′ and Z are as defined in Table 4.

240 compounds are described, designated compounds 73-1 to 73-240 respectively, of formula (1D) wherein D is N and X is O, and the values of A, R⁵, R⁶, R⁷, R⁷′, R⁸, R⁸′ and Z are as defined in Table 4.

240 compounds are described, designated compounds 74-1 to 74-240 respectively, of formula (1D) wherein D is N and X is S, and the values of A, R⁵, R⁶, R⁷, R⁷′, R⁸, R⁸′ and Z are as defined in Table 4.

240 compounds are described, designated compounds 75-1 to 75-240 respectively, of formula (1D) wherein D is CH and X is NH, and the values of A, R⁵, R⁶, R⁷, R⁷′, R⁸, R⁸′ and Z are as defined in Table 4.

240 compounds are described, designated compounds 76-1 to 76-240 respectively, of formula (1D) wherein D is CH and X is NMe, and the values of A, R⁵, R⁶, R⁷, R⁷′, R⁸, R⁸′ and Z are as defined in Table 4.

240 compounds are described, designated compounds 77-1 to 77-240 respectively, of formula (1D) wherein D is CH and X is O, and the values of A, R⁵, R⁶, R⁷, R⁷′, R⁸, R⁸′ and Z are as defined in Table 4.

240 compounds are described, designated compounds 78-1 to 78-240 respectively, of formula (1D) wherein D is CH and X is S, and the values of A, R⁵, R⁶, R⁷, R⁷′, R⁸, R⁸′ and Z are as defined in Table 4.

Table 5 below provides 84 compounds designated compounds 79-1 to 79-84 respectively, of formula (1E) wherein D is N and X is NH.

TABLE 5
(1E)

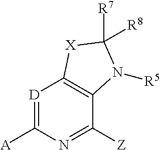

| Compound | Substituent Values | | | | |
|---|---|---|---|---|---|
| Number | A | R⁷ | R⁸ | R⁵ | Z |
| 79-1 | cyclopropyl | H | H | H | CO₂H |
| 79-2 | cyclopropyl | H | H | H | CO₂Me |
| 79-3 | cyclopropyl | H | H | Me | CO₂H |
| 79-4 | cyclopropyl | H | H | Me | CO₂Me |
| 79-5 | cyclopropyl | Me | H | H | CO₂H |
| 79-6 | cyclopropyl | Me | H | H | CO₂Me |
| 79-7 | cyclopropyl | Me | H | Me | CO₂H |
| 79-8 | cyclopropyl | Me | H | Me | CO₂Me |
| 79-9 | cyclopropyl | ═O | | H | CO₂H |
| 79-10 | cyclopropyl | ═O | | H | CO₂Me |
| 79-11 | cyclopropyl | ═O | | Me | CO₂H |
| 79-12 | cyclopropyl | ═O | | Me | CO₂Me |
| 79-13 | 4-chlorophenyl | H | H | H | CO₂H |
| 79-14 | 4-chlorophenyl | H | H | H | CO₂Me |
| 79-15 | 4-chlorophenyl | H | H | Me | CO₂H |
| 79-16 | 4-chlorophenyl | H | H | Me | CO₂Me |
| 79-17 | 4-chlorophenyl | Me | H | H | CO₂H |
| 79-18 | 4-chlorophenyl | Me | H | H | CO₂Me |
| 79-19 | 4-chlorophenyl | Me | H | Me | CO₂H |
| 79-20 | 4-chlorophenyl | Me | H | Me | CO₂Me |
| 79-21 | 4-chlorophenyl | ═O | | H | CO₂H |
| 79-22 | 4-chlorophenyl | ═O | | H | CO₂Me |
| 79-23 | 4-chlorophenyl | ═O | | Me | CO₂H |
| 79-24 | 4-chlorophenyl | ═O | | Me | CO₂Me |
| 79-25 | 4-chloro-3- | H | H | H | CO₂H |
| | fluorophenyl | | | | |
| 79-26 | 4-chloro-3- | H | H | H | CO₂Me |
| | fluorophenyl | | | | |
| 79-27 | 4-chloro-3- | H | H | Me | CO₂H |
| | fluorophenyl | | | | |
| 79-28 | 4-chloro-3- | H | H | Me | CO₂Me |
| | fluorophenyl | | | | |
| 79-29 | 4-chloro-3- | Me | H | H | CO₂H |
| | fluorophenyl | | | | |
| 79-30 | 4-chloro-3- | Me | H | H | CO₂Me |
| | fluorophenyl | | | | |
| 79-31 | 4-chloro-3- | Me | H | Me | CO₂H |
| | fluorophenyl | | | | |
| 79-32 | 4-chloro-3- | Me | H | Me | CO₂Me |
| | fluorophenyl | | | | |
| 79-33 | 4-chloro-3- | ═O | | H | CO₂H |
| | fluorophenyl | | | | |
| 79-34 | 4-chloro-3- | ═O | | H | CO₂Me |
| | fluorophenyl | | | | |
| 79-35 | 4-chloro-3- | ═O | | Me | CO₂H |
| | fluorophenyl | | | | |
| 79-36 | 4-chloro-3- | ═O | | Me | CO₂Me |
| | fluorophenyl | | | | |
| 79-37 | 4-chloro-2-fluoro-3- | H | H | H | CO₂H |
| | methoxyphenyl | | | | |
| 79-38 | 4-chloro-2-fluoro-3- | H | H | H | CO₂Me |
| | methoxyphenyl | | | | |
| 79-39 | 4-chloro-2-fluoro-3- | H | H | Me | CO₂H |
| | methoxyphenyl | | | | |
| 79-40 | 4-chloro-2-fluoro-3- | H | H | Me | CO₂Me |
| | methoxyphenyl | | | | |
| 79-41 | 4-chloro-2-fluoro-3- | Me | H | H | CO₂H |
| | methoxyphenyl | | | | |
| 79-42 | 4-chloro-2-fluoro-3- | Me | H | H | CO₂Me |
| | methoxyphenyl | | | | |
| 79-43 | 4-chloro-2-fluoro-3- | Me | H | Me | CO₂H |
| | methoxyphenyl | | | | |
| 79-44 | 4-chloro-2-fluoro-3- | Me | H | Me | CO₂Me |
| | methoxyphenyl | | | | |
| 79-45 | 4-chloro-2-fluoro-3- | Me | H | CH₂Ph | CO₂H |
| | methoxyphenyl | | | | |
| 79-46 | 4-chloro-2-fluoro-3- | Me | H | CH₂Ph | CO₂Me |
| | methoxyphenyl | | | | |
| 79-47 | 4-chloro-2-fluoro-3- | Me | H | CH₂(2,4- | CO₂H |
| | methoxyphenyl | | | dimethoxyphenyl) | |
| 79-48 | 4-chloro-2-fluoro-3- | Me | H | CH₂(2,4- | CO₂Me |
| | methoxyphenyl | | | dimethoxyphenyl) | |
| 79-49 | 4-chloro-2-fluoro-3- | ═O | | H | CO₂H |
| | methoxyphenyl | | | | |
| 79-50 | 4-chloro-2-fluoro-3- | ═O | | H | CO₂Me |
| | methoxyphenyl | | | | |
| 79-51 | 4-chloro-2-fluoro-3- | ═O | | Me | CO₂H |
| | methoxyphenyl | | | | |
| 79-52 | 4-chloro-2-fluoro-3- | ═O | | Me | CO₂Me |
| | methoxyphenyl | | | | |
| 79-53 | 4-chloro-3- | H | H | H | CO₂H |
| | dimethylamino-2- | | | | |
| | fluorophenyl | | | | |
| 79-54 | 4-chloro-3- | H | H | H | CO₂Me |
| | dimethylamino-2- | | | | |
| | fluorophenyl | | | | |
| 79-55 | 4-chloro-3- | H | H | Me | CO₂H |
| | dimethylamino-2- | | | | |
| | fluorophenyl | | | | |
| 79-56 | 4-chloro-3- | H | H | Me | CO₂Me |
| | dimethylamino-2- | | | | |
| | fluorophenyl | | | | |
| 79-57 | 4-chloro-3- | Me | H | H | CO₂H |
| | dimethylamino-2- | | | | |
| | fluorophenyl | | | | |
| 79-58 | 4-chloro-3- | Me | H | H | CO₂Me |
| | dimethylamino-2- | | | | |
| | fluorophenyl | | | | |
| 79-59 | 4-chloro-3- | Me | H | Me | CO₂H |
| | dimethylamino-2- | | | | |
| | fluorophenyl | | | | |
| 79-60 | 4-chloro-3- | Me | H | Me | CO₂Me |
| | dimethylamino-2- | | | | |
| | fluorophenyl | | | | |
| 79-61 | 4-chloro-3- | ═O | | H | CO₂H |
| | dimethylamino-2- | | | | |
| | fluorophenyl | | | | |
| 79-62 | 4-chloro-3- | ═O | | H | CO₂Me |
| | dimethylamino-2- | | | | |
| | fluorophenyl | | | | |
| 79-63 | 4-chloro-3- | ═O | | Me | CO₂H |
| | dimethylamino-2- | | | | |
| | fluorophenyl | | | | |
| 79-64 | 4-chloro-3- | ═O | | Me | CO₂Me |
| | dimethylamino-2- | | | | |
| | fluorophenyl | | | | |
| 79-65 | cyclopropyl | Me | Me | H | CO₂H |
| 79-66 | cyclopropyl | Me | Me | H | CO₂Me |
| 79-67 | cyclopropyl | Me | Me | Me | CO₂H |
| 79-68 | cyclopropyl | Me | Me | Me | CO₂Me |
| 79-69 | 4-chlorophenyl | Me | Me | H | CO₂H |
| 79-70 | 4-chlorophenyl | Me | Me | H | CO₂Me |
| 79-71 | 4-chlorophenyl | Me | Me | Me | CO₂H |
| 79-72 | 4-chlorophenyl | Me | Me | Me | CO₂Me |
| 79-73 | 4-chloro-3- | Me | Me | H | CO₂H |
| | fluorophenyl | | | | |
| 79-74 | 4-chloro-3- | Me | Me | H | CO₂Me |
| | fluorophenyl | | | | |
| 79-75 | 4-chloro-3- | Me | Me | Me | CO₂H |
| | fluorophenyl | | | | |
| 79-76 | 4-chloro-3- | Me | Me | Me | CO₂Me |
| | fluorophenyl | | | | |
| 79-77 | 4-chloro-2-fluoro-3- | Me | Me | H | CO₂H |
| | methoxyphenyl | | | | |
| 79-78 | 4-chloro-2-fluoro-3- | Me | Me | H | CO₂Me |
| | methoxyphenyl | | | | |
| 79-79 | 4-chloro-2-fluoro-3- | Me | Me | Me | CO₂H |
| | methoxyphenyl | | | | |
| 79-80 | 4-chloro-2-fluoro-3- | Me | Me | Me | CO₂Me |
| | methoxyphenyl | | | | |
| 79-81 | 4-chloro-3- | Me | Me | H | CO₂H |
| | dimethylamino-2- | | | | |
| | fluorophenyl | | | | |
| 79-82 | 4-chloro-3- | Me | Me | H | CO₂Me |
| | dimethylamino-2- | | | | |
| | fluorophenyl | | | | |
| 79-83 | 4-chloro-3- | Me | Me | Me | CO₂H |
| | dimethylamino-2- | | | | |
| | fluorophenyl | | | | |
| 79-84 | 4-chloro-3- | Me | Me | Me | CO₂Me |
| | dimethylamino-2- | | | | |
| | fluorophenyl | | | | |

84 compounds are described, designated compounds 80-1 to 80-84 respectively, of formula (1E) wherein D is N and X is NMe, and the values of A, R⁵, R⁷, R⁸ and Z are as defined in Table 5.

84 compounds are described, designated compounds 81-1 to 81-84 respectively, of formula (1E) wherein D is N and X is NCH₂Ph, and the values of A, R⁵, R⁷, R⁸ and Z are as defined in Table 5.

84 compounds are described, designated compounds 82-1 to 82-84 respectively, of formula (1E) wherein D is N and X is NCH₂(2-nitrophenyl), and the values of A, R⁵, R⁷, R⁸ and Z are as defined in Table 5.

84 compounds are described, designated compounds 83-1 to 83-84 respectively, of formula (1E) wherein D is N and X is NCH₂(2,4-dimethoxyphenyl), and the values of A, R⁵, R⁷, R⁸ and Z are as defined in Table 5.

84 compounds are described, designated compounds 84-1 to 84-84 respectively, of formula (1E) wherein D is N and X is NCH₂(2-furanyl), and the values of A, R⁵, R⁷, R⁸ and Z are as defined in Table 5.

84 compounds are described, designated compounds 85-1 to 85-84 respectively, of formula (1E) wherein D is CH and X is NH, and the values of A, R⁵, R⁷, R⁸ and Z are as defined in Table 5.

84 compounds are described, designated compounds 86-1 to 86-84 respectively, of formula (1E) wherein D is CH and X is NMe, and the values of A, R⁵, R⁷, R⁸ and Z are as defined in Table 5.

84 compounds are described, designated compounds 87-1 to 87-84 respectively, of formula (1E) wherein D is CH and X is NCH₂Ph, and the values of A, R⁵, R⁷, R⁸ and Z are as defined in Table 5.

84 compounds are described, designated compounds 88-1 to 88-84 respectively, of formula (1E) wherein D is CH and X is NCH₂(2-nitrophenyl), and the values of A, R⁵, R⁷, R⁸ and Z are as defined in Table 5.

84 compounds are described, designated compounds 89-1 to 89-84 respectively, of formula (1E) wherein D is CH and X is NCH₂(2,4-dimethoxyphenyl), and the values of A, R⁵, R⁷, R⁸ and Z are as defined in Table 5.

84 compounds are described, designated compounds 90-1 to 90-84 respectively, of formula (1E) wherein D is CH and X is NCH₂(2-furanyl), and the values of A, R⁵, R⁷, R⁸ and Z are as defined in Table 5.

Table 6 below provides 240 compounds designated compounds 91-1 to 91-240 respectively, of formula (1F) wherein D is N and X is NH.

TABLE 6
(1F)

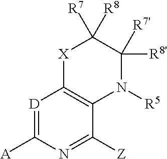

| Compound | Substituents Values | | | | | | |
|---|---|---|---|---|---|---|---|
| Number | A | R⁷ | R⁸ | R^{7′} | R^{8′} | R⁵ | Z |
| 91-1 | cyclopropyl | H | H | H | H | H | CO₂H |
| 91-2 | cyclopropyl | H | H | H | H | H | CO₂Me |
| 91-3 | cyclopropyl | H | H | H | H | Me | CO₂H |
| 91-4 | cyclopropyl | H | H | H | H | Me | CO₂Me |
| 91-5 | cyclopropyl | H | H | H | H | i-Pr | CO₂H |
| 91-6 | cyclopropyl | H | H | H | H | i-Pr | CO₂Me |
| 91-7 | cyclopropyl | H | H | H | H | CH₂Ph | CO₂H |
| 91-8 | cyclopropyl | H | H | H | H | CH₂Ph | CO₂Me |
| 91-9 | cyclopropyl | H | H | H | H | Ph | CO₂H |
| 91-10 | cyclopropyl | H | H | H | H | Ph | CO₂Me |
| 91-11 | cyclopropyl | H | H | H | Me | H | CO₂H |
| 91-12 | cyclopropyl | H | H | H | Me | H | CO₂Me |
| 91-13 | cyclopropyl | H | H | H | Me | Me | CO₂H |
| 91-14 | cyclopropyl | H | H | H | Me | Me | CO₂Me |
| 91-15 | cyclopropyl | H | H | Me | Me | H | CO₂H |
| 91-16 | cyclopropyl | H | H | Me | Me | H | CO₂Me |
| 91-17 | cyclopropyl | H | H | Me | Me | Me | CO₂H |
| 91-18 | cyclopropyl | H | H | Me | Me | Me | CO₂Me |
| 91-19 | cyclopropyl | H | Me | H | H | H | CO₂H |
| 91-20 | cyclopropyl | H | Me | H | H | H | CO₂Me |
| 91-21 | cyclopropyl | H | Me | H | H | Me | CO₂H |
| 91-22 | cyclopropyl | H | Me | H | H | Me | CO₂Me |
| 91-23 | cyclopropyl | H | Me | H | Me | H | CO₂H |
| 91-24 | cyclopropyl | H | Me | H | Me | H | CO₂Me |
| 91-25 | cyclopropyl | H | Me | H | Me | Me | CO₂H |
| 91-26 | cyclopropyl | H | Me | H | Me | Me | CO₂Me |
| 91-27 | cyclopropyl | H | Me | Me | Me | H | CO₂H |
| 91-28 | cyclopropyl | H | Me | Me | Me | H | CO₂Me |
| 91-29 | cyclopropyl | H | Me | Me | Me | Me | CO₂H |
| 91-30 | cyclopropyl | H | Me | Me | Me | Me | CO₂Me |
| 91-31 | cyclopropyl | Me | Me | H | H | H | CO₂H |
| 91-32 | cyclopropyl | Me | Me | H | H | H | CO₂Me |
| 91-33 | cyclopropyl | Me | Me | H | H | Me | CO₂H |
| 91-34 | cyclopropyl | Me | Me | H | H | Me | CO₂Me |
| 91-35 | cyclopropyl | Me | Me | H | Me | H | CO₂H |
| 91-36 | cyclopropyl | Me | Me | H | Me | H | CO₂Me |
| 91-37 | cyclopropyl | Me | Me | H | Me | Me | CO₂H |
| 91-38 | cyclopropyl | Me | Me | H | Me | Me | CO₂Me |
| 91-39 | cyclopropyl | Me | Me | Me | Me | H | CO₂H |
| 91-40 | cyclopropyl | Me | Me | Me | Me | H | CO₂Me |
| 91-41 | cyclopropyl | Me | Me | Me | Me | Me | CO₂H |
| 91-42 | cyclopropyl | Me | Me | Me | Me | Me | CO₂Me |
| 91-43 | cyclopropyl | H | Ph | H | Ph | H | CO₂H |
| 91-44 | cyclopropyl | H | Ph | H | Ph | H | CO₂Me |
| 91-45 | cyclopropyl | H | Ph | H | Ph | Me | CO₂H |
| 91-46 | cyclopropyl | H | Ph | H | Ph | Me | CO₂Me |
| 91-47 | cyclopropyl | H | (CH₂)₄ | | H | H | CO₂H |
| 91-48 | cyclopropyl | H | (CH₂)₄ | | H | H | CO₂Me |
| 91-49 | cyclopropyl | H | (CH₂)₄ | | H | Me | CO₂H |
| 91-50 | cyclopropyl | H | (CH₂)₄ | | H | Me | CO₂Me |
| 91-51 | cyclopropyl | ═O | | H | H | H | CO₂H |
| 91-52 | cyclopropyl | ═O | | H | H | H | CO₂Me |
| 91-53 | cyclopropyl | ═O | | H | H | Me | CO₂H |
| 91-54 | cyclopropyl | ═O | | H | H | Me | CO₂Me |
| 91-55 | cyclopropyl | ═O | | H | Me | H | CO₂H |
| 91-56 | cyclopropyl | ═O | | H | Me | H | CO₂Me |
| 91-57 | cyclopropyl | ═O | | H | Me | Me | CO₂H |
| 91-58 | cyclopropyl | ═O | | H | Me | Me | CO₂Me |
| 91-59 | cyclopropyl | ═O | | Me | Me | H | CO₂H |
| 91-60 | cyclopropyl | ═O | | Me | Me | H | CO₂Me |
| 91-61 | cyclopropyl | ═O | | Me | Me | Me | CO₂H |
| 91-62 | cyclopropyl | ═O | | Me | Me | Me | CO₂Me |
| 91-63 | cyclopropyl | H | H | ═O | | H | CO₂H |
| 91-64 | cyclopropyl | H | H | ═O | | H | CO₂Me |
| 91-65 | cyclopropyl | H | H | ═O | | Me | CO₂H |
| 91-66 | cyclopropyl | H | H | ═O | | Me | CO₂Me |
| 91-67 | cyclopropyl | H | Me | ═O | | H | CO₂H |
| 91-68 | cyclopropyl | H | Me | ═O | | H | CO₂Me |
| 91-69 | cyclopropyl | H | Me | ═O | | Me | CO₂H |
| 91-70 | cyclopropyl | H | Me | ═O | | Me | CO₂Me |
| 91-71 | cyclopropyl | Me | Me | ═O | | H | CO₂H |
| 91-72 | cyclopropyl | Me | Me | ═O | | H | CO₂Me |
| 91-73 | cyclopropyl | Me | Me | ═O | | Me | CO₂H |
| 91-74 | cyclopropyl | Me | Me | ═O | | Me | CO₂Me |
| 91-75 | cyclopropyl | ═O | | ═O | | H | CO₂H |
| 91-76 | cyclopropyl | ═O | | ═O | | H | CO₂Me |
| 91-77 | cyclopropyl | ═O | | ═O | | Me | CO₂H |
| 91-78 | cyclopropyl | ═O | | ═O | | Me | CO₂Me |
| 91-79 | cyclopropyl | ═O | | ═O | | CH₂Ph | CO₂H |
| 91-80 | cyclopropyl | ═O | | ═O | | CH₂Ph | CO₂Me |
| 91-81 | 4-chloro-3- | H | H | H | H | H | CO₂H |
| | fluorophenyl | | | | | | |
| 91-82 | 4-chloro-3- | H | H | H | H | H | CO₂Me |
| | fluorophenyl | | | | | | |
| 91-83 | 4-chloro-3- | H | H | H | H | Me | CO₂H |
| | fluorophenyl | | | | | | |
| 91-84 | 4-chloro-3- | H | H | H | H | Me | CO₂Me |
| | fluorophenyl | | | | | | |
| 91-85 | 4-chloro-3- | H | H | H | H | i-Pr | CO₂H |
| | fluorophenyl | | | | | | |
| 91-86 | 4-chloro-3- | H | H | H | H | i-Pr | CO₂Me |
| | fluorophenyl | | | | | | |
| 91-87 | 4-chloro-3- | H | H | H | H | CH₂Ph | CO₂H |
| | fluorophenyl | | | | | | |
| 91-88 | 4-chloro-3- | H | H | H | H | CH₂Ph | CO₂Me |
| | fluorophenyl | | | | | | |
| 91-89 | 4-chloro-3- | H | H | H | H | Ph | CO₂H |
| | fluorophenyl | | | | | | |
| 91-90 | 4-chloro-3- | H | H | H | H | Ph | CO₂Me |
| | fluorophenyl | | | | | | |
| 91-91 | 4-chloro-3- | H | H | H | Me | H | CO₂H |
| | fluorophenyl | | | | | | |
| 91-92 | 4-chloro-3- | H | H | H | Me | H | CO₂Me |
| | fluorophenyl | | | | | | |
| 91-93 | 4-chloro-3- | H | H | H | Me | Me | CO₂H |
| | fluorophenyl | | | | | | |
| 91-94 | 4-chloro-3- | H | H | H | Me | Me | CO₂Me |
| | fluorophenyl | | | | | | |
| 91-95 | 4-chloro-3- | H | H | Me | Me | H | CO₂H |
| | fluorophenyl | | | | | | |
| 91-96 | 4-chloro-3- | H | H | Me | Me | H | CO₂Me |
| | fluorophenyl | | | | | | |
| 91-97 | 4-chloro-3- | H | H | Me | Me | Me | CO₂H |
| | fluorophenyl | | | | | | |
| 91-98 | 4-chloro-3- | H | H | Me | Me | Me | CO₂Me |
| | fluorophenyl | | | | | | |
| 91-99 | 4-chloro-3- | H | Me | H | H | H | CO₂H |
| | fluorophenyl | | | | | | |
| 91-100 | 4-chloro-3- | H | Me | H | H | H | CO₂Me |
| | fluorophenyl | | | | | | |
| 91-101 | 4-chloro-3- | H | Me | H | H | Me | CO₂H |
| | fluorophenyl | | | | | | |
| 91-102 | 4-chloro-3- | H | Me | H | H | Me | CO₂Me |
| | fluorophenyl | | | | | | |
| 91-103 | 4-chloro-3- | H | Me | H | Me | H | CO₂H |
| | fluorophenyl | | | | | | |
| 91-104 | 4-chloro-3- | H | Me | H | Me | H | CO₂Me |
| | fluorophenyl | | | | | | |
| 91-105 | 4-chloro-3- | H | Me | H | Me | Me | CO₂H |
| | fluorophenyl | | | | | | |
| 91-106 | 4-chloro-3- | H | Me | H | Me | Me | CO₂Me |
| | fluorophenyl | | | | | | |
| 91-107 | 4-chloro-3- | H | Me | Me | Me | H | CO₂H |
| | fluorophenyl | | | | | | |
| 91-108 | 4-chloro-3- | H | Me | Me | Me | H | CO₂Me |
| | fluorophenyl | | | | | | |
| 91-109 | 4-chloro-3- | H | Me | Me | Me | Me | CO₂H |
| | fluorophenyl | | | | | | |
| 91-110 | 4-chloro-3- | H | Me | Me | Me | Me | CO₂Me |
| | fluorophenyl | | | | | | |
| 91-111 | 4-chloro-3- | Me | Me | H | H | H | CO₂H |
| | fluorophenyl | | | | | | |
| 91-112 | 4-chloro-3- | Me | Me | H | H | H | CO₂Me |
| | fluorophenyl | | | | | | |
| 91-113 | 4-chloro-3- | Me | Me | H | H | Me | CO₂H |
| | fluorophenyl | | | | | | |
| 91-114 | 4-chloro-3- | Me | Me | H | H | Me | CO₂Me |
| | fluorophenyl | | | | | | |
| 91-115 | 4-chloro-3- | Me | Me | H | Me | H | CO₂H |
| | fluorophenyl | | | | | | |
| 91-116 | 4-chloro-3- | Me | Me | H | Me | H | CO₂Me |
| | fluorophenyl | | | | | | |
| 91-117 | 4-chloro-3- | Me | Me | H | Me | Me | CO₂H |
| | fluorophenyl | | | | | | |
| 91-118 | 4-chloro-3- | Me | Me | H | Me | Me | CO₂Me |
| | fluorophenyl | | | | | | |
| 91-119 | 4-chloro-3- | Me | Me | Me | Me | H | CO₂H |
| | fluorophenyl | | | | | | |
| 91-120 | 4-chloro-3- | Me | Me | Me | Me | H | CO₂Me |
| | fluorophenyl | | | | | | |
| 91-121 | 4-chloro-3- | Me | Me | Me | Me | Me | CO₂H |
| | fluorophenyl | | | | | | |
| 91-122 | 4-chloro-3- | Me | Me | Me | Me | Me | CO₂Me |
| | fluorophenyl | | | | | | |
| 91-123 | 4-chloro-3- | H | Ph | H | Ph | H | CO₂H |
| | fluorophenyl | | | | | | |
| 91-124 | 4-chloro-3- | H | Ph | H | Ph | H | CO₂Me |
| | fluorophenyl | | | | | | |
| 91-125 | 4-chloro-3- | H | Ph | H | Ph | Me | CO₂H |
| | fluorophenyl | | | | | | |
| 91-126 | 4-chloro-3- | H | Ph | H | Ph | Me | CO₂Me |
| | fluorophenyl | | | | | | |
| 91-127 | 4-chloro-3- | H | (CH₂)₄ | | H | H | CO₂H |
| | fluorophenyl | | | | | | |
| 91-128 | 4-chloro-3- | H | (CH₂)₄ | | H | H | CO₂Me |
| | fluorophenyl | | | | | | |
| 91-129 | 4-chloro-3- | H | (CH₂)₄ | | H | Me | CO₂H |
| | fluorophenyl | | | | | | |
| 91-130 | 4-chloro-3- | H | (CH₂)₄ | | H | Me | CO₂Me |
| | fluorophenyl | | | | | | |
| 91-131 | 4-chloro-3- | ═O | | H | H | H | CO₂H |
| | fluorophenyl | | | | | | |
| 91-132 | 4-chloro-3- | ═O | | H | H | H | CO₂Me |
| | fluorophenyl | | | | | | |
| 91-133 | 4-chloro-3- | ═O | | H | H | Me | CO₂H |
| | fluorophenyl | | | | | | |
| 91-134 | 4-chloro-3- | ═O | | H | H | Me | CO₂Me |
| | fluorophenyl | | | | | | |
| 91-135 | 4-chloro-3- | ═O | | H | Me | H | CO₂H |
| | fluorophenyl | | | | | | |
| 91-136 | 4-chloro-3- | ═O | | H | Me | H | CO₂Me |
| | fluorophenyl | | | | | | |
| 91-137 | 4-chloro-3- | ═O | | H | Me | Me | CO₂H |
| | fluorophenyl | | | | | | |
| 91-138 | 4-chloro-3- | ═O | | H | Me | Me | CO₂Me |
| | fluorophenyl | | | | | | |
| 91-139 | 4-chloro-3- | ═O | | Me | Me | H | CO₂H |
| | fluorophenyl | | | | | | |
| 91-140 | 4-chloro-3- | ═O | | Me | Me | H | CO₂Me |
| | fluorophenyl | | | | | | |
| 91-141 | 4-chloro-3- | ═O | | Me | Me | Me | CO₂H |
| | fluorophenyl | | | | | | |
| 91-142 | 4-chloro-3- | ═O | | Me | Me | Me | CO₂Me |
| | fluorophenyl | | | | | | |
| 91-143 | 4-chloro-3- | H | H | ═O | | H | CO₂H |
| | fluorophenyl | | | | | | |
| 91-144 | 4-chloro-3- | H | H | ═O | | H | CO₂Me |
| | fluorophenyl | | | | | | |
| 91-145 | 4-chloro-3- | H | H | ═O | | Me | CO₂H |
| | fluorophenyl | | | | | | |
| 91-146 | 4-chloro-3- | H | H | ═O | | Me | CO₂Me |
| | fluorophenyl | | | | | | |
| 91-147 | 4-chloro-3- | H | Me | ═O | | H | CO₂H |
| | fluorophenyl | | | | | | |
| 91-148 | 4-chloro-3- | H | Me | ═O | | H | CO₂Me |
| | fluorophenyl | | | | | | |
| 91-149 | 4-chloro-3- | H | Me | ═O | | Me | CO₂H |
| | fluorophenyl | | | | | | |
| 91-150 | 4-chloro-3- | H | Me | ═O | | Me | CO₂Me |
| | fluorophenyl | | | | | | |
| 91-151 | 4-chloro-3- | Me | Me | ═O | | H | CO₂H |
| | fluorophenyl | | | | | | |
| 91-152 | 4-chloro-3- | Me | Me | ═O | | H | CO₂Me |
| | fluorophenyl | | | | | | |
| 91-153 | 4-chloro-3- | Me | Me | ═O | | Me | CO₂H |
| | fluorophenyl | | | | | | |
| 91-154 | 4-chloro-3- | Me | Me | ═O | | Me | CO₂Me |
| | fluorophenyl | | | | | | |
| 91-155 | 4-chloro-3- | ═O | | ═O | | H | CO₂H |
| | fluorophenyl | | | | | | |
| 91-156 | 4-chloro-3- | ═O | | ═O | | H | CO₂Me |
| | fluorophenyl | | | | | | |
| 91-157 | 4-chloro-3- | ═O | | ═O | | Me | CO₂H |
| | fluorophenyl | | | | | | |
| 91-158 | 4-chloro-3- | ═O | | ═O | | Me | CO₂Me |
| | fluorophenyl | | | | | | |
| 91-159 | 4-chloro-3- | ═O | | ═O | | CH₂Ph | CO₂H |
| | fluorophenyl | | | | | | |
| 91-160 | 4-chloro-3- | ═O | | ═O | | CH₂Ph | CO₂Me |
| | fluorophenyl | | | | | | |
| 91-161 | 4-chloro-2-fluoro-3- | H | H | H | H | H | CO₂H |
| | methoxyphenyl | | | | | | |
| 91-162 | 4-chloro-2-fluoro-3- | H | H | H | H | H | CO₂Me |
| | methoxyphenyl | | | | | | |
| 91-163 | 4-chloro-2-fluoro-3- | H | H | H | H | Me | CO₂H |
| | methoxyphenyl | | | | | | |
| 91-164 | 4-chloro-2-fluoro-3- | H | H | H | H | Me | CO₂Me |
| | methoxyphenyl | | | | | | |
| 91-165 | 4-chloro-2-fluoro-3- | H | H | H | H | i-Pr | CO₂H |
| | methoxyphenyl | | | | | | |
| 91-166 | 4-chloro-2-fluoro-3- | H | H | H | H | i-Pr | CO₂Me |
| | methoxyphenyl | | | | | | |
| 91-167 | 4-chloro-2-fluoro-3- | H | H | H | H | CH₂Ph | CO₂H |
| | methoxyphenyl | | | | | | |
| 91-168 | 4-chloro-2-fluoro-3- | H | H | H | H | CH₂Ph | CO₂Me |
| | methoxyphenyl | | | | | | |
| 91-169 | 4-chloro-2-fluoro-3- | H | H | H | H | Ph | CO₂H |
| | methoxyphenyl | | | | | | |
| 91-170 | 4-chloro-2-fluoro-3- | H | H | H | H | Ph | CO₂Me |
| | methoxyphenyl | | | | | | |
| 91-171 | 4-chloro-2-fluoro-3- | H | H | H | Me | H | CO₂H |
| | methoxyphenyl | | | | | | |
| 91-172 | 4-chloro-2-fluoro-3- | H | H | H | Me | H | CO₂Me |
| | methoxyphenyl | | | | | | |
| 91-173 | 4-chloro-2-fluoro-3- | H | H | H | Me | Me | CO₂H |
| | methoxyphenyl | | | | | | |
| 91-174 | 4-chloro-2-fluoro-3- | H | H | H | Me | Me | CO₂Me |
| | methoxyphenyl | | | | | | |
| 91-175 | 4-chloro-2-fluoro-3- | H | H | Me | Me | H | CO₂H |
| | methoxyphenyl | | | | | | |
| 91-176 | 4-chloro-2-fluoro-3- | H | H | Me | Me | H | CO₂Me |
| | methoxyphenyl | | | | | | |
| 91-177 | 4-chloro-2-fluoro-3- | H | H | Me | Me | Me | CO₂H |
| | methoxyphenyl | | | | | | |
| 91-178 | 4-chloro-2-fluoro-3- | H | H | Me | Me | Me | CO₂Me |
| | methoxyphenyl | | | | | | |
| 91-179 | 4-chloro-2-fluoro-3- | H | Me | H | H | H | CO₂H |
| | methoxyphenyl | | | | | | |
| 91-180 | 4-chloro-2-fluoro-3- | H | Me | H | H | H | CO₂Me |
| | methoxyphenyl | | | | | | |
| 91-181 | 4-chloro-2-fluoro-3- | H | Me | H | H | Me | CO₂H |
| | methoxyphenyl | | | | | | |
| 91-182 | 4-chloro-2-fluoro-3- | H | Me | H | H | Me | CO₂Me |
| | methoxyphenyl | | | | | | |
| 91-183 | 4-chloro-2-fluoro-3- | H | Me | H | Me | H | CO₂H |
| | methoxyphenyl | | | | | | |
| 91-184 | 4-chloro-2-fluoro-3- | H | Me | H | Me | H | CO₂Me |
| | methoxyphenyl | | | | | | |
| 91-185 | 4-chloro-2-fluoro-3- | H | Me | H | Me | Me | CO₂H |
| | methoxyphenyl | | | | | | |
| 91-186 | 4-chloro-2-fluoro-3- | H | Me | H | Me | Me | CO₂Me |
| | methoxyphenyl | | | | | | |
| 91-187 | 4-chloro-2-fluoro-3- | H | Me | Me | Me | H | CO₂H |
| | methoxyphenyl | | | | | | |
| 91-188 | 4-chloro-2-fluoro-3- | H | Me | Me | Me | H | CO₂Me |
| | methoxyphenyl | | | | | | |
| 91-189 | 4-chloro-2-fluoro-3- | H | Me | Me | Me | Me | CO₂H |
| | methoxyphenyl | | | | | | |
| 91-190 | 4-chloro-2-fluoro-3- | H | Me | Me | Me | Me | CO₂Me |
| | methoxyphenyl | | | | | | |
| 91-191 | 4-chloro-2-fluoro-3- | Me | Me | H | H | H | CO₂H |
| | methoxyphenyl | | | | | | |
| 91-192 | 4-chloro-2-fluoro-3- | Me | Me | H | H | H | CO₂Me |
| | methoxyphenyl | | | | | | |
| 91-193 | 4-chloro-2-fluoro-3- | Me | Me | H | H | Me | CO₂H |
| | methoxyphenyl | | | | | | |
| 91-194 | 4-chloro-2-fluoro-3- | Me | Me | H | H | Me | CO₂Me |
| | methoxyphenyl | | | | | | |
| 91-195 | 4-chloro-2-fluoro-3- | Me | Me | H | Me | H | CO₂H |
| | methoxyphenyl | | | | | | |
| 91-196 | 4-chloro-2-fluoro-3- | Me | Me | H | Me | H | CO₂Me |
| | methoxyphenyl | | | | | | |
| 91-197 | 4-chloro-2-fluoro-3- | Me | Me | H | Me | Me | CO₂H |
| | methoxyphenyl | | | | | | |
| 91-198 | 4-chloro-2-fluoro-3- | Me | Me | H | Me | Me | CO₂Me |
| | methoxyphenyl | | | | | | |
| 91-199 | 4-chloro-2-fluoro-3- | Me | Me | Me | Me | H | CO₂H |
| | methoxyphenyl | | | | | | |
| 91-200 | 4-chloro-2-fluoro-3- | Me | Me | Me | Me | H | CO₂Me |
| | methoxyphenyl | | | | | | |
| 91-201 | 4-chloro-2-fluoro-3- | Me | Me | Me | Me | Me | CO₂H |
| | methoxyphenyl | | | | | | |
| 91-202 | 4-chloro-2-fluoro-3- | Me | Me | Me | Me | Me | CO₂Me |
| | methoxyphenyl | | | | | | |
| 91-203 | 4-chloro-2-fluoro-3- | H | Ph | H | Ph | H | CO₂H |
| | methoxyphenyl | | | | | | |
| 91-204 | 4-chloro-2-fluoro-3- | H | Ph | H | Ph | H | CO₂Me |
| | methoxyphenyl | | | | | | |
| 91-205 | 4-chloro-2-fluoro-3- | H | Ph | H | Ph | Me | CO₂H |
| | methoxyphenyl | | | | | | |
| 91-206 | 4-chloro-2-fluoro-3- | H | Ph | H | Ph | Me | CO₂Me |
| | methoxyphenyl | | | | | | |
| 91-207 | 4-chloro-2-fluoro-3- | H | (CH₂)₄ | | H | H | CO₂H |
| | methoxyphenyl | | | | | | |
| 91-208 | 4-chloro-2-fluoro-3- | H | (CH₂)₄ | | H | H | CO₂Me |
| | methoxyphenyl | | | | | | |
| 91-209 | 4-chloro-2-fluoro-3- | H | (CH₂)₄ | | H | Me | CO₂H |
| | methoxyphenyl | | | | | | |
| 91-210 | 4-chloro-2-fluoro-3- | H | (CH₂)₄ | | H | Me | CO₂Me |
| | methoxyphenyl | | | | | | |
| 91-211 | 4-chloro-2-fluoro-3- | ═O | | H | H | H | CO₂H |
| | methoxyphenyl | | | | | | |
| 91-212 | 4-chloro-2-fluoro-3- | ═O | | H | H | H | CO₂Me |
| | methoxyphenyl | | | | | | |
| 91-213 | 4-chloro-2-fluoro-3- | ═O | | H | H | Me | CO₂H |
| | methoxyphenyl | | | | | | |
| 91-214 | 4-chloro-2-fluoro-3- | ═O | | H | H | Me | CO₂Me |
| | methoxyphenyl | | | | | | |
| 91-215 | 4-chloro-2-fluoro-3- | ═O | | H | Me | H | CO₂H |
| | methoxyphenyl | | | | | | |
| 91-216 | 4-chloro-2-fluoro-3- | ═O | | H | Me | H | CO₂Me |
| | methoxyphenyl | | | | | | |
| 91-217 | 4-chloro-2-fluoro-3- | ═O | | H | Me | Me | CO₂H |
| | methoxyphenyl | | | | | | |
| 91-218 | 4-chloro-2-fluoro-3- | ═O | | H | Me | Me | CO₂Me |
| | methoxyphenyl | | | | | | |
| 91-219 | 4-chloro-2-fluoro-3- | ═O | | Me | Me | H | CO₂H |
| | methoxyphenyl | | | | | | |
| 91-220 | 4-chloro-2-fluoro-3- | ═O | | Me | Me | H | CO₂Me |
| | methoxyphenyl | | | | | | |
| 91-221 | 4-chloro-2-fluoro-3- | ═O | | Me | Me | Me | CO₂H |
| | methoxyphenyl | | | | | | |
| 91-222 | 4-chloro-2-fluoro-3- | ═O | | Me | Me | Me | CO₂Me |
| | methoxyphenyl | | | | | | |
| 91-223 | 4-chloro-2-fluoro-3- | H | H | ═O | | H | CO₂H |
| | methoxyphenyl | | | | | | |
| 91-224 | 4-chloro-2-fluoro-3- | H | H | ═O | | H | CO₂Me |
| | methoxyphenyl | | | | | | |
| 91-225 | 4-chloro-2-fluoro-3- | H | H | ═O | | Me | CO₂H |
| | methoxyphenyl | | | | | | |
| 91-226 | 4-chloro-2-fluoro-3- | H | H | ═O | | Me | CO₂Me |
| | methoxyphenyl | | | | | | |
| 91-227 | 4-chloro-2-fluoro-3- | H | Me | ═O | | H | CO₂H |
| | methoxyphenyl | | | | | | |
| 91-228 | 4-chloro-2-fluoro-3- | H | Me | ═O | | H | CO₂Me |
| | methoxyphenyl | | | | | | |
| 91-229 | 4-chloro-2-fluoro-3- | H | Me | ═O | | Me | CO₂H |
| | methoxyphenyl | | | | | | |
| 91-230 | 4-chloro-2-fluoro-3- | H | Me | ═O | | Me | CO₂Me |
| | methoxyphenyl | | | | | | |
| 91-231 | 4-chloro-2-fluoro-3- | Me | Me | ═O | | H | CO₂H |
| | methoxyphenyl | | | | | | |
| 91-232 | 4-chloro-2-fluoro-3- | Me | Me | ═O | | H | CO₂Me |
| | methoxyphenyl | | | | | | |
| 91-233 | 4-chloro-2-fluoro-3- | Me | Me | ═O | | Me | CO₂H |
| | methoxyphenyl | | | | | | |
| 91-234 | 4-chloro-2-fluoro-3- | Me | Me | ═O | | Me | CO₂Me |
| | methoxyphenyl | | | | | | |
| 91-235 | 4-chloro-2-fluoro-3- | ═O | | ═O | | H | CO₂H |
| | methoxyphenyl | | | | | | |
| 91-236 | 4-chloro-2-fluoro-3- | ═O | | ═O | | H | CO₂Me |
| | methoxyphenyl | | | | | | |
| 91-237 | 4-chloro-2-fluoro-3- | ═O | | ═O | | Me | CO₂H |
| | methoxyphenyl | | | | | | |
| 91-238 | 4-chloro-2-fluoro-3- | ═O | | ═O | | Me | CO₂Me |
| | methoxyphenyl | | | | | | |
| 91-239 | 4-chloro-2-fluoro-3- | ═O | | ═O | | CH₂Ph | CO₂H |
| | methoxyphenyl | | | | | | |
| 91-240 | 4-chloro-2-fluoro-3- | ═O | | ═O | | CH₂Ph | CO₂Me |
| | methoxyphenyl | | | | | | |

240 compounds are described, designated compounds 92-1 to 92-240 respectively, of formula (1F) wherein D is N and X is NMe, and the values of A, R⁵, R⁷, R^{7′}, R⁸, R^{8′} and Z are as defined in Table 6.

240 compounds are described, designated compounds 93-1 to 93-240 respectively, of formula (1F) wherein D is N and X is Ni—Pr, and the values of A, R⁵, R⁷, R^{7′}, R⁸, R^{8′} and Z are as defined in Table 6.

240 compounds are described, designated compounds 94-1 to 94-240 respectively, of formula (1F) wherein D is N and X is NPh, and the values of A, R⁵, R⁷, R^{7′}, R⁸, R^{8′} and Z are as defined in Table 6.

240 compounds are described, designated compounds 95-1 to 95-240 respectively, of formula (1F) wherein D is N and X is NCH₂Ph, and the values of A, R⁵, R⁷, R^{7′}, R⁸, R^{8′} and Z are as defined in Table 6.

240 compounds are described, designated compounds 96-1 to 96-240 respectively, of formula (1F) wherein D is N and X is NCH₂(2-nitrophenyl), and the values of A, R⁵, R⁷, R^{7′}, R⁸, R^{8′} and Z are as defined in Table 6.

240 compounds are described, designated compounds 97-1 to 97-240 respectively, of formula (1F) wherein D is N and X is NCH₂(2-furanyl), and the values of A, R⁵, R⁷, R^{7′}, R⁸, R^{8′} and Z are as defined in Table 6.

240 compounds are described, designated compounds 98-1 to 98-240 respectively, of formula (1F) wherein D is CH and X is NH, and the values of A, R⁵, R⁷, R^{7′}, R⁸, R^{8′} and Z are as defined in Table 6.

240 compounds are described, designated compounds 99-1 to 99-240 respectively, of formula (1F) wherein D is CH and X is NMe, and the values of A, R⁵, R⁷, R^{7′}, R⁸, R^{8′} and Z are as defined in Table 6.

240 compounds are described, designated compounds 100-1 to 100-240 respectively, of formula (1F) wherein D is CH and X is Ni—Pr, and the values of A, R⁵, R⁷, R^{7′}, R⁸, R^{8′} and Z are as defined in Table 6.

240 compounds are described, designated compounds 101-1 to 101-240 respectively, of formula (1F) wherein D is CH and X is NPh, and the values of A, R⁵, R⁷, R^{7′}, R⁸, R^{8′} and Z are as defined in Table 6.

240 compounds are described, designated compounds 102-1 to 102-240 respectively, of formula (1F) wherein D is CH and X is NCH₂Ph, and the values of A, R⁵, R⁷, R^{7′}, R⁸, R^{8′} and Z are as defined in Table 6.

240 compounds are described, designated compounds 103-1 to 103-240 respectively, of formula (1F) wherein D is CH and X is NCH₂(2-nitrophenyl), and the values of A, R⁵, R⁷, R^{7′}, R⁸, R^{8′} and Z are as defined in Table 6.

240 compounds are described, designated compounds 104-1 to 104-240 respectively, of formula (1F) wherein D is CH and X is NCH₂(2-furanyl), and the values of A, R⁵, R⁷, R^{7′}, R⁸, R^{8′} and Z are as defined in Table 6.

Table 7 below provides 108 compounds designated compounds 105-1 to 105-108 respectively, of formula (1G) wherein D is N and X is NH.

TABLE 7
(1G)

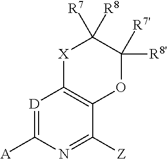

| Compound | Substituent Values | | | | | |
|---|---|---|---|---|---|---|
| Number | A | R⁷ | R⁸ | R^{7′} | R^{8′} | Z |
| 105-1 | cyclopropyl | H | H | H | H | CO₂H |
| 105-2 | cyclopropyl | H | H | H | H | CO₂Me |
| 105-3 | cyclopropyl | H | H | H | Me | CO₂H |
| 105-4 | cyclopropyl | H | H | H | Me | CO₂Me |
| 105-5 | cyclopropyl | H | H | Me | Me | CO₂H |
| 105-6 | cyclopropyl | H | H | Me | Me | CO₂Me |
| 105-7 | cyclopropyl | H | Me | H | H | CO₂H |
| 105-8 | cyclopropyl | H | Me | H | H | CO₂Me |
| 105-9 | cyclopropyl | H | Me | H | Me | CO₂H |
| 105-10 | cyclopropyl | H | Me | H | Me | CO₂Me |
| 105-11 | cyclopropyl | H | Me | Me | Me | CO₂H |
| 105-12 | cyclopropyl | H | Me | Me | Me | CO₂Me |
| 105-13 | cyclopropyl | Me | Me | H | H | CO₂H |
| 105-14 | cyclopropyl | Me | Me | H | H | CO₂Me |
| 105-15 | cyclopropyl | Me | Me | H | Me | CO₂H |
| 105-16 | cyclopropyl | Me | Me | H | Me | CO₂Me |
| 105-17 | cyclopropyl | Me | Me | Me | Me | CO₂H |
| 105-18 | cyclopropyl | Me | Me | Me | Me | CO₂Me |
| 105-19 | cyclopropyl | H | Ph | H | Ph | CO₂H |
| 105-20 | cyclopropyl | H | Ph | H | Ph | CO₂Me |
| 105-21 | cyclopropyl | H | (CH₂)₄ | | H | CO₂H |
| 105-22 | cyclopropyl | H | (CH₂)₄ | | H | CO₂Me |
| 105-23 | cyclopropyl | ═O | | H | H | CO₂H |
| 105-24 | cyclopropyl | ═O | | H | H | CO₂Me |
| 105-25 | cyclopropyl | ═O | | H | Me | CO₂H |
| 105-26 | cyclopropyl | ═O | | H | Me | CO₂Me |
| 105-27 | cyclopropyl | ═O | | Me | Me | CO₂H |
| 105-28 | cyclopropyl | ═O | | Me | Me | CO₂Me |
| 105-29 | cyclopropyl | H | H | ═O | | CO₂H |
| 105-30 | cyclopropyl | H | H | ═O | | CO₂Me |
| 105-31 | cyclopropyl | H | Me | ═O | | CO₂H |
| 105-32 | cyclopropyl | H | Me | ═O | | CO₂Me |
| 105-33 | cyclopropyl | Me | Me | ═O | | CO₂H |
| 105-34 | cyclopropyl | Me | Me | ═O | | CO₂Me |
| 105-35 | cyclopropyl | ═O | | ═O | | CO₂H |
| 105-36 | cyclopropyl | ═O | | ═O | | CO₂Me |
| 105-37 | 4-chloro-3-fluorophenyl | H | H | H | H | CO₂H |
| 105-38 | 4-chloro-3-fluorophenyl | H | H | H | H | CO₂Me |
| 105-39 | 4-chloro-3-fluorophenyl | H | H | H | Me | CO₂H |
| 105-40 | 4-chloro-3-fluorophenyl | H | H | H | Me | CO₂Me |
| 105-41 | 4-chloro-3-fluorophenyl | H | H | Me | Me | CO₂H |
| 105-42 | 4-chloro-3-fluorophenyl | H | H | Me | Me | CO₂Me |
| 105-43 | 4-chloro-3-fluorophenyl | H | Me | H | H | CO₂H |
| 105-44 | 4-chloro-3-fluorophenyl | H | Me | H | H | CO₂Me |
| 105-45 | 4-chloro-3-fluorophenyl | H | Me | H | Me | CO₂H |
| 105-46 | 4-chloro-3-fluorophenyl | H | Me | H | Me | CO₂Me |
| 105-47 | 4-chloro-3-fluorophenyl | H | Me | Me | Me | CO₂H |
| 105-48 | 4-chloro-3-fluorophenyl | H | Me | Me | Me | CO₂Me |
| 105-49 | 4-chloro-3-fluorophenyl | Me | Me | H | H | CO₂H |
| 105-50 | 4-chloro-3-fluorophenyl | Me | Me | H | H | CO₂Me |
| 105-51 | 4-chloro-3-fluorophenyl | Me | Me | H | Me | CO₂H |
| 105-52 | 4-chloro-3-fluorophenyl | Me | Me | H | Me | CO₂Me |
| 105-53 | 4-chloro-3-fluorophenyl | Me | Me | Me | Me | CO₂H |
| 105-54 | 4-chloro-3-fluorophenyl | Me | Me | Me | Me | CO₂Me |
| 105-55 | 4-chloro-3-fluorophenyl | H | Ph | H | Ph | CO₂H |
| 105-56 | 4-chloro-3-fluorophenyl | H | Ph | H | Ph | CO₂Me |
| 105-57 | 4-chloro-3-fluorophenyl | H | (CH₂)₄ | | H | CO₂H |
| 105-58 | 4-chloro-3-fluorophenyl | H | (CH₂)₄ | | H | CO₂Me |
| 105-59 | 4-chloro-3-fluorophenyl | ═O | | H | H | CO₂H |
| 105-60 | 4-chloro-3-fluorophenyl | ═O | | H | H | CO₂Me |
| 105-61 | 4-chloro-3-fluorophenyl | ═O | | H | Me | CO₂H |
| 105-62 | 4-chloro-3-fluorophenyl | ═O | | H | Me | CO₂Me |
| 105-63 | 4-chloro-3-fluorophenyl | ═O | | Me | Me | CO₂H |
| 105-64 | 4-chloro-3-fluorophenyl | ═O | | Me | Me | CO₂Me |
| 105-65 | 4-chloro-3-fluorophenyl | H | H | ═O | | CO₂H |
| 105-66 | 4-chloro-3-fluorophenyl | H | H | ═O | | CO₂Me |
| 105-67 | 4-chloro-3-fluorophenyl | H | Me | ═O | | CO₂H |
| 105-68 | 4-chloro-3-fluorophenyl | H | Me | ═O | | CO₂Me |
| 105-69 | 4-chloro-3-fluorophenyl | Me | Me | ═O | | CO₂H |
| 105-70 | 4-chloro-3-fluorophenyl | Me | Me | ═O | | CO₂Me |
| 105-71 | 4-chloro-3-fluorophenyl | ═O | | ═O | | CO₂H |
| 105-72 | 4-chloro-3-fluorophenyl | ═O | | ═O | | CO₂Me |
| 105-73 | 4-chloro-2-fluoro-3- | H | H | H | H | CO₂H |
| | methoxyphenyl | | | | | |
| 105-74 | 4-chloro-2-fluoro-3- | H | H | H | H | CO₂Me |
| | methoxyphenyl | | | | | |
| 105-75 | 4-chloro-2-fluoro-3- | H | H | H | Me | CO₂H |
| | methoxyphenyl | | | | | |
| 105-76 | 4-chloro-2-fluoro-3- | H | H | H | Me | CO₂Me |
| | methoxyphenyl | | | | | |
| 105-77 | 4-chloro-2-fluoro-3- | H | H | Me | Me | CO₂H |
| | methoxyphenyl | | | | | |
| 105-78 | 4-chloro-2-fluoro-3- | H | H | Me | Me | CO₂Me |
| | methoxyphenyl | | | | | |
| 105-79 | 4-chloro-2-fluoro-3- | H | Me | H | H | CO₂H |
| | methoxyphenyl | | | | | |
| 105-80 | 4-chloro-2-fluoro-3- | H | Me | H | H | CO₂Me |
| | methoxyphenyl | | | | | |
| 105-81 | 4-chloro-2-fluoro-3- | H | Me | H | Me | CO₂H |
| | methoxyphenyl | | | | | |
| 105-82 | 4-chloro-2-fluoro-3- | H | Me | H | Me | CO₂Me |
| | methoxyphenyl | | | | | |
| 105-83 | 4-chloro-2-fluoro-3- | H | Me | Me | Me | CO₂H |
| | methoxyphenyl | | | | | |
| 105-84 | 4-chloro-2-fluoro-3- | H | Me | Me | Me | CO₂Me |
| | methoxyphenyl | | | | | |
| 105-85 | 4-chloro-2-fluoro-3- | Me | Me | H | H | CO₂H |
| | methoxyphenyl | | | | | |
| 105-86 | 4-chloro-2-fluoro-3- | Me | Me | H | H | CO₂Me |
| | methoxyphenyl | | | | | |
| 105-87 | 4-chloro-2-fluoro-3- | Me | Me | H | Me | CO₂H |
| | methoxyphenyl | | | | | |
| 105-88 | 4-chloro-2-fluoro-3- | Me | Me | H | Me | CO₂Me |
| | methoxyphenyl | | | | | |
| 105-89 | 4-chloro-2-fluoro-3- | Me | Me | Me | Me | CO₂H |
| | methoxyphenyl | | | | | |
| 105-90 | 4-chloro-2-fluoro-3- | Me | Me | Me | Me | CO₂Me |
| | methoxyphenyl | | | | | |
| 105-91 | 4-chloro-2-fluoro-3- | H | Ph | H | Ph | CO₂H |
| | methoxyphenyl | | | | | |
| 105-92 | 4-chloro-2-fluoro-3- | H | Ph | H | Ph | CO₂Me |
| | methoxyphenyl | | | | | |
| 105-93 | 4-chloro-2-fluoro-3- | H | (CH₂)₄ | | H | CO₂H |
| | methoxyphenyl | | | | | |
| 105-94 | 4-chloro-2-fluoro-3- | H | (CH₂)₄ | | H | CO₂Me |
| | methoxyphenyl | | | | | |
| 105-95 | 4-chloro-2-fluoro-3- | ═O | | H | H | CO₂H |
| | methoxyphenyl | | | | | |
| 105-96 | 4-chloro-2-fluoro-3- | ═O | | H | H | CO₂Me |
| | methoxyphenyl | | | | | |
| 105-97 | 4-chloro-2-fluoro-3- | ═O | | H | Me | CO₂H |
| | methoxyphenyl | | | | | |
| 105-98 | 4-chloro-2-fluoro-3- | ═O | | H | Me | CO₂Me |
| | methoxyphenyl | | | | | |
| 105-99 | 4-chloro-2-fluoro-3- | ═O | | Me | Me | CO₂H |
| | methoxyphenyl | | | | | |
| 105-100 | 4-chloro-2-fluoro-3- | ═O | | Me | Me | CO₂Me |
| | methoxyphenyl | | | | | |
| 105-101 | 4-chloro-2-fluoro-3- | H | H | ═O | | CO₂H |
| | methoxyphenyl | | | | | |
| 105-102 | 4-chloro-2-fluoro-3- | H | H | ═O | | CO₂Me |
| | methoxyphenyl | | | | | |
| 105-103 | 4-chloro-2-fluoro-3- | H | Me | ═O | | CO₂H |
| | methoxyphenyl | | | | | |
| 105-104 | 4-chloro-2-fluoro-3- | H | Me | ═O | | CO₂Me |
| | methoxyphenyl | | | | | |
| 105-105 | 4-chloro-2-fluoro-3- | Me | Me | ═O | | CO₂H |
| | methoxyphenyl | | | | | |
| 105-106 | 4-chloro-2-fluoro-3- | Me | Me | ═O | | CO₂Me |
| | methoxyphenyl | | | | | |
| 105-107 | 4-chloro-2-fluoro-3- | ═O | | ═O | | CO₂H |
| | methoxyphenyl | | | | | |
| 105-108 | 4-chloro-2-fluoro-3- | ═O | | ═O | | CO₂Me |
| | methoxyphenyl | | | | | |

108 compounds are described, designated compounds 106-1 to 106-108 respectively, of formula (1G) wherein D is N and X is NMe, and the values of A, R⁷, R^{7′}, R⁸, R^{8′} and Z are as defined in Table 7.

108 compounds are described, designated compounds 107-1 to 107-108 respectively, of formula (1G) wherein D is N and X is Ni—Pr, and the values of A, R⁷, R^{7′}, R⁸, R^{8′} and Z are as defined in Table 7.

108 compounds are described, designated compounds 108-1 to 108-108 respectively, of formula (1G) wherein D is N and X is NPh, and the values of A, R⁷, R^{7′}, R⁸, R^{8′} and Z are as defined in Table 7.

108 compounds are described, designated compounds 109-1 to 109-108 respectively, of formula (1G) wherein D is N and X is NCH₂Ph, and the values of A, R⁷, R^{7′}, R⁸, R^{8′} and Z are as defined in Table 7.

108 compounds are described, designated compounds 110-1 to 110-108 respectively, of formula (1G) wherein D is N and X is NCH₂(2-nitrophenyl), and the values of A, R⁷, R^{7′}, R⁸, R^{8′} and Z are as defined in Table 7.

108 compounds are described, designated compounds 111-1 to 111-108 respectively, of formula (1G) wherein D is N and X is NCH₂(2-furanyl), and the values of A, R⁷, R^{7′}, R⁸, R^{8′} and Z are as defined in Table 7.

108 compounds are described, designated compounds 112-1 to 112-108 respectively, of formula (1G) wherein D is CH and X is NH, and the values of A, R⁷, R^{7′}, R⁸, R^{8′} and Z are as defined in Table 7.

108 compounds are described, designated compounds 113-1 to 113-108 respectively, of formula (1G) wherein D is CH and X is NMe, and the values of A, R⁷, R^{7′}, R⁸, R^{8′} and Z are as defined in Table 7.

108 compounds are described, designated compounds 114-1 to 114-108 respectively, of formula (1G) wherein D is CH and X is Ni—Pr, and the values of A, R⁷, R^{7′}, R⁸, R^{8′} and Z are as defined in Table 7.

108 compounds are described, designated compounds 115-1 to 115-108 respectively, of formula (1G) wherein D is CH and X is NPh, and the values of A, R⁷, R^{7′}, R⁸, R^{8′} and Z are as defined in Table 7.

108 compounds are described, designated compounds 116-1 to 116-108 respectively, of formula (1G) wherein D is CH and X is NCH₂Ph, and the values of A, R⁷, R^{7′}, R⁸, R^{8′} and Z are as defined in Table 7.

108 compounds are described, designated compounds 117-1 to 117-108 respectively, of formula (1G) wherein D is CH and X is NCH₂(2-nitrophenyl), and the values of A, R⁷, R^{7′}, R⁸, R^{8′} and Z are as defined in Table 7.

108 compounds are described, designated compounds 118-1 to 118-108 respectively, of formula (1G) wherein D is CH and X is NCH₂(2-furanyl), and the values of A, R⁷, R^{7′}, R⁸, R^{8′} and Z are as defined in Table 7.

Table 8 below provides 210 compounds designated compounds 119-1 to 119-210 respectively, of formula (1H) wherein D is N and X is NH.

TABLE 8
(1H)

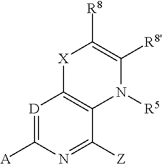

| Compound | Substituent Values | | | | |
|---|---|---|---|---|---|
| Number | A | R⁸ | R^{8′} | R⁵ | Z |
| 119-1 | cyclopropyl | H | H | H | CO₂H |
| 119-2 | cyclopropyl | H | H | H | CO₂Me |
| 119-3 | cyclopropyl | H | H | Me | CO₂H |
| 119-4 | cyclopropyl | H | H | Me | CO₂Me |
| 119-5 | cyclopropyl | H | H | i-Pr | CO₂H |
| 119-6 | cyclopropyl | H | H | i-Pr | CO₂Me |
| 119-7 | cyclopropyl | H | H | CH₂Ph | CO₂H |
| 119-8 | cyclopropyl | H | H | CH₂Ph | CO₂Me |
| 119-9 | cyclopropyl | H | H | Ph | CO₂H |
| 119-10 | cyclopropyl | H | H | Ph | CO₂Me |
| 119-11 | cyclopropyl | H | Me | H | CO₂H |
| 119-12 | cyclopropyl | H | Me | H | CO₂Me |
| 119-13 | cyclopropyl | H | Me | Me | CO₂H |
| 119-14 | cyclopropyl | H | Me | Me | CO₂Me |
| 119-15 | cyclopropyl | Me | H | H | CO₂H |
| 119-16 | cyclopropyl | Me | H | H | CO₂Me |
| 119-17 | cyclopropyl | Me | H | Me | CO₂H |
| 119-18 | cyclopropyl | Me | H | Me | CO₂Me |
| 119-19 | cyclopropyl | Me | Me | H | CO₂H |
| 119-20 | cyclopropyl | Me | Me | H | CO₂Me |
| 119-21 | cyclopropyl | Me | Me | Me | CO₂H |
| 119-22 | cyclopropyl | Me | Me | Me | CO₂Me |
| 119-23 | cyclopropyl | (CH₂)₄ | | H | CO₂H |
| 119-24 | cyclopropyl | (CH₂)₄ | | H | CO₂Me |
| 119-25 | cyclopropyl | (CH₂)₄ | | Me | CO₂H |
| 119-26 | cyclopropyl | (CH₂)₄ | | Me | CO₂Me |
| 119-27 | cyclopropyl | CH═CH—CH═CH | | H | CO₂H |
| 119-28 | cyclopropyl | CH═CH—CH═CH | | H | CO₂Me |
| 119-29 | cyclopropyl | CH═CH—CH═CH | | Me | CO₂H |
| 119-30 | cyclopropyl | CH═CH—CH═CH | | Me | CO₂Me |
| 119-31 | cyclopropyl | CH═CMe—CH═CH | | H | CO₂H |
| 119-32 | cyclopropyl | CH═CMe—CH═CH | | H | CO₂Me |
| 119-33 | cyclopropyl | CH═CMe—CH═CH | | Me | CO₂H |
| 119-34 | cyclopropyl | CH═CMe—CH═CH | | Me | CO₂Me |
| 119-35 | cyclopropyl | CH═CH—CMe═CH | | H | CO₂H |
| 119-36 | cyclopropyl | CH═CH—CMe═CH | | H | CO₂Me |
| 119-37 | cyclopropyl | CH═CH—CMe═CH | | Me | CO₂H |
| 119-38 | cyclopropyl | CH═CH—CMe═CH | | Me | CO₂Me |
| 119-39 | cyclopropyl | CH═CMe—CMe═CH | | H | CO₂H |
| 119-40 | cyclopropyl | CH═CMe—CMe═CH | | H | CO₂Me |
| 119-41 | cyclopropyl | CH═CMe—CMe═CH | | Me | CO₂H |
| 119-42 | cyclopropyl | CH═CMe—CMe═CH | | Me | CO₂Me |
| 119-43 | cyclopropyl | CMe═CMe—CH═CH | | H | CO₂H |
| 119-44 | cyclopropyl | CMe═CMe—CH═CH | | H | CO₂Me |
| 119-45 | cyclopropyl | CMe═CMe—CH═CH | | Me | CO₂H |
| 119-46 | cyclopropyl | CMe═CMe—CH═CH | | Me | CO₂Me |
| 119-47 | cyclopropyl | CH═CH—CMe═CMe | | H | CO₂H |
| 119-48 | cyclopropyl | CH═CH—CMe═CMe | | H | CO₂Me |
| 119-49 | cyclopropyl | CH═CH—CMe═CMe | | Me | CO₂H |
| 119-50 | cyclopropyl | CH═CH—CMe═CMe | | Me | CO₂Me |
| 119-51 | cyclopropyl | CH═CMe—CCl═CH | | H | CO₂H |
| 119-52 | cyclopropyl | CH═CMe—CCl═CH | | H | CO₂Me |
| 119-53 | cyclopropyl | CH═CMe—CCl═CH | | Me | CO₂H |
| 119-54 | cyclopropyl | CH═CMe—CCl═CH | | Me | CO₂Me |
| 119-55 | cyclopropyl | CH═CCl—CMe═CH | | H | CO₂H |
| 119-56 | cyclopropyl | CH═CCl—CMe═CH | | H | CO₂Me |
| 119-57 | cyclopropyl | CH═CCl—CMe═CH | | Me | CO₂H |
| 119-58 | cyclopropyl | CH═CCl—CMe═CH | | Me | CO₂Me |
| 119-59 | cyclopropyl | CH═CCl—CCl═CH | | H | CO₂H |
| 119-60 | cyclopropyl | CH═CCl—CCl═CH | | H | CO₂Me |
| 119-61 | cyclopropyl | CH═CCl—CCl═CH | | Me | CO₂H |
| 119-62 | cyclopropyl | CH═CCl—CCl═CH | | Me | CO₂Me |
| 119-63 | cyclopropyl | C(NO₂)═CH—CH═CH | | H | CO₂H |
| 119-64 | cyclopropyl | C(NO₂)═CH—CH═CH | | H | CO₂Me |
| 119-65 | cyclopropyl | C(NO₂)═CH—CH═CH | | Me | CO₂H |
| 119-66 | cyclopropyl | C(NO₂)═CH—CH═CH | | Me | CO₂Me |
| 119-67 | cyclopropyl | CH═CH—CH═C(NO₂) | | H | CO₂H |
| 119-68 | cyclopropyl | CH═CH—CH═C(NO₂) | | H | CO₂Me |
| 119-69 | cyclopropyl | CH═CH—CH═C(NO₂) | | Me | CO₂H |
| 119-70 | cyclopropyl | CH═CH—CH═C(NO₂) | | Me | CO₂Me |
| 119-71 | 4-chloro-3-fluorophenyl | H | H | H | CO₂H |
| 119-72 | 4-chloro-3-fluorophenyl | H | H | H | CO₂Me |
| 119-73 | 4-chloro-3-fluorophenyl | H | H | Me | CO₂H |
| 119-74 | 4-chloro-3-fluorophenyl | H | H | Me | CO₂Me |
| 119-75 | 4-chloro-3-fluorophenyl | H | H | i-Pr | CO₂H |
| 119-76 | 4-chloro-3-fluorophenyl | H | H | i-Pr | CO₂Me |
| 119-77 | 4-chloro-3-fluorophenyl | H | H | CH₂Ph | CO₂H |
| 119-78 | 4-chloro-3-fluorophenyl | H | H | CH₂Ph | CO₂Me |
| 119-79 | 4-chloro-3-fluorophenyl | H | H | Ph | CO₂H |
| 119-80 | 4-chloro-3-fluorophenyl | H | H | Ph | CO₂Me |
| 119-81 | 4-chloro-3-fluorophenyl | H | Me | H | CO₂H |
| 119-82 | 4-chloro-3-fluorophenyl | H | Me | H | CO₂Me |
| 119-83 | 4-chloro-3-fluorophenyl | H | Me | Me | CO₂H |
| 119-84 | 4-chloro-3-fluorophenyl | H | Me | Me | CO₂Me |
| 119-85 | 4-chloro-3-fluorophenyl | Me | H | H | CO₂H |
| 119-86 | 4-chloro-3-fluorophenyl | Me | H | H | CO₂Me |
| 119-87 | 4-chloro-3-fluorophenyl | Me | H | Me | CO₂H |
| 119-88 | 4-chloro-3-fluorophenyl | Me | H | Me | CO₂Me |
| 119-89 | 4-chloro-3-fluorophenyl | Me | Me | H | CO₂H |
| 119-90 | 4-chloro-3-fluorophenyl | Me | Me | H | CO₂Me |
| 119-91 | 4-chloro-3-fluorophenyl | Me | Me | Me | CO₂H |
| 119-92 | 4-chloro-3-fluorophenyl | Me | Me | Me | CO₂Me |
| 119-93 | 4-chloro-3-fluorophenyl | (CH₂)₄ | | H | CO₂H |
| 119-94 | 4-chloro-3-fluorophenyl | (CH₂)₄ | | H | CO₂Me |
| 119-95 | 4-chloro-3-fluorophenyl | (CH₂)₄ | | Me | CO₂H |
| 119-96 | 4-chloro-3-fluorophenyl | (CH₂)₄ | | Me | CO₂Me |
| 119-97 | 4-chloro-3-fluorophenyl | CH═CH—CH═CH | | H | CO₂H |
| 119-98 | 4-chloro-3-fluorophenyl | CH═CH—CH═CH | | H | CO₂Me |
| 119-99 | 4-chloro-3-fluorophenyl | CH═CH—CH═CH | | Me | CO₂H |
| 119-100 | 4-chloro-3-fluorophenyl | CH═CH—CH═CH | | Me | CO₂Me |
| 119-101 | 4-chloro-3-fluorophenyl | CH═CMe—CH═CH | | H | CO₂H |
| 119-102 | 4-chloro-3-fluorophenyl | CH═CMe—CH═CH | | H | CO₂Me |
| 119-103 | 4-chloro-3-fluorophenyl | CH═CMe—CH═CH | | Me | CO₂H |
| 119-104 | 4-chloro-3-fluorophenyl | CH═CMe—CH═CH | | Me | CO₂Me |
| 119-105 | 4-chloro-3-fluorophenyl | CH═CH—CMe═CH | | H | CO₂H |
| 119-106 | 4-chloro-3-fluorophenyl | CH═CH—CMe═CH | | H | CO₂Me |
| 119-107 | 4-chloro-3-fluorophenyl | CH═CH—CMe═CH | | Me | CO₂H |
| 119-108 | 4-chloro-3-fluorophenyl | CH═CH—CMe═CH | | Me | CO₂Me |
| 119-109 | 4-chloro-3-fluorophenyl | CH═CMe—CMe═CH | | H | CO₂H |
| 119-110 | 4-chloro-3-fluorophenyl | CH═CMe—CMe═CH | | H | CO₂Me |
| 119-111 | 4-chloro-3-fluorophenyl | CH═CMe—CMe═CH | | Me | CO₂H |
| 119-112 | 4-chloro-3-fluorophenyl | CH═CMe—CMe═CH | | Me | CO₂Me |
| 119-113 | 4-chloro-3-fluorophenyl | CMe═CMe—CH═CH | | H | CO₂H |
| 119-114 | 4-chloro-3-fluorophenyl | CMe═CMe—CH═CH | | H | CO₂Me |
| 119-115 | 4-chloro-3-fluorophenyl | CMe═CMe—CH═CH | | Me | CO₂H |
| 119-116 | 4-chloro-3-fluorophenyl | CMe═CMe—CH═CH | | Me | CO₂Me |
| 119-117 | 4-chloro-3-fluorophenyl | CH═CH—CMe═CMe | | H | CO₂H |
| 119-118 | 4-chloro-3-fluorophenyl | CH═CH—CMe═CMe | | H | CO₂Me |
| 119-119 | 4-chloro-3-fluorophenyl | CH═CH—CMe═CMe | | Me | CO₂H |
| 119-120 | 4-chloro-3-fluorophenyl | CH═CH—CMe═CMe | | Me | CO₂Me |
| 119-121 | 4-chloro-3-fluorophenyl | CH═CMe—CCl═CH | | H | CO₂H |
| 119-122 | 4-chloro-3-fluorophenyl | CH═CMe—CCl═CH | | H | CO₂Me |
| 119-123 | 4-chloro-3-fluorophenyl | CH═CMe—CCl═CH | | Me | CO₂H |
| 119-124 | 4-chloro-3-fluorophenyl | CH═CMe—CCl═CH | | Me | CO₂Me |
| 119-125 | 4-chloro-3-fluorophenyl | CH═CCl—CMe═CH | | H | CO₂H |
| 119-126 | 4-chloro-3-fluorophenyl | CH═CCl—CMe═CH | | H | CO₂Me |
| 119-127 | 4-chloro-3-fluorophenyl | CH═CCl—CMe═CH | | Me | CO₂H |
| 119-128 | 4-chloro-3-fluorophenyl | CH═CCl—CMe═CH | | Me | CO₂Me |
| 119-129 | 4-chloro-3-fluorophenyl | CH═CCl—CCl═CH | | H | CO₂H |
| 119-130 | 4-chloro-3-fluorophenyl | CH═CCl—CCl═CH | | H | CO₂Me |
| 119-131 | 4-chloro-3-fluorophenyl | CH═CCl—CCl═CH | | Me | CO₂H |
| 119-132 | 4-chloro-3-fluorophenyl | CH═CCl—CCl═CH | | Me | CO₂Me |
| 119-133 | 4-chloro-3-fluorophenyl | C(NO₂)═CH—CH═CH | | H | CO₂H |
| 119-134 | 4-chloro-3-fluorophenyl | C(NO₂)═CH—CH═CH | | H | CO₂Me |
| 119-135 | 4-chloro-3-fluorophenyl | C(NO₂)═CH—CH═CH | | Me | CO₂H |
| 119-136 | 4-chloro-3-fluorophenyl | C(NO₂)═CH—CH═CH | | Me | CO₂Me |
| 119-137 | 4-chloro-3-fluorophenyl | CH═CH—CH═C(NO₂) | | H | CO₂H |
| 119-138 | 4-chloro-3-fluorophenyl | CH═CH—CH═C(NO₂) | | H | CO₂Me |
| 119-139 | 4-chloro-3-fluorophenyl | CH═CH—CH═C(NO₂) | | Me | CO₂H |
| 119-140 | 4-chloro-3-fluorophenyl | CH═CH—CH═C(NO₂) | | Me | CO₂Me |
| 119-141 | 4-chloro-2-fluoro-3- | H | H | H | CO₂H |
| | methoxyphenyl | | | | |
| 119-142 | 4-chloro-2-fluoro-3- | H | H | H | CO₂Me |
| | methoxyphenyl | | | | |
| 119-143 | 4-chloro-2-fluoro-3- | H | H | Me | CO₂H |
| | methoxyphenyl | | | | |
| 119-144 | 4-chloro-2-fluoro-3- | H | H | Me | CO₂Me |
| | methoxyphenyl | | | | |
| 119-145 | 4-chloro-2-fluoro-3- | H | H | i-Pr | CO₂H |
| | methoxyphenyl | | | | |
| 119-146 | 4-chloro-2-fluoro-3- | H | H | i-Pr | CO₂Me |
| | methoxyphenyl | | | | |
| 119-147 | 4-chloro-2-fluoro-3- | H | H | CH₂Ph | CO₂H |
| | methoxyphenyl | | | | |
| 119-148 | 4-chloro-2-fluoro-3- | H | H | CH₂Ph | CO₂Me |
| | methoxyphenyl | | | | |
| 119-149 | 4-chloro-2-fluoro-3- | H | H | Ph | CO₂H |
| | methoxyphenyl | | | | |
| 119-150 | 4-chloro-2-fluoro-3- | H | H | Ph | CO₂Me |
| | methoxyphenyl | | | | |
| 119-151 | 4-chloro-2-fluoro-3- | H | Me | H | CO₂H |
| | methoxyphenyl | | | | |
| 119-152 | 4-chloro-2-fluoro-3- | H | Me | H | CO₂Me |
| | methoxyphenyl | | | | |
| 119-153 | 4-chloro-2-fluoro-3- | H | Me | Me | CO₂H |
| | methoxyphenyl | | | | |
| 119-154 | 4-chloro-2-fluoro-3- | H | Me | Me | CO₂Me |
| | methoxyphenyl | | | | |
| 119-155 | 4-chloro-2-fluoro-3- | Me | H | H | CO₂H |
| | methoxyphenyl | | | | |
| 119-156 | 4-chloro-2-fluoro-3- | Me | H | H | CO₂Me |
| | methoxyphenyl | | | | |
| 119-157 | 4-chloro-2-fluoro-3- | Me | H | Me | CO₂H |
| | methoxyphenyl | | | | |
| 119-158 | 4-chloro-2-fluoro-3- | Me | H | Me | CO₂Me |
| | methoxyphenyl | | | | |
| 119-159 | 4-chloro-2-fluoro-3- | Me | Me | H | CO₂H |
| | methoxyphenyl | | | | |
| 119-160 | 4-chloro-2-fluoro-3- | Me | Me | H | CO₂Me |
| | methoxyphenyl | | | | |
| 119-161 | 4-chloro-2-fluoro-3- | Me | Me | Me | CO₂H |
| | methoxyphenyl | | | | |
| 119-162 | 4-chloro-2-fluoro-3- | Me | Me | Me | CO₂Me |
| | methoxyphenyl | | | | |
| 119-163 | 4-chloro-2-fluoro-3- | (CH₂)₄ | | H | CO₂H |
| | methoxyphenyl | | | | |
| 119-164 | 4-chloro-2-fluoro-3- | (CH₂)₄ | | H | CO₂Me |
| | methoxyphenyl | | | | |
| 119-165 | 4-chloro-2-fluoro-3- | (CH₂)₄ | | Me | CO₂H |
| | methoxyphenyl | | | | |
| 119-166 | 4-chloro-2-fluoro-3- | (CH₂)₄ | | Me | CO₂Me |
| | methoxyphenyl | | | | |
| 119-167 | 4-chloro-2-fluoro-3- | CH═CH—CH═CH | | H | CO₂H |
| | methoxyphenyl | | | | |
| 119-168 | 4-chloro-2-fluoro-3- | CH═CH—CH═CH | | H | CO₂Me |
| | methoxyphenyl | | | | |
| 119-169 | 4-chloro-2-fluoro-3- | CH═CH—CH═CH | | Me | CO₂H |
| | methoxyphenyl | | | | |
| 119-170 | 4-chloro-2-fluoro-3- | CH═CH—CH═CH | | Me | CO₂Me |
| | methoxyphenyl | | | | |
| 119-171 | 4-chloro-2-fluoro-3- | CH═CMe—CH═CH | | H | CO₂H |
| | methoxyphenyl | | | | |
| 119-172 | 4-chloro-2-fluoro-3- | CH═CMe—CH═CH | | H | CO₂Me |
| | methoxyphenyl | | | | |
| 119-173 | 4-chloro-2-fluoro-3- | CH═CMe—CH═CH | | Me | CO₂H |
| | methoxyphenyl | | | | |
| 119-174 | 4-chloro-2-fluoro-3- | CH═CMe—CH═CH | | Me | CO₂Me |
| | methoxyphenyl | | | | |
| 119-175 | 4-chloro-2-fluoro-3- | CH═CH—CMe═CH | | H | CO₂H |
| | methoxyphenyl | | | | |
| 119-176 | 4-chloro-2-fluoro-3- | CH═CH—CMe═CH | | H | CO₂Me |
| | methoxyphenyl | | | | |
| 119-177 | 4-chloro-2-fluoro-3- | CH═CH—CMe═CH | | Me | CO₂H |
| | methoxyphenyl | | | | |
| 119-178 | 4-chloro-2-fluoro-3- | CH═CH—CMe═CH | | Me | CO₂Me |
| | methoxyphenyl | | | | |
| 119-179 | 4-chloro-2-fluoro-3- | CH═CMe—CMe═CH | | H | CO₂H |
| | methoxyphenyl | | | | |
| 119-180 | 4-chloro-2-fluoro-3- | CH═CMe—CMe═CH | | H | CO₂Me |
| | methoxyphenyl | | | | |
| 119-181 | 4-chloro-2-fluoro-3- | CH═CMe—CMe═CH | | Me | CO₂H |
| | methoxyphenyl | | | | |
| 119-82 | 4-chloro-2-fluoro-3- | CH═CMe—CMe═CH | | Me | CO₂Me |
| | methoxyphenyl | | | | |
| 119-183 | 4-chloro-2-fluoro-3- | CMe═CMe—CH═CH | | H | CO₂H |
| | methoxyphenyl | | | | |
| 119-184 | 4-chloro-2-fluoro-3- | CMe═CMe—CH═CH | | H | CO₂Me |
| | methoxyphenyl | | | | |
| 119-185 | 4-chloro-2-fluoro-3- | CMe═CMe—CH═CH | | Me | CO₂H |
| | methoxyphenyl | | | | |
| 119-186 | 4-chloro-2-fluoro-3- | CMe═CMe—CH═CH | | Me | CO₂Me |
| | methoxyphenyl | | | | |
| 119-187 | 4-chloro-2-fluoro-3- | CH═CH—CMe═CMe | | H | CO₂H |
| | methoxyphenyl | | | | |
| 119-188 | 4-chloro-2-fluoro-3- | CH═CH—CMe═CMe | | H | CO₂Me |
| | methoxyphenyl | | | | |
| 119-189 | 4-chloro-2-fluoro-3- | CH═CH—CMe═CMe | | Me | CO₂H |
| | methoxyphenyl | | | | |
| 119-190 | 4-chloro-2-fluoro-3- | CH═CH—CMe═CMe | | Me | CO₂Me |
| | methoxyphenyl | | | | |
| 119-191 | 4-chloro-2-fluoro-3- | CH═CMe—CCl═CH | | H | CO₂H |
| | methoxyphenyl | | | | |
| 119-192 | 4-chloro-2-fluoro-3- | CH═CMe—CCl═CH | | H | CO₂Me |
| | methoxyphenyl | | | | |
| 119-193 | 4-chloro-2-fluoro-3- | CH═CMe—CCl═CH | | Me | CO₂H |
| | methoxyphenyl | | | | |
| 119-194 | 4-chloro-2-fluoro-3- | CH═CMe—CCl═CH | | Me | CO₂Me |
| | methoxyphenyl | | | | |
| 119-195 | 4-chloro-2-fluoro-3- | CH═CCl—CMe═CH | | H | CO₂H |
| | methoxyphenyl | | | | |
| 119-196 | 4-chloro-2-fluoro-3- | CH═CCl—CMe═CH | | H | CO₂Me |
| | methoxyphenyl | | | | |
| 119-197 | 4-chloro-2-fluoro-3- | CH═CCl—CMe═CH | | Me | CO₂H |
| | methoxyphenyl | | | | |
| 119-198 | 4-chloro-2-fluoro-3- | CH═CCl—CMe═CH | | Me | CO₂Me |
| | methoxyphenyl | | | | |
| 119-199 | 4-chloro-2-fluoro-3- | CH═CCl—CCl═CH | | H | CO₂H |
| | methoxyphenyl | | | | |
| 119-200 | 4-chloro-2-fluoro-3- | CH═CCl—CCl═CH | | H | CO₂Me |
| | methoxyphenyl | | | | |
| 119-201 | 4-chloro-2-fluoro-3- | CH═CCl—CCl═CH | | Me | CO₂H |
| | methoxyphenyl | | | | |
| 119-202 | 4-chloro-2-fluoro-3- | CH═CCl—CCl═CH | | Me | CO₂Me |
| | methoxyphenyl | | | | |
| 119-203 | 4-chloro-2-fluoro-3- | C(NO₂)═CH—CH═CH | | H | CO₂H |
| | methoxyphenyl | | | | |
| 119-204 | 4-chloro-2-fluoro-3- | C(NO₂)═CH—CH═CH | | H | CO₂Me |
| | methoxyphenyl | | | | |
| 119-205 | 4-chloro-2-fluoro-3- | C(NO₂)═CH—CH═CH | | Me | CO₂H |
| | methoxyphenyl | | | | |
| 119-206 | 4-chloro-2-fluoro-3- | C(NO₂)═CH—CH═CH | | Me | CO₂Me |
| | methoxyphenyl | | | | |
| 119-207 | 4-chloro-2-fluoro-3- | CH═CH—CH═C(NO₂) | | H | CO₂H |
| | methoxyphenyl | | | | |
| 119-208 | 4-chloro-2-fluoro-3- | CH═CH—CH═C(NO₂) | | H | CO₂Me |
| | methoxyphenyl | | | | |
| 119-209 | 4-chloro-2-fluoro-3- | CH═CH—CH═C(NO₂) | | Me | CO₂H |
| | methoxyphenyl | | | | |
| 119-210 | 4-chloro-2-fluoro-3- | CH═CH—CH═C(NO₂) | | Me | CO₂Me |
| | methoxyphenyl | | | | |

210 compounds are described, designated compounds 120-1 to 120-210 respectively, of formula (1H) wherein D is N and X is NMe, and the values of A, R⁵, R⁸, R^{8′} and Z are as defined in Table 8.

210 compounds are described, designated compounds 121-1 to 121-210 respectively, of formula (1H) wherein D is CH and X is NH, and the values of A, R⁵, R⁸, R^{8′} and Z are as defined in Table 8.

210 compounds are described, designated compounds 122-1 to 122-210 respectively, of formula (1H) wherein D is CH and X is NMe, and the values of A, R⁵, R⁸, R^{8′} and Z are as defined in Table 8.

Table 9 below provides 72 compounds designated compounds 123-1 to 123-72 respectively, of formula (1J) wherein D is N.

TABLE 9

| | |
|---|---|
| | (1J) |
| 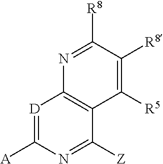 | |

| Compound | Substituent Values | | | | |
|---|---|---|---|---|---|
| Number | A | R⁸ | R^{8′} | R⁵ | Z |
| 123-1 | cyclopropyl | H | H | H | CO₂H |
| 123-2 | cyclopropyl | H | H | H | CO₂Me |
| 123-3 | cyclopropyl | H | H | Me | CO₂H |
| 123-4 | cyclopropyl | H | H | Me | CO₂Me |
| 123-5 | cyclopropyl | H | Me | H | CO₂H |
| 123-6 | cyclopropyl | H | Me | H | CO₂Me |
| 123-7 | cyclopropyl | H | Me | Me | CO₂H |
| 123-8 | cyclopropyl | H | Me | Me | CO₂Me |
| 123-9 | cyclopropyl | Me | H | H | CO₂H |
| 123-10 | cyclopropyl | Me | H | H | CO₂Me |
| 123-11 | cyclopropyl | Me | H | Me | CO₂H |
| 123-12 | cyclopropyl | Me | H | Me | CO₂Me |
| 123-13 | cyclopropyl | Me | Me | H | CO₂H |
| 123-14 | cyclopropyl | Me | Me | H | CO₂Me |
| 123-15 | cyclopropyl | Me | Me | Me | CO₂H |
| 123-16 | cyclopropyl | Me | Me | Me | CO₂Me |
| 123-17 | cyclopropyl | (CH₂)₄ | | H | CO₂H |
| 123-18 | cyclopropyl | (CH₂)₄ | | H | CO₂Me |
| 123-19 | cyclopropyl | (CH₂)₄ | | Me | CO₂H |
| 123-20 | cyclopropyl | (CH₂)₄ | | Me | CO₂Me |
| 123-21 | cyclopropyl | CH═CH—CH═CH | | H | CO₂H |
| 123-22 | cyclopropyl | CH═CH—CH═CH | | H | CO₂Me |
| 123-23 | cyclopropyl | CH═CH—CH═CH | | Me | CO₂H |
| 123-24 | cyclopropyl | CH═CH—CH═CH | | Me | CO₂Me |
| 123-25 | 4-chloro-3-fluorophenyl | H | H | H | CO₂H |
| 123-26 | 4-chloro-3-fluorophenyl | H | H | H | CO₂Me |
| 123-27 | 4-chloro-3-fluorophenyl | H | H | Me | CO₂H |
| 123-28 | 4-chloro-3-fluorophenyl | H | H | Me | CO₂Me |
| 123-29 | 4-chloro-3-fluorophenyl | H | Me | H | CO₂H |
| 123-30 | 4-chloro-3-fluorophenyl | H | Me | H | CO₂Me |
| 123-31 | 4-chloro-3-fluorophenyl | H | Me | Me | CO₂H |
| 123-32 | 4-chloro-3-fluorophenyl | H | Me | Me | CO₂Me |
| 123-33 | 4-chloro-3-fluorophenyl | Me | H | H | CO₂H |
| 123-34 | 4-chloro-3-fluorophenyl | Me | H | H | CO₂Me |
| 123-35 | 4-chloro-3-fluorophenyl | Me | H | Me | CO₂H |
| 123-36 | 4-chloro-3-fluorophenyl | Me | H | Me | CO₂Me |
| 123-37 | 4-chloro-3-fluorophenyl | Me | Me | H | CO₂H |
| 123-38 | 4-chloro-3-fluorophenyl | Me | Me | H | CO₂Me |
| 123-39 | 4-chloro-3-fluorophenyl | Me | Me | Me | CO₂H |
| 123-40 | 4-chloro-3-fluorophenyl | Me | Me | Me | CO2Me |
| 123-41 | 4-chloro-3-fluorophenyl | (CH₂)₄ | | H | CO₂H |
| 123-42 | 4-chloro-3-fluorophenyl | (CH₂)₄ | | H | CO₂Me |
| 123-43 | 4-chloro-3-fluorophenyl | (CH₂)₄ | | Me | CO₂H |
| 123-44 | 4-chloro-3-fluorophenyl | (CH₂)₄ | | Me | CO₂Me |
| 123-45 | 4-chloro-3-fluorophenyl | CH═CH—CH═CH | | H | CO₂H |
| 123-46 | 4-chloro-3-fluorophenyl | CH═CH—CH═CH | | H | CO₂Me |
| 123-47 | 4-chloro-3-fluorophenyl | CH═CH—CH═CH | | Me | CO₂H |
| 123-48 | 4-chloro-3-fluorophenyl | CH═CH—CH═CH | | Me | CO₂Me |
| 123-49 | 4-chloro-2-fluoro-3- | H | H | H | CO₂H |
| | methoxyphenyl | | | | |
| 123-50 | 4-chloro-2-fluoro-3- | H | H | H | CO₂Me |
| | methoxyphenyl | | | | |
| 123-51 | 4-chloro-2-fluoro-3- | H | H | Me | CO₂H |
| | methoxyphenyl | | | | |
| 123-52 | 4-chloro-2-fluoro-3- | H | H | Me | CO₂Me |
| | methoxyphenyl | | | | |
| 123-53 | 4-chloro-2-fluoro-3- | H | Me | H | CO₂H |
| | methoxyphenyl | | | | |
| 123-54 | 4-chloro-2-fluoro-3- | H | Me | H | CO₂Me |
| | methoxyphenyl | | | | |
| 123-55 | 4-chloro-2-fluoro-3- | H | Me | Me | CO₂H |
| | methoxyphenyl | | | | |
| 123-56 | 4-chloro-2-fluoro-3- | H | Me | Me | CO₂Me |
| | methoxyphenyl | | | | |
| 123-57 | 4-chloro-2-fluoro-3- | Me | H | H | CO₂H |
| | methoxyphenyl | | | | |
| 123-58 | 4-chloro-2-fluoro-3- | Me | H | H | CO₂Me |
| | methoxyphenyl | | | | |
| 123-59 | 4-chloro-2-fluoro-3- | Me | H | Me | CO₂H |
| | methoxyphenyl | | | | |
| 123-60 | 4-chloro-2-fluoro-3- | Me | H | Me | CO₂Me |
| | methoxyphenyl | | | | |
| 123-61 | 4-chloro-2-fluoro-3- | Me | Me | H | CO₂H |
| | methoxyphenyl | | | | |
| 123-62 | 4-chloro-2-fluoro-3- | Me | Me | H | CO₂Me |
| | methoxyphenyl | | | | |
| 123-63 | 4-chloro-2-fluoro-3- | Me | Me | Me | CO₂H |
| | methoxyphenyl | | | | |
| 123-64 | 4-chloro-2-fluoro-3- | Me | Me | Me | CO₂Me |
| | methoxyphenyl | | | | |

| | | | | |
|---|---|---|---|---|
| 123-65 | 4-chloro-2-fluoro-3- | (CH₂)₄ | H | CO₂H |
| | methoxyphenyl | | | |
| 123-66 | 4-chloro-2-fluoro-3- | (CH₂)₄ | H | CO₂Me |
| | methoxyphenyl | | | |
| 123-67 | 4-chloro-2-fluoro-3- | (CH₂)₄ | Me | CO₂H |
| | methoxyphenyl | | | |
| 123-68 | 4-chloro-2-fluoro-3- | (CH₂)₄ | Me | CO₂Me |
| | methoxyphenyl | | | |
| 123-69 | 4-chloro-2-fluoro-3- | CH═CH—CH═CH | H | CO₂H |
| | methoxyphenyl | | | |
| 123-70 | 4-chloro-2-fluoro-3- | CH═CH—CH═CH | H | CO₂Me |
| | methoxyphenyl | | | |
| 123-71 | 4-chloro-2-fluoro-3- | CH═CH—CH═CH | Me | CO₂H |
| | methoxyphenyl | | | |
| 123-72 | 4-chloro-2-fluoro-3- | CH═CH—CH═CH | Me | CO₂Me |
| | methoxyphenyl | | | |

72 compounds are described, designated compounds 124-1 to 124-72 respectively, of formula (1J) wherein D is CH, and the values of A, R⁵, R⁸, R^{8′} and Z are as defined in Table 9.

Table 10 below provides 96 compounds designated compounds 125-1 to 125-96 respectively, of formula (1K) wherein D is N.

TABLE 10

| | |
|---|---|
| | (1K) |
| 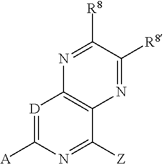 | |

| Compound | Substituent Values | | | |
|---|---|---|---|---|
| Number | A | R⁸ | R^{8′} | Z |
| 125-1 | cyclopropyl | H | H | CO₂H |
| 125-2 | cyclopropyl | H | H | CO₂Me |
| 125-3 | cyclopropyl | H | Me | CO₂H |
| 125-4 | cyclopropyl | H | Me | CO₂Me |
| 125-5 | cyclopropyl | Me | H | CO₂H |
| 125-6 | cyclopropyl | Me | H | CO₂Me |
| 125-7 | cyclopropyl | Me | Me | CO₂H |
| 125-8 | cyclopropyl | Me | Me | CO₂Me |
| 125-9 | cyclopropyl | (CH₂)₄ | | CO₂H |
| 125-10 | cyclopropyl | (CH₂)₄ | | CO₂Me |
| 125-11 | cyclopropyl | CH═CH—CH═CH | | CO₂H |
| 125-12 | cyclopropyl | CH═CH—CH═CH | | CO₂Me |
| 125-13 | cyclopropyl | CH═CMe—CH═CH | | CO₂H |
| 125-14 | cyclopropyl | CH═CMe—CH═CH | | CO₂Me |
| 125-15 | cyclopropyl | CH═CH—CMe═CH | | CO₂H |
| 125-16 | cyclopropyl | CH═CH—CMe═CH | | CO₂Me |
| 125-17 | cyclopropyl | CH═CMe—CMe═CH | | CO₂H |
| 125-18 | cyclopropyl | CH═CMe—CMe═CH | | CO₂Me |
| 125-19 | cyclopropyl | CMe═CMe—CH═CH | | CO₂H |
| 125-20 | cyclopropyl | CMe═CMe—CH═CH | | CO₂Me |
| 125-21 | cyclopropyl | CH═CH—CMe═CMe | | CO₂H |
| 125-22 | cyclopropyl | CH═CH—CMe═CMe | | CO₂Me |
| 125-23 | cyclopropyl | CH═CMe—CCl═CH | | CO₂H |
| 125-24 | cyclopropyl | CH═CMe—CCl═CH | | CO₂Me |
| 125-25 | cyclopropyl | CH═CCl—CMe═CH | | CO₂H |
| 125-26 | cyclopropyl | CH═CCl—CMe═CH | | CO₂Me |
| 125-27 | cyclopropyl | CH═CCl—CCl═CH | | CO₂H |
| 125-28 | cyclopropyl | CH═CCl—CCl═CH | | CO₂Me |
| 125-29 | cyclopropyl | C(NO₂)═CH—CH═CH | | CO₂H |
| 125-30 | cyclopropyl | C(NO₂)═CH—CH═CH | | CO₂Me |
| 125-31 | cyclopropyl | CH═CH—CH═C(NO₂) | | CO₂H |
| 125-32 | cyclopropyl | CH═CH—CH═C(NO₂) | | CO₂Me |
| 125-33 | 4-chloro-3-fluorophenyl | H | H | CO₂H |
| 125-34 | 4-chloro-3-fluorophenyl | H | H | CO₂Me |
| 125-35 | 4-chloro-3-fluorophenyl | H | Me | CO₂H |
| 125-36 | 4-chloro-3-fluorophenyl | H | Me | CO₂Me |
| 125-37 | 4-chloro-3-fluorophenyl | Me | H | CO₂H |
| 125-38 | 4-chloro-3-fluorophenyl | Me | H | CO₂Me |
| 125-39 | 4-chloro-3-fluorophenyl | Me | Me | CO₂H |
| 125-40 | 4-chloro-3-fluorophenyl | Me | Me | CO₂Me |
| 125-41 | 4-chloro-3-fluorophenyl | (CH₂)₄ | | CO₂H |
| 125-42 | 4-chloro-3-fluorophenyl | (CH₂)₄ | | CO₂Me |
| 125-43 | 4-chloro-3-fluorophenyl | CH═CH—CH═CH | | CO₂H |
| 125-44 | 4-chloro-3-fluorophenyl | CH═CH—CH═CH | | CO₂Me |
| 125-45 | 4-chloro-3-fluorophenyl | CH═CMe—CH═CH | | CO₂H |
| 125-46 | 4-chloro-3-fluorophenyl | CH═CMe—CH═CH | | CO₂Me |
| 125-47 | 4-chloro-3-fluorophenyl | CH═CH—CMe═CH | | CO₂H |
| 125-48 | 4-chloro-3-fluorophenyl | CH═CH—CMe═CH | | CO₂Me |
| 125-49 | 4-chloro-3-fluorophenyl | CH═CMe—CMe═CH | | CO₂H |
| 125-50 | 4-chloro-3-fluorophenyl | CH═CMe—CMe═CH | | CO₂Me |
| 125-51 | 4-chloro-3-fluorophenyl | CMe═CMe—CH═CH | | CO₂H |
| 125-52 | 4-chloro-3-fluorophenyl | CMe═CMe—CH═CH | | CO₂Me |
| 125-53 | 4-chloro-3-fluorophenyl | CH═CH—CMe═CMe | | CO₂H |
| 125-54 | 4-chloro-3-fluorophenyl | CH═CH—CMe═CMe | | CO₂Me |
| 125-55 | 4-chloro-3-fluorophenyl | CH═CMe—CCl═CH | | CO₂H |
| 125-56 | 4-chloro-3-fluorophenyl | CH═CMe—CCl═CH | | CO₂Me |
| 125-57 | 4-chloro-3-fluorophenyl | CH═CCl—CMe═CH | | CO₂H |
| 125-58 | 4-chloro-3-fluorophenyl | CH═CCl—CMe═CH | | CO₂Me |
| 125-59 | 4-chloro-3-fluorophenyl | CH═CCl—CCl═CH | | CO₂H |
| 125-60 | 4-chloro-3-fluorophenyl | CH═CCl—CCl═CH | | CO₂Me |
| 125-61 | 4-chloro-3-fluorophenyl | C(NO₂)═CH—CH═CH | | CO₂H |
| 125-62 | 4-chloro-3-fluorophenyl | C(NO₂)═CH—CH═CH | | CO₂Me |
| 125-63 | 4-chloro-3-fluorophenyl | CH═CH—CH═C(NO₂) | | CO₂H |
| 125-64 | 4-chloro-3-fluorophenyl | CH═CH—CH═C(NO₂) | | CO₂Me |
| 125-65 | 4-chloro-2-fluoro-3- | H | H | CO₂H |
| | methoxyphenyl | | | |
| 125-66 | 4-chloro-2-fluoro-3- | H | H | CO₂Me |
| | methoxyphenyl | | | |
| 125-67 | 4-chloro-2-fluoro-3- | H | Me | CO₂H |
| | methoxyphenyl | | | |
| 125-68 | 4-chloro-2-fluoro-3- | H | Me | CO₂Me |
| | methoxyphenyl | | | |
| 125-69 | 4-chloro-2-fluoro-3- | Me | H | CO₂H |
| | methoxyphenyl | | | |
| 125-70 | 4-chloro-2-fluoro-3- | Me | H | CO₂Me |
| | methoxyphenyl | | | |
| 125-71 | 4-chloro-2-fluoro-3- | Me | Me | CO₂H |
| | methoxyphenyl | | | |
| 125-72 | 4-chloro-2-fluoro-3- | Me | Me | CO₂Me |
| | methoxyphenyl | | | |

| | | | |
|---|---|---|---|
| 125-73 | 4-chloro-2-fluoro-3- | (CH₂)₄ | CO₂H |
| | methoxyphenyl | | |
| 125-74 | 4-chloro-2-fluoro-3- | (CH₂)₄ | CO₂Me |
| | methoxyphenyl | | |
| 125-75 | 4-chloro-2-fluoro-3- | CH═CH—CH═CH | CO₂H |
| | methoxyphenyl | | |
| 125-76 | 4-chloro-2-fluoro-3- | CH═CH—CH═CH | CO₂Me |
| | methoxyphenyl | | |
| 125-77 | 4-chloro-2-fluoro-3- | CH═CMe—CH═CH | CO₂H |
| | methoxyphenyl | | |
| 125-78 | 4-chloro-2-fluoro-3- | CH═CMe—CH═CH | CO₂Me |
| | methoxyphenyl | | |
| 125-79 | 4-chloro-2-fluoro-3- | CH═CH—CMe═CH | CO₂H |
| | methoxyphenyl | | |
| 125-80 | 4-chloro-2-fluoro-3- | CH═CH—CMe═CH | CO₂Me |
| | methoxyphenyl | | |
| 125-81 | 4-chloro-2-fluoro-3- | CH═CMe—CMe═CH | CO₂H |
| | methoxyphenyl | | |
| 125-82 | 4-chloro-2-fluoro-3- | CH═CMe—CMe═CH | CO₂Me |
| | methoxyphenyl | | |
| 125-83 | 4-chloro-2-fluoro-3- | CMe═CMe—CH═CH | CO₂H |
| | methoxyphenyl | | |
| 125-84 | 4-chloro-2-fluoro-3- | CMe═CMe—CH═CH | CO₂Me |
| | methoxyphenyl | | |
| 125-85 | 4-chloro-2-fluoro-3- | CH═CH—CMe═CMe | CO₂H |
| | methoxyphenyl | | |
| 125-86 | 4-chloro-2-fluoro-3- | CH═CH—CMe═CMe | CO₂Me |
| | methoxyphenyl | | |
| 125-87 | 4-chloro-2-fluoro-3- | CH═CMe—CCl═CH | CO₂H |
| | methoxyphenyl | | |
| 125-88 | 4-chloro-2-fluoro-3- | CH═CMe—CCl═CH | CO₂Me |
| | methoxyphenyl | | |
| 125-89 | 4-chloro-2-fluoro-3- | CH═CCl—CMe═CH | CO₂H |
| | methoxyphenyl | | |
| 125-90 | 4-chloro-2-fluoro-3- | CH═CCl—CMe═CH | CO₂Me |
| | methoxyphenyl | | |
| 125-91 | 4-chloro-2-fluoro-3- | CH═CCl—CCl═CH | CO₂H |
| | methoxyphenyl | | |
| 125-92 | 4-chloro-2-fluoro-3- | CH═CCl—CCl═CH | CO₂Me |
| | methoxyphenyl | | |
| 125-93 | 4-chloro-2-fluoro-3- | C(NO)₂═CH—CH═CH | CO₂H |
| | methoxyphenyl | | |
| 125-94 | 4-chloro-2-fluoro-3- | C(NO₂)═CH—CH═CH | CO₂Me |
| | methoxyphenyl | | |
| 125-95 | 4-chloro-2-fluoro-3- | CH═CH—CH═C(NO₂) | CO₂H |
| | methoxyphenyl | | |
| 125-96 | 4-chloro-2-fluoro-3- | CH═CH—CH═C(NO₂) | CO₂Me |
| | methoxyphenyl | | |

96 compounds are described, designated compounds 126-1 to 126-96 respectively, of formula (1K) wherein D is CH, and the values of A, R⁸, R^{8′} and Z are as defined in Table 10.

Table 11 below provides 36 compounds designated compounds 127-1 to 127-36 respectively, of formula (1L) wherein D is N and X is NH.

TABLE 11

| | |
|---|---|
| | (1L) |
| 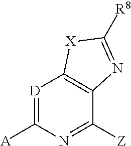 | |

| Com- | | | |
|---|---|---|---|
| pound | Substituent Values | | |
| Number | A | R⁸ | Z |
| 127-1 | Cl | H | CO₂H |
| 127-2 | Cl | H | CO₂Me |
| 127-3 | Cl | H | CO₂ⁿPr |
| 127-4 | Cl | Me | CO₂H |
| 127-5 | Cl | Me | CO₂Me |
| 127-6 | Cl | Me | CO₂ⁿPr |
| 127-7 | cyclopropyl | H | CO₂H |
| 127-8 | cyclopropyl | H | CO₂Me |
| 127-9 | cyclopropyl | H | CO₂ⁿPr |
| 127-10 | cyclopropyl | Me | CO₂H |
| 127-11 | cyclopropyl | Me | CO₂Me |
| 127-12 | cyclopropyl | Me | CO₂ⁿPr |
| 127-13 | 4-chlorophenyl | H | CO₂H |
| 127-14 | 4-chlorophenyl | H | CO₂Me |
| 127-15 | 4-chlorophenyl | H | CO₂ⁿPr |
| 127-16 | 4-chlorophenyl | Me | CO₂H |
| 127-17 | 4-chlorophenyl | Me | CO₂Me |
| 127-18 | 4-chlorophenyl | Me | CO₂ⁿPr |
| 127-19 | 4-chloro-3-fluorophenyl | H | CO₂H |
| 127-20 | 4-chloro-3-fluorophenyl | H | CO₂Me |
| 127-21 | 4-chloro-3-fluorophenyl | H | CO₂ⁿPr |
| 127-22 | 4-chloro-3-fluorophenyl | Me | CO₂H |
| 127-23 | 4-chloro-3-fluorophenyl | Me | CO₂Me |
| 127-24 | 4-chloro-3-fluorophenyl | Me | CO₂ⁿPr |
| 127-25 | 4-chloro-2-fluoro-3-methoxyphenyl | H | CO₂H |
| 127-26 | 4-chloro-2-fluoro-3-methoxyphenyl | H | CO₂Me |
| 127-27 | 4-chloro-2-fluoro-3-methoxyphenyl | H | CO₂ⁿPr |
| 127-28 | 4-chloro-2-fluoro-3-methoxyphenyl | Me | CO₂H |
| 127-29 | 4-chloro-2-fluoro-3-methoxyphenyl | Me | CO₂Me |
| 127-30 | 4-chloro-2-fluoro-3-methoxyphenyl | Me | CO₂ⁿPr |
| 127-31 | 4-chloro-3-dimethylamino-2-fluorophenyl | H | CO₂H |
| 127-32 | 4-chloro-3-dimethylamino-2-fluorophenyl | H | CO₂Me |
| 127-33 | 4-chloro-3-dimethylamino-2-fluorophenyl | H | CO₂ⁿPr |
| 127-34 | 4-chloro-3-dimethylamino-2-fluorophenyl | Me | CO₂H |
| 127-35 | 4-chloro-3-dimethylamino-2-fluorophenyl | Me | CO₂Me |
| 127-36 | 4-chloro-3-dimethylamino-2-fluorophenyl | Me | CO₂ⁿPr |

36 compounds are described, designated compounds 128-1 to 128-36 respectively, of formula (1L) wherein D is N and X is NMe, and the values of A, R⁸ and Z are as defined in Table 11.

36 compounds are described, designated compounds 129-1 to 129-36 respectively, of formula (1L) wherein D is N and X is NCH₂Ph, and the values of A, R⁸ and Z are as defined in Table 11.

36 compounds are described, designated compounds 130-1 to 130-36 respectively, of formula (1L) wherein D is N and X is NCH₂(2-nitrophenyl), and the values of A, R⁸ and Z are as defined in Table 11.

36 compounds are described, designated compounds 131-1 to 131-36 respectively, of formula (1L) wherein D is N and X is NCH₂(2,4-dimethoxyphenyl), and the values of A, R⁸ and Z are as defined in Table 11.

36 compounds are described, designated compounds 132-1 to 132-36 respectively, of formula (1L) wherein D is N and X is NCH₂(2-furanyl), and the values of A, R⁸ and Z are as defined in Table 11.

36 compounds are described, designated compounds 133-1 to 133-36 respectively, of formula (1L) wherein D is CH and X is NH, and the values of A, R⁸ and Z are as defined in Table 11.

36 compounds are described, designated compounds 134-1 to 134-36 respectively, of formula (1L) wherein D is CH and X is NMe, and the values of A, R⁸ and Z are as defined in Table 11.

36 compounds are described, designated compounds 135-1 to 135-36 respectively, of formula (1L) wherein D is CH and X is NCH₂Ph, and the values of A, R⁸ and Z are as defined in Table 11.

36 compounds are described, designated compounds 136-1 to 136-36 respectively, of formula (1L) wherein D is CH and X is NCH₂(2-nitrophenyl), and the values of A, R⁸ and Z are as defined in Table 11.

36 compounds are described, designated compounds 137-1 to 137-36 respectively, of formula (1L) wherein D is CH and X is NCH₂(2,4-dimethoxyphenyl), and the values of A, R⁸ and Z are as defined in Table 11.

36 compounds are described, designated compounds 138-1 to 138-36 respectively, of formula (1L) wherein D is CH and X is NCH₂(2-furanyl), and the values of A, R⁸ and Z are as defined in Table 11.

General methods for the production of compounds of formula (I) are described below. Unless otherwise stated in the text, the substituents A, D, E, R⁵, R⁶, R⁷, R⁸, X, Y and Z, and the number n are as defined hereinbefore. The abbreviation LG as used herein refers to any suitable leaving group. Preferred leaving groups are halogen, sulphonate (preferably tosylate), and sulphone groups. The groups R′ as used herein are optional substituents and are, independently of each other, alkyl or substituted alkyl groups. The abbreviation M as used herein refers to a metal or metalloid derivative. Preferred groups M are boronic acids and esters, trialkylstannanes and halomagnesium species (Grignard reagents).

Compounds of formula (I) in which Y is a carbon atom and n=1 may be prepared from compounds of formula (A) as shown in reaction scheme 1.

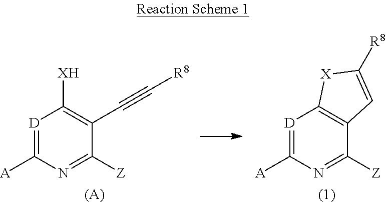

For example a compound of formula (I), in which X is an oxygen atom, may be prepared by reacting a pyridone or pyrimidone in the presence of a suitable base (for example an organic base, such as triethylamine), optionally in a suitable solvent, as described in, for example, Chem. Pharm. Bull., 1982, 30(7), 2417.

Compounds of formula (A) may be prepared from compounds of formula (B) as shown in reaction scheme 2.

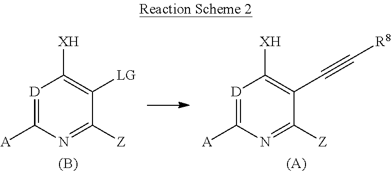

For example, a compound of formula (A) may be prepared from a compound of formula (B) in which LG is a halogen atom or sulphonate by Sonogashira reaction with an alkyne of formula (C) in the presence of a copper source (for example, a copper(I) salt, such as copper(I) iodide), a palladium catalyst (for example bis(triphenylphosphine)palladium(II) dichloride) and a suitable base (for example an organic base, such as triethylamine), optionally in a suitable solvent, as described in, for example, Chem. Pharm. Bull., 1982, 30(7), 2417; as shown in reaction scheme 3.

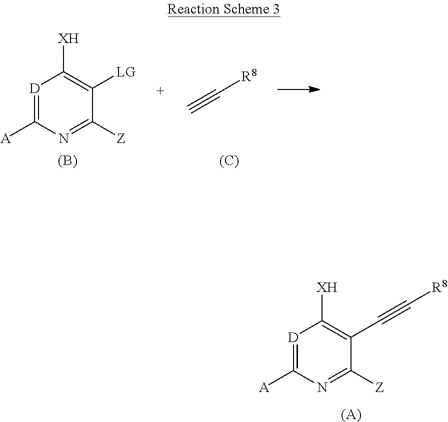

Alternatively (see reaction scheme 4) a compound of formula (A) may be prepared from a compound of formula (B) in which LG is a halogen atom or sulphonate by reaction with a suitable metal or metalloid alkyne derivative (D) (for example a boronic acid or ester, a trialkyltin derivative, a zinc derivative or a Grignard reagent) in the presence of a suitable base (for example an inorganic base, such as potassium phosphate or caesium fluoride), a metal source (for example a palladium source, such as Pd(OAc)₂) and, optionally, a ligand for the metal (for example a phosphine ligand, such as PCy₃.HBF₄) in a suitable solvent (for example a single solvent, such as dimethylformamide, or a mixed solvent system, such as a mixture of dimethoxyethane and water or toluene and water). The metal catalyst and ligands may also be added as a single, pre-formed, complex (for example a palladium/phosphine complex, such as bis(triphenylphosphine)palladium dichloride or [1,1′-bis(diphenylphosphino)ferrocene]palladium dichloride dichloromethane adduct). Such reactions are well known in the literature and are described in, for example, WO2009/046090.

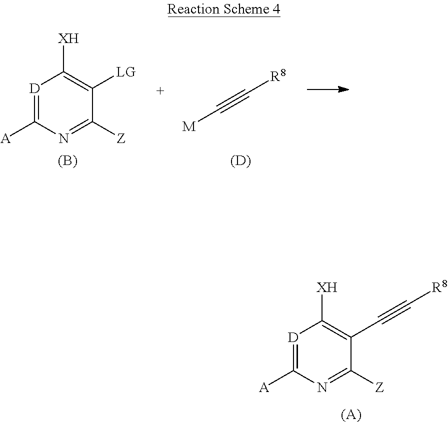

Compounds of formula (B) may be prepared as described in, for example, WO2009/081112.

Alternatively, compounds of formula (A1), which are compounds of formula A in which R⁸ is a hydrogen atom, may be prepared from compounds of formula (E) as shown in reaction scheme 5.

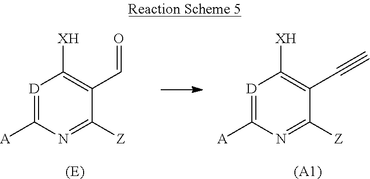

There are many ways in which this transformation may be performed known in the literature, for example as described in Synlett., 1996, 521; Tetrahedron Lett., 1972, 36, 3769; J. Org. Chem., 2000, 65, 1889.

Compounds of formula (E) may be prepared as described in, for example, WO2009/046090.

Alternatively, compounds of formula A may be prepared from compounds of formula (F) as shown in reaction scheme 6.

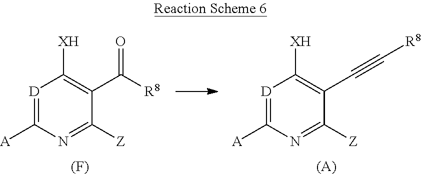

This transformation may be performed, for example, as described in J. Org. Chem., 1982, 47, 1837.

Compounds of formula (F) may be prepared as described in, for example, WO2009/046090.

Alternatively, compounds of formula (E) and formula (F) may be prepared from compounds of formula (G), as shown in reaction scheme 7.

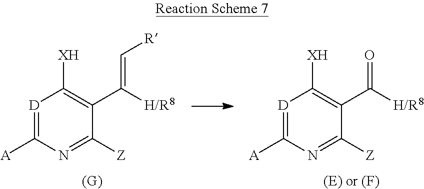

For example, a compound of formula (E) or (F) may be prepared by the reaction of a compound of formula (G) with ozone in a suitable solvent, for example dichloromethane, followed by in situ treatment of the resulting ozonide with a suitable reducing agent, for example triphenylphosphine or dimethyl sulphide.

Alternatively, a compound of formula (G) may be treated with metallic oxidising agents, for example, osmium tetroxide and sodium periodate, optionally in the presence of a further stoichiometric oxidant, for example, an amine N-oxide such as N-methylmorpholine N-oxide, to produce a compound of formula (E) or (F).

Compounds of formula (G) are described in, for example, WO2009/081112.

Compounds of formula (I) may also be prepared from compounds of formula (B), as shown in reaction scheme 8.

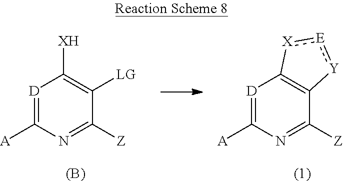

For example, a compound of formula (I) in which Y is a carbon atom and n=1 may be prepared from a compound of formula (B) by reaction with an allenyl metal or metalloid reagent (H) (for example an allenyl stannane or allenyl boronic acid) in the presence of a suitable base (for example an inorganic base, such as potassium phosphate or caesium fluoride), a metal source (for example a palladium source, such as Pd(OAc)₂) and, optionally, a ligand for the metal (for example a phosphine ligand, such as PCy₃.HBF₄) in a suitable solvent (for example a single solvent, such as dimethylformamide, or a mixed solvent system, such as a mixture of dimethoxyethane and water or toluene and water). The metal catalyst and ligands may also be added as a single, pre-formed, complex (for example a palladium/phosphine complex, such as bis(triphenylphosphine)palladium dichloride or [1,1′-bis(diphenylphosphino)ferrocene]palladium dichloride dichloromethane adduct), as shown in reaction scheme 9.

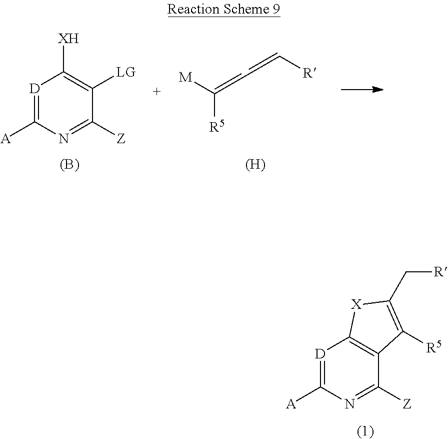

Alternatively, as shown in reaction scheme 10, a compound of formula (I) in which Y is a carbon atom and n=1 may be prepared from a compound of formula (B) in which LG is a halogen atom or sulphonate by Sonogashira reaction with an alkyne of formula (C) in the presence of a copper source (for example, a copper(I) salt, such as copper(I) iodide), a palladium catalyst (for example bis(triphenylphosphine)palladium(II) dichloride) and a suitable base (for example an organic base, such as triethylamine), optionally in a suitable solvent, as described in, for example, Chem. Pharm. Bull., 1982, 30(7), 2417.

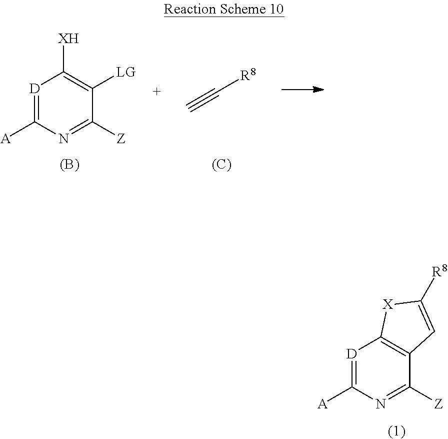

Compounds of formula (I) in which Y is carbon may also be prepared from compounds of formula (J), as shown in reaction scheme 11.

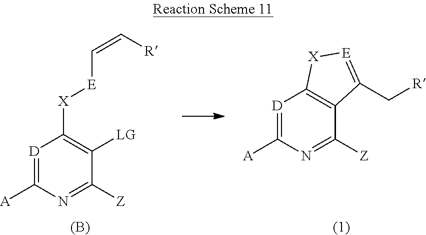

For example a compound of formula (J) may be treated with a suitable base (for example an inorganic base, such as sodium acetate), a metal source (for example a palladium source, such as Pd(OAc)₂) and, optionally, a ligand for the metal (for example a phosphine ligand, such as PCy₃.HBF₄) in a suitable solvent (for example dimethylacetamide). The metal catalyst and ligands may also be added as a single, pre-formed, complex (for example a palladium/phosphine or palladium/N-heterocyclic carbene complex, such as a PEPPSI complex). Such methods are described in, for example, J. Chem. Soc., Perkin 1, 1979, 771; Tetrahedron Lett., 1987, 28(44), 5291.

Compounds of formula (J) may be prepared from compounds of formula (K), in which LG and LG′ may be the same or different leaving groups, by reaction with a nucleophile of formula (L), optionally in the presence of a base (for example an organic base, such as triethylamine, or an inorganic base, such as potassium carbonate), in a suitable solvent, for example dichloroethane, as shown in reaction scheme 12.

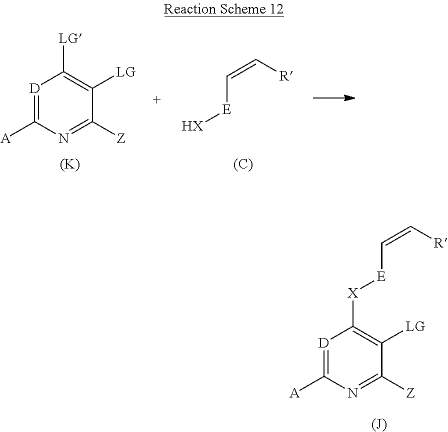

Compounds of formula (K) may be prepared using methods known in the literature, for example as described in WO2009/081112.

Alternatively, compounds of formula (J) may be prepared from compounds of formula (B) by reaction with suitable alkylating agents of formula (M), as shown in reaction scheme 13.

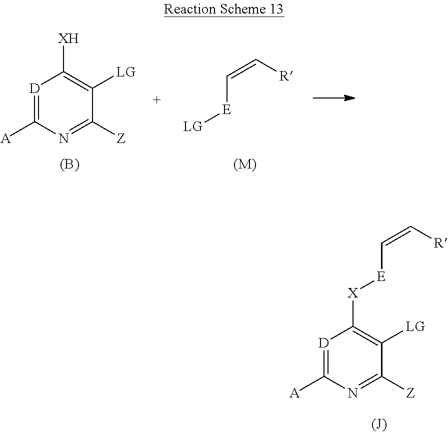

Compounds of formula (I) in which Y is carbon may also be prepared from compounds of formula (N), as shown in reaction scheme 14.

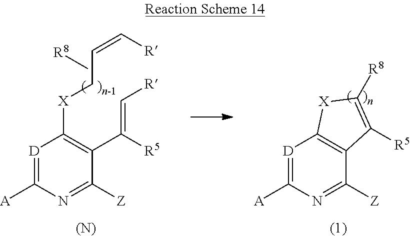

For example a compound of formula (I) may be prepared by treating a compound of formula (N) with an olefin metathesis catalyst, for example a ruthenium or molybdenum complex, such as 1,3-bis-(2,4,6-trimethylphenyl)-2-(imidazolidinylidene)(dichlorophenylmethylene)(tricyclohexylphosphine)ruthenium.

Compounds of formula (N) may be prepared from compounds of formula (J), as shown in reaction scheme 15.

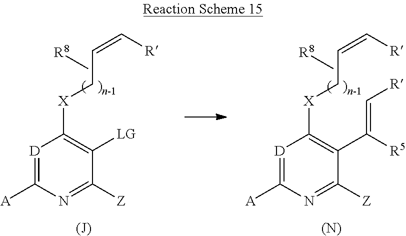

For example, such a transformation may be carried out by reaction with a metal or metalloid derivative of formula (O) (for example a boronic acid, boronate ester or stannane) in the presence of a base (for example an inorganic base, such as potassium phosphate or caesium fluoride, or an organic base, such as triethylamine), a metal source (for example, a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakis(triphenylphosphine), bis(triphenylphosphine)palladium dichloride or [1,1-bis(diphenylphosphino)ferrocene]palladium dichloride), as shown in reaction scheme 16.

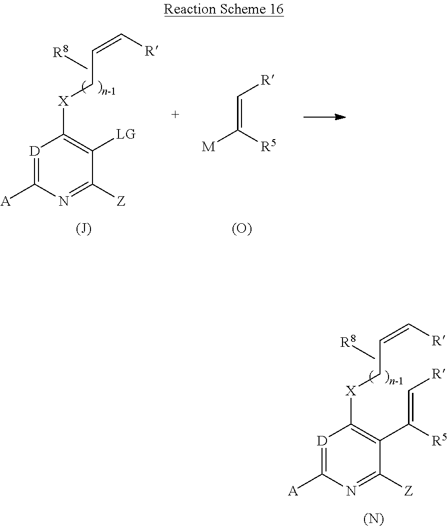

Alternatively, compounds of formula (N) may be prepared from compounds of formula (P) by reaction with a nucleophile of formula (L), optionally in the presence of a base (for example an organic base, such as triethylamine, or an inorganic base, such as potassium carbonate), in a suitable solvent, for example dichloroethane, as shown in reaction scheme 17.

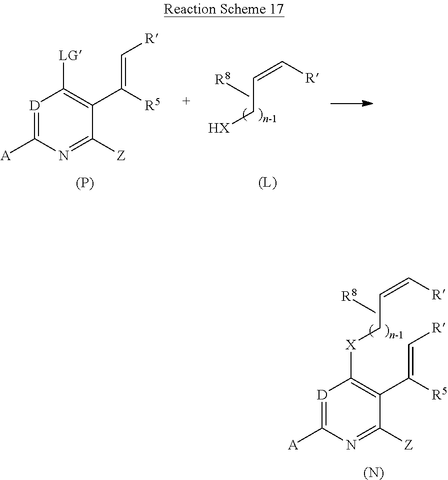

Compounds of formula (P) may be prepared from compounds of formula (K), in which LG′ is a leaving group or the precursor to a leaving group (for example an alkylthio group that can be converted into an alkylsulphonyl leaving group by oxidation), as shown in reaction scheme 18.

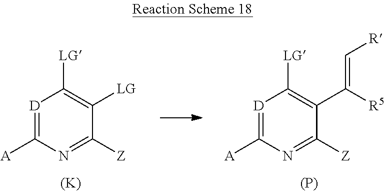

For example, such a transformation may be carried out by reaction with a metal or metalloid derivative of formula (O) (for example a boronic acid, boronate ester or stannane) in the presence of a base (for example an inorganic base, such as potassium phosphate or caesium fluoride, or an organic base, such as triethylamine), a metal source (for example, a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakis(triphenylphosphine), bis(triphenylphosphine)palladium dichloride or [1,1′-bis(diphenylphosphino)ferrocene]palladium dichloride), as shown in reaction scheme 19.

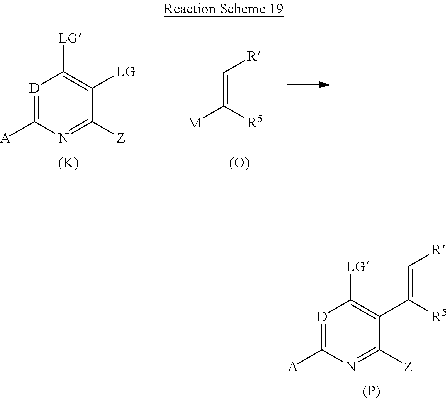

Compounds of formula (N) may also be prepared from compounds of formula (G) by reaction with suitable alkylating agents of formula (M), as shown in reaction scheme 20.

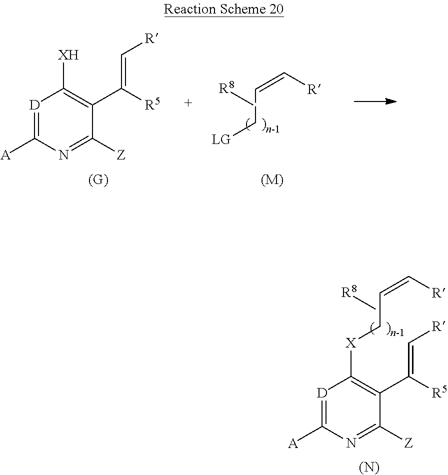

Compounds of formula (I) in which X is NH, Y is carbon and n=1 may be prepared from compounds of formula (Q), as shown in reaction scheme 21.

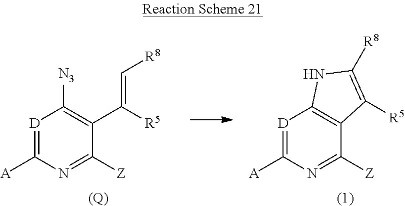

For example, an azide of formula (Q) may be heated in an inert solvent (for example a haloarene such as dibromobenzene) to produce a compound of formula (I), for example as described in Chem. Pharm. Bull., 1982, 30(7), 2417.

Alternatively an azide of formula (Q) may be converted to a compound of formula (I) by photolysis in a suitable solvent (for example, trifluoroacetic acid), for example as described in Chem. Pharm. Bull., 1989, 37(11), 2933.

Compounds of formula (Q) may be prepared from compounds of formula (P), as shown in reaction scheme 22.

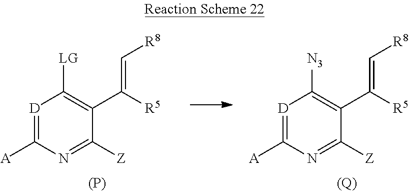

For example, a compound of formula (Q) may be prepared by treating a compound of formula (P) with a source of azide (for example an inorganic azide, such as sodium azide) in a suitable solvent (for example, ethanol or dimethylformamide).

Alternatively, compounds of formula (Q) may be prepared from compounds of formula (R), as shown in reaction scheme 23.

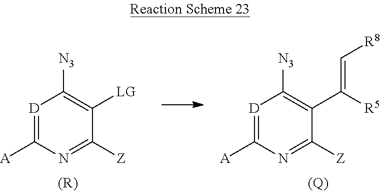

For example, such a transformation may be carried out by reaction with a metal or metalloid derivative of formula (O) (for example a boronic acid, boronate ester or stannane) in the presence of a base (for example an inorganic base, such as potassium phosphate or caesium fluoride, or an organic base, such as triethylamine), a metal source (for example, a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as bis(triphenylphosphine)palladium dichloride or [1,1′-bis(diphenylphosphino)ferrocene]palladium dichloride), as shown in reaction scheme 24.

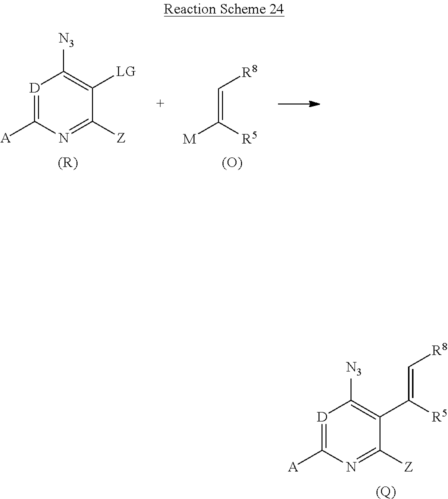

Compounds of formula (R) may be prepared from compounds of formula (K) as shown in reaction scheme 25.

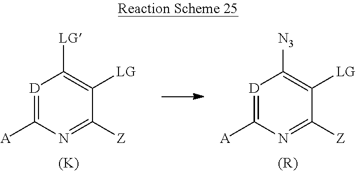

For example, a compound of formula (R) may be prepared by treating a compound of formula (K) with a source of azide (for example an inorganic azide, such as sodium azide) in a suitable solvent (for example, ethanol or dimethylformamide).

Compounds of formula (I) may be prepared from compounds of formula (S), as shown in reaction scheme 26.

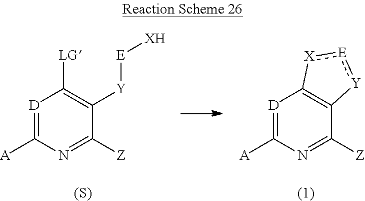

For example, a compound of formula (I) may be prepared from a compound of formula (S) by reaction with a base (for example an organic base, such as triethylamine, or an inorganic base, such as potassium carbonate), in a suitable solvent, for example dichloroethane.

As an additional example a compound of formula (I) may be prepared from a compound of formula (S) by treatment with a suitable catalyst (for example a metal catalyst, such as a palladium source) and optionally a suitable ligand (for example a phosphine ligand, such as Josiphos) in a suitable solvent.

Compounds of formula (S) may be prepared from compounds of formula (K), in which LG′ is a leaving group or the precursor to a leaving group (for example an alkylthio group that can be converted into an alkylsulphonyl leaving group by oxidation), as shown in reaction scheme 27.

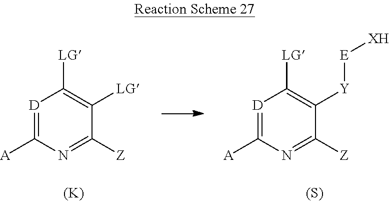

For example, a compound of formula (S) in which Y is a heteroatom may be prepared by treating a compound of formula (K) with a nucleophile of formula (T), as shown in reaction scheme 28.

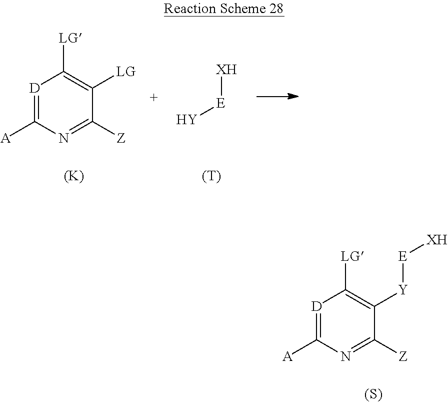

As an example a compound of formula (S) may be prepared from a compound of formula (K) and a nucleophile of formula (T) by treatment with a suitable catalyst (for example a metal catalyst, such as a palladium source) and optionally a suitable ligand (for example a phosphine ligand, such as Josiphos) in a suitable solvent.

As a further example, a compound of formula (S) in which Y is a carbon atom may be prepared by reaction of a compound of formula (K) with a metal or metalloid derivative of formula (U) (for example a boronic acid, boronate ester or stannane) in the presence of a base (for example an inorganic base, such as potassium phosphate or caesium fluoride, or an organic base, such as triethylamine), a metal source (for example, a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakis(triphenylphosphine), bis(triphenylphosphine)palladium dichloride or [1,1-bis(diphenylphosphino)ferrocene]palladium dichloride), as shown in reaction scheme 29.

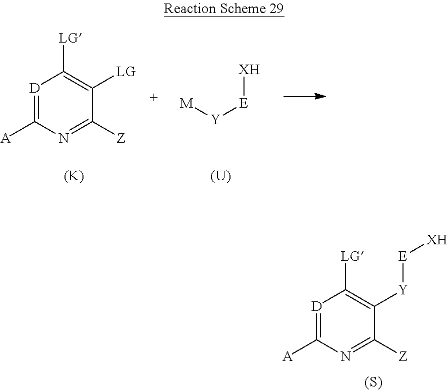

Compounds of formula (I) may be prepared from compounds of formula (V), as shown in reaction scheme 30.

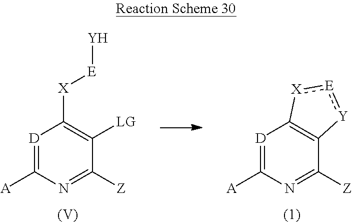

For example, a compound of formula (I) may be prepared from a compound of formula (V) by reaction with a base (for example an organic base, such as triethylamine, or an inorganic base, such as potassium carbonate), in a suitable solvent, for example dichloroethane.

As an additional example a compound of formula (I) may be prepared from a compound of formula (V) by treatment with a suitable catalyst (for example a metal catalyst, such as a palladium source) and optionally a suitable ligand (for example a phosphine ligand, such as Josiphos) in a suitable solvent.

Compounds of formula (V) may be prepared from compounds of formula (K), in which LG′ is a leaving group and may be the same as or different to LG, as shown in reaction scheme 31.

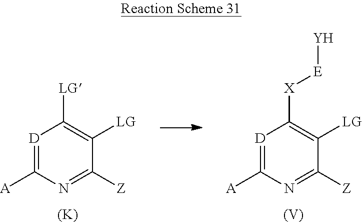

For example, a compound of formula (V) may be prepared by treating a compound of formula (K) with a nucleophile of formula (T), optionally in the presence of a base (for example an organic base, such as triethylamine, or an inorganic base, such as potassium carbonate), in a suitable solvent, for example dichloroethane, as shown in reaction scheme 32.

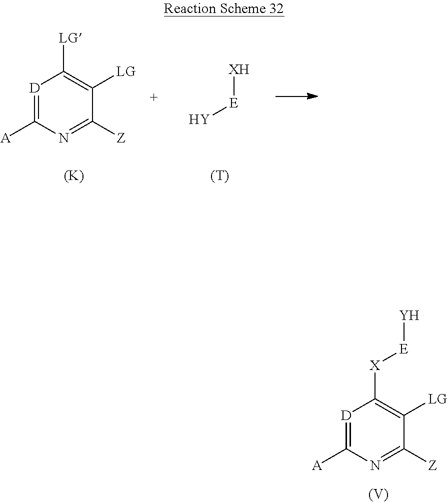

As an example a compound of formula (V) may be prepared from a compound of formula (K) and a nucleophile of formula (T) by treatment with a suitable catalyst (for example a metal catalyst, such as a palladium source) and optionally a suitable ligand (for example a phosphine ligand, such as Josiphos) in a suitable solvent.

Compounds of formula (I) may be prepared from compounds of formula (W), as shown in reaction scheme 33.

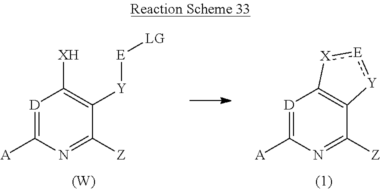

For example, a compound of formula (I) may be prepared from a compound of formula (W) by reaction with a base (for example an organic base, such as triethylamine, or an inorganic base, such as potassium carbonate), in a suitable solvent, for example dichloroethane.

Compounds of formula (W) may be prepared from compounds of formula (X), as shown in reaction scheme 34.

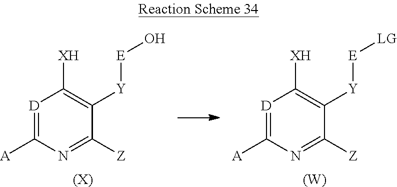

For example, a compound of formula (W) may be prepared by treating a compound of formula (X) with an activating reagent (for example, a sulphonyl chloride such as tosyl chloride or mesyl chloride) in the presence of a base (for example an organic base such as triethylamine).

Compounds of formula (X) may be prepared from compounds of formula (Y), as shown in reaction scheme 35.

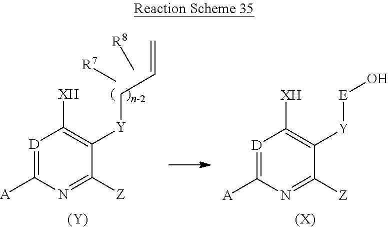

For example (as shown in reaction scheme 36) a compound of formula (X) may be prepared by hydroboration of a compound of formula (G), using conditions that are well known in the literature.

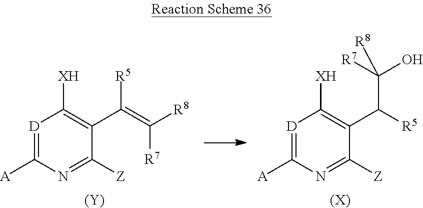

Alternatively, compounds of formula (X) may be prepared by reduction of compounds of formula (Z), as shown in reaction scheme 37.

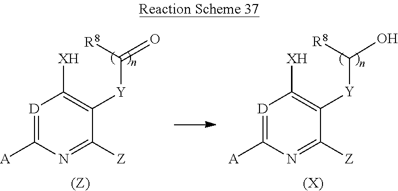

For example, this transformation may be achieved by the reaction of a compound of formula (Z) with a suitable reducing agent, for example a metal hydride such as sodium borohydride.

Compounds of formula (Z) in which Y is carbon and n=1 may be prepared from compounds of formula (A), as shown in reaction scheme 38.

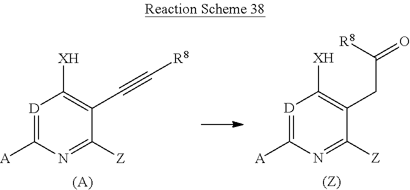

For example, a compound of formula (Z) may be prepared by treating a compound of formula (A) with a metal salt (for example a gold or mercury salt, such as mercury (II) sulphate) optionally in the presence of an acid (for example an inorganic acid such as sulphuric acid).

Compounds of formula (Z) may also be made by oxidation of compounds of formula (X), as shown in reaction scheme 39.

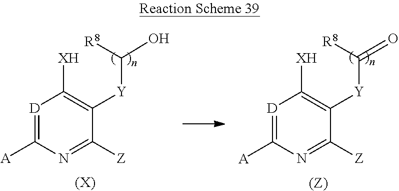

For example, a compound of formula (Z) may be prepared by treating a compound of formula (X) with an oxidising agent, using methods that are well known in the literature.

Compounds of formula (Z) in which Y is a carbon and n=1 may be prepared from compounds of formula (B) by reaction with a silyl enol ether (AA), as shown in reaction scheme 40.

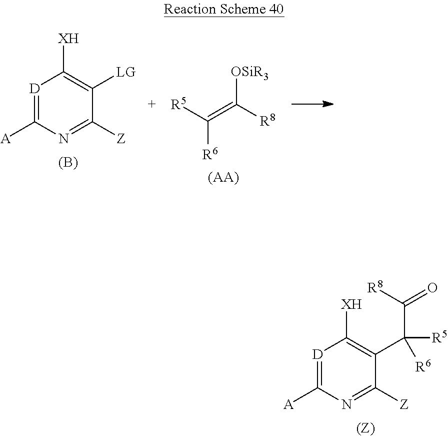

For example a compound of formula (Z) may be prepared by reacting a silyl enol ether (AA) with a compound of formula (B) in the presence of a metal source (for example a palladium source such as Pd₂(dba)₃), a second metal (for example a zinc salt, such as zinc difluoride) and, optionally, a ligand for the metal (for example a phosphine ligand, such as S-Phos) in a suitable solvent (for example dimethyl formamide), as described in, for example, Tetrahedron Lett., 2007, 48, 1213.

Compounds of formula (I) may be prepared from compounds of formula (X), as shown in reaction scheme 38.

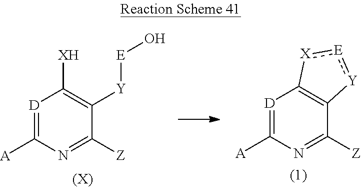

For example, a compound of formula (I) may be prepared by treating a compound of formula (X) as described in, for example, J. Het. Chem., 1996, 33, 229.

Compounds of formula (I) in which X is nitrogen may be prepared from compounds of formula (Z) in which X is nitrogen, as shown in reaction scheme 42.

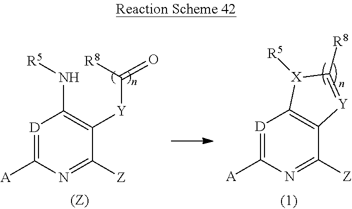

For example, a compound of formula (I) may be prepared by treating a compound of formula (Z) with an acid (for example an organic acid, such as para-toluene sulphonic acid) as described in, for example, J. Org. Chem., 2007, 72(13), 4596 and WO2004/000843.

Compounds of formula (I) may be prepared from compounds of formula (BB), as shown in reaction scheme 43.

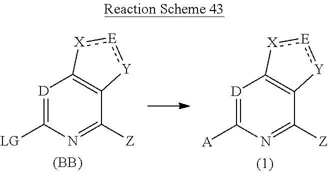

For example, a compound of formula (I), in which A is a ring linked to the bicyclic ring system through a nitrogen atom, may be prepared by reaction of a compound of formula (BB) with A-H (for example pyrrole), optionally in the presence of a suitable base (for example an amine base, such as triethylamine), in a suitable solvent (for example an alcohol, such as methanol)—see reaction scheme 44 below. The reaction may be performed at ambient temperature or preferably, at an elevated temperature. This transformation may also be performed in the presence of a suitable metal (for example palladium) catalyst, optionally complexed by any suitable ligands (for example phosphine ligands, such as Josiphos).

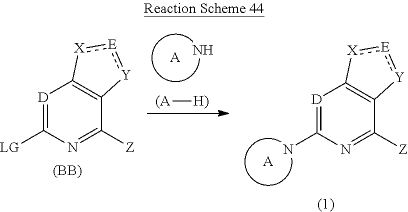

In a second example (see reaction scheme 45) a compound of formula (I), in which A is a group attached through a carbon atom, may be prepared by reacting a suitable metal or metalloid derivative A-M (for example a boronic acid or ester, a trialkyltin derivative, a zinc derivative or a Grignard reagent) with a compound of formula (BB) in the presence of a suitable base (for example an inorganic base, such as potassium phosphate or caesium fluoride, or an organic base, such as triethylamine), a metal source (for example a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakis(triphenylphosphine), bis(triphenylphosphine)palladium dichloride or [1,1′-bis(diphenylphosphino)ferrocene]palladium dichloride).

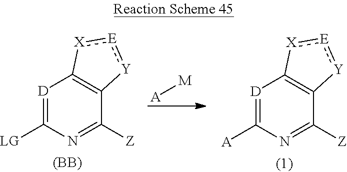

As an additional example, a compound of formula (I) in which A is an alkenyl group may be prepared using a Heck reaction in which the group A component containing the double bond may be reacted with a compound of formula (AA) in the presence of a suitable metal catalyst (for example a palladium derivative, such as palladium acetate), optionally with a suitable ligand for the metal, and a suitable base (for example an inorganic base, such as potassium phosphate) in a suitable solvent (for example N-methylpyrrolidone), as shown in reaction scheme 46.

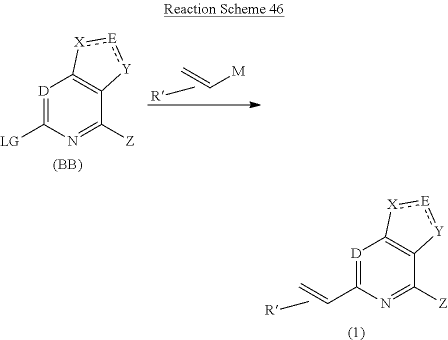

Alternatively, compounds of formula (I) may be prepared from compounds of formula (CC), wherein M represents a suitable metal or metalloid derivative (for example a boronic acid or ester, a trialkyltin group, a suitably substituted silyl group, a zinc derivative or a magnesium halide), by reaction with a compound A-LG—see reaction scheme 47 below.

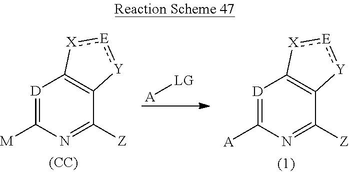

For example, a compound of formula (I) may be prepared from a compound of formula (CC) in which M is a boronic acid group by reaction with a compound A-LG in the presence of a metal catalyst (for example a palladium derivative such as Pd₂(dba)₃), optionally with a suitable ligand (for example a phosphine such as X-Phos) and a base (for example an inorganic base, such as potassium phosphate or caesium fluoride) in a suitable solvent.

Compounds of formula (CC) may be prepared from other compounds of formula (CC) using a transmetallation reaction. For example, a compound of formula (CC) wherein M is a boronic acid may be prepared from a compound of formula (CC) where M is a magnesium halide by reaction with a trialkylboronate, followed by hydrolysis (for example under acidic conditions).

Alternatively compounds of formula (CC) may be prepared from compounds of formula (BB) (reaction scheme 48).

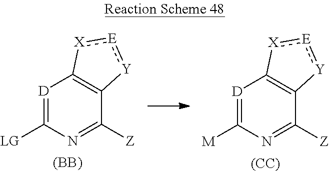

For example, a compound of formula (CC) wherein M is a boronate ester or a trialkylstannane may be prepared from a compound of formula (BB) by treating it with a suitable M-containing reagent (for example pinacolborane, bispinacolatodiboron, hexa-alkyldi-tin) in the presence of a metal catalyst (for example a palladium species, such as bis(diphenylphosphine)palladium dichloride) in a suitable solvent (for example dioxane).

Alternatively, a compound of formula (CC) where M is a magnesium halide may be prepared from a compound of formula (BB) by treatment with a suitable Grignard reagent (for example an isopropylmagnesium halide such as isopropylmagnesium chloride) in a suitable solvent.

Compounds of formula (BB) may be prepared from compounds of formula (DD), in which LG′ is a leaving group and may be the same as or different to LG, as shown in reaction scheme 49.

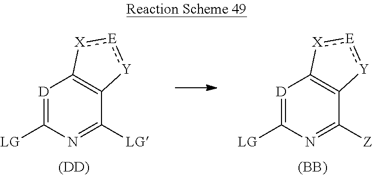

For example a compound of formula (BB) in which Z is CO₂R′ may be prepared from a compound of formula (DD) by reaction with an alcohol R′OH and carbon monoxide in the presence of a suitable metal catalyst (for example a palladium reagent, such as bis(triphenylphosphine)palladium dichloride) and a suitable base (for example an organic base, such as triethylamine), see reaction scheme 50. It may conveniently be conducted under an atmosphere of carbon monoxide gas at atmospheric or raised pressure.

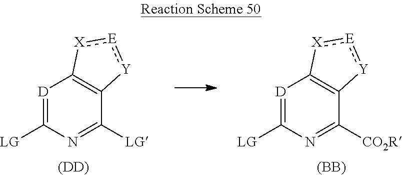

Compounds of formula (DD) in which LG and LG′ are the same, may be prepared from compounds of formula (EE) by reaction with a suitable reagent (for example a phosphoryl halide or sulphonyl anhydride) as shown in reaction scheme 51.

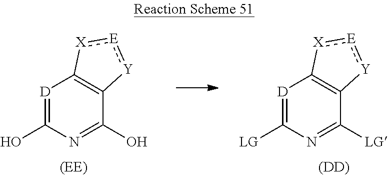

For example, a compound of formula (DD) in which LG and LG′ are halogen atoms may be prepared by reaction of a compound of formula (EE) with a halogenating agent (for example a phosphoryl halide such as phosphorus oxychloride) in the presence of a suitable base (for example an organic base, such as N,N-diethylaniline).

Compounds of formula (EE) may be prepared from compounds of formula (FF), in which G is a leaving group or an amine and J is an alkoxy or amino group, as shown in reaction scheme 52.

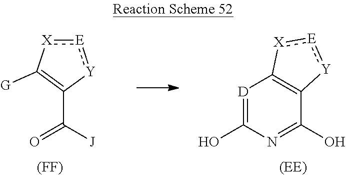

For example, as shown in reaction scheme 53, a compound of formula (EE) in which D is a nitrogen atom, may be prepared by the reaction of a compound of formula (FF) in which G and J are both NH₂, with a chloroformate in the presence of a base (for example an organic base, such as pyridine), as described in, for example, Nucleosides and Nucleotides, 1994, 13(5), 1135.

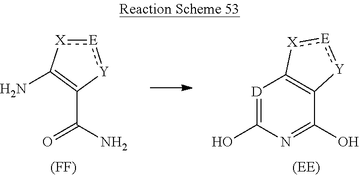

Compounds of formula (FF) are known in the literature, or may be made using procedures known in the literature.

Compounds of formula (I) may be prepared from different compounds of formula (I) by the conversion of any of the substituents A, D, E, X, Y and Z, into a different group A, D, E, X, Y and Z, using techniques well known to the skilled man.

For example, an unsaturated group A (for example an alkene or cycloalkene) may be reduced to form a saturated group (for example an alkyl or cycloalkyl group). When A is an unsaturated ring it may be oxidised to form an aromatic ring under standard conditions.

A second example is the conversion of a compound in which A is a halogen atom (for example, chlorine) into a compound in which A is a substituted phenyl ring. Such a conversion may be performed by reacting a suitable metal or metalloid derivative A-M (for example a boronic acid or ester, a trialkyltin derivative, a zinc derivative or a Grignard reagent) with a compound of formula (I) in which A=Cl in the presence of a suitable base (for example an inorganic base, such as potassium phosphate or caesium fluoride, or an organic base, such as triethylamine), a metal source (for example a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakis(triphenylphosphine), bis(triphenylphosphine)palladium dichloride or [1,1′-bis(diphenylphosphino)ferrocene]palladium dichloride).

A further example is the conversion of a compound in which D is C-Halogen (for example C—Br or C—Cl) into a compound in which D is a carbon atom attached to a carbon-based group, for example an alkyl or alkenyl group. Such a transformation may be carried out by reaction with a metal or metalloid derivative of the alkyl or alkenyl group (for example a boronic acid or boronate ester) in the presence of a base (for example an inorganic base, such as potassium phosphate or caesium fluoride, or an organic base, such as triethylamine), a metal source (for example a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example as a palladium/phosphine complex, such as palladium tetrakis(triphenylphosphine), bis(triphenylphosphine)palladium dichloride or [1,1′-bis(diphenylphosphino)ferrocene]palladium dichloride).

A further compound of formula (I) may be prepared from a compound of formula (I) in which R⁴ is H by reaction with a suitable reagent R⁴-LG in which LG is a leaving group such as a halogen atom. Examples of such reagents R⁴-LG are alkyl halides and acid anhydrides. See reaction scheme 54.

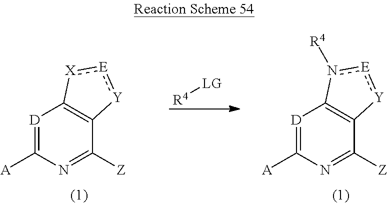

In an additional example a compound of formula (I) in which Z is a carboxylic acid may be prepared from a compound of formula (I) in which Z is a carboxylate ester, by hydrolysis under basic or acidic conditions, for example by treatment with aqueous sodium hydroxide. Alternatively this transformation may be achieved by treatment of the ester with a nucleophile (for example an alkyl thiolate) in a suitable solvent (both shown schematically in reaction scheme 55 below).

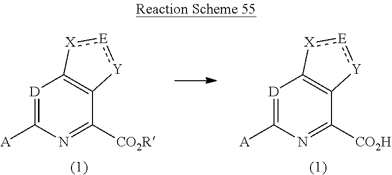

A compound of formula (I) in which Z is a carboxylate ester may be prepared directly from a compound of formula (I) in which Z is a carboxylic acid by esterification under standard conditions, for example by treatment with an alcohol R′OH and an acid catalyst (for example, thionyl chloride). Alternatively, this transformation may be achieved by first preparing an activated derivative of the acid group (for example an acyl halide) followed by reaction with an alcohol.

Other derivatives of the acid group in compounds of formula (I) in which Z is a carboxylic acid may be prepared by standard methods found in the literature. For example a compound of formula (I) in which Z is an amide group may be prepared from a compound of formula (I) in which Z is a carboxylic acid by treatment with a suitable coupling reagent (for example a carbodiimide such as dicyclohexylcarbodiimide) and an amine R′₂NH, optionally with a additive (for example dimethylaminopyridine), in a suitable solvent (for example dimethylformamide). Alternatively, this transformation may be performed by first preparing an activated derivative of the carboxylic acid group (for example an acyl halide such as an acid chloride), and then treating the activated derivative with an amine R′₂NH. Again, both transformations are shown schematically in reaction scheme 56 below.

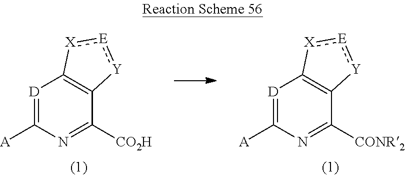

Compounds of formula (Z) in which Y is a carbon may be prepared from compounds of formula (GG) by reaction with an oxidising agent, as shown in reaction scheme 57.

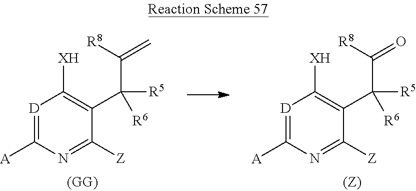

For example a compound of formula (Z) may be prepared by reacting an alkene (GG) with ozone followed by a reducing agent, for example dimethyl sulphide.

Compounds of formula (GG) may be prepared from compounds of formula (B) by reaction with an organometallic reagent (HH), as shown in reaction scheme 58.

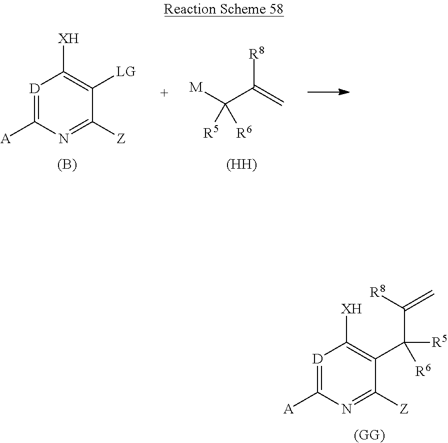

For example a compound of formula (GG) may be prepared by reacting an organometallic reagent, for example an organostannane or organoboron reagent, (HH) with a compound of formula (B) in the presence of a metal source (for example a palladium source such as Pd₂(dba)₃), and, optionally, a ligand for the metal (for example a phosphine ligand, such as S-Phos) in a suitable solvent (for example dimethyl formamide).

Compounds of formula (Z) in which Y is a carbon may also be prepared from compounds of formula (JJ) by hydrolysis, as shown in reaction scheme 59.

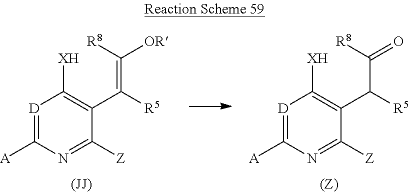

For example a compound of formula (Z) may be prepared by reacting an alkene (GG) with ozone followed by a reducing agent, for example dimethyl sulphide.

Compounds of formula (JJ) may be prepared from compounds of formula (B) by reaction with an organometallic reagent (KK), as shown in reaction scheme 60.

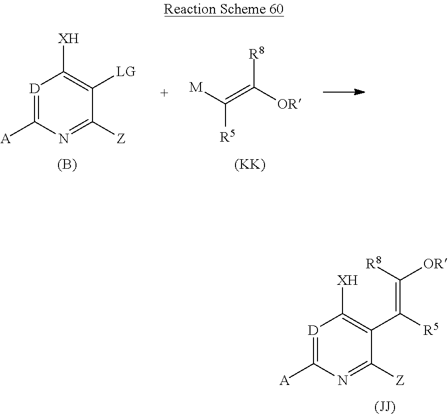

For example a compound of formula (JJ) may be prepared by reacting an organometallic reagent, for example an organostannane or organoboron reagent, (KK) with a compound of formula (B) in the presence of a metal source (for example a palladium source such as Pd₂(dba)₃), and, optionally, a ligand for the metal (for example a phosphine ligand, such as S-Phos) in a suitable solvent (for example dimethyl formamide). Compounds of formula (1) may be prepared from compounds of formula (LL) by reaction with a suitable bifunctional reagent (MM), as shown in reaction scheme 61.

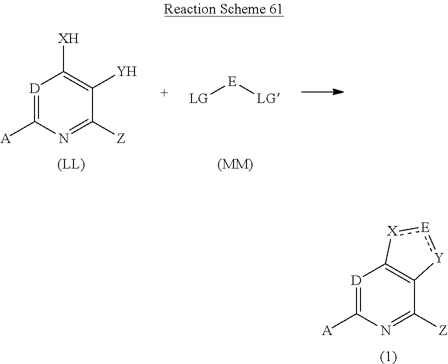

For example a compound of formula (1) may be prepared by reacting a compound of formula (LL) with an aldehyde or a bis acid chloride, optionally in the opresence of an acid, such as toluene sulphonic acid, or a base, such as triethylamine.

Compounds of formula (LL) may be prepared from compounds of formula (B) as shown in reaction scheme 62.

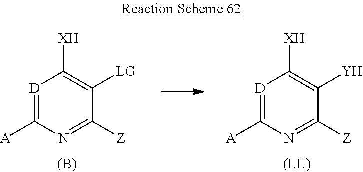

For example, a compound of formula (LL) in which Y is a heteroatom may be prepared by treating a compound of formula (B) with a nucleophile of formula (NN), as shown in reaction scheme 63.

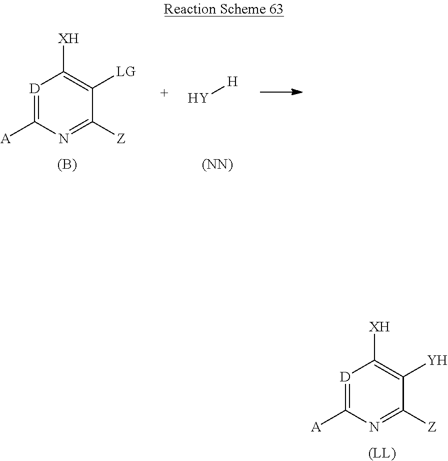

As an example a compound of formula (LL) may be prepared from a compound of formula (B) and a nucleophile of formula (NN) by treatment with a suitable catalyst (for example a metal catalyst, such as a palladium source) and optionally a suitable ligand (for example a phosphine ligand, such as Josiphos) in a suitable solvent.

One skilled in the art will realise that it is often possible to alter the order in which the transformations described above are conducted, or to combine them in alternative ways to prepare a wide range of compounds of formula (I). All such variations are contemplated within the scope of the invention.

The skilled man will also be aware that some reagents will be incompatible with certain values or combinations of the substituents A, D, E, X, Y and Z, and the number n as defined herein, and any additional steps, such as protection and/or deprotection steps, which are necessary to achieve the desired transformation will be clear to the skilled man.

Compounds of formula (I) may be used in unmodified form, i.e. as obtainable from synthesis, but preferably are formulated in any suitable manner using formulation adjuvants, such as carriers, solvents and surface-active substances, for example, as described hereinafter.

The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, suspension concentrates, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. The formulations can be in the form of concentrates which are diluted prior to use, although ready-to-use formulations can also be made. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules usually have a diameter of from 0.1 to 500 microns. Typically, they will contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other known polymers. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol (PEG), propionic acid, propyl lactate, propylene carbonate, propylene glycol, propylene glycol methyl ether, p-xylene, toluene, triethyl phosphate, Methylene glycol, xylenesulfonic acid, paraffin, mineral oil, trichloroethylene, perchloroethylene, amyl acetate, methanol, ethanol, isopropanol, and alcohols of higher molecular weight, such as amyl alcohol, tetrahydrofurfuryl alcohol, hexanol, octanol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in CFR 180.1001. (c) & (d).

A large number of surface-active substances may advantageously be used in the formulations, especially in those formulations designed to be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood N.J., 1981.

Further adjuvants that can usually be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and also liquid and solid fertilisers.

The compositions according to the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhône-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈₋₂₂ fatty acids, especially the methyl derivatives of C₁₂₋₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Another preferred adjuvant is Adigor® (Syngenta AG) which is a methylated rapeseed oil-based adjuvant.

The application and action of the oil additives can be further improved by combination with surface-active substances, such as non-ionic, anionic or cationic surfactants. Examples of suitable anionic, non-ionic and cationic surfactants are listed on pages 7 and 8 of WO97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂₋₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 10 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE®(Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic lattices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

Herbicidal compositions of the invention generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. Whereas commercial products will preferably be formulated as concentrates, the end user will normally employ dilute formulations.

Examples of preferred formulation types and their typical compositions are given below (% is percent by weight). Wettable powders as described herein are one particularly preferred type of formulation for use in the invention. In other preferred embodiments, in particular where the compound/composition/formulation of the invention is intended for use on turf, granular (inert or fertiliser) formulations as described herein are particularly suitable.

Emulsifiable Concentrates:

active ingredient: 1 to 95%, preferably 60 to 90%

surface-active agent: 1 to 30%, preferably 5 to 20%

liquid carrier: 1 to 80%, preferably 1 to 35%

Dusts:

active ingredient: 0.1 to 10%, preferably 0.1 to 5%

solid carrier: 99.9 to 90%, preferably 99.9 to 99%

Suspension Concentrates:

active ingredient: 5 to 75%, preferably 10 to 50%

water: 94 to 24%, preferably 88 to 30%

surface-active agent: 1 to 40%, preferably 2 to 30%

Wettable Powders:

active ingredient: 0.5 to 90%, preferably 1 to 80%

surface-active agent: 0.5 to 20%, preferably 1 to 15%

solid carrier: 5 to 95%, preferably 15 to 90%

Granules:

active ingredient: 0.1 to 30%, preferably 0.1 to 15%

solid carrier: 99.5 to 70%, preferably 97 to 85%

The following Examples further illustrate, but do not limit, the invention.

Formulation Examples for herbicides of formula (I) (%=% by weight)

F1. Emulsifiable concentrates

| | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| N-methyl pyrrolidone | — | — | 10% | 20% |
| arom. hydrocarbon mixture | 85% | 78% | 55% | 16% |
| (C₉-C₁₂) | | | | |

Emulsions of any desired concentration can be obtained from such concentrates by dilution with water.

F2. Solutions

| | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol MW 400 | 20% | 10% | — | — |
| NMP | — | — | 30% | 10% |
| arom. hydrocarbon mixture | 75% | 60% | — | — |
| (C₉-C₁₂) | | | | |

The solutions are suitable for use in the form of microdrops.

F3. Wettable powders

| | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

F4. Coated granules

| | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride and applied to the carrier by spraying, and the solvent is then evaporated off in vacuo.

F5. Coated granules

| | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is uniformly applied, in a mixer, to the carrier moistened with polyethylene glycol. Non-dusty coated granules are obtained in this manner.

F6. Extruder granules

| | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

F7. Dusts

| | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

F8. Suspension concentrates

| | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

Compounds of the invention (as well as mixtures and/or formulations containing the same) find utility as herbicides, and may thus be employed in methods of controlling plant growth. Such methods involve applying to the plants or to the locus thereof a herbicidally effective amount of said compound, or composition comprising the same (or mixture as described hereinafter). The invention thus also relates to a method of inhibiting plant growth which comprises applying to the plants or to the locus thereof a herbicidally effective amount of a compound of formula (I), composition, or mixture of the invention. In particular the invention provides a method of controlling weeds in crops of useful plants, which comprising applying to said weeds or the locus of said weeds, or to said crop of useful plants, a compound of formula I or a composition or mixture containing the same.

The term “locus” as used herein includes not only areas where weeds may already be growing, but also areas where weeds have yet to emerge, and also to areas under cultivation with respect to crops of useful plants. Areas under cultivation include land on which the crop plants are already growing and land intended for cultivation with such crop plants.

A compound, composition, and/or mixture of the invention may be used in a pre-emergence application and/or in a post-emergence application in order to mediate its effect.

Crops of useful plants in which compounds of formula (I), as well as formulations and/or mixtures containing the same, may be used according to the invention include perennial crops, such as citrus fruit, grapevines, nuts, oil palms, olives, pome fruit, stone fruit and rubber, and annual arable crops, such as cereals, for example barley and wheat, cotton, oilseed rape, maize, rice, soy beans, sugar beet, sugar cane, sunflowers, ornamentals and vegetables, especially cereals and maize.

Compounds of formula (I), formulations and/or mixtures containing the same may also be used on turf, pasture, rangeland, rights of way etc. In particular they may be used on golf-courses, lawns, parks, sports-fields, race-courses and the like.

Crops are to be understood as also including those crops which have been rendered tolerant to herbicides or classes of herbicides (e.g. ALS-, GS-, EPSPS-, PPO- and HPPD-inhibitors and synthetic auxins) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavour).

The term “weeds” as used herein means any undesired plant, and thus includes not only agronomically important weeds as described below, but also volunteer crop plants.

Compounds of formula (I) may be used against a large number of agronomically important weeds. The weeds that may be controlled include both monocotyledonous and dicotyledonous weeds, such as, for example, Alisma spp, Leptochloa chinensis, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus and especially Cyperus iria, Abutilon, Sida, Xanthium, Amaranthus, Chenopodium, Ipomoea, Chrysanthemum, Galium, Viola, Veronica, Bidens, Euphorbia, Ischaemum, Polygonum, Helianthus, Panicum, Eriochloa, Brachiaria, Cenchrus, Commelina, Spermacoce, Senna, Tridax, Richardia, Chamaesyce, and Conyza spp.

The rates of application of compounds of formula (I) may vary within wide limits and depend on the nature of the soil, the method of application (pre- or post-emergence; seed dressing; application to the seed furrow; no tillage application etc.), the crop plant, or weed to be controlled, the prevailing climatic conditions, and other factors governed by the method of application, the time of application and the target crop. The compounds of formula I according to the invention are generally applied at a rate of from 10 to 2000 g/ha, especially from 25 to 1000 g/ha.

Any method of application to weeds/crop of useful plant, or locus thereof, which is routinely used in agriculture may be used, for example application by spray or broadcast method typically after suitable dilution of a compound of formula (I) (whether said compound is formulated and/or in combination with one or more further active ingredients and/or safeners, as described herein).

The compounds of formula (I) according to the invention can also be used in combination with other active ingredients, e.g. other herbicides, and/or insecticides, and/or acaricides, and/or nematocides, and/or molluscicides, and/or fungicides, and/or plant growth regulators. Such mixtures, and the use of such mixtures to control weeds and/or undesired plant growth form yet further aspects of the invention. For the avoidance of doubt, mixtures of invention also include mixtures of two or more different compounds of formula (I).

Where a compound of formula (I) is combined with at least one additional herbicide, the following mixtures of the compound of formula (I) are particularly preferred. Compound of formula (I)+acetochlor, compound of formula (I)+acifluorfen, compound of formula (I)+acifluorfen-sodium, compound of formula (I)+aclonifen, compound of formula (I)+acrolein, compound of formula (I)+alachlor, compound of formula (I)+alloxydim, compound of formula (I)+allyl alcohol, compound of formula (I)+ametryn, compound of formula (I)+amicarbazone, compound of formula (I)+amidosulfuron, compound of formula (I)+aminocyclopyrachlor, compound of formula (I)+aminopyralid, compound of formula (I)+amitrole, compound of formula (I)+ammonium sulfamate, compound of formula (I)+anilofos, compound of formula (I)+asulam, compound of formula (I)+atrazine, formula (I)+aviglycine, formula (I)+azafenidin, compound of formula (I)+azimsulfuron, compound of formula (I)+BCPC, compound of formula (I)+beflubutamid, compound of formula (I)+benazolin, formula (I)+bencarbazone, compound of formula (I)+benfluralin, compound of formula (I)+benfuresate, compound of formula (I)+bensulfuron, compound of formula (I)+bensulfuron-methyl, compound of formula (I)+bensulide, compound of formula (I)+bentazone, compound of formula (I)+benzfendizone, compound of formula (I)+benzobicyclon, compound of formula (I)+benzofenap, compound of formula (I)+bifenox, compound of formula (I)+bilanafos, compound of formula (I)+bispyribac, compound of formula (I)+bispyribac-sodium, compound of formula (I)+borax, compound of formula (I)+bromacil, compound of formula (I)+bromobutide, formula (I)+bromophenoxim, compound of formula (I)+bromoxynil, compound of formula (I)+butachlor, compound of formula (I)+butafenacil, compound of formula (I)+butamifos, compound of formula (I)+butralin, compound of formula (I)+butroxydim, compound of formula (I)+butylate, compound of formula (I)+cacodylic acid, compound of formula (I)+calcium chlorate, compound of formula (I)+cafenstrole, compound of formula (I)+carbetamide, compound of formula (I)+carfentrazone, compound of formula (I)+carfentrazone-ethyl, compound of formula (I)+CDEA, compound of formula (I)+CEPC, compound of formula (I)+chlorflurenol, compound of formula (I)+chlorflurenol-methyl, compound of formula (I)+chloridazon, compound of formula (I)+chlorimuron, compound of formula (I)+chlorimuron-ethyl, compound of formula (I)+chloroacetic acid, compound of formula (I)+chlorotoluron, compound of formula (I)+chlorpropham, compound of formula (I)+chlorsulfuron, compound of formula (I)+chlorthal, compound of formula (I)+chlorthal-dimethyl, compound of formula (I)+cinidon-ethyl, compound of formula (I)+cinmethylin, compound of formula (I)+cinosulfuron, compound of formula (I)+cisanilide, compound of formula (I)+clethodim, compound of formula (I)+clodinafop, compound of formula (I)+clodinafop-propargyl, compound of formula (I)+clomazone, compound of formula (I)+clomeprop, compound of formula (I)+clopyralid, compound of formula (I)+cloransulam, compound of formula (I)+cloransulam-methyl, compound of formula (I)+CMA, compound of formula (I)+4-CPB, compound of formula (I)+CPMF, compound of formula (I)+4-CPP, compound of formula (I)+CPPC, compound of formula (I)+cresol, compound of formula (I)+cumyluron, compound of formula (I)+cyanamide, compound of formula (I)+cyanazine, compound of formula (I)+cycloate, compound of formula (I)+cyclosulfamuron, compound of formula (I)+cycloxydim, compound of formula (I)+cyhalofop, compound of formula (I)+cyhalofop-butyl, compound of formula (I)+2,4-D, compound of formula (I)+3,4-DA, compound of formula (I)+daimuron, compound of formula (I)+dalapon, compound of formula (I)+dazomet, compound of formula (I)+2,4-DB, compound of formula (I)+3,4-DB, compound of formula (I)+2,4-DEB, compound of formula (I)+desmedipham, formula (I)+desmetryn, compound of formula (I)+dicamba, compound of formula (I)+dichlobenil, compound of formula (I)+ortho-dichlorobenzene, compound of formula (I)+para-dichlorobenzene, compound of formula (I)+dichlorprop, compound of formula (I)+dichlorprop-P, compound of formula (I)+diclofop, compound of formula (I)+diclofop-methyl, compound of formula (I)+diclosulam, compound of formula (I)+difenzoquat, compound of formula (I)+difenzoquat metilsulfate, compound of formula (I)+diflufenican, compound of formula (I)+diflufenzopyr, compound of formula (I)+dimefuron, compound of formula (I)+dimepiperate, compound of formula (I)+dimethachlor, compound of formula (I)+dimethametryn, compound of formula (I)+dimethenamid, compound of formula (I)+dimethenamid-P, compound of formula (I)+dimethipin, compound of formula (I)+dimethylarsinic acid, compound of formula (I)+dinitramine, compound of formula (I)+dinoterb, compound of formula (I)+diphenamid, formula (I)+dipropetryn, compound of formula (I)+diquat, compound of formula (I)+diquat dibromide, compound of formula (I)+dithiopyr, compound of formula (I)+diuron, compound of formula (I)+DNOC, compound of formula (I)+3,4-DP, compound of formula (I)+DSMA, compound of formula (I)+EBEP, compound of formula (I)+endothal, compound of formula (I)+EPTC, compound of formula (I)+esprocarb, compound of formula (I)+ethalfluralin, compound of formula (I)+ethametsulfuron, compound of formula (I)+ethametsulfuron-methyl, formula (I)+ethephon, compound of formula (I)+ethofumesate, compound of formula (I)+ethoxyfen, compound of formula (I)+ethoxysulfuron, compound of formula (I)+etobenzanid, compound of formula (I)+fenoxaprop, compound of formula (I)+fenoxaprop-P, compound of formula (I)+fenoxaprop-ethyl, compound of formula (I)+fenoxaprop-P-ethyl, compound of formula (I)+fentrazamide, compound of formula (I)+ferrous sulfate, compound of formula (I)+flamprop-M, compound of formula (I)+flazasulfuron, compound of formula (I)+florasulam, compound of formula (I)+fluazifop, compound of formula (I)+fluazifop-butyl, compound of formula (I)+fluazifop-P, compound of formula (I)+fluazifop-P-butyl, formula (I)+fluazolate, compound of formula (I)+flucarbazone, compound of formula (I)+flucarbazone-sodium, compound of formula (I)+flucetosulfuron, compound of formula (I)+fluchloralin, compound of formula (I)+flufenacet, compound of formula (I)+flufenpyr, compound of formula (I)+flufenpyr-ethyl, formula (I)+flumetralin, compound of formula (I)+flumetsulam, compound of formula (I)+flumiclorac, compound of formula (I)+flumiclorac-pentyl, compound of formula (I)+flumioxazin, formula (I)+flumipropin, compound of formula (I)+fluometuron, compound of formula (I)+fluoroglycofen, compound of formula (I)+fluoroglycofen-ethyl, formula (I)+fluoxaprop, formula (I)+flupoxam, formula (I)+flupropacil, compound of formula (I)+flupropanate, compound of formula (I)+flupyrsulfuron, compound of formula (I)+flupyrsulfuron-methyl-sodium, compound of formula (I)+flurenol, compound of formula (I)+fluridone, compound of formula (I)+flurochloridone, compound of formula (I)+fluroxypyr, compound of formula (I)+flurtamone, compound of formula (I)+fluthiacet, compound of formula (I)+fluthiacet-methyl, compound of formula (I)+fomesafen, compound of formula (I)+foramsulfuron, compound of formula (I)+fosamine, compound of formula (I)+glufosinate, compound of formula (I)+glufosinate-ammonium, compound of formula (I)+glyphosate, compound of formula (I)+halosulfuron, compound of formula (I)+halosulfuron-methyl, compound of formula (I)+haloxyfop, compound of formula (I)+haloxyfop-P, compound of formula (I)+HC-252, compound of formula (I)+hexazinone, compound of formula (I)+imazamethabenz, compound of formula (I)+imazamethabenz-methyl, compound of formula (I)+imazamox, compound of formula (I)+imazapic, compound of formula (I)+imazapyr, compound of formula (I)+imazaquin, compound of formula (I)+imazethapyr, compound of formula (I)+imazosulfuron, compound of formula (I)+indanofan, compound of formula (I)+iodomethane, compound of formula (I)+iodosulfuron, compound of formula (I)+iodosulfuron-methyl-sodium, compound of formula (I)+ioxynil, compound of formula (I)+isoproturon, compound of formula (I)+isouron, compound of formula (I)+isoxaben, compound of formula (I)+isoxachiortole, compound of formula (I)+isoxaflutole, formula (I)+isoxapyrifop, compound of formula (I)+karbutilate, compound of formula (I)+lactofen, compound of formula (I)+lenacil, compound of formula (I)+linuron, compound of formula (I)+MAA, compound of formula (I)+MAMA, compound of formula (I)+MCPA, compound of formula (I)+MCPA-thioethyl, compound of formula (I)+MCPB, compound of formula (I)+mecoprop, compound of formula (I)+mecoprop-P, compound of formula (I)+mefenacet, compound of formula (I)+mefluidide, compound of formula (I)+mesosulfuron, compound of formula (I)+mesosulfuron-methyl, compound of formula (I)+mesotrione, compound of formula (I)+metam, compound of formula (I)+metamifop, compound of formula (I)+metamitron, compound of formula (I)+metazachlor, compound of formula (I)+methabenzthiazuron, formula (I)+methazole, compound of formula (I)+methylarsonic acid, compound of formula (I)+methyldymron, compound of formula (I)+methyl isothiocyanate, compound of formula (I)+metobenzuron, formula (I)+metobromuron, compound of formula (I)+metolachlor, compound of formula (I)+S-metolachlor, compound of formula (I)+metosulam, compound of formula (I)+metoxuron, compound of formula (I)+metribuzin, compound of formula (I)+metsulfuron, compound of formula (I)+metsulfuron-methyl, compound of formula (I)+MK-616, compound of formula (I)+molinate, compound of formula (I)+monolinuron, compound of formula (I)+MSMA, compound of formula (I)+naproanilide, compound of formula (I)+napropamide, compound of formula (I)+naptalam, formula (I)+NDA-402989, compound of formula (I)+neburon, compound of formula (I)+nicosulfuron, formula (I)+nipyraclofen, formula (I)+n-methyl glyphosate, compound of formula (I)+nonanoic acid, compound of formula (I)+norflurazon, compound of formula (I)+oleic acid (fatty acids), compound of formula (I)+orbencarb, compound of formula (I)+orthosulfamuron, compound of formula (I)+oryzalin, compound of formula (I)+oxadiargyl, compound of formula (I)+oxadiazon, compound of formula (I)+oxasulfuron, compound of formula (I)+oxaziclomefone, compound of formula (I)+oxyfluorfen, compound of formula (I)+paraquat, compound of formula (I)+paraquat dichloride, compound of formula (I)+pebulate, compound of formula (I)+pendimethalin, compound of formula (I)+penoxsulam, compound of formula (I)+pentachlorophenol, compound of formula (I)+pentanochlor, compound of formula (I)+pentoxazone, compound of formula (I)+pethoxamid, compound of formula (I)+petrolium oils, compound of formula (I)+phenmedipham, compound of formula (I)+phenmedipham-ethyl, compound of formula (I)+picloram, compound of formula (I)+picolinafen, compound of formula (I)+pinoxaden, compound of formula (I)+piperophos, compound of formula (I)+potassium arsenite, compound of formula (I)+potassium azide, compound of formula (I)+pretilachlor, compound of formula (I)+primisulfuron, compound of formula (I)+primisulfuron-methyl, compound of formula (I)+prodiamine, compound of formula (I)+profluazol, compound of formula (I)+profoxydim, formula (I)+prohexadione-calcium, compound of formula (I)+prometon, compound of formula (I)+prometryn, compound of formula (I)+propachlor, compound of formula (I)+propanil, compound of formula (I)+propaquizafop, compound of formula (I)+propazine, compound of formula (I)+propham, compound of formula (I)+propisochlor, compound of formula (I)+propoxycarbazone, compound of formula (I)+propoxycarbazone-sodium, compound of formula (I)+propyzamide, compound of formula (I)+prosulfocarb, compound of formula (I)+prosulfuron, compound of formula (I)+pyraclonil, compound of formula (I)+pyraflufen, compound of formula (I)+pyraflufen-ethyl, formula (I)+pyrasulfotole, compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyributicarb, compound of formula (I)+pyridafol, compound of formula (I)+pyridate, compound of formula (I)+pyriftalid, compound of formula (I)+pyriminobac, compound of formula (I)+pyriminobac-methyl, compound of formula (I)+pyrimisulfan, compound of formula (I)+pyrithiobac, compound of formula (I)+pyrithiobac-sodium, formula (I)+pyroxasulfone, formula (I)+pyroxulam, compound of formula (I)+quinclorac, compound of formula (I)+quinmerac, compound of formula (I)+quinoclamine, compound of formula (I)+quizalofop, compound of formula (I)+quizalofop-P, compound of formula (I)+quizalofop-ethyl, compound of formula (I)+quizalofop-P-ethyl, compound of formula (I)+rimsulfuron, compound of formula (I)+saflufenacil, compound of formula (I)+sethoxydim, compound of formula (I)+siduron, compound of formula (I)+simazine, compound of formula (I)+simetryn, compound of formula (I)+SMA, compound of formula (I)+sodium arsenite, compound of formula (I)+sodium azide, compound of formula (I)+sodium chlorate, compound of formula (I)+sulcotrione, compound of formula (I)+sulfentrazone, compound of formula (I)+sulfometuron, compound of formula (I)+sulfometuron-methyl, compound of formula (I)+sulfosate, compound of formula (I)+sulfosulfuron, compound of formula (I)+sulfuric acid, compound of formula (I)+tar oils, compound of formula (I)+2,3,6-TBA, compound of formula (I)+TCA, compound of formula (I)+TCA-sodium, formula (I)+tebutam, compound of formula (I)+tebuthiuron, formula (I)+tefuryltrione, compound of formula 1+tembotrione, compound of formula (I)+tepraloxydim, compound of formula (I)+terbacil, compound of formula (I)+terbumeton, compound of formula (I)+terbuthylazine, compound of formula (I)+terbutryn, compound of formula (I)+thenylchlor, compound of formula (I)+thiazafluron, compound of formula (I)+thiazopyr, compound of formula (I)+thifensulfuron, compound of formula (I)+thiencarbazone, compound of formula (I)+thifensulfuron-methyl, compound of formula (I)+thiobencarb, compound of formula (I)+tiocarbazil, compound of formula (I)+topramezone, compound of formula (I)+tralkoxydim, compound of formula (I)+tri-allate, compound of formula (I)+triasulfuron, compound of formula (I)+triaziflam, compound of formula (I)+tribenuron, compound of formula (I)+tribenuron-methyl, compound of formula (I)+tricamba, compound of formula (I)+triclopyr, compound of formula (I)+trietazine, compound of formula (I)+trifloxysulfuron, compound of formula (I)+trifloxysulfuron-sodium, compound of formula (I)+trifluralin, compound of formula (I)+triflusulfuron, compound of formula (I)+triflusulfuron-methyl, compound of formula (I)+trifop, compound of formula (I)+trifop-methyl, compound of formula (I)+trihydroxytriazine, compound of formula (I)+trinexapac-ethyl, compound of formula (I)+tritosulfuron, compound of formula (I)+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6), compound of formula (I)+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), compound of formula (I)+4-hydroxy-3-[[2-(3-methoxypropyl)-6-(difluoromethyl)-3-pyridinyl]carbonyl]-bicyclo[3.2.1]oct-3-en-2-one, and compound of formula (I)+4-amino-3-chloro-6-(4-chloro-2-fluoro-3-methoxyphenyl)-pyridine-2-carboxylic acid (CAS RN 943832-60-8).

Whilst two-way mixtures of a compound of formula (I) and another herbicide are explicitly disclosed above, the skilled man will appreciate that the invention extends to three-way, and further multiple combinations comprising the above two-way mixtures.

In preferred embodiments a compound of formula (I) is combined with an acetolactate synthase inhibitor, (e.g. one or more of florasulam, metsulfuron, thifensulfuron, tribenuron, triasulfuron, flucarbazone, flupyrsulfuron, iodosulfuron, mesosulfuron, propoxicarbazone, sulfosulfuron, pyroxsulam and tritosulfuron, as well as salts or esters thereof), a synthetic auxin herbicide [e.g. one or more of aminocyclopyrachlor, aminopyralid, clopyralid, 2,4-D, 2,4-DB, dicamba, dichlorprop, fluroxypyr, MCPA, MCPB, mecoprop, mecoprop-P and 4-amino-3-chloro-6-(4-chloro-2-fluoro-3-methoxyphenyl)-pyridine-2-carboxylic acid (CAS RN 943832-60-8)], an ACCase-inhibiting herbicide (e.g. one or more of phenylpyrazolin; pinoxaden; an aryloxyphenoxypropionic herbicide such as clodinafop, cyhalofop, diclofop, fenoxaprop, fluazifop, haloxyfop, quizalofop, trifop and mixtures thereof, as well as the isomers thereof, for example, fenoxaprop-P, fluazifop-P, haloxyfop-P, quizalofop-P; and a cyclohexanedione herbicide such as alloxydim, butroxydim, clethodim, cycloxydim, profoxydim, sethoxydim, tepraloxydim and tralkoxydim, as well as salts or esters thereof), an auxin transport inhibitor such as a semicarbazone (e.g. diflufenzopyr, in particular the sodium salt) or phthalamate compound (e.g. naptalam), and/or an EPSPS inhibitor such as glyphosate.

Particularly preferred mixture partners for compounds of formula (I) are: florasulam, iodosulfuron-methyl-sodium, mesosulfuron-methyl, metsulfuron-methyl, thifensulfuron, triasulfuron, tribenuron-methyl or pyroxsulam; dicamba, fluroxypyr, MCPA, mecoprop, mecoprop-P or 4-amino-3-chloro-6-(4-chloro-2-fluoro-3-methoxyphenyl)-pyridine-2-carboxylic acid (CAS RN 943832-60-8); clodinafop-propargyl, cyhalofop-butyl, diclofop-methyl, fenoxaprop-ethyl, fenoxaprop-P-ethyl, fluazifop-butyl, fluazifop-P-butyl, haloxyfop-methyl, haloxyfop-P-methyl, pinoxaden, propaquizafop, quizalofop-ethyl, quizalofop-P-ethyl, tralkoxydim, trifop-methyl, diflufenzopyr-Na, naptalam, and glyphosate.

For the avoidance of doubt, even if not explicitly stated above, the mixing partners of the compound of formula (I) may also be in the form of any suitable agrochemically acceptable ester or salt, as mentioned e.g. in The Pesticide Manual, Thirteenth Edition, British Crop Protection Council, 2003.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

The compounds of formula (I) according to the invention can also be used in combination with one or more safeners. Likewise, mixtures of a compound of formula (I) according to the invention with one or more further active ingredients, in particular with one or more further herbicides, can also be used in combination with one or more safeners. Where a compound of formula (I) is combined with a safener, the following combinations of the compound of formula (I) and the safener are particularly preferred. Compound of formula (I)+AD 67 (MON 4660), compound of formula (I)+benoxacor, compound of formula (I)+cloquintocet-mexyl, compound of formula (I)+cyometrinil and a compound of formula (I)+the corresponding (Z) isomer of cyometrinil, compound of formula (I)+cyprosulfamide (CAS RN 221667-31-8), compound of formula (I)+dichlormid, compound of formula (I)+fenchlorazole-ethyl, compound of formula (I)+fenclorim, compound of formula (I)+flurazole, compound of formula (I)+fluxofenim, compound of formula (I)+furilazole and a compound of formula (I)+the corresponding R isomer or furilazome, compound of formula (I)+isoxadifen-ethyl, compound of formula (I)+mefenpyr-diethyl, compound of formula (I)+oxabetrinil, compound of formula (I)+naphthalic anhydride (CAS RN 81-84-5), compound of formula (I)+N-isopropyl-4-(2-methoxy-benzoylsulfamoyl)-benzamide (CAS RN 221668-34-4) and a compound of formula (I) N-(2-methoxybenzoyl)-4-[(methylaminocarbonyl)amino]benzenesulfonamide.

Particularly preferred safeners for use in the invention are cloquintocet-mexyl, cyprosulfamide, fenchlorazole-ethyl, mefenpyr-diethyl and N-(2-methoxybenzoyl)-4-[(methylaminocarbonyl)amino]benzenesulfonamide. The safeners of the compound of formula (I) may also be in the form of esters or salts, as mentioned e.g. in The Pesticide Manual, 13^{th} Edition supra. The reference to cloquintocet-mexyl also applies to a lithium, sodium, potassium, calcium, magnesium, aluminium, iron, ammonium, quaternary ammonium, sulfonium or phosphonium salt thereof as disclosed in W002/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

Preferred mixtures of a compound of formula (I) with further herbicides and safeners include: a compound of formula (I)+pinoxaden+cloquintocet-mexyl, a compound of formula (I)+clodinafop+cloquintocet-mexyl, and a compound of formula (I)+clodinafop-propargyl+cloquintocet-mexyl.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

### Example 1

### Synthesis of 2-(4-chloro-3-dimethylamino-2-fluorophenyl)-4-methoxycarbonyl-6-methyl-7H-pyrrolo[2,3-d]pyrimidine (Compound 1-118)

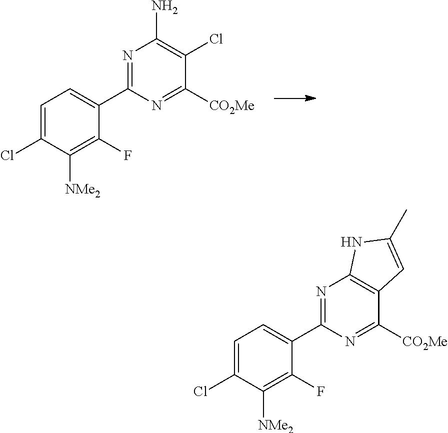

A solution of 4-amino-5-chloro-2-(4-chloro-3-dimethylamino-2-fluorophenyl)-6-methoxycarbonylpyrirnidine (prepared as described in WO2007/082076) (585 mg, 1.63 mmol), allenyltributylstannane (0.58 ml, 2.0 mmol) and tetrakis(triphenylphosphine)palladium (376 mg, 0.326 mmol) in dimethyl sulphoxide (11 ml) was heated in a microwave reactor at 170° C. for 40 minutes, then allowed to cool. A saturated solution of potassium fluoride in methanol (24 ml) was added and the resulting mixture stirred at ambient temperature for 2 hours, then allowed to stand for a further 16 hours. The mixture was filtered through Celite®, the solid washed with methanol and the filtrate evaporated under reduced pressure. The residue was extracted with ether and ethyl acetate and the combined organic extracts washed with brine, dried over magnesium sulphate, filtered and absorbed onto silica. Purification using a FractionLynx hplc provided 2-(4-chloro-3-dimethylamino-2-fluorophenyl)-4-methoxycarbonyl-6-methyl-7H-pyrrolo[2,3-d]pyrimidine as a yellow gum (45 mg, 8%).

Characterising data for the compound are as follows:

¹H NMR (400 MHz, CDCl₃) δ 9.90 (1H, br s), 7.70 (1H, t), 7.20 (1H, dd), 6.80 (1H, m), 4.10 (3H, s), 2.90 (6H, s), 2.40 (3H, s) ppm.

Further examples of compounds that were prepared using this method are listed below in Table 12.

TABLE 12
Compounds made according to the method described in Example 1 above.

| Compound | | | ¹H NMR (400 MHz, |
|---|---|---|---|
| Number | Name | Structure | CDCl₃) δ |
| 1-66 | 2-(4-chloro-2- fluoro-3- methoxyphenyl)-4- methoxycarbonyl- 6-methyl-7H- pyrrolo[2,3- d]pyrimidine | 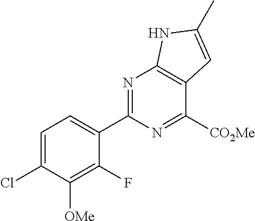 | 9.30 (1H, br s), 7.80 (1H, t), 7.20 (1H, t), 6.80 (1H, m), 4.10 (3H, s), 4.00 (3H, s), 2.50 (3H, s) ppm |

### Example 2

### Alternative synthesis of 2-(4-chloro-2-fluoro-3-methoxyphenyl)-4-methoxycarbonyl-6-methyl-7H-pyrrolo[2,3-d]pyrimidine (Compound 1-66)

### 2.1 Preparation of 5-chloro-2-(4-chloro-2-fluoro-3-methoxyphenyl)-6-methoxycarbonyl-4-thiomethyl-pyrimidine

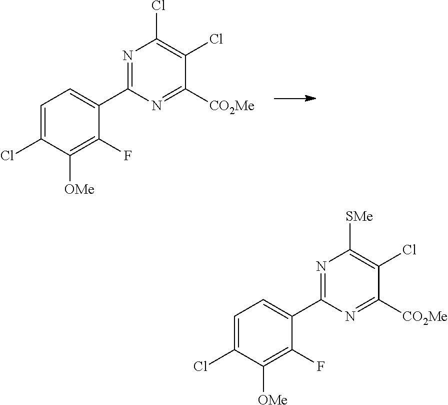

Sodium methanethiolate (290 mg, 4.1 mmol) was added to a solution of 2-(4-chloro-2-fluoro-3-methoxyphenyl)-4,5-dichloro-6-methoxycarbonyl-pyrimidine (prepared as described in WO2009/081112) (1.00 g, 2.73 mmol) in methanol (20 ml) and the resulting mixture stirred at ambient temperature for 1 hour, then evaporated under reduced pressure. The residue was extracted with ethyl acetate and the extract washed with water and brine, dried over magnesium sulphate, filtered and evaporated under reduced pressure to provide 5-chloro-2-(4-chloro-2-fluoro-3-methoxyphenyl)-6-methoxycarbonyl-4-thiomethyl-pyrimidine as pale yellow solid (800 mg, 77%).

Characterising data for the compound are as follows:

¹H NMR (400 MHz, CDCl₃) δ 7.80 (1H, t), 7.20 (1H, dd), 4.05 (3H, s), 4.00 (3H, s), 2.70 (3H, s) ppm.

Further examples of compounds that were prepared using this method are listed below in Table 13.

TABLE 13
Compounds made according to the method described in Example 2.1 above.

| Name | Structure | Melting point ° C. |
|---|---|---|
| 5-Chloro-2- cyclopropyl-6- methoxycarbonyl- 4-thiomethyl- pyrimidine | 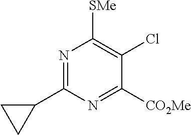 | 74-75 |

### 2.2 Preparation of (Z)-2-(4-chloro-2-fluoro-3-methoxyphenyl)-6-methoxycarbonyl-5-(prop-1-enyl)-4-thiomethyl-pyrimidine

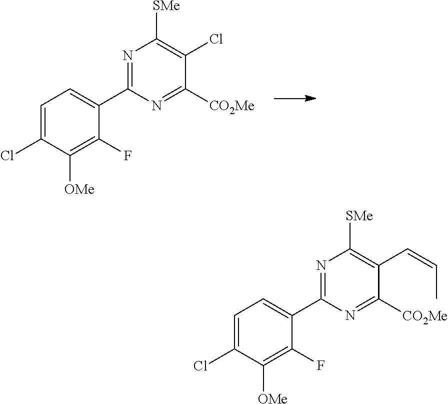

A mixture of 5-chloro-2-(4-chloro-2-fluoro-3-methoxyphenyl)-6-methoxycarbonyl-4-thiomethyl-pyrimidine (200 mg, 0.56 mmol), cis-propenyl boronic acid (72 mg, 0.84 mmol), [1,1′-bis(diphenylphosphino)-ferrocene]dichloropalladium (II) complex with dichloromethane (1:1) (46 mg, 0.056 mmol), caesium fluoride (170 mg, 1.12 mmol), dimethoxyethane (2.4 ml) and water (2.4.ml) was heated in a microwave reactor at 150° C. for 20 minutes, then allowed to cool. Water was added and the resulting mixture extracted with dichloromethane. The organic extracts were washed with brine, dried over magnesium sulphate, filtered and evaporated under reduced pressure. The residue was purified by automated flash chromatography (Presearch Combiflash Rf) on silica, using hexane:thyl acetate (4:1) as eluent, to provide (Z)-2-(4-chloro-2-fluoro-3-methoxyphenyl)-6-methoxycarbonyl-5-(prop-1-enyl)-4-thiomethyl-pyrimidine as a pale yellow solid (100 mg, 49%).

Characterising data for the compound are as follows:

¹H NMR (400 MHz, CDCl₃) δ 7.90 (1H, t), 7.30 (1H, dd), 6.30 (1H, d), 6.10 (1H, m), 4.00 (3H, s), 3.90 (3H, s), 2.60 (3H, s), 1.60 (3H, d) ppm.

Further examples of compounds that were prepared using this method are listed below in Table 14.

TABLE 14
Compounds made according to the method described in Example 2.2 above.

| | | ¹H NMR (400 MHz, |
|---|---|---|
| Name | Structure | CDCl₃) δ |
| 2-Cyclopropyl-5- ethenyl-6- methoxycarbonyl- 4-thiomethyl- pyrimidine | 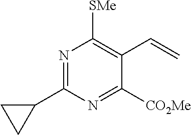 | 6.68 (1H, m), 5.55 (2H, m), 3.90 (3H, s), 2.50 (3H, s), 2.23 (1H, m), 1.20 (2H, m), 1.08 (2H, m) ppm |

### 2.3 Preparation of (Z)-4-azido-2-(4-chloro-2-fluoro-3-methoxyphenyl)-6-methoxycarbonyl-5-(prop-1-enyl)-pyrimidine

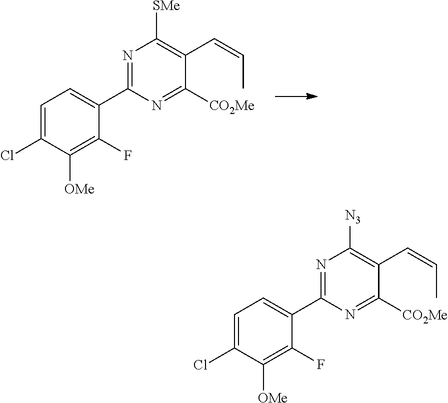

3-Chloroperbenzoic acid (168 mg, 1.0 mmol) was added to a stirred suspension of (Z)-2-(4-chloro-2-fluoro-3-methoxyphenyl)-6-methoxycarbonyl-5-(prop-1-enyl)-4-thiomethyl-pyrimidine (100 mg, 0.39 mmol) in dichloromethane and stirring continued for 2 hours. The reaction mixture was washed with water and brine, dried over magnesium sulphate, filtered and evaporated under reduced pressure. The residue was dissolved in methanol (5 ml) and sodium azide (255 mg, 3.0 mmol) added. The resulting mixture was stirred at ambient temperature for 16 hours, and then concentrated under reduced pressure. The residue was suspended in dichloromethane, washed with water and brine, dried over magnesium sulphate, filtered and evaporated under reduced pressure. The residue was purified by automated flash chromatography (Presearch Combiflash Rf) on silica, using hexane:ethyl acetate (4:1) as eluent, to provide (Z)-4--azido-2-(4-chloro-2-fluoro-3-methoxyphenyl)-6-methoxycarbonyl-5-(prop-1-enyl)-pyrimidine as a pale yellow oil (80 mg, 81%).

Characterising data for the compound are as follows:

¹H NMR (400 MHz, CDCl₃) δ 7.80 (1H, t), 7.30 (1H, m), 6.30 (1H, dd), 6.00 (1H, m), 4.00 (2×3H, s), 1.60 (3H, d) ppm.

Further examples of compounds that were prepared using this method are listed below in Table 15.

TABLE 15
Compounds made according to the method described in Example 2.3 above.

| Name | Structure | Melting Point ° C. |
|---|---|---|
| 4-Azido-2- cyclopropyl-5- ethenyl-6- methoxycarbonyl- pyrimidine | 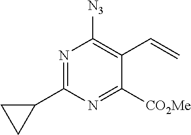 | 120-130 (dec.) |

### 2.4 Preparation of 2-(4-chloro-2-fluoro-3-methoxyphenyl)-4-methoxycarbonyl-6-methyl-7H-pyrrolo[2,3-d]pyrimidine (Compound 1-66)

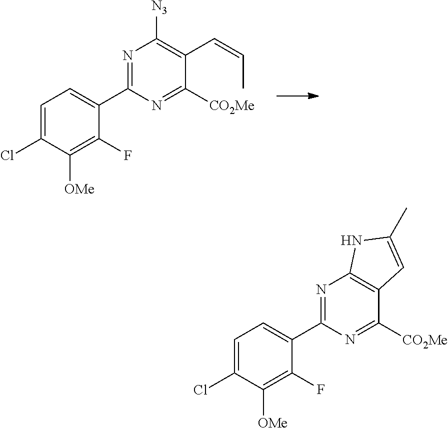

A solution of (Z)-4-azido-2-(4-chloro-2-fluoro-3-methoxyphenyl)-6-methoxycarbonyl-5-(prop-1-enyl)-pyrimidine (80 mg, 0.21 mmol) in 1,2-dichlorobenzene (3 ml) was heated at 156° C. for 1 hour, then allowed to cool. The reaction mixture was filtered through a silica column, eluting first with hexane, then with hexane:ethyl acetate (3:2) to provide the crude product, which was further purified by automated flash chromatography (Presearch Combiflash Rf) on silica, with hexane, then ethyl acetate in hexane (0-40% gradient) to provide 2-(4-chloro-2-fluoro-3-methoxyphenyl)-4-methoxycarbonyl-6-methyl-7H-pyrrolo[2,3-d]pyrimidine as a yellow solid (20 mg, 27%).

Characterising data for the compound are as follows:

¹H NMR (400 MHz, CDCl₃) δ 9.30 (1H, br s), 7.80 (1 H, t), 7.20 (1H, t), 6.80 (1H, m), 4.10 (3H, s), 4.00 (3H, s), 2.50 (3H, s) ppm.

Further examples of compounds that were prepared using this method are listed below in Table 16.

TABLE 16
Compounds made according to the method described in Example 2.4 above.

| Compound | | | Melting |
|---|---|---|---|
| Number | Name | Structure | Point ° C. |
| 1-2 | 2-Cyclopropyl-4- methoxycarbonyl- 7H-pyrrolo[2,3- d]pyrimidine | 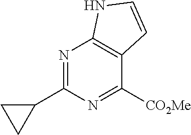 | 162-165 |

### Example 3

### Synthesis of 2-(4-chloro-2-fluoro-3-methoxyphenyl)-4-methoxycarbonyl-5-methyl-7H-pyrrolo[2,3-d]pyrimidine (Compound 1-64)

### 3.1 Preparation of 5-chloro-2-(4-chloro-2-fluoro-3-methoxyphenyl)-6-methoxycarbonyl-4(prop-2-enylamino)-pyrimidine

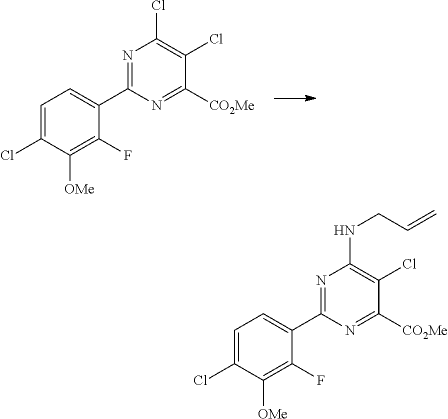

A solution of 2-(4-chloro-2-fluoro-3-methoxyphenyl)-4,5-dichloro-6-methoxycarbonylpyrimidine (prepared as described in WO2009/081112) (364 mg, 1.0 mmol), allylamine (0.15 ml, 2.0 mmol) and triethylamine (0.26 ml, 2.0 mmol) in dichloroethane (3.5 ml) was stirred at ambient temperature for 4 hours. Water was added and the mixture extracted with dichloromethane. The organic extract was washed with brine, dried over magnesium sulphate, filtered and evaporated under reduced pressure. The residue was purified by automated flash chromatography (Presearch Combiflash Rf) on silica, using ethyl acetate in hexane (20% to 40% gradient) as eluent to provide 5-chloro-2-(4-chloro-2-fluoro-3-methoxyphenyl)-6-methoxycarbonyl-4-(prop-2-enylamino)-pyrimidine as a yellow solid (330 mg, 85%). Characterising data for the compound are as follows:

¹H NMR (400 MHz, CDCl₃) δ 7.70 (1H, t), 7.20 (1H, dd), 6.00 (1H, m), 5.80 (1H, br s), 5.30 (2H, qd), 4.30 (2H, m), 4.00 (2×3H, s) ppm.

Further examples of compounds that were prepared using this method are listed below in Table 17.

TABLE 17
Compounds made according to the method described in Example 3.1 above.

| | | ¹H NMR (400 MHz, |
|---|---|---|
| Name | Structure | CDCl₃) δ |
| 5-Chloro-2-(4-chloro-2-fluoro-3- methoxyphenyl)-6- methoxycarbonyl-4-(N-methyl- N-prop-2-enyl-amino)- pyrimidine | 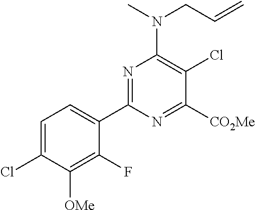 | 7.70 (1H, t), 7.20 (1H. dd), 5.90 (1H, m), 5.30 (1H, m), 5.20 (1H, m), 4.30 (2H, d), 4.00 (2x 3H, s), 3.30 (3H, s) ppm |
| 5-Chloro-2-(4-chloro-2-fluoro-3- methoxyphenyl)-6- methoxycarbonyl-4-(N- phenylmethyl-N-prop-2-enyl- amino)-pyrimidine | 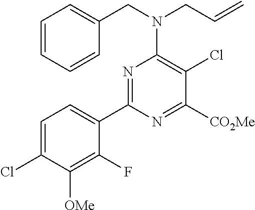 | 7.70 (1H, t), 7.30 (5H, m), 7.20 (1H, dd), 6.00 (1H, m), 5.30 (2H, q), 5.00 (2H, s), 4.30 (2H, d), 4.00 (2x 3H, s) ppm |
| 4-(But-1-en-3-ylamino)-5- chloro-2-(4-chloro-2-fluoro-3- methoxyphenyl)-6- methoxycarbonyl-pyrimidine | 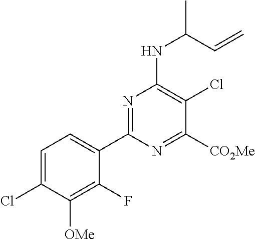 | 7.70 (1H, t), 7.20 (1H, dd), 5.90 (1H, dq), 5.60 (1H, br s), 5.20 (2H, qd), 5.00 (1H, m), 4.00 (2x 3H, s), 1.40 (3H, d) ppm |
| 5-Chloro-2-(4-chloro-2-fluoro-3- methoxyphenyl)-6- methoxycarbonyl-4-(1- phenylprop-2-en-1-ylamino)- pyrimidine | 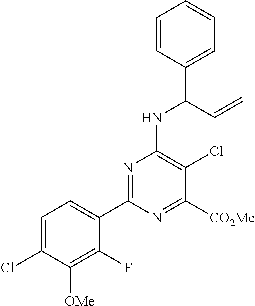 | 7.60 (1H, t), 7.40 (5H, m), 7.20 (1H, dd), 6.10 (1H, m), 6.00 (2H, br s), 5.30 (2H, dd), 4.00 (2x 3H, s) ppm |
| 5-Chloro-2-(4-chloro-2-fluoro-3- methoxyphenyl)-6- methoxycarbonyl-4-(prop-2- enylthio)-pyrimidine | 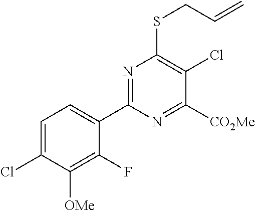 | 7.81 (1H, t), 7.26 (1H, dd) 5.98 (1H, m), 5.37 (1H, dq), 5.20 (1H, d), 4.04 (3H, s), 4.02 (3H, s), 3.97 (2H, d) ppm |
| 5-Chloro-2-(4-chloro-2-fluoro-3- methoxyphenyl)-4-(2,2- dimethyl-but-3-en-1-ylamino)-6- nethoxycarbonyl-pyrimidine | 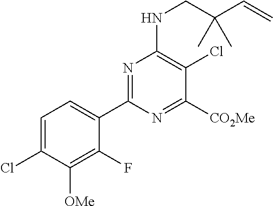 | 7.70 (1H, t), 7.20 (1H, d), 5.80 (1H, dd), 5.70 (1H, br s), 5.20 (2H, m), 4.00 (2x 3H, s), 3.50 (2H, d), 1.10 (6H, s) ppm |
| 5-Chloro-2-cyclopropyl-6- methoxycarbonyl-4-(prop-2- enylamino)-pyrimidine | 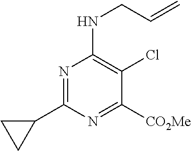 | 5.90 (1H, m) 5.60 (1H, br s), 5.20 (2H, m), 4.10 (2H, m), 4.00 (3H, s), 2.10 (1H, m), 1.10 (2H, m), 0.90 (2H, m) ppm |
| 5-Chloro-2-cyclopropyl-6- methoxycarbonyl-4-(2- nitrophenyl-methylamino)- pyrimidine | 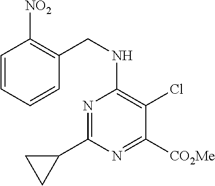 | 8.10 (1H, d), 7.61, (2H, m), 7.48 (1H, t), 6.46 (1H, br t), 4.98 (2H, d), 3.96 (3H, s), 2.10 (1H, quintet), 0.99 (4H, d) ppm |
| 4-(2-Amino-1,2-diphenyl- ethylamino)-5-chloro-2- cyclopropyl-6-methoxycarbonyl- pyrimidine | 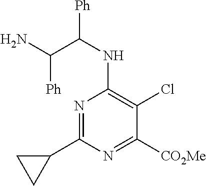 | 7.29 (3H, m), 7.23 (3H, m), 7.11 (2H, d), 7.03 (2H, m), 6.70 (1H, br d), 5.16 (1H, t), 4.38 (1H, m), 3.95 (3H, s), 1.95 (1H, quintet), 0.98 (1H, m), 0.89 (1H, m), 0.75 (1H, m), 0.49 (1H, m) ppm (NH₂ not observed) |
| 4-(trans-2-Amino- cyclohexylamino)-5-chloro-2-(4- chloro-2-fluoro-3- methoxyphenyl)-6- methoxycarbonyl-pyrimidine | 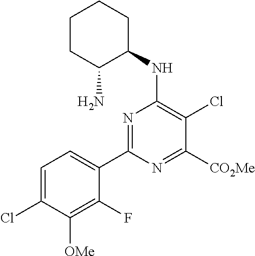 | 7.86 (1H, br s), 7.20 (1H, dd), 6.09 (1H, br s), 4.16 (1H, br s), 4.00 (3H, s), 3.97 (3H, s), 2.74 (1H, br s), 2.03 (3H, m), 1.72 (3H, m), 1.34 (4H, m) ppm |
| 5-Chloro-2-(4-chloro-2-fluoro-3- methoxyphenyl)-6- methoxycarbonyl-4- phenylmethylamino-pyrimidine | 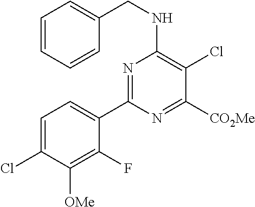 | 7.70 (1H, dd), 7.36 (4H, m), 7.33 (1H, m), 7.20 (1H, dd), 6.04 (1H, br t), 4.81 (2H, d), 4.01 (3H, s), 4.00 (3H, s) ppm |
| 5-Chloro-2-(4-chloro-2-fluoro-3- methoxyphenyl)-4-[N-(2- hydroxyethyl)-N-methyl-amino)- 6-methoxycarbonyl-pyrimidine | 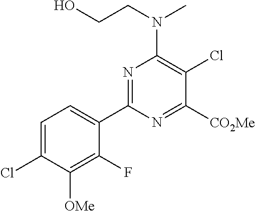 | 7.69 (1H, dd), 7.21 (1H, d), 3.98 (2x3H, s), 3.95 (2H, m), 3.89 (2H, m), 3.43 (3H, s), 2.81 (1H, br s) ppm |
| 4-(3-Amino-butan-2-yl-amino)- 5-chloro-2-(4-chloro-2-fluoro-3- methoxyphenyl)-6- methoxycarbonyl-pyrimidine | 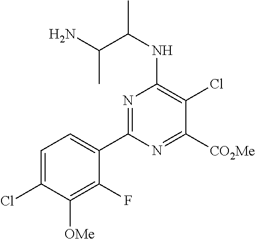 | 7.69 (1H, dd), 7.20 (1H, dd), 6.33 (1H, br s), 4.30 (1H, m), 3.99 (2x3H, s), 3.12 (1H, m), 1.19 (3H, d), 1.16 (3H, d) ppm |
| 5-Chloro-2-(4-chloro-2-fluoro-3- methoxyphenyl)-4-(2,4- dimethoxyphenyl-methylamino)- 6-methoxycarbonyl-pyrimidine | 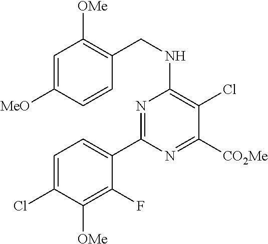 | 7.73 (1H, dd), 7.28 (1H, d), 7.22 (1H, dd), 6.49 (1H, s), 6.44 (1H, d), 6.30 (1H, br t), 4.72 (2H, d), 4.02 (3H, s), 3.97 (3H, s), 3.88 (3H, s), 3.80 (3H, s) ppm |

### 3.2 Preparation of 2-(4-chloro-2-fluoro-3-methoxyphenyl)-4-methoxycarbonyl-5-methyl-7H-pyrrolo[2,3-d]pyrimidine (Compound 1-64)

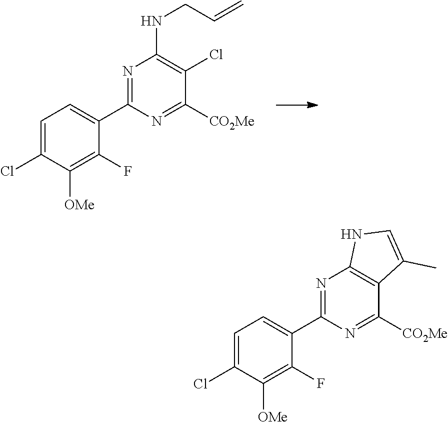

A mixture of 5-chloro-2-(4-chloro-2-fluoro-3-methoxyphenyl)-6-methoxycarbonyl-4-(prop-2-enylamino)-pyrimidine (200 mg, 0.52 mmol), [1,3-bis(2,6-diisopropylphenyl)imidazol-2-ylidend3-chloropyridyl)palladium(II) dichloride (36 mg, 0.052 mmol), sodium acetate (64 mg, 0.78 mmol) and dimethyl acetamide (4 ml) was heated in a microwave reactor at 150° C. for 30 minutes, then allowed to cool, water added and the mixture extracted with ethyl acetate. The organic extract was washed with water and brine, dried over magnesium sulphate, filtered and evaporated under reduced pressure. The residue was purified by automated flash chromatography (Presearch Combiflash Rf) on silica, with ethyl acetate in hexane (20% to 40% gradient) as eluent, to provide 2-(4-chloro-2-fluoro-3-methoxyphenyl)-4-methoxycarbonyl-5-methyl-7H-pyrrolo[2,3-d]pyrimidine as a yellow solid (106 mg, 59%).

Characterising data for the compound are as follows:

¹H NMR (400 MHz, CDCl₃) δ 7.78 (1H, t), 7.30 (1H, dd), 7.23 (1H, br s), 4.10 (3H, s), 4.03 (3H, s), 2.45 (3H, s) ppm (NH not observed).

Further examples of compounds that were prepared using this method are listed below in Table 18.

TABLE 18
Compounds made according to the method described in Example 3.2 above.

| Compound | | | ¹H NMR (400 MHz, |
|---|---|---|---|
| Number | Name | Structure | CDCl₃) δ |
| 2-64 | 2-(4-Chloro-2-fluoro-3- methoxyphenyl)-5,7- dimethyl-4- methoxycarbonyl-7H- pyrrolo[2,3-d]pyrimidine | 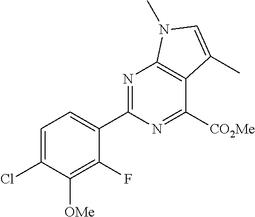 | 7.80 (1H, t), 7.20 (1H, dd), 7.10 (1H, d), 4.10 (3H, s), 4.00 (3H, s), 3.90 (3H, s), 2.50 (3H, s) ppm |
| 6-64 | 2-(4-Chloro-2-fluoro-3- methoxyphenyl)-4- methoxycarbonyl-5- methyl-7-phenylmethyl- 7H-pyrrolo[2,3- d]pyrimidine | 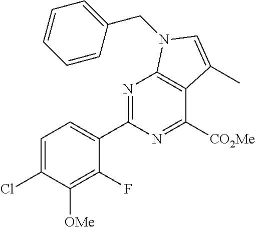 | 7.85 (1H, t), 7.30 (6H, m), 7.12 (1H, d), 5.48 (2H, s), 4.07 (3H, s), 4.03 (3H, s), 2.40 (3H, s) ppm |
| 1-68 | 2-(4-Chloro-2-fluoro-3- methoxyphenyl)-5,6- dimethyl-4- methoxycarbonyl-7H- pyrrolo[2,3-d]pyrimidine | 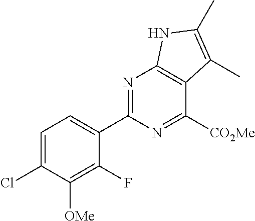 | 8.70 (1H, br s), 7.80 (1H, t), 7.20 (1H, dd), 4.10 (3H, s), 4.00 (3H, s), 2.50 (3H, s), 2.40 (3H, s) ppm |
| 1-108 | 2-(4-Chloro-2-fluoro-3- methoxyphenyl)-4- methoxycarbonyl-5- methyl-6-phenyl-7H- pyrrolo[2,3-d]pyrimidine | 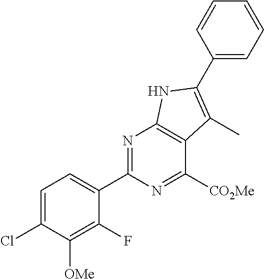 | 9.40 (1H, br s), 7.80 (1H, t), 7.50 (5H, m), 7.20 (1H, dd), 4.10 (3H, s), 4.00 (3H, s), 2.50 (3H, s) ppm |
| 21-64 | 2-(4-Chloro-2-fluoro-3- methoxyphenyl)-4- methoxycarbonyl-5- methyl-thieno[2,3- d]pyrimidine | 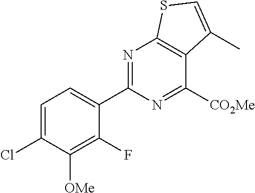 | 7.80 (1H, t), 7.30 (1H, dd), 6.00 (1H, m), 4.00 (2x 3H, s), 2.60 (3H, s) ppm |
| 71-180 | 2-(4-Chloro-2-fluoro-3- methoxyphenyl)-6,6- dimethyl-4- methoxycarbonyl-5- methylene-5,6,7,8- tetrahydro-pyrido[2,3- d]pyrimidine | 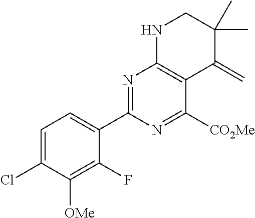 | 7.60 (1H, t), 7.20 (1H, dd), 5.70 (1H, br s), 5.20 (2H, s), 4.00 (3H, s), 3.90 (3H, s), 3.20 (2H, d), 1.20 (6H, s) ppm |
| 1-124 | 2-Chloro-4- methoxycarbonyl-5- methyl-7H-pyrrolo[2,3- d]pyrimidine | 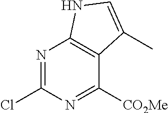 | 7.40 (1H, s) 4.00 (3H, s) 2.40 (3H, s) ppm (NH not observed) (nmr run in CD₃OD) |
| 1-4 | 2-Cyclopropyl-4- methoxycarbonyl-5- methyl-7H-pyrrolo[2,3- d]pyrimidine | 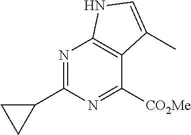 | 10.20 (1H, br s), 7.20 (1H, s), 4.00 (3H, s), 2.40 (1H, m), 2.35 (3H, s), 1.20 (4H, m) ppm |

### Example 4

### Synthesis of 2-(4-chloro-3-fluorophenyl)-5,6-dimethyl-4-methoxycarbonyl-7H-pyrrolo[2,3-d]pyrimidine (Compound 1-24)

### 4.1 Preparation of 2,5-dichloro-6-methoxycarbonyl-4-(prop-2-enylamino)-pyrimidine

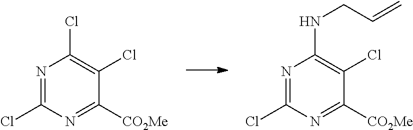

A solution of 6-methoxycarbony-2,4,5-trichloropyrimidine (prepared as described in WO2009/081112) (1000 mg, 4.0 mmol), allylamine (0.45 ml, 6.0 mmol) and triethylamine (1.1 ml, 8.0 mmol) in dimethoxyethane (10 ml) was stirred at ambient temperature for 2 hours. Water was added and the mixture extracted with ethyl acetate. The organic extract was washed with brine, dried over magnesium sulphate, filtered and evaporated under reduced pressure. The residue was purified by automated flash chromatography (Presearch Combiflash Rf) on silica, using ethyl acetate in hexane (0% to 40% gradient) as eluent to provide 2,5-dichloro-6-methoxycarbonyl-4-(prop-2-enylamino)-pyrimidine as an off-white solid (1000 mg, 87%).

Characterising data for the compound are as follows:

¹H NMR (400 MHz, CDCl₃) δ 6.00 (2H, br m), 5.30 (2H, m), 4.20 (2H, m), 4.00 (3H, s) ppm.

Further examples of compounds that were prepared using this method are listed below in Table 19.

TABLE 19
Compounds made according to the method described in Example 4.1 above.

| | | ¹H NMR (400 MHz, |
|---|---|---|
| Name | Structure | CDCl₃) δ |
| 4-(But-1-en-3-ylamino)-2,5- dichloro-6-methoxycarbonyl- pyrimidine | 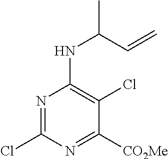 | 5.90 (1H, dq), 5.80 (1H, br s), 5.30 (1H, d), 5.20 (1H, td), 4.90 (1H, m), 4.00 (3H, s), 1.40 (3H, d) ppm |
| 2,5-Dichloro-4-(2-furanyl- methylamino)-6- methoxycarbonyl-pyrimidine | 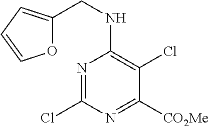 | 7.40 (1H, s), 6.36 (2H, m), 6.19 (1H, br t), 4.72 (2H, d), 3.97 (3H, s) ppm |
| 4-Cyclopropylmethylamino-2,5- dichloro-6-methoxycarbonyl- pyrimidine | 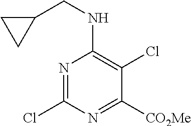 | 5.98 (1H, br s), 3.97 (3H, s), 3.39 (2H, dd), 1.11 (1H, m), 0.63 (2H, m), 0.32 (2H, m) ppm |
| 2,5-Dichloro-6- methoxycarbonyl-4- phenylmethylamino-pyrimidine | 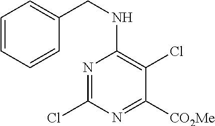 | 7.37 (5H, m), 6.14 (1H, br t), 4.73 (2H, d), 3.97 (3H, s) ppm |
| 2,5-Dichloro-6- methoxycarbonyl-4-(2- nitrophenyl-methylamino)- pyrimidine | 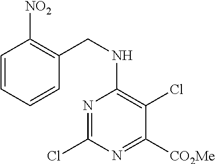 | 8.13 (1H, dd), 7.76 (1H, dd), 7.67 (1H, d), 7.52 (1H, td), 6.87 (1H, br t), 5.01 (2H, d), 3.95 (3H, s) ppm |
| 4-(3-Chloropyrid-2-yl- methylamino)-2,5-dichloro-6- methoxycarbonyl-pyrimidine | 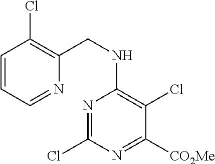 | 8.64 (1H, br t), 8.43 (1H, dd), 7.91 (1H, m), 7.33 (1H, dd), 4.74 (2H, d), 3.86 (3H, s) ppm (nmr run in d₆-DMSO) |
| 4-(2-Amino-1,2-diphenyl- ethylamino)-2,5-dichloro-6- methoxycarbonyl-pyrimidine | 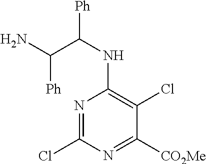 | 7.24 (6H, m), 7.05 (2H, d), 6.99 (2H, d), 5.37 (1H, t), 4.42 (1H, m), 3.96 (3H, s) ppm (NH and NH₂ not observed) |

### 4.2 Preparation of 2-(4-chloro-3-fluorophenyl)-5,6-dimethyl-4-methoxycarbonyl-7H-pyrrolo[2,3-d]pyrimidine (Compound 1-24)

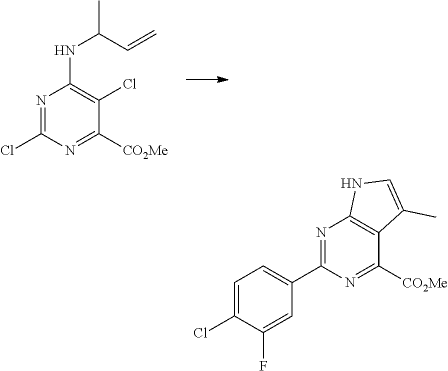

A mixture of 4-(but-1-en-3-ylamino)-2,5-dichloro-6-methoxycarbonyl-pyrimidine (276 mg, 1.0 mmol), 4-chloro-3-fluorophenylboronic acid (210 mg, 1.2 mmol), tris(dibenzylideneacetone)dipalladium(0) (46 mg, 0.05 mmol), tri-t-butylphosphine tetrafluoroboric acid complex (29 mg, 0.10 mmol), caesium carbonate (652 mg, 2.0 mmol), dioxane (6 ml) and dimethylformamide (2 ml) was heated in a microwave reactor at 150° C. for 20 minutes, then allowed to cool. Dichloromethane was added and the mixture washed with water, dried over magnesium sulphate, filtered and evaporated under reduced pressure. The residue was purified by automated flash chromatography (Presearch Combiflash Rf) on silica, with ethyl acetate in hexane (0% to 40% gradient) as eluent, followed by further purification using a FractionLynx hplc, to provide 2-(4-chloro-3-fluorophenyl)-5,6-dimethyl-4-methoxycarbonyl-7H-pyrrolo[2,3-d]pyrimidine as a yellow solid (49 mg, 15%).

Characterising data for the compound are as follows:

¹H NMR (400 MHz, CDCl₃) δ 8.47 (1H, br s), 8.28 (1H, dd), 8.23 (1H, dd), 7.46 (1H, dd), 4.09 (3H, s), 2.46 (3H, s), 2.32 (3H, s) ppm.

Further examples of compounds that were prepared using this method are listed below in Table 20.

TABLE 20
Compounds made according to the method described in Example 4.2 above.

| Compound | | | ¹H NMR (400 MHz, |
|---|---|---|---|
| Number | Name | Structure | CDCl₃) δ |
| 1-20 | 2-(4-Chloro-3- fluorophenyl)-4- methoxycarbonyl-5- methyl-7H-pyrrolo[2,3- d]pyrimidine | 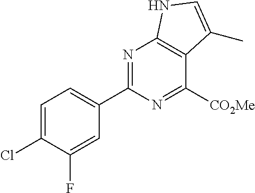 | 8.70 (1H, br s), 8.30 (1H, dd), 8.20 (1H, dd), 7.50 (1H, t), 7.20 (1H, m), 4.10 (3H, s), 2.40 (3H, s) ppm |
| 1-116 | 2-(4-Chloro-3- dimethylamino-2- fluorophenyl)-4- methoxycarbonyl-5- methyl-7H-pyrrolo[2,3- d]pyrimidine | 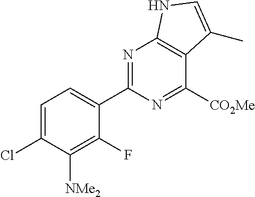 | 10.60 (1H, br s), 7.70 (1H t), 7.30 (1H, dd), 7.20 (1H, br s), 4.10 (3H, s), 2.90 (6H, s), 2.40 (3H, s) ppm |
| 1-120 | 2-(4-Chloro-3- dimethylamino-2- fluorophenyl)-5,6- dimethyl-4- methoxycarbonyl-7H- pyrrolo[2,3-d]pyrimidine | 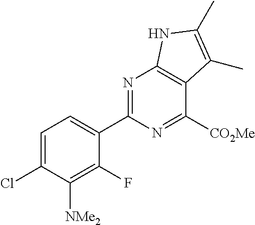 | 9.70 (1H, br s), 7.70 (1H, t), 7.30 (1H, m), 4.10 (3H, s), 2.90 (6H, s), 2.30 (3H, s), 2.25 (3H, d) ppm |
| 22-20 | 6-(4-Chloro-3- fluorophenyl)-4- methoxycarbonyl-3- methyl-1H-pyrrolo[3,2- c]pyridine | 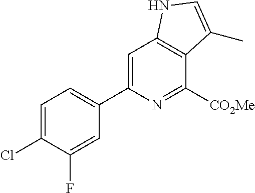 | 8.40 (1H, br s), 7.90 (1H, dd), 7.70 (2H, m), 7.40 (1H, t), 7.10 (1H, br s), 4.10 (3H, s), 2.40 (3H, s) ppm |

### Example 5

### Synthesis of 6-(4-chloro-2-fluoro-3-methoxyphenyl)-4-methoxycarbonyl-3-methyl-1H-pyrrolo[3,2-c]pyridine (Compound 22-64)

### 5.1 Preparation of 2,5-dichloro-6-methoxycarbonyl-4-(prop-2-enylamino)-pyridine

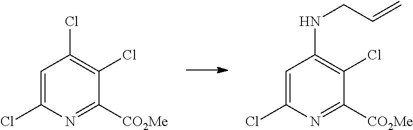

A solution of 6-methoxycarbonyl-2,4,5-trichloro-pyridine (1000 mg, 4.0 mmol), allylamine (240 mg, 4.8 mmol) and triethylamine (1.1 ml, 8.0 mmol) in dimethylformamide (10 ml) was heated at 100° C. for 3 hours, allowed to cool to ambient temperature, water added and the mixture extracted with ethyl acetate. The organic extract was washed with brine, dried over magnesium sulphate, filtered and evaporated under reduced pressure. The residue was purified by automated flash chromatography (Presearch Combiflash Rf) on silica, using ethyl acetate in hexane (20% to 40% gradient) as eluent to provide 2,5-dichloro-6-methoxycarbonyl-4-(prop-2-enylamino)-pyridine as an off-white solid (680 mg, 65%).

Characterising data for the compound are as follows:

¹H NMR (400 MHz, CDCl₃) δ 6.60 (1H, s), 5.90 (1H, m), 5.40 (1H, br s), 5.30 (2H, m), 4.00 (3H, s), 3.90 (2H, m) ppm.

Further examples of compounds that were prepared using this method are listed below in Table 21.

TABLE 21
Compounds made according to the method
described in Example 5.1 above.

| | | ¹H NMR (400 |
|---|---|---|
| Name | Structure | MHz, CDCl₃) δ |
| 2,5-Dichloro- 4-(furan-2- ylmethylamino)- 6-methoxy- carbonyl- pyridine | 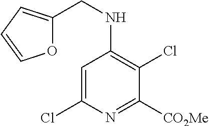 | 7.42 (m, 1H), 6.72 (s, 1H), 6.37 (m, 1H), 6.32 (m, 1H), 5.55 (br. s, 1H), 4.44 (d, 2H), 3.96 (s, 3H) ppm |

### 5.2 Preparation of 5-chloro-2-(4-chloro-2-fluoro-3-methoxyphenyl)-6-methoxycarbonyl-4-(prop-2-enylamino)-pyridine

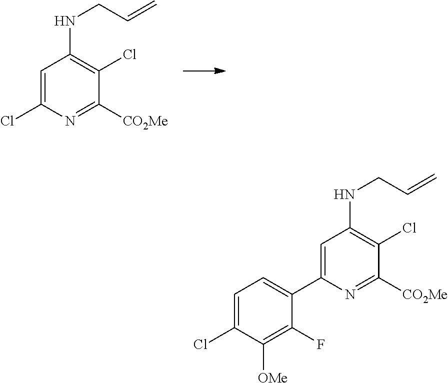

A mixture of 2,5-dichloro-6-methoxycarbonyl-4-(prop-2-enylamino)-pyridine (260 mg, 1.0 mmol), 4-chloro-2-fluoro-3-methoxyphenylboronic acid 1,3-propanediol ester (290 mg, 1.2 mmol), [1,1′-bis(diphenylphosphino)-ferrocene] dichloropalladium (II) complex with dichloromethane (1:1) (82 mg, 0.1 mmol) and caesium fluroide (300 mg, 2.0 mmol), dimethoxyethane (7 ml) and water (7 ml) was heated in a microwave reactor at 140° C. for 40 minutes, allowed to cool to ambient temperature and dichloromethane added. The resulting mixture was washed with water, dried over magnesium sulphate, filtered and evaporated under reduced pressure. The residue was purified by automated flash chromatography (Presearch Combiflash Rf) on silica, using ethyl acetate:hexane (1:4) as eluent to provide 5-chloro-2-(4-chloro-2-fluoro-3-methoxyphenyl)-6-methoxycarbonyl-4-(prop-2-enylamino)-pyridine as a colourless oil (110 mg, 29%).

Characterising data for the compound are as follows:

¹H NMR (400 MHz, CDCl₃) δ 7.60 (1H, t), 7.20 (1H, dd), 7.00 (1H, s), 5.90 (1H, m), 5.20 (3H, m), 4.00 (8H, m) ppm.

Further examples of compounds that were prepared using this method are listed below in Table 22.

TABLE 22
Compounds made according to the method described in Example 5.2 above.

| | | ¹H NMR (400 MHz, |
|---|---|---|
| Name | Structure | CDCl₃) δ |
| 5-Chloro-2-(4-chloro-2- fluoro-3-methoxyphenyl)- 4-(furan-2-ylmethylamino)-6- methoxycarbonyl-pyridine | 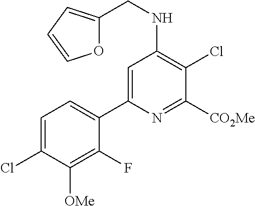 | 7.64 (1H, t), 7.41 (1H, s), 7.25 (1H, m), 7.17 (1H, m), 6.37 (1H, m), 6.34 (1H, m), 5.49 (1H, m), 4.50 (2H, d), 4.00 (3H, s), 3.99 (3H, s) ppm |
| 5-Chloro-2-(4-chloro-3- fluorophenyl)-4-(furan- 2-ylmethylamino)-6- methoxycarbonyl- pyrimidine | 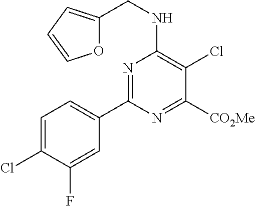 | 8.17 (2H, m), 7.46 (1H, m), 7.40 (1H, d), 6.35 (2H, m), 6.02 (1H, br t), 4.83 (2H, d), 4.02 (3H, s) ppm |
| 5-Chloro-2-(4-chloro-3- fluorophenyl)-4- cyclopropylmethylamino- 6-methoxycarbonyl- pyrimidine | 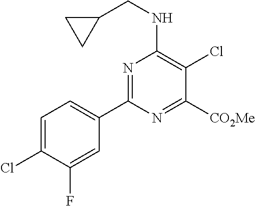 | 8.14 (2H, m), 7.45 (1H, dd), 5.83 (1H, br t), 4.02 (3H, s), 3.50 (2H, dd), 1.18 (1H, m), 0.63 (2H, m), 0.35 (2H, m) ppm |
| 5-Chloro-2-(4-chloro-3- fluorophenyl)-6- methoxycarbonyl-4- phenylmethylamino- pyrimidine | 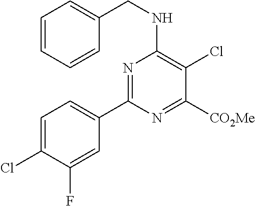 | 8.14 (2H, m), 7.45 (1H, t), 7.39 (4H, m), 7.34 (1H, m), 6.05 (1H, br t), 4.84 (2H, d), 4.02 (3H, s) ppm |
| 5-Chloro-2-(4-chloro-3- fluorophenyl)-6- methoxycarbonyl-4-(2- nitrophenyl-methylamino)- pyrimidine | 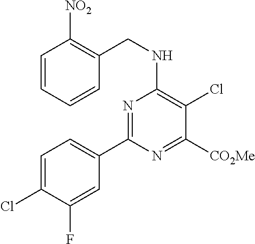 | 8.12 (3H, m), 7.70 (1H, dd), 7.63 (1H, td), 7.48 (2H, m), 6.62 (1H, br t), 5.14 (2H, d), 4.02 (3H, s) ppm |
| 5-Chloro-2-(4-chloro-3- fluorophenyl)-4-(3- chloropyrid-2-yl-methyl- amino)-6-methoxycarbonyl- pyrimidine | 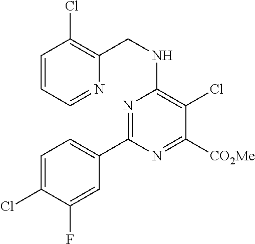 | 8.55 (1H, dd), 8.22 (2H, m), 7.78 (1H, dd), 7.65 (1H, br t), 7.48 (1H, dd), 7.28 (1H, m), 4.96 (2H, d), 4.04 (3H, s) ppm |
| 4-(2-Amino-1,2- diphenyl-ethylamino)-5- chloro-2-(4-chloro-2- fluoro-3-methoxyphenyl)- 6-methoxycarbonyl- pyrimidine | 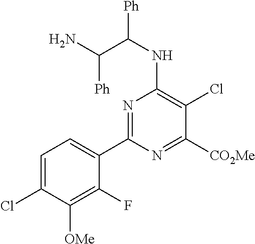 | 7.42 (1H, dd), 7.24 (4H, m), 7.09 (5H, m), 6.95 (2H, m), 5.43 (1H, m), 4.45 (1H, m), 3.97 (3H, s), 3.95 (3H, s) ppm (NH and NH₂ not observed) |
| 5-Chloro-2-(4-chloro-2- fluoro-3-methoxyphenyl)- 6-methoxycarbonyl-4-(2- nitrophenyl-methylamino)- pyrimidine | 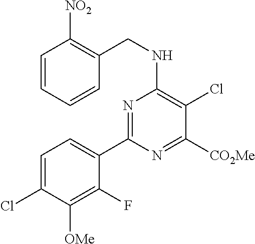 | 8.13 (1H, m), 7.75 (1H, m), 7.69 (1H, m), 7.61 (1H, t), 7.49 (1H, m), 7.22 (1H, dd), 6.71 (1H, br t), 5.10 (2H, d), 4.01 (3H, s), 3.97 (3H, s) ppm |
| 5-Chloro-2-(4-chloro-2- fluoro-3-methoxyphenyl)- 4-cyclopropylmethylamino- 6-methoxycarbonyl- pyrimidine | 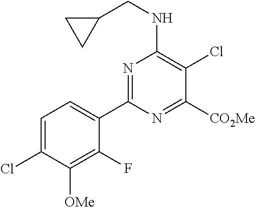 | 7.70 (1H, dd), 7.20 (1H, dd), 5.84 (1H, br t), 3.99 (2x 3H, s), 3.45 (2H, m), 1.16 (1H, m), 0.61 (2H, m), 0.33 (2H, m) ppm |

### 5.3 Preparation of 6-(4-chloro-2-fluoro-3-methoxyphenyl)-4-methoxycarbonyl-3-methyl-1H-pyrrolo[3,2-c]pyridine (Compound 22-64)

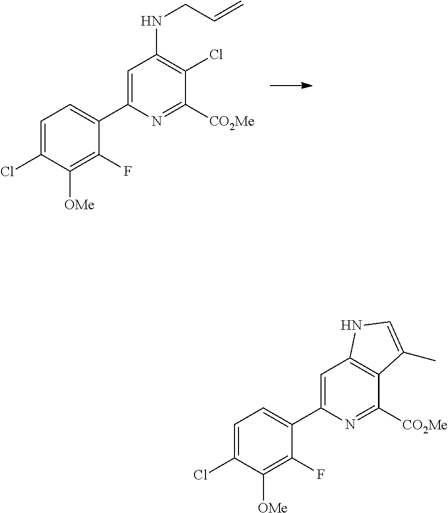

A mixture of 5-chloro-2-(4-chloro-2-fluoro-3-methoxyphenyl)-6-methoxycarbonyl-4-(prop-2-enylamino)-pyridine (110 mg, 0.286 mmol), tris(dibenzylideneacetone)dipalladium(0) (13 mg, 0.014 mmol), tri-t-butylphosphine tetrafluoroboric acid complex (8 mg, 0.028 mmol), caesium carbonate (186 mg, 0.57 mmol), dioxane (3.5 ml) and dimethylformamide (1 ml) was heated in a microwave reactor at 150° C. for 20 minutes, then allowed to cool and ethyl acetate added. The resulting mixture was washed with water, dried over magnesium sulphate, filtered and evaporated under reduced pressure. The residue was purified by automated flash chromatography (Presearch Combiflash Rf) on silica, with ethyl acetate in hexane (0% to 40% gradient) as eluent, to provide 6-(4-chloro-2-fluoro-3-methoxyphenyl)-4-methoxyca rbonyl-3-methyl-1H-pyrrolo[3,2-c]pyridine as an off-white solid (35 mg, 35%).

Characterising data for the compound are as follows:

¹H NMR (400 MHz, CDCl₃) δ 8.60 (1H, br s), 7.90 (1H, d), 7.80 (1H, t), 7.20 (1H, dd), 7.10 (1H, m), 4.10 (3H, s), 4.00 (3H, s), 2.40 (3H, s) ppm.

### Example 6

### Synthesis of 2-cyclopropyl-4-methoxycarbonyl-6-methyl-7H-pyrrolo[2,3-d]pyrimidine (Compound 1-6)

### 6.1 Preparation of 4-amino-2-cyclopropyl-6-methoxycarbonyl-5-(2-methylprop-2-enyl)-pyrimidine

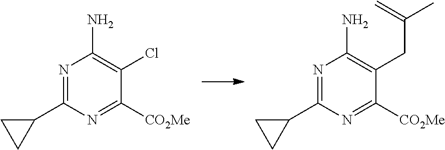

A mixture of 4-amino-5-chloro-2-cyclopropyl-6-methoxycarbonylpyrimidine (prepared as described in WO2010/092339; 227 mg, 1.0 mmol), (1-tributylstannyl)-2-methyl-prop-2-ene (414 mg, 1.2 mmol), bis-(tri-t-butylphosphine)palladium (26 mg, 0.05 mmol) and degassed dimethylformamide (10 ml) was heated in a microwave reactor at 160° C. for 20 minutes, then allowed to cool and ethyl acetate added. The mixture was washed with water and brine, dried over magnesium sulphate, filtered and evaporated under reduced pressure. The residue was purified by automated flash chromatography (Presearch Combiflash Rf) on silica, with ethyl acetate in hexane (0% to 40% gradient) as eluent, to provide 4-am ino-2-cyclopropyl-6-methoxycarbonyl-5-(2-methylprop-2-enyl)-pyrimidine as a white solid (210 mg, 85%).

Characterising data for the compound are as follows:

¹H NMR (400 MHz, CDCl₃) δ 5.10 (2H, br s), 4.90 (1H, m), 4.80 (1H, m), 3.90 (3H. S), 3.40 (2H, s), 2.10 (1H, m), 1.70 (3H, s), 1.00 (4H, m) ppm.

Further examples of compounds that were prepared using this method are listed below in Table 23.

TABLE 23
Compounds made according to the method described in Example 6.1 above.
Characteristic data is ¹H NMR (400 MHz, CDCl₃) δ or mass ion.

| Name | Structure | Characteristic data |
|---|---|---|
| 4-Amino-2-(4-chloro-2- fluoro-3-methoxyphenyl)- 6-methoxycarbonyl-5-(2- methylprop-2-enyl)- pyridine | 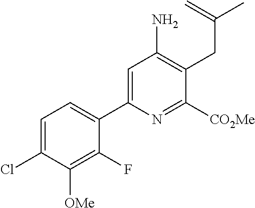 | MH⁺ 365, 367 |
| 4-Amino-2-(4-chloro-2- fluoro-3-methoxyphenyl)- 6-methoxycarbonyl-5-(2- methylprop-2-enyl)- pyrimidine | 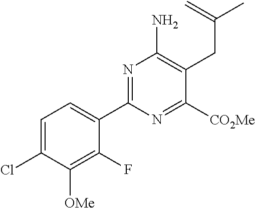 | 7.64 (1H, dd), 7.22 (1H, d), 5.34 (2H, br s), 4.97 (1H, s), 4.85 (1H, s), 4.00 (3H, s), 3.96 (3H, s), 3.50 (2H, s), 1.78 (3H, s) ppm |
| 2-(4-Chloro-3- fluorophenyl)-4-(furan- 2-ylmethylamino)-6- methoxycarbonyl-5-(2- methylprop-2-enyl)- pyrimidine | 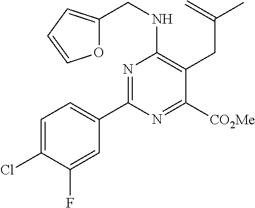 | 8.20 (2H, m), 7.45 (1H, dd), 7.38 (1H, d), 6.33 (1H, m), 6.27 (1H, m), 5.60 (1H, br t), 4.95 (1H, m), 4.80 (3H, m), 3.98 (3H, s), 3.49 (2H, s), 1.73 (3H, s) ppm |
| 2-(4-Chloro-3- fluorophenyl)-6- methoxycarbonyl-5-(2- methylprop-2-enyl)-4- (2-nitrophenyl- methylamino)- pyrimidine | 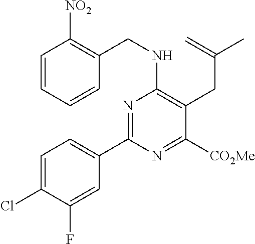 | 8.11 (3H, m), 7.69 (1H, dd), 7.59 (1H, t), 7.45 (2H, m), 6.18 (1H, br t), 5.10 (2H, d), 4.94 (1H, s), 4.81 (1H, s), 3.96 (3H, s), 3.44 (2H, s), 1.72 (3H, s) ppm |
| 2-Cyclopropyl-6- methoxycarbonyl-5-(2- methylprop-2-enyl)-4- (2-nitrophenyl- methylamino)- pyrimidine | 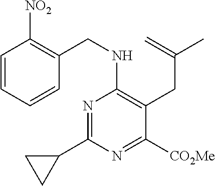 | 8.06 (1H, d), 7.59 (2H, m), 7.44 (1H, m), 5.97 (1H, br t), 4.90 (3H, m), 4.75 (1H, s), 3.91 (3H, s), 3.36 (2H, s), 2.09 (1H, m), 1.59 (3H, s), 0.95 (4H, m) ppm |
| 2-(4-Chloro-2-fluoro-3- methoxyphenyl)-6- methoxycarbonyl-5-(2- methylprop-2-enyl)-4- (2-nitrophenyl- methylamino)- pyrimidine | 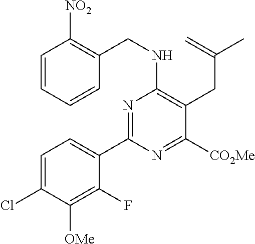 | MH⁺ 501, 503 |
| 2-(4-Chloro-2-fluoro-3- methoxyphenyl)-4- cyclopropylmethylamino- 6-methoxycarbonyl-5- (2-methylprop-2-enyl)- pyrimidine | 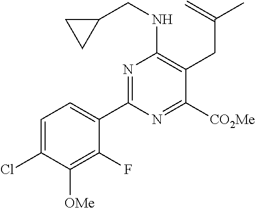 | 7.72 (1H, dd), 7.20 (1H, dd), 5.45 (1H, br t), 4.99 (1H, m), 4.90 (1H, m), 3.99 (3H, s), 3.97 (3H, s), 3.50 (2H, s), 3.40 (2H, m), 1.76 (3H, s), 1.10 (1H, m), 0.54 (2H, m), 0.25 (2H, m) ppm |
| 2-(4-Chloro-2-fluoro-3- methoxyphenyl)-6- methoxycarbonyl-5-(2- methylprop-2-enyl)-4- phenylmethylamino- pyrimidine | 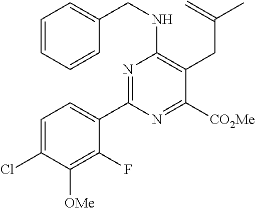 | 7.69 (1H, dd), 7.32 (5H, m), 7.19 (1H, dd), 5.61 (1H, br t), 4.92 (1H, m), 4.78 (3H, m), 3.99 (3H, s), 3.96 (3H, s), 3.49 (2H, s), 2.73 (3H, s) ppm |

### 6.2 Preparation of 2-cyclopropyl-4-methoxycarbonyl-6-methyl-7H-pyrrolo[2,3-d]pyrimidine (Compound 1-6)

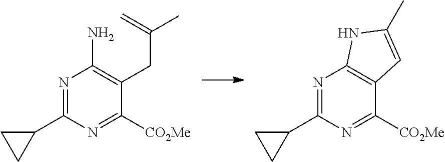

Ozone was bubbled through a solution of 4-amino-2-cyclopropyl-6-methoxycarbonyl-5-(2-methylprop-2-enyl)-pyrimidine (190 mg, 0.77 mmol) in dichloromethane (40 ml) at −78° C. until a blue colour persisted in the reaction vessel. Oxygen was then bubbled through the reaction mixture until the blue colour disappeared, dimethyl sulphide (2 ml) was added and the mixture was allowed to warm to room temperature and stirred for 3 hours The solution was evaporated under reduced pressure and the residue purified by automated flash chromatography (Presearch Combiflash Rf) on silica, with ethyl acetate in hexane (0% to 40% gradient) as eluent, followed by further purification using a Fraction Lynx hplc, to provide 2-cyclopropyl-4-methoxycarbonyl-6-methyl-7H-pyrrolo[2,3-d]pyrimidine as a pale yellow solid (29 mg, 16%).

Characterising data for the compound are as follows:

¹H NMR (400 MHz, CDCl₃) δ 8.80 (1H, br s), 6.70 (1H, s), 4.10 (3H, s), 2.50 (3H, s), 2.40 (1H, m), 1.10 (4H, m) ppm.

Further examples of compounds that were prepared using this method are listed below in Table 24.

TABLE 24
Compounds made according to the method described in Example 6.2 above.
Characteristic data is melting point (° C.) or ¹H NMR (400 MHz, CDCl₃) δ

| Compound | | | |
|---|---|---|---|
| Number | Name | Structure | Characteristic data |
| 5-66 | 2-(4-Chloro-2-fluoro-3- methoxyphenyl)-7- cyclopropylmethyl-4- methoxycarbonyl-6- methyl-7H-pyrrolo[2,3- d]pyrimidine | 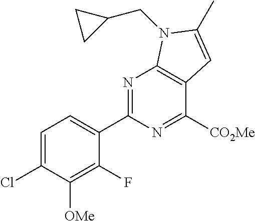 | 120 |
| 6-66 | 2-(4-Chloro-2-fluoro-3- methoxyphenyl)-4- methoxycarbonyl-6- methyl-7-phenylmethyl- 7H-pyrrolo[2,3- d]pyrimidine | 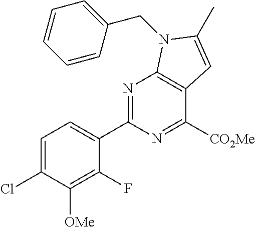 | 7.86 (1H, dd), 7.27 (4H, m), 7.16 (2H, m), 6.86 (1H, s), 5.57 (2H, s), 4.09 (3H, s), 4.02 (3H, s), 2.44 (3H, s) ppm |
| 8-6 | 2-Cyclopropyl-4- methoxycarbonyl-6- methyl-7-(2-nitrophenyl- methyl)-7H-pyrrolo[2,3- d]pyrimidine | 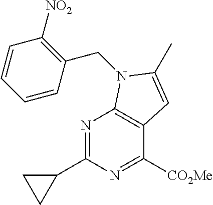 | 8.20 (1H, m), 7.44 (2H, m), 6.82 (1H, s), 6.39 (1H, m), 5.82 (2H, s), 4.08 (3H, s), 2.39 (1H, quintet), 2.32 (3H, s), 1.05 (2H, m), 0.98 (2H, m) ppm |
| 8-22 | 2-(4-Chloro-3- fluorophenyl)-4- methoxycarbonyl-6- methyl-7-(2- nitrophenylmethyl)-7H- pyrrolo[2,3-d]pyrimidine | 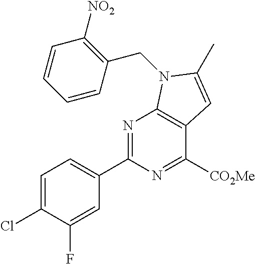 | 8.26 (3H, m), 7.46 (3H, m), 6.95 (1H, s), 6.44 (1H, m), 6.00 (2H, s), 4.13 (3H, s), 2.40 (3H, s) ppm |
| 8-66 | 2-(4-Chloro-2-fluoro-3- methoxyphenyl)-4- methoxycarbonyl-6- methyl-7-(2-nitrophenyl- methyl)-7H-pyrrolo[2,3- d]pyrimidine | 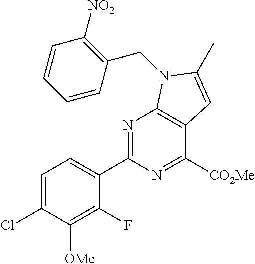 | 198 |
| 1-66 | 2-(4-chloro-2-fluoro-3- methoxyphenyl)-4- methoxycarbonyl-6- methyl-7H-pyrrolo[2,3- d]pyrimidine | 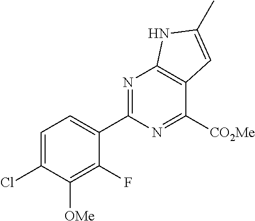 | 9.30 (1H, br s), 7.80 (1H, t), 7.20 (1H, t), 6.80 (1H, m), 4.10 (3H, s), 4.00 (3H, s), 2.50 (3H, s) ppm |
| 22-66 | 6-(4-Chloro-2-fluoro-3- methoxyphenyl)-4- methoxycarbonyl-2- methyl-1H-pyrrolo[3,2- c]pyridine | 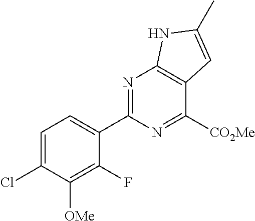 | 8.04 (1H, s), 7.26 (1H, m), 7.12 (1H, m), 7,10 (1H, br s), 6.86 (1H, s), 4.11 (3H, s), 3.92 (3H, s), 2.58 (3H, s) ppm |

### Example 7

### Synthesis of 7-carboxymethyl-2-(4-chloro-3-fluorophenyl)-4-methoxycarbonyl-6-methyl-7H-pyrrolo[2,3-d]pyrimidine (Compound 4-22)

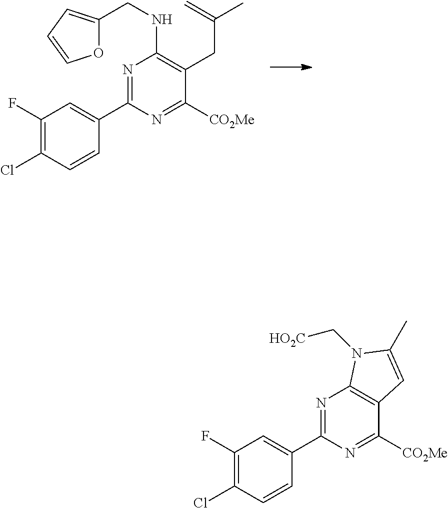

Ozone was bubbled through a solution of 2-(4-chloro-3-fluorophenyl)-4-(2-furanylmethylamino)-6-methoxycarbonyl-5-(2-methylprop-2-enyl)-pyrimidine (150 mg, 0.36 mmol) in dichloromethane (40 ml) at −78° C. until a blue colour persisted in the reaction vessel. Oxygen was then bubbled through the reaction mixture until the blue colour disappeared, dimethyl sulphide (2 ml) was added and the mixture was allowed to warm to room temperature and stirred for 3 hours The solution was evaporated under reduced pressure and the residue purified using a FractionLynx hplc, to provide 7-carboxymethyl-2-(4-chloro-3-fluorophenyl)-4-methoxycarbonyl-6-methyl-7H-pyrrolo[2,3-d]pyrimidine as a yellow solid (5 mg, 4%).

Characterising data for the compound are as follows:

¹H NMR (400 MHz, CD₃OD) δ 8.34 (2H, m) 7.53 (1H, t) 6.74 (1H, s) 4.96 (2H, s) 3.97 (3H, s) 2.52 (3H, s) ppm (CO₂H not observed).

### Example 8

### Synthesis of 2-chloro-4-methoxycarbonyl-7H-pyrrolo[2,3-d]pyrimidine (Compound 1-122) and 4-carboxy-2-chloro-7H-pyrrolo[2,3-d]pyrimidine (Compound 1-121)

### 8.1 Preparation of 4-amino-2-chloro-5-(2-ethoxyethenyl)-6-methoxycarbonyl-pyrimidine

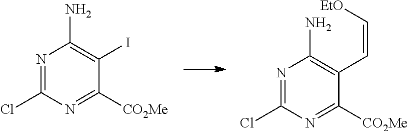

A mixture of 4-amino-2-chloro-5-iodo-6-methoxycarbonyl-pyrimidine (prepared as described in WO2009/046090; 475 mg, 1.5 mmol), 2-ethoxy-(1-tributylstannyl)-ethene (650 mg, 1.8 mmol), bis-(tri-t-butylphosphine)palladium (38 mg, 0.07 mmol) and degassed dimethylformamide (15 ml) was heated in a microwave reactor at 160° C. for 20 minutes, then allowed to cool and ethyl acetate added. The mixture was washed with water and brine, dried over magnesium sulphate, filtered and evaporated under reduced pressure. The residue was purified by automated flash chromatography (Presearch Combiflash Rf) on silica, with ethyl acetate in hexane (0% to 40% gradient) as eluent, to provide 4-amino-2-chloro-5-(2-ethoxyethenyl)-6-methoxycarbonyl-pyrimidine as an off-white solid (154 mg, 39%).

Characterising data for the compound are as follows:

¹H NMR (400 MHz, CDCl₃) δ 6.30 (1H, d), 5.60 (2H, br s), 5.40 (1H, d), 4.00 (2H, q), 3.90 (3H, s), 1.30 (3H, t) ppm.

Further examples of compounds that were prepared using this method are listed below in Table 25.

TABLE 25
Compounds made according to the method described in Example 8.1 above.

| | | ¹H NMR (400 MHz, |
|---|---|---|
| Name | Structure | CDCl₃) δ |
| 4-Amino-2-(4-chloro-2- fluoro-3-methoxyphenyl)- 5-(2-ethoxyethenyl)-6- methoxycarbonyl- pyrimidine | 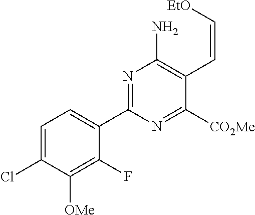 | 7.60 (1H, t), 7.20 (1H, dd), 6.30 (1H, d), 5.60 (2H, br s), 5.50 (1H, d), 4.10 (2H, q), 4.00 (3H, s), 3.90 (3H, s), 1.30 (3H, q) ppm |
| 2-(4-Chloro-2-fluoro-3- methoxyphenyl)-5-(2- ethoxyethenyl)-4-(2- furanylmethylamino)-6- methoxycarbonyl- pyridine | 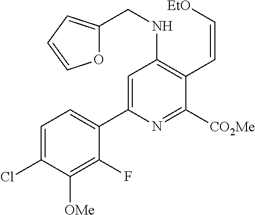 | MH⁺ 461, 463 |
| 2-(4-Chloro-3- fluorophenyl)-5-(2- ethoxyethenyl)-4-(2- furanylmethylamino)-6- methoxycarbonyl- pyrimidine | 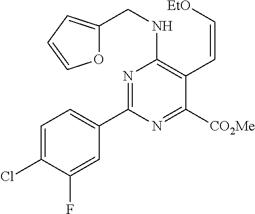 | MH⁺ 432, 434 |
| 2-(4-Chloro-3- fluorophenyl)-4- cyclopropylmethylamino- 5-(2-ethoxyethenyl)- 6-methoxycarbonyl- pyrimidine | 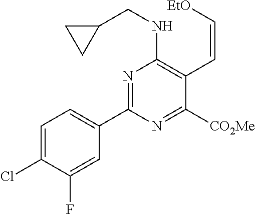 | 8.19 (2H, m), 7.43 (1H, dd), 6.35 (1H, d), 5.72 (1H, br t), 5.40 (1H, d), 3.99 (2H, q), 3.95 (3H, s), 3.49 (2H, m), 1.31 (3H, t), 1.14 (1H, m), 0.60 (2H, m), 0.32 (2H, m) ppm |
| 2-(4-Chloro-3- fluorophenyl)-5-(2- ethoxyethenyl)-6- methoxycarbonyl-4- phenylmethylamino- pyrimidine | 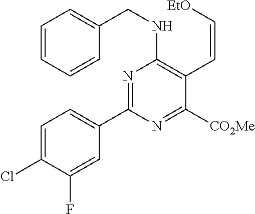 | 8.17 (2H, m), 7.39 (6H, m), 6.30 (1H, d), 5.94 (1H, br t), 5.40 (1H, d), 4.83(2H, d), 3.96 (3H, s), 3.87 (2H, q), 1.13 (3H, t) ppm |
| 2-(4-Chloro-3- fluorophenyl)-5-(2- ethoxyethenyl)-6- methoxycarbonyl-4-(2- nitrophenyl- methylamino)- pyrimidine | 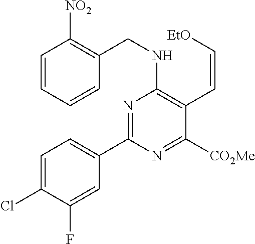 | 8.08 (3H, m), 7.72 (1H, dd), 7.59 (1H, m), 7.44 (2H, m), 6.41 (1H, br t), 6.36 (1H, d), 5.39 (1H, d), 5.14 (2H, d), 4.12 (2H, q), 3.94 (3H, s) 1.28 (3H, t) ppm |
| 2-(4-Chloro-3- fluorophenyl)-4-(3- chloropyrid-2-yl- methylamino)-5-(2- ethoxyethenyl)-6- methoxycarbonyl- pyrimidine | 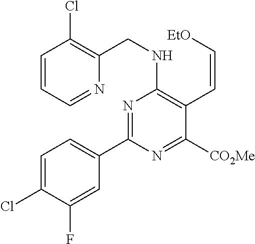 | 8.49 (1H, d), 8.26 (1H, m), 7.75 (1H, d), 7.62 (1H, br q), 7.46 (1H, t), 7.22 (2H, m), 6.44 (1H, d), 5.45 (1H, d), 4.99 (2H, d), 4.00 (2H, q), 3.99 (3H, s), 1.21 (3H, t) ppm |
| 2-Cyclopropyl-5-(2- ethoxyethenyl)-6- methoxycarbonyl-4-(2- nitrophenyl- methylamino)- pyrimidine | 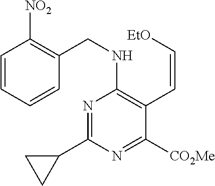 | 8.07 (1H, m), 7.64 (1H, m), 7.56 (1H, m), 7.43 (1H, m), 6.28 (1H, d), 6.22 (1H, br t), 5.35 (1H, d), 4.96 (2H, d), 3.91 (3H, s), 3.49 (2H, m), 2.09 (1H, m), 1.20 (3H, t), 0.91 (4H, m) ppm |

### 8.2 Preparation of 2-chloro-4-methoxycarbonyl-7H-pyrrolo[2,3-d]pyrimidine (Compound 1-122) and 4-carboxy-2-chloro-7H-pyrrolo[2,3-d]pyrimidine (Compound 1-121)

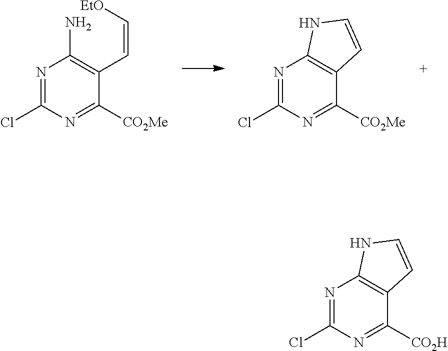

A mixture of 4-amino-2-chloro-5-(2-ethoxyethenyl)-6-methoxycarbonyl-pyrimidine (120 mg, 0.47 mmol) and hydrochloric acid (2N; 5 ml) was heated at reflux for 3 hours, cooled to ambient temperature and evaporated' under reduced pressure. The residue was purified using a FractionLynx hplc, to provide 2-chloro-4-methoxycarbonyl-7H-pyrrolo[2,3-d]pyrimidine (24 mg, 24%).

Characterising data for the compound are as follows:

MH⁺ 212, 214.

Also isolated was 4-carboxy-2-chloro-7H-pyrrolo[2,3-d]pyrimidine as an off-white solid (61 mg, 66%)

Characterising data for the compound are as follows:

¹H NMR (400 MHz, d₆-DMSO) δ 7.50 (1H, d), 6.80 (1H, d), 6.50 (1H, m) ppm (CO₂H not observed).

Further examples of compounds that were prepared using this method are listed below in Table 26.

TABLE 26
Compounds made according to the method described in Example 8.2 above.

| Compound | | | ¹H NMR (400 MHz, |
|---|---|---|---|
| Number | Name | Structure | CDCl₃) δ |
| 1-61 | 4-Carboxy-2-(4-chloro- 2-fluoro-3-methoxyphenyl)- 7H-pyrrolo[2,3- d]pyrimidine | 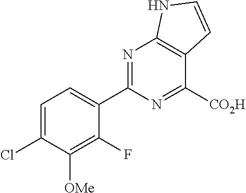 | 7.90 (1H, t), 7.80 (1H, m), 7.50 (1H, m), 6.90 (1H, m), 4.00 (3H, s) ppm (NH and CO₂H not observed) |
| 5-18 | 2-(4-Chloro-3- fluorophenyl)-7- cyclopropylmethyl-4- methoxycarbonyl-7H- pyrrolo[2,3-d]pyrimidine | 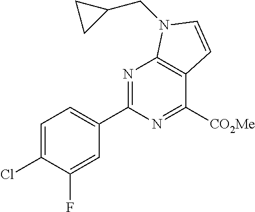 | 8.38 (2H, m), 7.55 (1H, d), 7.52 (1H, dd), 7.07 (1H, d), 4.24 (2H, d), 4.11 (3H, s), 1.22 (1H, m), 0.69 (2H, m), 0.51 (2H, m) ppm |
| 6-18 | 2-(4-Chloro-3- fluorophenyl)-4- methoxycarbonyl-7- phenylmethyl-7H- pyrrolo[2,3-d]pyrimidine | 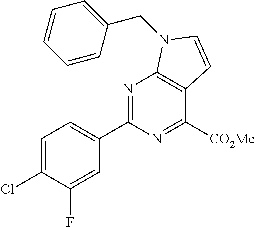 | 8.39 (2H, m), 7.52 (1H, dd), 7.34 (5H, m), 7.29 (1H, m), 7.07 (1H, d), 5.58 (2H, s), 4.12 (3H, s) ppm |
| 8-1 | 4-Carboxy-2- cyclopropyl-7-(2- nitrophenyl-methyl)-7H- pyrrolo[2,3-d]pyrimidine | 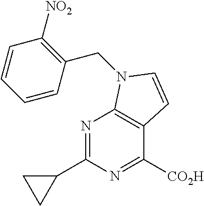 | 8.08 (1H, d), 7.56 (1H, t), 7.51 (1H, t), 7.42 (1H, br s), 6.75 (1H, br s), 6.67 (1H, m), 5.70 (2H, s), 4.08 (1H, br s), 2.13 (1H, m), 0.89 (4H, m) ppm (nmr run in d₆-DMSO) |
| 8-2 | 2-Cyclopropyl-4- methoxycarbonyl-7-(2- nitrophenyl-methyl)-7H- pyrrolo[2,3-d]pyrimidine | 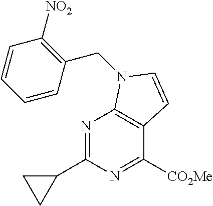 | 8.13 (1H, d), 7.48 (2H, m), 7.31 (1H, d), 7.03 (1H, d), 6.94 (1H, dd), 5.81 (2H, s), 4.07 (3H, s), 2.45 (1H, quintet), 1.12 (2H, m), 1.05 (2H, m) ppm |
| 8-18 | 2-(4-Chloro-3- fluorophenyl)-4- methoxycarbonyl-7-(2- nitrophenyl-methyl)-7H- pyrrolo[2,3-d]pyrimidine | 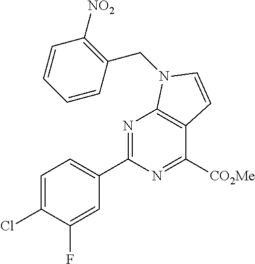 | 8.32 (2H, m), 8.17 (1H, dd), 7.50 (4H, m), 7.15 (1H, d), 7.00 (1H, m), 5.98 (2H, s), 4.13 (3H, s) pm |
| 11-18 | 2-(4-Chloro-3- fluorophenyl)-7-(2- furanyl-methyl)-4- methoxycarbonyl-7H- pyrrolo[2,3-d]pyrimidine | 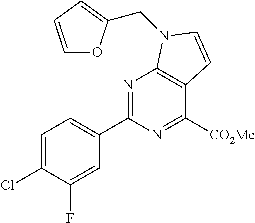 | 8.40 (2H, m), 7.52 (1H, dd), 7.45 (1H, d), 7.40 (1H, m), 7.06 (1H, d), 6.42 (1H, d), 6.37 (1H, m), 5.54 (2H, s), 4.11 (3H, s) ppm |
| 14-18 | 2-(4-Chloro-3- fluorophenyl)-7-(3- chloropyrid-2-yl- methyl)-4-methoxy- carbonyl-7H-pyrrolo[2,3- d]pyrimidine | 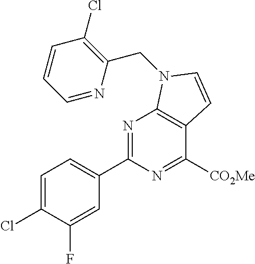 | MH⁺ 431, 433, 435 |
| 32-62 | 6-(4-Chloro-2-fluoro-3- methoxyphenyl)-1-(2- furanyl-methyl)-4- methoxycarbonyl-1H- pyrrolo[3,2-c]pyridine | 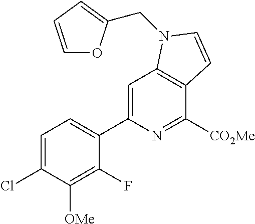 | 7.98 (1H, s), 7.83 (1H, t), 7.37 (2H, m), 7.28 (1H, dd), 7.22 (1H, d), 6.34 (2H, s), 5.33 (2H, s), 4.06 (3H, s), 4.00 (3H, s) ppm |

### Example 9

### Synthesis of 2-chloro-7-(2-furanylmethyl)-4-methoxycarbonyl-6-methyl-7H-pyrrolo[2,3-d]pyrimidine (Compound 11-126)

### 9.1 Preparation of 2-chloro-4-(2-furanylmethylamino)-5-iodo-6-methoxycarbonyl-pyrimidine

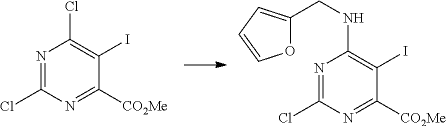

A solution of 2,4-dichloro-5-iodo-6-methoxycarbonyl-pyrimidine (prepared as described in WO2009/046090; 1.0 g, 3.0 mmol), furfurylamine (0,44 g, 4.5 mmol) and triethylamine (0.83 ml, 6.0 mmol) in dichloromethane (10 ml) was stirred at ambient temperature for 1 hour. Dichloromethane was added, the resulting solution washed with water and brine, dried over magnesium sulphate, filtered and evaporated under reduced pressure. The residue was purified by automated flash chromatography (Presearch Combiflash Rf) on silica, with ethyl acetate in hexane (0% to 40% gradient) as eluent, to provide 2-chloro-4-(2-furanylmethylarnino)-5-iodo-6-methoxycarbonyl-pyrimidine as a white solid (898 mg, 76%).

Characterising data for the compound are as follows:

¹H NMR (400 MHz, CDCl₃) δ 7.40 (1H, d), 6.30 (2H, m), 6.20 (1H, br s), 4.70 (2H, d), 4.00 (3H, s) ppm.

### 9.2 Preparation of 2-chloro-7-(2-furanylmethyl)-4-methoxycarbonyl-6-methyl-7H-pyrrolo[2,3-d]pyrimidine (Compound 11-126)

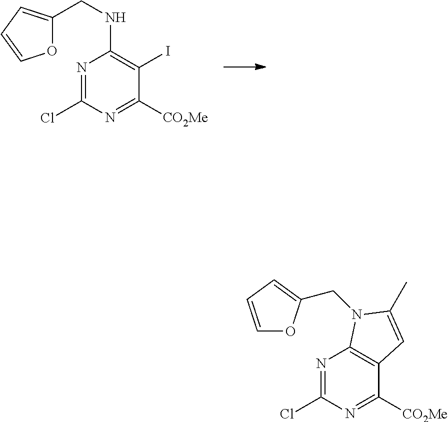

A mixture of 2-chloro-4-(2-furanylmethylamino)-5-iodo-6-methoxycarbonyl-pyrimidine (377 mg, 0.96 mmol), (1-tributylstannyl)-allene (378 mg, 1.15 mmol), bis-(tri-t-butylphosphine)palladium (24 mg, 0.05 mmol) and degassed dimethylformamide (9 ml) was heated in a microwave reactor at 160° C. for 20 minutes, then allowed to cool and ethyl acetate added. The mixture was washed with water and brine, dried over magnesium sulphate, filtered and evaporated under reduced pressure. The residue was purified by automated flash chromatography (Presearch Combiflash Rf) on silica, with ethyl acetate in hexane (0% to 40% gradient) as eluent, followed by further purification using a FractionLynx hplc, to provide 2-chloro-7-(2-furanylmethyl)-4-methoxycarbonyl-6-methyl-7H-pyrrolo[2,3-d]pyrimidine as an off-white solid (75 mg, 26%).

Characterising data for the compound are as follows:

¹H NMR (400 MHz, CDCl₃) δ 7.30 (1H, m), 6.80 (1H, m), 6.20 (2H, m), 5.40 (2H, s), 4.10 (3H, s), 2.50 (3H, s) ppm.

Further examples of compounds that were prepared using this method are listed below in Table 27.

TABLE 27
Compounds made according to the method described in Example 9.2 above.

| Compound | | | ¹H NMR (400 MHz, |
|---|---|---|---|
| Number | Name | Structure | CDCl₃) δ |
| 1-126 | 2-Chloro-4-methoxy- carbonyl-6-methyl-7H- pyrrolo[2,3-d]pyrimidine | 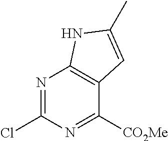 | 6.80 (1H, s), 5.70 (1H, br s), 4.10 (3H, s), 2.60 (3H, s) ppm |

### Example 10

### Synthesis of 6-chloro-4-methoxycarbonyl-1H-pyrrolo[3,2-c]pyridine (Compound 22-122)

### 10.1 Preparation of 4-methoxycarbonyl-5-oxy-1H-pyrrolo[3,2-c]pyridine

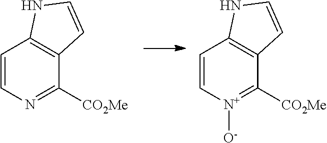

A mixture of 4-methoxycarbonyl-1H-pyrrolo[3,2-c]pyridine (0.50 g, 2.8 mmol), 3-chloroperoxybenzoic acid (0.82 g, 2.8 mmol) and chloroform (10 ml) was stirred at ambient temperature for 4 hours. The reaction mixture was purified by automated flash chromatography (Presearch Combiflash Rf) on silica, with methanol in dichloromethane (0% to 10% gradient) as eluent to provide 4-methoxycarbonyl-5-oxy-1H-pyrrolo[3,2-c]pyridine as an orange foam (220 mg, 40%).

Characterising data for the compound are as follows:

¹H NMR (400 MHz, CDCl₃) δ 12.93 (1H, s), 8.02 (1H, d), 7.55 (2H, m), 6.56 (1H, m), 4.03 (3H, s) ppm.

### 10.2 Preparation of 6-chloro-4-methoxycarbonyl-1H-pyrrolo[3,2-c]pyridine (Compound 22-122)

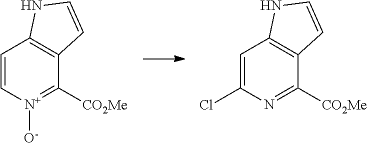

A solution of 4-methoxycarbonyl-5-oxy-1H-pyrrolo[3,2-c]pyridine (220 mg, 1.15 mmol) in phosphorous oxychloride (5 ml) was heated at reflux for 4 hours, then allowed to cool to ambient temperature. The mixture was concentrated under reduced pressure, ice added and the resulting mixture extracted with dichloromethane. The combined organic extracts were evaporated under reduced pressure and the residue purified using a FractionLynx hplc, to provide 6-chloro-4-methoxycarbonyl-1H-pyrrolo[3,2-c]pyridine as a white solid (24 mg, 10%).

Characterising data for the compound are as follows:

¹H NMR (400 MHz, CD₃OD) δ 7.63 (1H, s), 7.55 (1H, d), 7.08 (1H, m), 4.60 (1H, br s), 4.02 (3H, s) ppm.

### Example 11

### Synthesis of 3,6-dichloro-4-methoxycarbonyl-1H-pyrrolo[3,2-c]pyridine (Compound 22-132)

### 11.1 Preparation of 3-chloro-4-methoxycarbonyl-1H-pyrrolo[3,2-c]pyridine

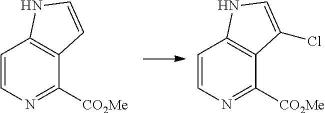

A solution of 4-methoxycarbonyl-1H-pyrrolo[3,2-c]pyridine (0.25 g, 1.4 mmol) and N-chlorosuccinimide (0.23 g, 1.7 mmol) in dimethylformamide (5 ml) was stirred at ambient temperature for 18 hours, then poured into water. The resulting mixture was extracted with diethyl ether and the combined organic phases evaporated under reduced pressure to provide a yellow solid. This was purified using a FractionLynx hplc, to provide 3-chloro-4-methoxycarbonyl-1H-pyrrolo[3,2-c]pyridine as a solid (74 mg, 25%).

Characterising data for the compound are as follows:

¹H NMR (400 MHz, CD₃OD) δ 8.22 (1H, d), 7.57 (1H, d), 7.56 (1H, s), 4.60 (1H, br s), 4.02 (3H, s) ppm.

### 11.2 Preparation of 3-chloro-4-methoxycarbonyl-5-oxy-1 H-pyrrolo[3,2-c]pyridine

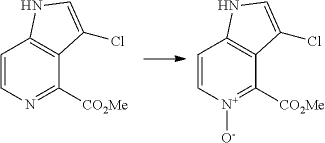

A mixture of 3-chloro-4-methoxycarbonyl-1H-pyrrolo[3,2-c]pyridine (1.16 g, 5.5 mmol), 3-chloroperoxybenzoic acid (1.90 g, 6.6 mmol) and chloroform (10 ml) was stirred at ambient temperature for 16 hours. The reaction mixture was purified by automated flash chromatography (Presearch Combiflash Rf) on silica, with methanol in dichloromethane (0% to 10% gradient) as eluent to provide 3-chloro-4-methoxycarbonyl-5-oxy-1H-pyrrolo[3,2-c]pyridine as an off-white solid (560 mg, 45%).

Characterising data for the compound are as follows:

¹H NMR (400 MHz, CD₃OD) δ 8.12 (1H, d), 7.70 (1H, s), 7.66 (1H, d), 4.60 (1H, br s), 4.07 (3H, s) ppm.

### 11.3 Preparation of 3,6-dichloro-4-methoxycarbonyl-1H-pyrrolo[3,2-c]pyridine Compound 22-132)

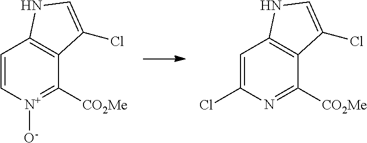

A solution of 3-chloro-4-methoxycarbonyl-5-oxy-1H-pyrrolo[3,2-c]pyridine (520 mg, 5.5 mmol) in phosphorous oxychloride (20 ml) was heated at reflux for 4 hours, then allowed to cool to ambient temperature. The mixture was concentrated under reduced pressure, warm water added and the resulting mixture extracted with ethyl acetate. The combined organic extracts were dried over magnesium sulphate, filtered and evaporated under reduced pressure and the residue purified using a FractionLynx hplc, to provide 3,6-dichloro-4-methoxycarbonyl-1H-pyrrolo[3,2-c]pyridine as a white solid (23 mg, 4%).

Characterising data for the compound are as follows:

¹H NMR (400 MHz, d₆-DMSO) δ 7.86 (1H, s), 7.69 (1H, s), 3.94 (3H, s) ppm (NH not observed).

### Example 12

### Synthesis of 6-chloro-4-methoxycarbonyl-3-methyl-1H-pyrrolo[3,2-c]pyridine (Compound 22-124)

### 12.1 Preparation of 5-bromo-2-chloro-6-methoxycarbonyl-4-(prop-2-enylamino)-pyridine

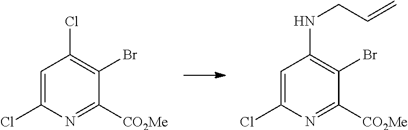

Triethylamine (4.1 ml, 30 mmol), followed by allylamine (0.84 g, 14.7 mmol), were added to a solution of 5-bromo-2,4-dichloro-6-methoxycarbonyl-pyridine (4.20 g, 14.7 mmol) in anhydrous dimethylformamide (50 ml). The resulting reaction mixture was heated at 100° C. for 2 hours, then allowed to cool to ambient temperature and evaporated under reduced pressure to yield an orange coloured oil which was purified by automated flash chromatography (Presearch Combiflash Rf) on silica, with ethyl acetate in isohexane (0% to 70% gradient) as eluent to provide 5-bromo-2-chloro-6-methoxycarbonyl-4-(prop-2-enylamino)-pyridine as a white solid (2.44 g, 54%).

Characterising data for the compound are as follows:

¹H NMR (400 MHz, CDCl₃) δ 6.53 (1H, s), 5.96-5.85 (1H, m), 5.45 (1H, br s), 5.32 (1H, s), 5.28 (1 H, d), 3.97 (3H, s), 3.92 (2H, m) ppm.

### 12.2 Preparation of 6-chloro-4-methoxycarbonyl-3-methyl-1H-pyrrolo[3,2-c]pyridine (Compound 22-124)

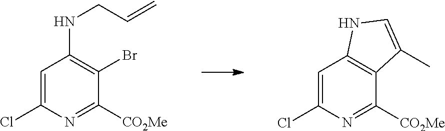

Tetrakis(triphenylphosphine) palladium(0) (1.06 g, 0.91 mmol), followed by degassed dimethylformamide (100 ml), were added to a mixture of 5-bromo-2-chloro-6-methoxycarbonyl-4-(prop-2-enylamino)-pyridine (5.57 g, 18.3 mmol) and finely ground sodium acetate (2.25 g, 27.4 mmol) The mixture was purged with nitrogen and then heated at 105° C. under an atmosphere of nitrogen for 20 hours. Additional tetrakis(triphenylphosphine) palladium(0) (1.06 g, 0.91 mmol) was added and heating continued for a further 24 hours. The reaction mixture was allowed to cool to ambient temperature, poured into water and the resulting mixture extracted with diethyl ethyl. The combined organic phases were combined, washed with brine, dried over magnesium sulphate, filtered and evaporated to yield a yellow solid, which was purified by automated flash chromatography (Presearch Combiflash Rf) on silica, with ethyl acetate in isohexane (0% to 80% gradient) as eluent to provide 6-chloro-4-methoxycarbonyl-3-methyl-1H-pyrrolo[3,2-c]pyridine as a yellow solid (1.92 g, 47%).

Characterising data for the compound are as follows:

¹H NMR (400 MHz, CD₃OD) δ 7.51 (1H, s), 7.28 (1H, s), 4.00 (3H, s), 2.33 (3H, s) ppm (NH not observed).

### Example 13

### Alternative synthesis of 6-(4-chloro-2-fluoro-3-methoxyphenyl)-4-methoxycarbonyl-3-methyl-1H-pyrrolo[3,2-c]pyridine (Compound 22-64)

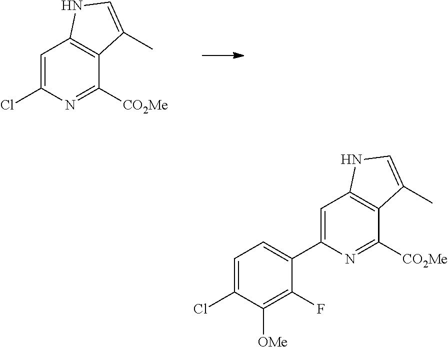

A mixture of 6-chloro-4-methoxycarbonyl-3-methyl-1H-pyrrolo[3,2-c]pyridine (1.00 g, 4.45 mmol), 4-chloro-2-fluoro-3-methoxyphenylboronic acid 1,3-propanediol ester (1.42 g, 5.8 mmol), [1,1′-bis(diphenylphosphino)-ferrocene] dichloropalladium (II) complex with dichloromethane (1:1) (182 mg, 0.22 mmol) and caesium fluoride (1.35 g, 8.9 mmol), dimethylformamide (12 ml) and water (4 ml) was heated in a microwave reactor at 115° C. for 3 hours, allowed to cool to ambient temperature and water added. The resulting mixture was extracted with dichloromethane and the combined organic extracts dried over magnesium sulphate, filtered and evaporated under reduced pressure. The residue was purified by automated flash chromatography (Presearch Combiflash Rf) on silica, with ethyl acetate in isohexane (0% to 60% gradient) to provide 6-(4-chloro-2-fluoro-3-methoxyphenyl)-4-methoxycarbonyl-3-methyl-1H-pyrrolo[3,2-c]pyridine as a beige solid (595 mg, 38%).

Characterising data for the compound are as follows:

¹H NMR (400 MHz, CD₃OD) δ 7.85 (1H, d), 7.65 (1H, t), 7.33 (2H, m), 4.03 (3H, s), 3.99 (3H, s), 2.37 (3H, s) ppm (NH not observed).

Further examples of compounds that were prepared using this method are listed below in Table 28.

TABLE 28
Compounds made according to the method described in Example 13 above. Characteristic data is
melting point (° C.), ¹H NMR (400 MHz, CDCl₃) δ or mass ion

| Compound | | | |
|---|---|---|---|
| Number | Name | Structure | Characteristic data |
| 1-22 | 2-(4-Chloro-3- fluorophenyl)-4- methoxycarbonyl-6- methyl-7H-pyrrolo[2,3- d]pyrimidine | 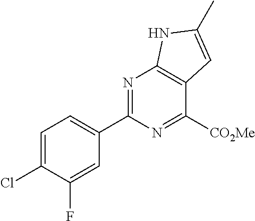 | 8.90 (1H, br s), 8.30 (2H, m), 7.50 (1H, t), 6.80 (1H, s), 4.10 (3H, s), 2.50 (3H, s) ppm |
| 11-22 | 2-(4-Chloro-3- fluorophenyl)-7-(2- furanylmethyl)-4- methoxycarbonyl-6- methyl-7H-pyrrolo[2,3- d]pyrimidine | 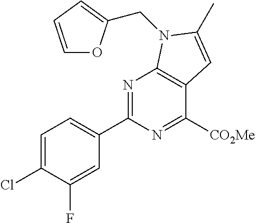 | MH⁺ 400, 402 |
| 11-66 | 2-(4-Chloro-2-fluoro-3- methoxyphenyl)-7-(2- furanylmethyl)-4- methoxycarbonyl-6- methyl-7H-pyrrolo[2,3- d]pyrimidine | 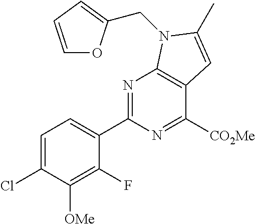 | 130 |
| 22-62 | 6-(4-Chloro-2-fluoro-3- methoxyphenyl)-4- methoxycarbonyl-1H- pyrrolo[3,2-c]pyridine | 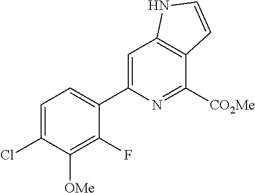 | 7.97 (1H, m), 7.70 (1H, t), 7.60 (1H, d), 7.32 (1H, dd), 7.11 (1H, d), 4.60 (1H, br s), 4.05 (3H, s), 3.98 (3H, s) ppm (nmr run in CD₃OD) |

### Example 14

### Synthesis of 2-(4-chloro-2-fluoro-3-methoxyphenyl)-5,8-dimethyl-4-methoxycarbonyl-5,6,7,8-tetrahydropteridine (Compound 92-164)

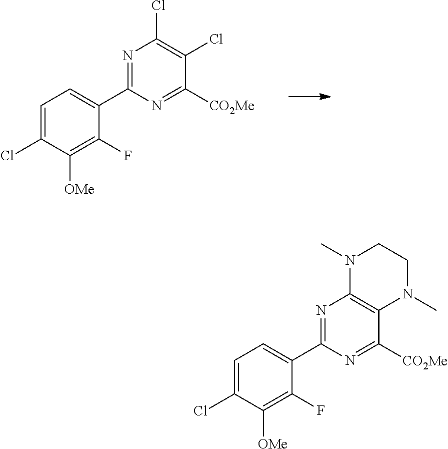

A solution of 2-(4-chloro-2-fluoro-3-methoxyphenyl)-4,5-dichloro-6-methoxycarbonyl-pyrimidine (prepared as described in WO2009/081112, 1.50 g. 4.15 mmol), N,N′-dimethyl-ethane-1,2-diamine (0.5.ml, 4.1 mmol) and triethylamine (0.6 ml, 4.2 mmol) in dichloromethane (20 ml) was stirred at ambient temperature for 45 minute and then evaporated under reduced pressure. The residue was purified by automated flash chromatography (Presearch Combiflash Rf) on silica, with methanol in dichloromethane (0% to 10% gradient)as eluent, followed by further purification using a FractionLynx hplc, to provide 2-(4-chloro-2-fluoro-3-methoxyphenyl)-5,8-dimethyl-4-methoxycarbonyl-5,6,7,8-tetrahydropteridine as a white solid (310 mg, 20%).

Characterising data for the compound are as follows:

M.p. 97° C.;

¹H NMR (400 MHz, CDCl₃) δ 7.68 (1H, dd), 7.16 (1H, m), 3.97 (3H, s), 3.96 (3H, s), 3.54 (2H, m), 3.42 (2H, m), 3.23 (3H, s), 2.88 (3H, s) ppm.

Further examples of compounds that were prepared using this method are listed below in Table 29.

TABLE 29
Compounds made according to the method described in Example 14 above.

| Compound | | | |
|---|---|---|---|
| Number | Name | Structure | Melting point (° C.) |
| 93-166 | 2-(4-Chloro-2-fluoro-3- methoxyphenyl)-5,8- diisopropyl-4-methoxy- carbonyl-5,6,7,8- tetrahydropteridine | 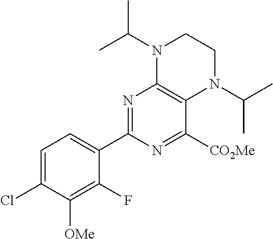 | 135 |
| 95-168 | 2-(4-Chloro-2-fluoro-3- methoxyphenyl)-5,8- dibenzyl-4-methoxy- carbonyl-5,6,7,8- tetrahydropteridine | 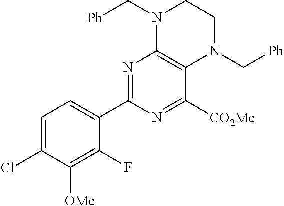 | 146 |

### Example 15

### Synthesis of 2-(4-chloro-2-fluoro-3-methoxyphenyl)-5,8-diphenyl-4-methoxycarbonyl-5,6,7,8-tetrahydropteridine (Compound 94-170)

### 15.1 Preparation of 5-chloro-2-(4-chloro-2-fluoro-3-methoxyphenyl)-4-(N,N′-diphenyl-2-amino-ethylamino)-6-methoxycarbonyl-pyrimidine

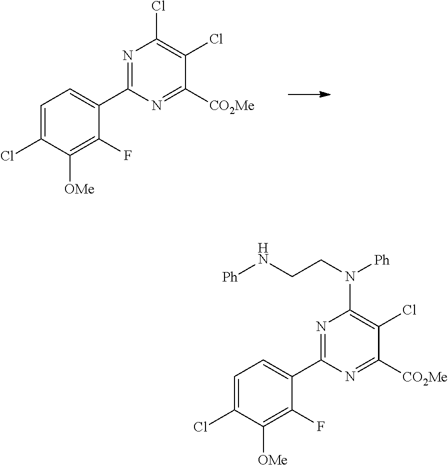

A mixture of 2-(4-chloro-2-fluoro-3-methoxyphenyl)-4,5-dichloro-6-methoxycarbonyl-pyrimidine (prepared as described in WO2009/081112; 360 mg, 1.0 mmol), tris(dibenzylideneacetone)dipalladium(0) (90 mg, 0.10 mmol), Xantphos (60 mg, 0.10 mmol), sodium carbonate (130 mg, 1.2 mmol), water (10 drops) and dimethoxyethane (5 ml) was heated in a microwave reactor at 140° C. for 75 minutes, then allowed to cool and ethyl acetate added. The mixture was washed with water and brine, dried over magnesium sulphate, filtered and evaporated under reduced pressure. The residue was purified by automated flash chromatography (Presearch Combiflash Rf) on silica, with ethyl acetate in hexane (10% to 40% gradient) as eluent, to provide 5-chloro-2-(4-chloro-2-fluoro-3-methoxyphenyl)-4-(N,N′-diphenyl-2-amino-ethylamino)-6-methoxycarbonyl-pyrimidine as a solid (100 mg, 19%).

Characterising data for the compound are as follows:

MH⁺ 541, 543, 545;

### 15.2 Preparation of 2-(4-chloro-2-fluoro-3-methoxyphenyl)-5,8-diphenyl-4-methoxycarbonyl-5,6,7,8-tetrahydropteridine (Compound 94-170)

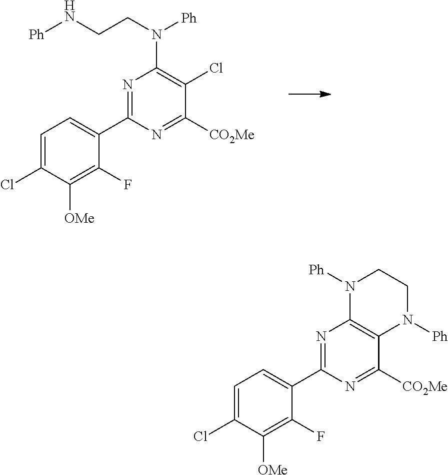

A mixture of 5-chloro-2-(4-chloro-2-fluoro-3-methoxyphenyl)-4-(N,N′-diphenyl-2-amino-ethylamino)-6-methoxycarbonyl-pyrimidine (190 mg, 0.35 mmol), tris(dibenzylideneacetone)dipalladium(0) (30 mg, 0.035 mmol), Xantphos (20 mg, 0.035 mmol), sodium carbonate (50 mg, 0.46 mmol), water (10 drops) and dimethoxyethane (4 ml) was heated in a microwave reactor at 140° C. for 75 minutes, then allowed to cool and ethyl acetate added. The mixture was washed with water and brine, dried over magnesium sulphate, filtered and evaporated under reduced pressure. The residue was purified by automated flash chromatography (Presearch Combiflash Rf) on silica, with ethyl acetate in hexane (10% to 30% gradient) as eluent, followed by further purification using a FractionLynx hplc, to provide 2-(4-chloro-2-fluoro-3-methoxyphenyl)-5,8-diphenyl-4-methoxycarbonyl-5,6,7,8-tetrahydropteridine as a yellow solid (4 mg, 2%).

Characterising data for the compound are as follows:

¹H NMR (400 MHz, CDCl₃) δ 7.68 (1H, m), 7.56 (1H, m), 7.45 (3H, m), 7.30 (4H, m), 7.16 (1H, dd), 7.09 (2H, d), 3.95 (3H, s), 3.94 (3H, s), 3.32 (2H, m), 3.06 (2H, m) ppm.

Further examples of compounds that were prepared using this method are listed below in Table 30.

TABLE 30
Compounds made according to the method described in Example 15.2 above.
Characteristic data is melting point (° C.) or ¹H NMR (400 MHz, CDCl₃) δ

| Compound | | | |
|---|---|---|---|
| Number | Name | Structure | Characteristic data |
| 91-44 | 2-cyclopropyl-6,7- diphenyl-4-methoxycarbonyl- 5,6,7,8-tetrahydropteridine | 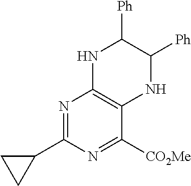 | 7.41 (1H, m), 7.15 (6H, m), 6.82 (2H, d), 6.76 (2H, d), 5.68 (1H, br s), 4.87 (2H, m), 3.95 (3H, s), 2.12 (1H, m), 0.90 (4H, m) ppm |
| 91-184 | 2-(4-Chloro-2-fluoro-3- methoxyphenyl)-6,7- dimethyl-4-methoxy- carbonyl-5,6,7,8- tetrahydropteridine | 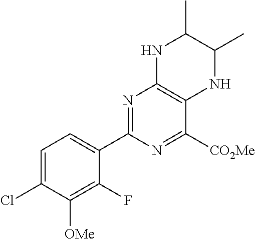 | 110 |
| 91-203 | 4-Carboxy-2-(4-chloro- 2-fluoro-3-methoxyphenyl)- 6,7-diphenyl-5,6,7,8- tetrahydropteridine | 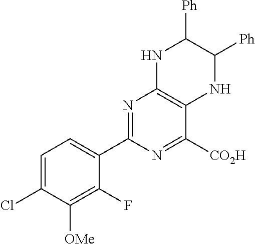 | 230 |
| 91-204 | 2-(4-Chloro-2-fluoro-3- methoxyphenyl)-6,7- diphenyl-4-methoxycarbonyl- 5,6,7,8-tetrahydropteridine | 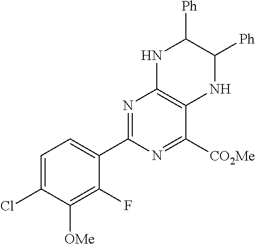 | 7.77 (1H, m), 7.66 (1H, dd), 7.21 (3H, m), 7.14 (4H, m), 6.83 (2H, d), 6.79 (2H, d), 5.92 (1H, br s), 4.98 (2H, m), 4.01 (3H, s), 3.96 (3H, s) ppm |
| 91-207 | (5aR,9aR)-4-Carboxy- 2-(4-chloro-2-fluoro-3- methoxyphenyl)- 5,5a,6,7,8,9,9a,10- octahydro-benzo[g]pteridine | 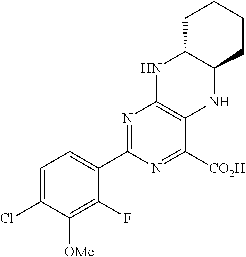 | 236 |
| 106-74 | 2-(4-Chloro-2-fluoro-3- methoxyphenyl)-4- methoxycarbonyl-8- methyl-7,8-dihydro-6H- pyrimido[5,4-b][1,4]oxazine | 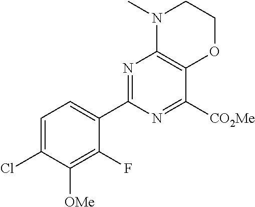 | 117 |

### Example 16

### Synthesis of 8-benzyl-2-(4-chloro-2-fluoro-3-methoxyphenyl)-4-methoxycarbonyl-5,6,7,8-tetrahydropteridine (Compound 95-162)

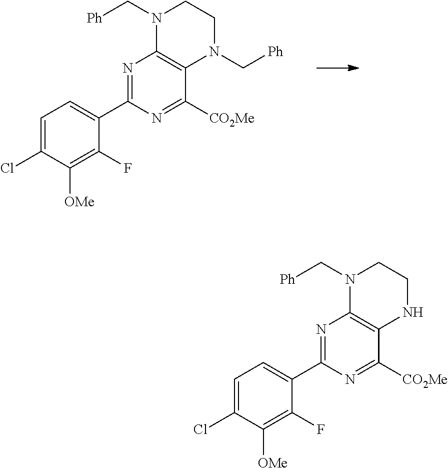

Hydrochloric acid (2N; 3 drops) and palladium (5% on carbon; 32 mg) moistened with water (3 drops) were added to a solution of 2-(4-chloro-2-fluoro-3-methoxyphenyl)-5,8-dibenzyl-4-methoxycarbonyl-5,6,7,8-tetrahydropteridine (prepared as described in example 14; 190 mg, 0.36 mmol) in methanol (10 ml). The mixture was hydrogenated under 4 bar of hydrogen for 6 hours then filtered through Celite®, the filtrate evaporated under reduced pressure and the residue purified using a FractionLynx hplc, to provide 8-benzyl-2-(4-chloro-2-fluoro-3-methoxyphenyl)-4-methoxycarbonyl-5,6,7,8-tetrahydropteridine as a yellow solid (30 mg, 19%).

Characterising data for the compound are as follows:

¹H NMR (400 MHz, CDCl₃) δ 7.70 (1H, t), 7.60 (1H, br s), 7.30 (5H, m), 7.10 (1H, d), 5.00 (2H, s), 4.00 (3H, s), 3.95 (3H, s), 3.50 (4H, s) ppm.

### Example 17

### Synthesis of 2-(4-chloro-2-fluoro-3-methoxyphenyl)-4-methoxycarbonyl-5,10-dihydro-benzo[g]pteridine (Compound 119-168) and 2-(4-chloro-2-fluoro-3-methoxyphenyl)-4-methoxycarbonyl-benzo[g]pteridine (Compound 125.76)

### 17.1 Preparation of 4-(2-amino-phenylamino)-5-chloro-2-(4-chloro-2-fluoro-3-methoxyphenyl)-4-methoxycarbonyl-pyrimidine

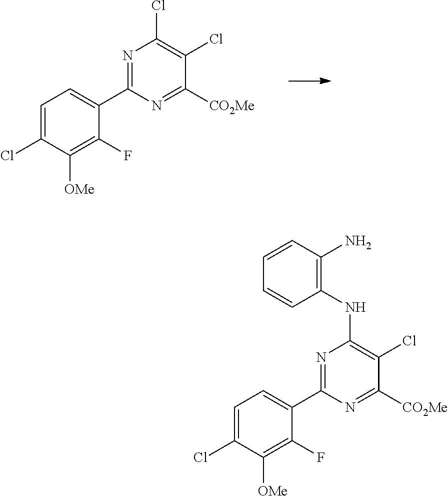

2-Amino-aniline (0.13 g, 1.2 mmol), followed by triethylamine (0.15 ml, 1.3 mmol), were added to a solution of 2-(4-chloro-2-fluoro-3-methoxyphenyl)-4,5-dichloro-6-methoxycarbonyl-pyrimidine (prepared as described in WO2009/081112; 365 mg, 1.0 mmol) in dimethylsulphoxide (6 ml) was heated at 90° C. for 3 hours, then allowed to cool to ambient temperature. The mixture was poured into water and the dark yellow solid removed by filtration and washed with cold dichloromethane to provide 4-(2-amino-phenylamino)-5-chloro-2-(4-chloro-2-fluoro-3-methoxyphenyl)-4-methoxycarbonyl-pyrimidine as a yellow solid (400 mg, 92%).

Characterising data for the compound are as follows:

M.p. 162-164° C.;

¹H NMR (400 MHz, CDCl₃) δ 7.62 (1H, t), 7.59 (1H, d), 7.4(1H, br s), 7.17 (1H, d), 7.12 (1H, t), 6.90 (2H, dd), 4.03 (3H, s), 3.97 (3H, s), 3.70 (2H, br s) ppm.

Further examples of compounds that were prepared using this method are listed below in Table 31.

TABLE 31
Compounds made according to the method described in Example
17.1 above.

| | | Melting point |
|---|---|---|
| Name | Structure | ° C. |
| 4-(2-Amino- phenylamino)-5-chloro- 2-cyclopropyl-4- methoxycarbonyl- pyrimidine | 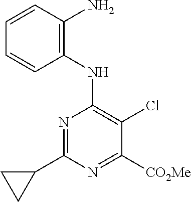 | 164-168 |

### 17.2 Preparation of 2-(4-chloro-2-fluoro-3-methoxyphenyl)-4-methoxycarbonyl-5,10-dihydro-benzo[g]pteridine (Compound 119-168) and 2-(4-chloro-2-fluoro-3-methoxyphenyl)-4-methoxycarbonyl-benzo[g]pteridine (Compound 125-76)

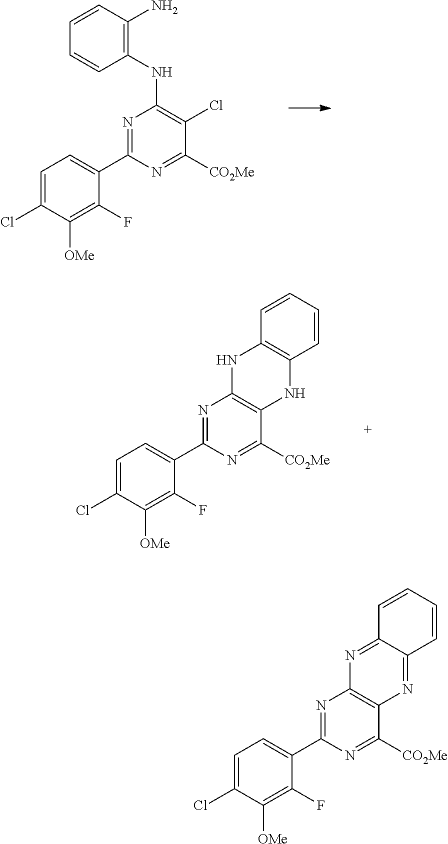

A mixture of 4-(2-amino-phenylamino)-5-chloro-2-(4-chloro-2-fluoro-3-methoxyphenyl)-4-methoxycarbonyl-pyrimidine (170 mg, 0.4 mmol), tris(dibenzylideneacetone)dipalladium(0) (25 mg, 0.03 mmol), Xantphos (45 mg, 0.08 mmol), sodium carbonate (70 mg, 0.65 mmol), water (5 drops) and dimethoxyethane (3 ml) was heated in a microwave reactor at 150° C. for 60 minutes, then allowed to cool to ambient temperature. The mixture was evaporated under reduced pressure and the residue dissolved in water and extracted with ethyl acetate. The combined organic extracts were washed with water and brine, dried over magnesium sulphate, filtered and evaporated under reduced pressure. The residue was purified by chromatography on silica, with 20% ethyl acetate in hexane as eluent, to provide 2-(4-chloro-2-fluoro-3-methoxyphenyl)-4-methoxycarbonyl-5,10-dihydro-benzo[g]pteridine as a yellow solid (50 mg, 32%).

Characterising data for the compound are as follows:

¹H NMR (400 MHz, CDCl₃) δ 8.70 (1H, br s), 7.48 (1H, t), 7.14 (1H, d), 6.88 (1H, br s), 6.62 (1H, t), 6.58 (1H, t), 6.34 (1H, d), 6.14 (1H, d), 3.97 (3H, s), 3.92 (3H, s) ppm.

Also isolated was 2-(4-chloro-2-fluoro-3-methoxyphenyl)-4-methoxycarbonyl-benzo[g]pteridine as a yellow solid (20 mg, 13%).

Characterising data for the compound are as follows:

¹H NMR (400 MHz, CDCl₃) δ 8.40 (2H, t), 8.30 (1H, t), 8.18 (1H, t), 8.00 (1H, t), 7.35 (1H, d), 4.25 (3H, s), 4.10 (3H, s) ppm.

Further examples of compounds that were prepared using this method are listed below in Table 32.

TABLE 32
Compounds made according
to the method described in Example 17.2 above.

| | | | Melting |
|---|---|---|---|
| Compound | | | point |
| Number | Name | Structure | ° C. |
| 119-28 | 2-Cyclo- propyl-4- methoxy- carbonyl-5,10- dihydro- benzo[g] pteridine | 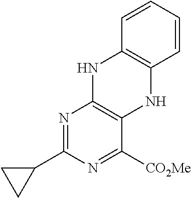 | 217-219 |

### Example 18

### Synthesis of 2-(4-chloro-2-fluoro-3-methoxyphenyl)-7,9-dibenzyl-6-methoxycarbonyl-8-methyl-8,9-dihydro-7H-purine (Compound 81-46)

### 18.1 Preparation of 2-(4-chloro-2-fluoro-3-methoxyphenyl)-4,5-di(benzylamino)-6-methoxycarbonyl-pyrimidine

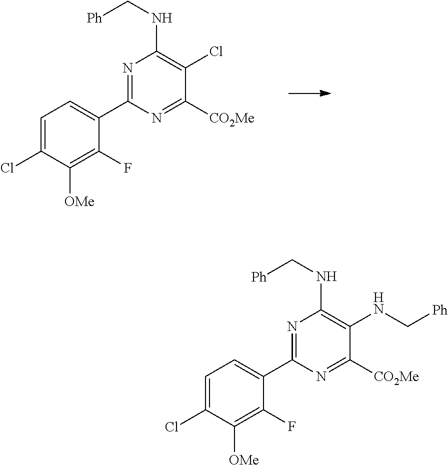

A mixture of 4-benzylamino-5-chloro-2-(4-chloro-2-fluoro-3-methoxyphenyl)-6-methoxycarbonyl-pyrimidine (prepared as described in example 3.1; 330 mg, 0.75 mmol), tris(dibenzylideneacetone)dipalladium(0) (70 mg, 0.075 mmol), Xantphos (50 mg, 0.075 mmol), sodium carbonate (95 mg, 0.90 mmol), water (10 drops) and dimethoxyethane (5 ml) was heated in a microwave reactor at 140° C. for 75 minutes, then allowed to cool and ethyl acetate added. The mixture was washed with water and brine, dried over magnesium sulphate, filtered and evaporated under reduced pressure. The residue was purified by automated flash chromatography (Presearch Combiflash Rf) on silica, with ethyl acetate in hexane (10% to 30% gradient) as eluent, to provide 2-(4-chloro-2-fluoro-3-methoxyphenyl)-4,5-di(benzylamino)-6-methoxyca rbonyl-pyrim idine as a solid (100 mg, 26%).

Characterising data for the compound are as follows:

¹H NMR (400 MHz, CDCl₃) δ 7.70 (1H, dd), 7.30 (6H, m), 7.23 (5H, m), 6.28 (1H, br t), 5.88 (1H, br t), 4.74 (2H, d), 4.15 (2H, d), 3.99 (3H, s), 3.87 (3H, s) ppm.

Further examples of compounds that were prepared using this method are listed below in Table 33.

TABLE 33
Compounds made according to the method described in Example
18.1 above.

| | | ¹H NMR (400 MHz, |
|---|---|---|
| Name | Structure | CDCl₃) δ |
| 2-(4-Chloro-2-fluoro-3- methoxyphenyl)-4,5- bis(2,4- dimethoxyphenyl- methylamino)-6- methoxycarbonyl- pyrimidine | 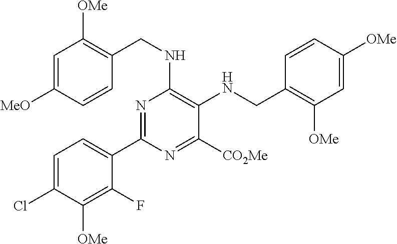 | 7.72 (1H, dd), 7.31 (1H, m), 7.19 (1H, m), 7.05 (1H, d), 6.63 (1H, br t), 6.45 (2H, m), 6.40 (2H, m), 6.08 (1H, t), 4.72 (2H, d), 4.03 (2H, d), 4.01 (3H, s), 3.86 (3H, s), 3.80 (3H, s), 3.79 (3H, s), 3.78 (3H, s), 3.63 (3H, s) ppm |
| 2-(4-Chloro-3- fluorophenyl)-4,5- di(benzylamino)-6- methoxycarbonyl- pyrimidine | 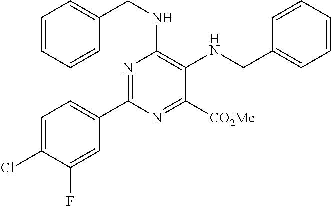 | 8.13 (2H, dd), 7.41 (1H, dd), 7.30 (8H, m), 7.20 (2H, d), 6.18 (1H, br t), 5.92 (1H, br t), 4.78 (2H, d), 4.13 (2H, d), 3.87 (3H, s) ppm |
| 2-(4-Chloro-2-fluoro-3- methoxyphenyl)-4,5- bis(2,4- dimethoxyphenyl- methylamino)-6-(n- propoxycarbonyl)- pyrimidine | 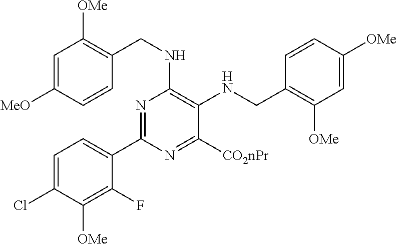 | 7.78 (1H, dd), 7.33 (1H, dd), 7.19 (1H, m), 7.04 (1H, dd), 6.61 (1H, br t), 6.40 (4H, m), 6.09 (1H, br t), 4.74 (2H, d), 4.50 (2H, d), 4.22 (2H, m), 4.01 (3H, s), 3.86 (3H, s), 3.80 (3H, s), 3.79 (3H, s), 3.78 (3H, s), 1.79 (2H, m), 1.00 (3H, t) ppm |

### 18.2 Preparation of 2-(4-chloro-2-fluoro-3-methoxyphenyl)-7,9-dibenzyl-6-methoxycarbonyl-8-methyl-8,9-dihydro-7H-purine (Compound 81-46)

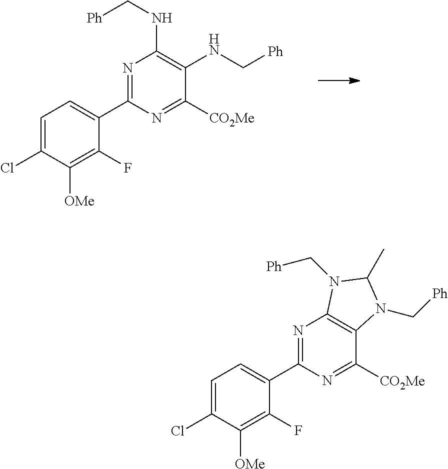

A solution of 2-(4-chloro-2-fluoro-3-methoxyphenyl)-4,5-di(benzylamino)-6-methoxycarbonyl-pyrimidine (100 mg, 0.20 mmol) and acetaldehyde (0.02 ml; 0.36 mmol) in ethanol (5 ml) was heated at reflux for 6 hours, then allowed to cool and evaporated under reduced pressure. The residue was purified using a FractionLynx hplc, to provide 2-(4-chloro-2-fluoro-3-methoxyphenyl)-7,9-dibenzyl-6-methoxycarbonyl-8-methyl-8,9-dihydro-7H-purine as a yellow oil (28 mg, 27%).

Characterising data for the compound are as follows:

¹H NMR (400 MHz, CDCl₃) δ 7.69 (1H, dd), 7.25 (8H, m), 7.16 (1H, d), 7.10 (2H, d), 5.24 (1H, q), 5.19 (2H, dd), 4.56 (1H, d), 4.37 (1H, d), 3.98 (3H, s), 3.81 (3H, s), 1.39 (3H, d) ppm.

Further examples of compounds that were prepared using this method are listed below in Table 34.

TABLE 34
Compounds made according to the method described in Example
18.2 above.

| Compound | | | ¹H NMR (400 MHz, |
|---|---|---|---|
| Number | Name | Structure | CDCl₃) δ |
| 83-48 | 2-(4-Chloro-2-fluoro-3- methoxyphenyl)-7,9- bis(2,4- dimethoxyphenyl- methyl)-6- methoxycarbonyl-8- methyl-8,9-dihydro-7H- purine | 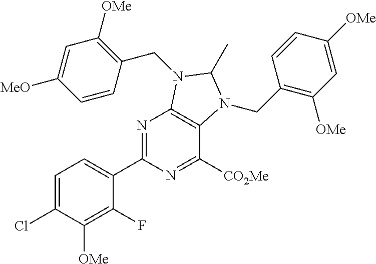 | 7.68 (1H, dd), 7.22 (1H, dd), 7.15 (1H, d), 6.92 (1H, d), 6.37 (2H, m), 6.30 (2H, m), 5.17 (1H, q), 5.14 (1H, d), 4.96 (1H, d), 4.34 (2H, dd), 3.98 (3H, s), 3.84 (3H, s), 3.79 (3H, s), 3.76 (3H, s) 3.67 (3H, s), 3.48 (3H, s), 1.47 (3H, d) ppm |

### Example 19

### Synthesis of 2-(4-chloro-3-fluorophenyl)- 5,8-dibenzyl-6,7-dioxo-4-methoxycarbonyl-5,6,7,8-tetrahydropteridine (Compound 95-160)

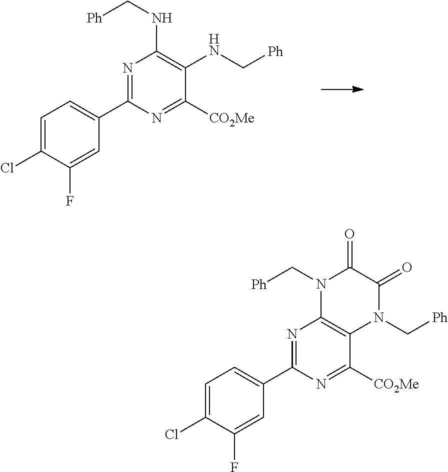

A solution of 2-(4-chloro-3-fluorophenyl)-4,5-di(benzylamino)-6-methoxycarbonyl-pyrimidine (prepared as described in example 18.1; 60 mg, 0.13 mmol) in dichlorobenzene (4 ml) was added to a stirred solution of oxalyl chloride (0.02 ml; 0.18 mmol) in dichlorobenzene (2 ml) at 60° C. The reaction mixture was heated at 160° C. for 1 hour, then allowed to cool and ethyl acetate added. The mixture was washed with water and brine, dried over magnesium sulphate, filtered and evaporated under reduced pressure. The residue was purified by automated flash chromatography (Presearch Combiflash Rf) on silica, with ethyl acetate in hexane (10% to 40% gradient) as eluent, to provide 2-(4-chloro-3-fluorophenyl)-5,8-dibenzyl-6,7-dioxo-4-methoxycarbonyl-5,6,7,8-tetrahydropteridine as a yellow solid (46 mg, 54%).

Characterising data for the compound are as follows:

M.p. 211° C.;

¹H NMR (400 MHz, CDCl₃) δ 8.12 (2H, m), 7.55 (3H, m), 7.30 (6H, m), 7.06 (2H, dd), 5.72 (2H, s), 5.48 (2H, s), 3.57 (3H, s) ppm.

### Example 20

### Synthesis of 4-carboxy-2-(4-chloro-2-fluoro-3-methoxyphenyl)-pyrido[2,3-d]pyrimidine (Compound 123-49)

### 20.1 Preparation of 2-chloro-4-(2-furanyl)-pyrido[2,3-d]pyrimidine

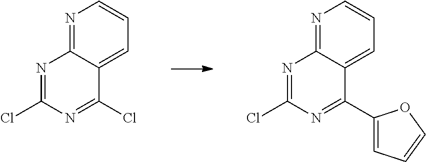

A mixture of 2,4-dichloro-pyrido[2,3-d]pyrimidine (200 mg, 1.0 mmol), 2-(tributylstannyl)-furan (0.35 ml, 1.1 mmol), bis-(triphenylphosphine)palladium dichloride (35 mg, 0.05 mmol) and degassed dimethylformamide (10 ml) was heated in a microwave reactor at 60° C. for 20 minutes, then allowed to cool and ethyl acetate added. The mixture was washed with water and brine, dried over magnesium sulphate, filtered and evaporated under reduced pressure. The residue was purified by automated flash chromatography (Presearch Combiflash Rf) on silica, with methanol in dichloromethane (0% to 10% gradient) as eluent, to provide 2-chloro-4-(2-furanyl)-pyrido[2,3-d]pyrimidine as a yellow solid (217 mg, 94%).

Characterising data for the compound are as follows:

¹H NMR (400 MHz, CDCl₃) δ 9.40 (1H, dd), 9.30 (1H, m), 7.80 (1H, m), 7,70 (1H, m), 7.60 (1H, dd), 6.70 (1H, m) ppm.

### 20.2 Preparation of 2-(4-chloro-2-fluoro-3-methoxyphenyl)-4-(2-furanyl)-pyrido[2,3-d]pyrimidine

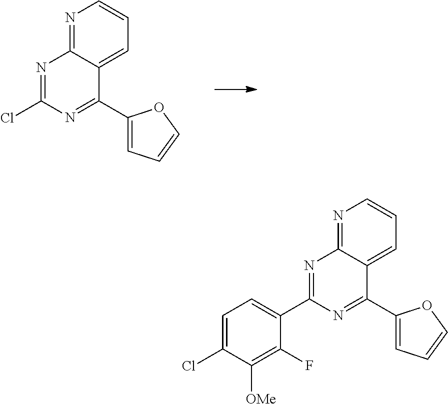

A mixture of 2-chloro-4-(2-furanyl)-pyrido[2,3-d]pyrimidine (217 mg, 0.94 mmol), 4-chloro-2-fluoro-3-methoxyphenylboronic acid 1,3-propanediol ester (275 mg, 1.1 mmol), [1,1′-bis(diphenylphosphino)-ferrocene] dichloropalladium (II) complex with dichloromethane (1:1) (77 mg, 0.09 mmol) and caesium fluoride (283 mg, 1.9 mmol), dimethoxyethane (5 ml) and water (5 ml) was heated in a microwave reactor at 140° C. for 20 minutes, allowed to cool to ambient temperature and extracted with ethyl acetate. The organic extract was washed with brine, dried over magnesium sulphate, filtered and evaporated under reduced pressure. The residue was purified by automated flash chromatography (Presearch Combiflash Rf) on silica, with methanol in dichloromethane (0% to 10% gradient) to provide 2-(4-chloro-2-fluoro-3-methoxyphenyl)-4-(2-furanyl)-pyrido[2,3-d]pyrimidine as a brown solid (137 mg, 39%).

Characterising data for the compound are as follows:

¹H NMR (400 MHz, CDCl₃) δ 9.40 (1H, dd), 9.30 (1H, m), 8.10 (1H, t), 7.90 (1H, m), 7.80 (1H, m), 7.70 (1H, dd), 7.30 (1H, dd), 6.70 (1H, m), 4.00 (3H, s) ppm.

### 20.3 Preparation of 4-carboxy-2-(4-chloro-2-fluoro-3-methoxyphenyl)-pyrido[2,3-d]pyrimidine (Compound 123-49)

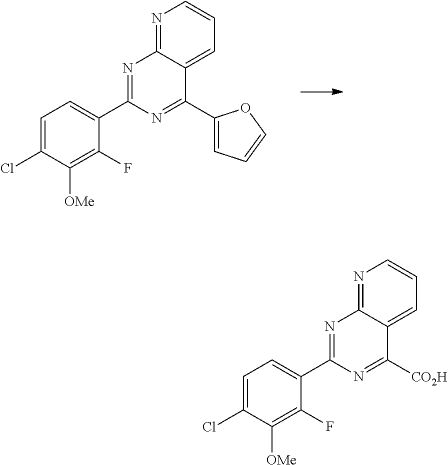

Ozone was bubbled through a solution of 2-(4-chloro-2-fluoro-3-methoxyphenyl)-4-(2-furanyl)-pyrido[2,3-d]pyrimidine (137 mg, 0.37 mmol) in dichloromethane (40 ml) at −78° C. until a blue colour persisted in the reaction vessel. Oxygen was then bubbled through the reaction mixture until the blue colour disappeared, dimethyl sulphide (2 ml) was added and the mixture was allowed to warm to room temperature and stirred for 1 hour. The solution was evaporated under reduced pressure to provide 4-carboxy-2-(4-chloro-2-fluoro-3-methoxyphenyl)-pyrido[2,3-d]pyrimidine.

Characterising data for the compound are as follows:

[M−H]⁻ 332, 334

### Example 21

### Synthesis of 2-(4-chloro-2-fluoro-3-methoxyphenyl)-4-methoxycarbonyl-pyrido[2,3-d]pyrimidine (Compound 123-50)

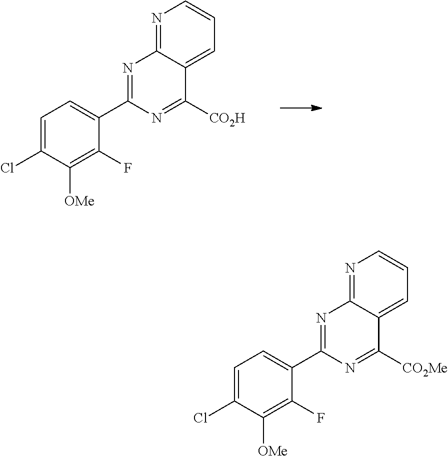

Trimethylsilyldiazomethane (2M in hexane; 0.48 ml, 0.89 mmol) was added to a stirred solution of 4-carboxy-2-(4-chloro-2-fluoro-3-methoxyphenyl)-pyrido[2,3-d]pyrimidine (prepared as described in example 20; 267 mg, 0.80 mmol) in methanol (5 ml) and dichloromethane (20 ml) and the reaction mixture stirred at ambient temperature for 30 minutes. Glacial acetic acid (0.1 ml) was added, the mixture evaporated under reduced pressure and the residue dissolved in ethyl acetate. The solution was washed with water, aqueous sodium hydrogen carbonate and brine, dried over magnesium sulphate, filtered and evaporated under reduced pressure. The residue was purified by automated flash chromatography (Presearch Combiflash Rf) on silica, with methanol in dichloromethane (0% to 10% gradient) to provide 2-(4-chloro-2-fluoro-3-methoxyphenyl)-4-methoxycarbonyl-pyrido[2,3-d]pyrimidine as a brown solid (25 mg, 9%).

Characterising data for the compound are as follows:

M.p. 160-162° C.;

¹H NMR (400 MHz, CDCl₃) δ 9.37 (1H, m), 9.12 (1H, dd), 8.06 (1H, dd), 7.70 (1H, dd), 7.33 (1H, dd), 4.17 (3H, s), 4.07 (3H, s) ppm.

### Example 22

### Synthesis of 4-carboxy-2-(4-chloro-2-fluoro-3-methoxyphenyl)-5-methyl-7H-pyrrolo[2,3-d]pyrimidine (Compound 1-63)

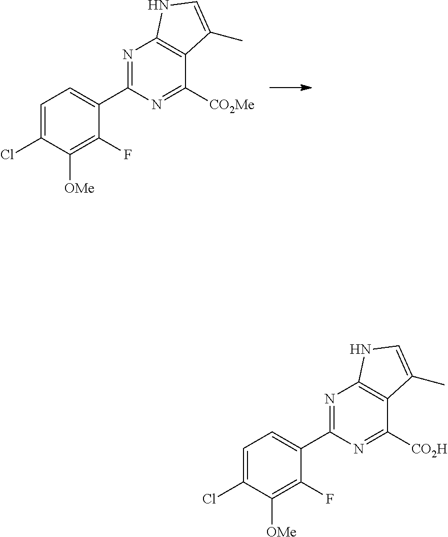

A mixture of 2-(4-chloro-2-fluoro-3-methoxyphenyl)-4-methoxycarbonyl-5-methyl-7H-pyrrolo[2,3-d]pyrimidine (prepared as described in example 5; 260 mg, 0.75 mmol), sodium hydroxide (80 mg, 2.0 mmol), tetrahydrofuran (30 ml) and water (20 ml) was stirred at ambient temperature for 3 hours and then allowed to stand for 12 hours. The reaction mixture was acidified to pH 1-2 and extracted with ethyl acetate. The combined organic extracts were dried over magnesium sulphate, filtered and evaporated under reduced pressure to provide 4-carboxy-2-(4-chloro-2-fluoro-3-methoxyphenyl)-5-methyl-7H-pyrrolo[2,3-d]pyrimidine as a yellow solid (88 mg, 35%).

Characterising data for the compound are as follows:

¹H NMR (500 MHz, d₆-DMSO) δ 12.40 (1H, s), 7.80 (1H, t), 7.60 (1H, br s), 7.40 (1H, d), 4.00 (3H, s), 2.30 (3H, s) ppm (CO₂H not observed).

Further examples of compounds that were prepared using this method are listed below in Table 35.

TABLE 35
Compounds made according to the method described in Example 22
above. Characteristic data is melting point (° C.) or ¹H NMR (400 MHz,
CDC₃) δ

| Compound | | | |
|---|---|---|---|
| Number | Name | Structure | Characteristic data |
| 1-3 | 4-Carboxy-2- cyclopropyl-5-methyl- 7H-pyrrolo[2,3- d]pyrimidine | 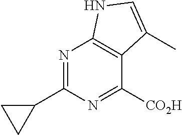 | 204 (dec.) |
| 1-65 | 4-Carboxy-2-(4-chloro- 2-fluoro-3- methoxyphenyl-6- methyl-7H-pyrrolo[2,3- d]pyrimidine | 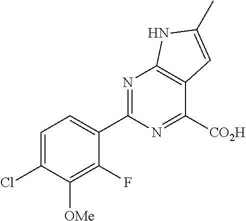 | 7.80 (1H, t), 7.40 (1H, d), 6.60 (1H, s), 4.00 (3H, s), 2.50 (3H, s) ppm (NH and CO₂H not observed) (nmr run in d₆- DMSO) |
| 5-21 | 4-Carboxy-2-(4-chloro- 3-fluorophenyl)-7- cyclopropylmethyl-6- methyl-7H-pyrrolo[2,3- d]pyrimidine | 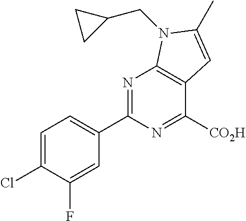 | 8.23 (1H, dd), 8.16 (1H, dd), 7.71 (1H, t), 7.05 (1H, s), 4.34 (2H, d), 2.71 (3H s), 1.36 (1H, m), 0.60 (4H, m) ppm (CO₂H not observed) (nmr run in CD₃OD) |
| 5-65 | 4-Carboxy-2-(4-chloro- 2-fluoro-3- methoxyphenyl)-7- cyclopropymethyl-6- methyl-7H-pyrrolo[2,3- d]pyrimidine | 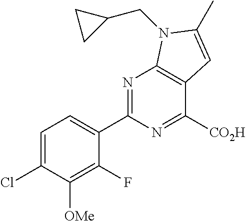 | 7.86 (1H, dd), 7.29 (1H, m), 7.01 (1H, s), 4.22 (2H, d), 4.00 (3H, s), 2.63 (3H, s), 1.26 (1H, m), 0.56 (4H, m) ppm (CO₂H not observed) |
| 6-17 | 4-Carboxy-2-(4-chloro- 3-fluorophenyl)-7- phenylmethyl-7H- pyrrolo[2,3-d]pyrimidine | 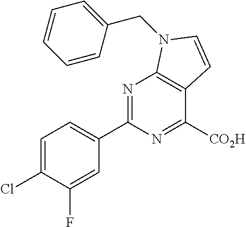 | 8.51 (1H, dd), 8.43 (1H, dd), 7.97 (1H, d), 7.95 (1H, d), 7.79 (1H, t), 7.36 (4H, m), 6.99 (1H, d), 5.6 2(2H, s) ppm (CO₂H not observed) (nmr run in d₆-DMSO) |
| 6-21 | 4-Carboxy-2-(4-chloro- 3-fluorophenyl)-6- methyl-7-phenylmethyl- 7H-pyrrolo[2,3- d]pyrimidine | 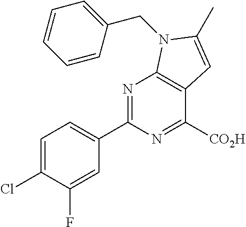 | 8.28 (2H, t), 7.51 (1H, t), 7.30 (3H, m), 7.14 (2H, d), 7.03 (1H, s), 5.60 (2H, s), 2.45 (3H, s) ppm (CO₂H not observed) |
| 6-65 | 4-Carboxy-2-(4-chloro- 2-fluoro-3- methoxyphenyl)-6- methyl-7-phenylmethyl- 7H-pyrrolo[2,3- d]pyrimidine | 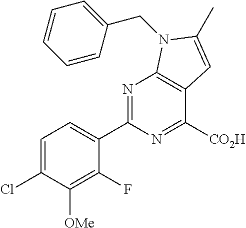 | 165 |
| 8-5 | 4-Carboxy-2- cyclopropyl-6-methyl-7- (2-nitrophenyl-methyl)- 7H-pyrrolo[2,3- d]pyrimidine | 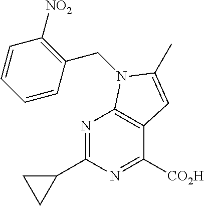 | 8.12 (1H, d), 7.52 (2H, m), 6.69 (1H, s), 6.35 (1H, d), 5.76 (2H, m), 2.27 (3H, s), 2.15 (1H, quintet), 0.88 (4H, m) ppm (CO₂H not observed) (nmr run in d₆-DMSO) |
| 8-17 | 4-Carboxy-2-(4-chloro- 3-fluorophenyl)-7-(2- nitropheny-methyl)-7H- pyrrolo[2,3-d]pyrimidine | 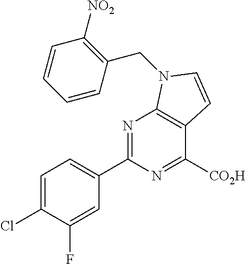 | 8.35 (1H, dd), 8.27 (1H, dd), 8.09 (1H, dd), 7.83 (1H, d), 7.68 (1 H, t), 7.59 (1 H, m), 7.51 (1 H, m), 6.98 (1H, d), 6.89 (1H, d), 5.92 (2H, s) ppm (CO₂H not observed) (nmr run in d₆-DMSO) |
| 8-21 | 4-Carboxy-2-(4-chloro- 3-fluorophenyl)-6- methyl-7-(2- nitrophenyl-methyl)-7H- pyrrolo[2,3-d]pyrimidine | 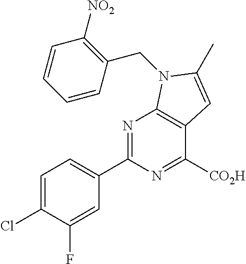 | 8.18 (3H, m), 7.60 (1H, t), 7.51 (2H, m), 6.70 (1H, s), 6.39 (1H, dd), 5.87 (2H, s), 2.29 (3H, s) ppm (CO₂H not observed) (nmr run in d₆- DMSO) |
| 8-65 | 4-Carboxy-2-(4-chloro- 2-fluoro-3- methoxyphenyl)-6- methyl-7-(2- nitrophenyl-methyl)-7H- pyrrolo[2,3-d]pyrimidine | 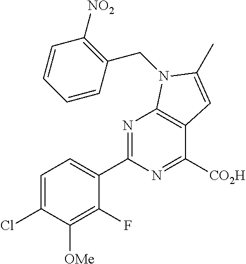 | 187 |
| 11-21 | 4-Carboxy-2-(4-chloro- 3-fluorophenyl)-7-(2- furanyl-methyl)-6- methyl-7H-pyrrolo[2,3- d]pyrimidine | 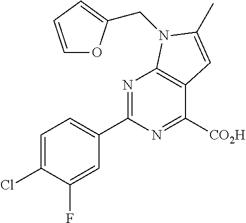 | 8.41 (1H, dd), 8.34 (1H, d), 7.69 (1H, t), 7.52 (1H, d), 6.66 (1H, br s), 6.40 (1H, m), 6.36 (1H, m), 5.55 (2H, s), 2.50 (3H, s) ppm (CO₂H not observed) (nmr run in d₆- DMSO) |
| 12-63 | 4-Carboxy-2-(4-chloro- 2-fluoro-3- methoxyphenyl )-7-(5- trifluoromethyl-furan-2- yl-methyl)-5-methyl-7H- pyrrolo[2, 3-d]pyrimidine | 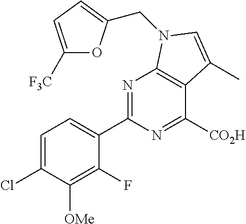 | 158 |
| 14-17 | 4-Carboxy-2-(4-chloro- 3-fluorophenyl)-7-(3- chloropyrid-2-yl- methyl)-7H-pyrrolo[2,3- d]pyrimidine | 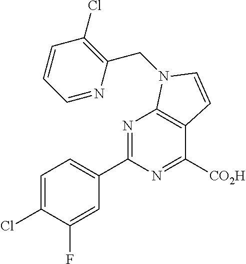 | 8.41 (1H, d), 8.28 (2H, m), 8.02 (1H, s), 7.76 (1H, dd), 7.62 (1H, m), 7.53 (1H, m), 7.29 (1H, m), 5.84 (2H, s) ppm (CO₂H not observed) |
| 22-63 | 4-Carboxy-6-(4-chloro- 2-fluoro-3- methoxyphenyl)-3- methyl-1H-pyrrolo[3,2- c]pyridine | 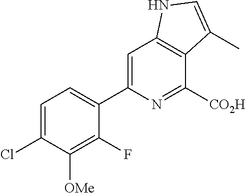 | 11.66 (1H, s), 7.91 (1H, m), 7.86 (1H, t), 7.44 (2H, m), 3.95 (3H, s), 2.34 (3H, s) ppm (CO₂H not observed) (nmr run in d₆-DMSO) |
| 91-183 | 4-Carboxy2-(4-chloro- 2-fluoro-3- methoxyphenyl)-6,7- dimethyl-5,6,7,8- tetrahydropteridine | 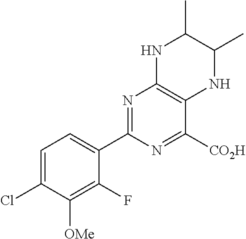 | 8.10 (1H, brs), 7.65 (1H, t), 7.49 (1H, br s), 7.34 (1H, dd), 3.90 (3H, s), 3.62 (2H, m), 1.10 (2 × 3H, s) ppm (CO₂H not observed) (nmr run in d₆-DMSO) |
| 92-163 | 4-Carboxy-2-(4-chloro- 2-fluoro-3- methoxyphenyl)-5,8- dimethyl-5,6,7,8- tetrahydropteridine | 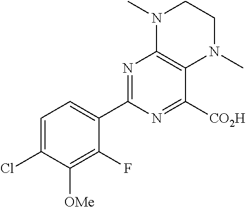 | 148 |
| 93-165 | 4-Carboxy-2-(4-chloro- 2-fluoro-3- methoxyphenyl)-5,8- diisopropyl-5,6,7,8- tetrahydropteridine | 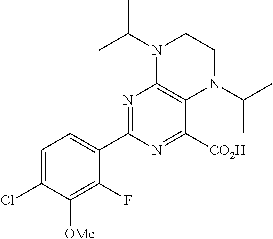 | 125 |

### Example 23

### Synthesis of 6-(4-chloro-2-fluoro-3-methoxyphenyl)-4-ethoxycarbonyl-3-methyl-1H-pyrrolo[3,2-c]pyridine (Compound 22-75)

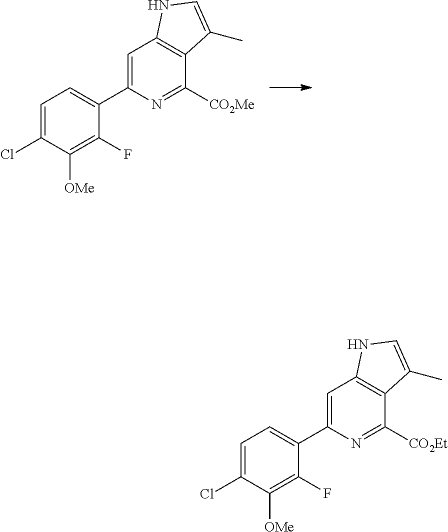

1-Hydroxy-3-isothionato-1,1,3,3-tetrabutyl-distannoxane (16 mg, 0.029 mmol) was added to a suspension of 6-(4-chloro-2-fluoro-3-methoxyphenyl)-4-methoxycarbonyl-3-methyl-1H-pyrrolo[3,2-c]pyridine (prepared as described in example 5; 200 mg, 0.58 mmol) in toluene (5 ml). Ethanol (0.67 ml, 11.5 mmol) was added and the resulting mixture heated in a microwave reactor at 170° C. for 1 hour, then cooled and ethyl acetate and water added. The organic phase was evaporated under reduced pressure and the residue purified using a FractionLynx hplc, to provide 6-(4-chloro-2-fluoro-3-methoxyphenyl)-4-ethoxycarbonyl-3-methyl-1H-pyrrolo[3,2-c]pyridine (98 mg, 47%).

Characterising data for the compound are as follows:

¹H NMR (400 MHz, CDCl₃) δ 8.50 (1H, br. s), 7.87 (1H, m), 7.84 (1H, d), 7.26 (1H, m), 7.16 (1H, m), 4.55 (2H, q), 3.99 (3H, s), 2.42 (3H, s), 1.49 (3H, t) ppm.

Further examples of compounds that were prepared using this method are listed below in Table 36.

TABLE 36
Compounds made according to the method described in Example 23
above. Characteristic data is melting point (°C.) or ¹H NMR (400 MHz,
CDCl₃) δ

| Compound | | | |
|---|---|---|---|
| Number | Name | Structure | Characteristic data |
| 1-75 | 2-(4-Chloro-2-fluoro-3- methoxyphenyl)-4- ethoxycarbonyl-5- methyl-7H-pyrrolo[2,3- d]pyrimidine | 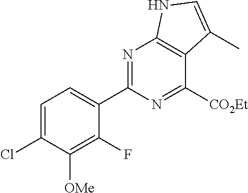 | 172-174 |
| 1-77 | 2-(4-Chloro-2-fluoro-3- methoxyphenyl)-5- methyl-4-(n- octyloxycarbonyl)-7H- pyrrolo[2,3-d]pyrimidine | 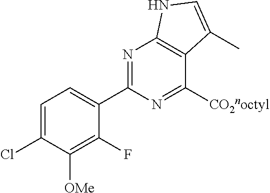 | 9.64 (1H, br s), 7.82 (1H, dd), 7.28 (1H, m), 7.22 (1H, m), 4.50 (2H, t), 4.03 (3H, s), 2.43 (3H, d), 1.85 (2H, quintet), 1.48 (2H, m), 1.30 (8H, m), 0.89 (3H, t) ppm |
| 1-78 | 2-(4-Chloro-2-fluoro-3- methoxyphenyl)-4- isopropoxycarbonyl-5- methyl-7H-pyrrolo[2,3- d]pyrimidine | 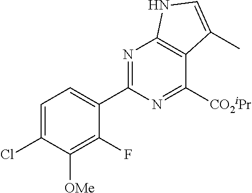 | 154-156 |
| 1-79 | 2-(4-Chloro-2-fluoro-3- methoxyphenyl)-4- (hept-2-yloxycarbonyl)- 5-methyl-7H- pyrrolo[2,3-d]pyrimidine | 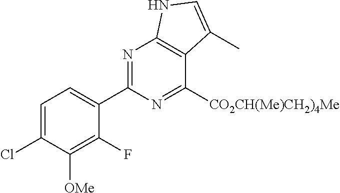 | 9.43 (1H, br s), 7.86 (1H, dd), 7.28 (1H, d), 7.22 (1H, t), 5.32 (1H, m), 4.03 (3H, s), 2.44 (3H, s), 1.85 (1H, m), 1.69 (1H, m), 1.47 (3H, d), 1.35 (3H, m), 0.90 (3H, m) ppm |

0

| | | | |
|---|---|---|---|
| 1-80 | 2-(4-Chloro-2-fluoro-3- methoxyphenyl)-5- methyl-4-(prop-2- enyloxycarbonyl)-7H- pyrrolo[2,3-d]pyrimidine | 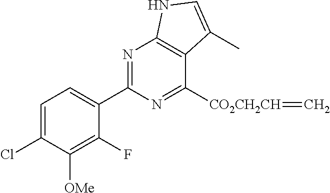 | 10.55 (1H, br s), 7.81 (1H, dd), 7.30 (1H, m), 7.22 (1H, m), 6.10 (1H, m), 5.51 (1H, dd), 5.36 (1H, dd), 5.01 (2H, m), 4.04 (3H, s), 2.44 (3H, s) ppm |
| 1-82 | 2-(4-Chloro-2-fluoro-3- methoxyphenyl)-4-[(2- ethoxy)- ethoxycarbonyl]-5- methyl-7H-pyrrolo[2,3- d]pyrimidine | 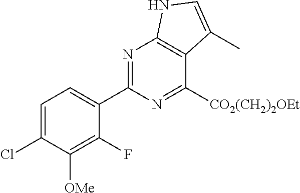 | 9.24 (1H, br s), 7.82 (1H, m), 7.26 (1H, m), 7.22 (1H, m), 4.63 (2H, m), 4.04 (3H, s), 3.85 (2H, 2.43 (3H, s), 1.24 (3H, t), ppm |
| 1-83 | 4-[(2-n-Butoxy)- ethoxycarbonyl]-2-(4- chloro-2-fluoro-3- methoxyphenyl)-5- methy-7H-pyrrolo[2,3- d]pyrimidine | 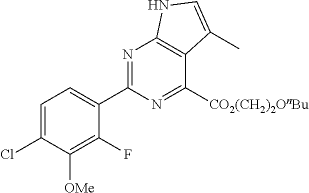 | 9.39 (1H, br s), 7.82 (1H, dd), 7.26 (1H, m), 7.21 (1H, m), 4.63 (2H, m), 4.02 (3H, s), 3.85 (2H, m), 3.53 (2H, t), 2.44 (3H, s), 1.60 (2H, m), 1.37 (2H, sextet), 0.91 (3H, t) ppm |
| 1-84 | 4-[(1-n-Butoxy)-prop-2- yloxycarbonyl]-2-(4- chloro-2-fluoro-3- methoxyphenyl)-5- methyl-7H-pyrrolo[2,3- d]pyrimidne | 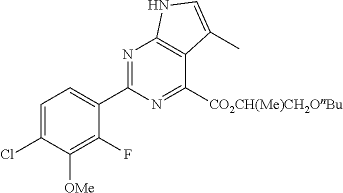 | 9.80 (1H, br s), 7.84 (1H, dd), 7.27 (1H, m), 7.20 (1H, t), 5.49 (1H, sextet), 4.02 (3H, s), 3.68 (2H, ddd), 3.53 (2H, m), 2.42 (3H, d), 1.56 (2H, m), 1.49 (3H, d), 1.37 (2H, sextet), 0.90 (3H, s) ppm |
| 1-85 | 2-(4-Chloro-2-fluoro-3- methoxyphenyl)-4-{2- [(2-methoxy)-ethoxy]- ethoxycarbonyl}-5- methyl-7H-pyrrolo[2,3- d]pyrimidine | 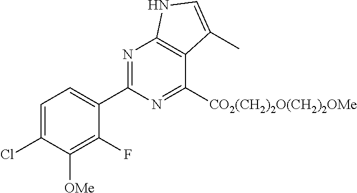 | 10.05 (1H, br s), 7.81 (1H, dd), 7.28 (1H, m), 7.19 (1H, t), 4.69 (2H, m), 4.04 (3H, s), 3.93 (2H, m), 3.75 (2H, m), 3.59 (2H, m), 3.39 (3H, s), 2.41 (3H, d) ppm |
| 1-86 | 4-Benzyloxycarbonyl-2- (4-chloro-2-fluoro-3- methoxyphenyl)-5- methyl-7H-pyrrolo[2,3- d]pyrimidine | 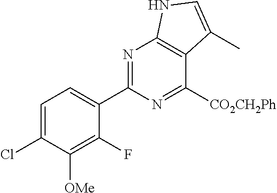 | 10.20 (1H, br s), 7.81 (1H, dd), 7.53 (2H, m), 7.40 (3H m), 7.29 (1H, dd) 7.19 (1H, m), 5.54 (2H, s), 4.04 (3H, s) 2.31 (3H, d) ppm |
| 1-87 | 2-(4-Chloro-2-fluoro-3- methoxyphenyl)-4-(2- furanyl- methoxycarbonyl)-5- methy-7H-pyrrolo[2,3- d]pyrimidine | 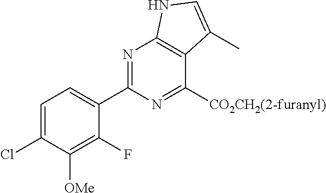 | 9.40 (1H, br s), 7.81 (1H, dd), 7.45 (1H, d), 7.28 (1H, m), 7.20 (1H, m), 6.59 (1H, m), 6.40 (1H, m), 5.46 (2H, s), 4.03 (3H, s), 2.33 (3H, d) ppm |
| 1-88 | 2-(4-Chloro-2-fluoro-3- methoxyphenyl)-5- methyl-4- (tetrahydrofuran-2-yl- methoxycarbonyl)-7H- pyrrolo[2,3-d]pyrimidine | 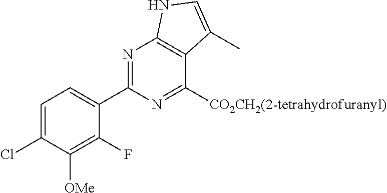 | 9.49 (1H, br s), 7.82 (1H, dd), 7.28 (1H, m), 7.20 (1H, t), 4.55 (1H, dd), 4.46 (1H, m), 4.35 (1H, m), 4.04 (3H, s), 3.96 (1H, m), 3.84 (1H, m), 2.42 (3H, s), 2.10 (1H, m), 1.90 (2H, m), 1.80 (1H, m) ppm |
| 22-77 | 6-(4-Chloro-2-fiuoro-3- methoxyphenyl)-3- methyl)-4-(n- octyloxycarbonyl)1H- pyrrolo[3,2-c]pyridine | 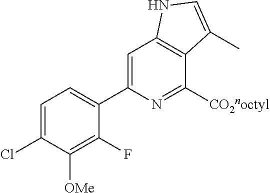 | 8.40 (1H, br s), 7.88 (2H, m), 7.26 (1H, dd), 7.16 (1H, m), 4.48 (2H, t), 3.99 (3H, s), 2.42 (3H, s), 1.85 (2H, m), 1.49 (2H, m), 1.32 (8H, m), 0.89 (3H, m) ppm |
| 22-78 | 6-(4-Chloro-2-fluoro-3- methoxyphenyl)-4- isopropoxycarbonyl-3- methyl-1H-pyrrolo[3,2- c]pyridne | 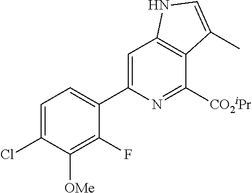 | 8.35 (1H, s), 7.86 (2H, m), 7.26 (1H, m), 7.14 (1H, m), 5.41 (1H, sept), 3.98 (3H, s), 2.42 (3H, s), and 1.48 (6H, d) ppm |
| 22-79 | 6-(4-Chloro-2-fluoro-3- methoxyphenyl)-4- (hept-2-yloxycarbonyl)- 3-methyl-1H- pyrrolo[3,2-c]pyridine | 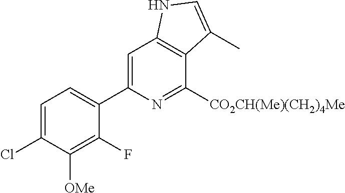 | 8.67 (1H, br s), 7.89 (1H, d), 7.85 m), (1H, m), 7.23 (1H, dd), 7.12 (1H, m), 5.31 (1H, m), 3.97 (3H, s), 2.41 (3H, s), 1.85 (1H, m), 1.66 (1H, m), 1.49 (2H, m), 1.45 (3H, d), 1.33 (4H, m), 0.90 (3H, m) ppm |
| 22-80 | 6-(4-Chloro-2-fluoro-3- methoxyphenyl)-3- methyl-4-(prop-2- enyloxycarbonyl)-1H- pyrrolo[3,2-c]pyridine | 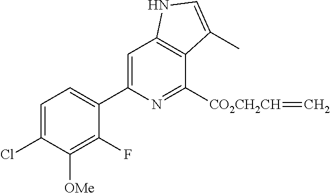 | 8.47 (1H, br s), 7.88 (1H, m), 7.85 (1H, d), 7.26 (1H, m), 7.16 (1H, m), 6.13 (1H, m), 5.50 (1H, dd), 5.34 (1H, dd), 4.98 (2H, m), 3.99 (3H, s), 2.42 (3H, s) ppm |
| 22-82 | 6-(4-Chloro-2-fluoro-3- methoxyphenyl)-4-[(2- ethoxy)- ethoxycarbonyl]-3- methyl-1H-pyrrolo[3,2- c]pyridine | 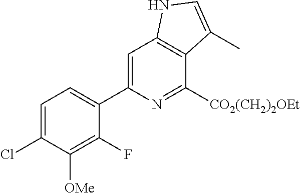 | 8.50 (1H, br s), 7.86 (2H, m), 7.26 (1H, m), 7.13 (1H, m), 4.63 (2H, m), 3.99 (3H, s), 3.86 (2H, m), 3.62 (2H, q), 2.41 (3H, s), 1.24 (3H, t) ppm |
| 22-83 | 4-[(2-n-Butoxy- ethoxycarbonyl]-6-(4- chloro-2-fluoro-3- methoxyphenyl)-3- methyl-1H-pyrrolo[3,2- c]pyridine | 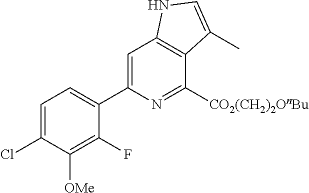 | 8.55 (1H, br s), 7.85 (2H, m), 7.26 (1H, m), 7.12 (1H, m), 4.63 (2H, m), 3.99 (3H, s), 3.85 (2H, m), 3.55 (2H, m), 2.40 (3H, s), 1.59 (2H, m), 1.43-1.32 (2H, m), 0.91 (3H, t) ppm |
| 22-84 | 4-[(1-n-Butoxy)-prop-2- yloxycarbonyl]-6-(4- chloro-2-fluoro-3- methoxyphenyl)-3- methyl-1H-pyrrolo[3,2- c]pyridine | 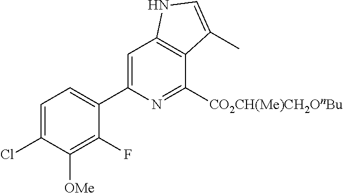 | 8.68 (1H, br s), 7.85 (2H, m), 7.23 (1H, dd), 7.09 (1H, s), 5.49 (1H, m), 3.98 (3H, s) 3.68 (2H, m), 3.55 (2H, m), 2.39 (3H, s), 1.57 (2H, m), 1.48 (3H, d), 1.38 (2H, m), 0.90 (3H, t) ppm |
| 22-85 | 6-(4-Chloro-2-fluoro-3- methoxyphenyl)-4-{2- [(2-methoxy)-ethoxy]- ethoxycarbonyl}-3- methyl-1H-pyrrolo[3,2- c]pyridine | 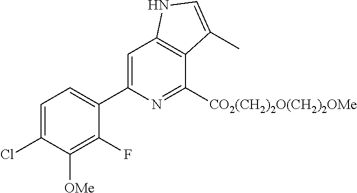 | 8.63 (1H, br s), 7.83 (2H, m), 7.24 (1H, dd), 7.10 (1H, m), 4.64 (2H, m), 3.99 (3H, s), 3.94 (2H, m), 3.74 (2H, m), 3.59 (2H, m), 3.37 (3H, s), 2.37 (3H, s) ppm |
| 22-86 | 4-Benzyloxycarbonyl-6- (4-chloro-2-fluoro-3- methoxyphenyl)-3- methyl-1H-pyrrolo[3,2- c]pyridine | 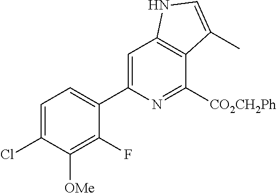 | 8.45 (1H, br s), 7.87 (2H, m), 7.54 (2H, m), 7.36 (3H, m), 7.26 (1H, m), 7.13 (1H, m), 5.53 (2H, s), 3.99 (3H, s), 2.31 (3H, s) ppm |
| 22-87 | 6-(4-Chloro-2-fluoro-3- methoxyphenyl)-4-(2- furanyl- methoxycarbonyl)-3- methyl-1H-pyrrolo[3,2- c]pyridine | 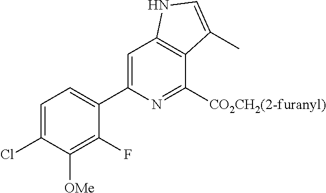 | 8.52 (1H, br s), 7.86 (2H, m), 7.45 (1H, m), 7.26 (1H, m), 7.13 (1H, m), 6.55 (1H, m), 6.39 (1H, m), 5.47 (2H, s), 3.98 (3H, s), 2.33 (3H, s) ppm |
| 22-88 | 6-(4-Chloro-2-fluoro-3- methoxyphenyl)-3- methyl-4- (tetrahydrofuran-2-yl- methoxycarbonyl)-1H- pyrrolo[3,2-c]pyridine | 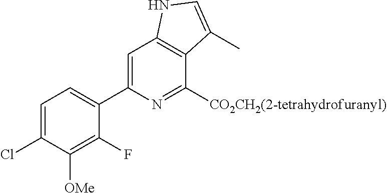 | 8.71 (1H, br s), 7.84 (2H, m), 7.24 (1H, dd), 7.06 (1H, s), 4.55 (1H, m), 4.41 (2H, m), 3.97 (4H, m), 3.86 (1H, m), 2.36 (3H, s), 2.03 (3H, m), 1.80 (1H, m) ppm |
| — | 5-Chloro-2-(4-chloro-2- fluoro-3- methoxyphenyl)-4-(2,4- dimethoxyphenyl- methylamino)-6-(n- propoxycarbonyl)- pyrimidine | 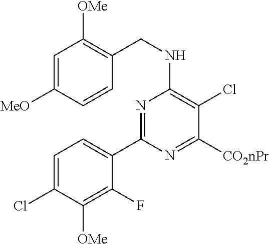 | 7.80 (1H, t), 7.30 (1H, m), 7.20 (1H, m), 6.50 (1H, m), 6.40 (1H, dd), 6.30 (1H, br s), 4.70 (2H, d), 4.30 (2H, t), 4.10 (3H, s), 3.90 (3H, s), 3.80 (3H, s), 1.80 (2H, sept), 1.00 (3H, t) ppm |

### Example 24

### Synthesis of 2-(4-chloro-2-fluoro-3-methoxyphenyl)-4-methoxycarbonyl-5-methyl-7-methylsulphonyl-7H-pyrrolo[2,3-d]pyrimidine (Compound 18-64)

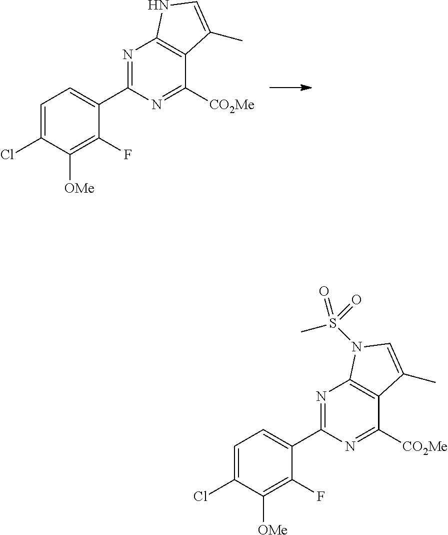

Triethylamine (0.26 ml, 1.9 mmol) was added to a suspension of 2-(4-chloro-2-fluoro-3-methoxyphenyl)-4-methoxycarbonyl-5-methyl-7H-pyrrolo[2,3-d]pyrimidine (prepared as described in example 3; 350 mg, 1.0 mmol) in dichloromethane (12 ml) and the resulting mixture cooled to 0° C. After stirring for 10 mins, methanesulphonyl chloride (0.12 ml, 1.5 mmol) was added and the reaction mixture stirred for 10 minutes at 0° C., then warmed to ambient temperature and stirred for a further 1 hour. The reaction mixture was evaporated under reduced pressure and the residue purified by automated flash chromatography (Presearch Combiflash Rf) on silica, with ethyl acetate in isohexane (10% to 40% gradient) as eluent, followed by further purification using a FractionLynx hplc, to provide 2-(4-chloro-2-fluoro-3-methoxyphenyl)-4-methoxycarbonyl-5-methyl-7-methylsulphonyl-7H-pyrrolo[2,3-d]pyrimidine as an off-white solid (151 mg, 35%).

Characterising data for the compound are as follows:

¹H NMR (400 MHz, CDCl₃) δ 8.00 (1H, m), 7.60 (1H, m), 7.30 (1H, m), 4.10 (3H, s), 4.00 (3H, s), 3.70 (3H, s), 2.40 (3H, s) ppm.

### Example 25

### Synthesis of 2-(4-chloro-2-fluoro-3-methoxyphenyl)-7-ethoxymethyl-4-methoxycarbonyl-5-methyl-7H-pyrrolo[2,3-d]pyrimidine (Compound 3-64) and 4-carboxy-2-(4-chloro-2-fluoro-3-methoxyphenyl)-7-ethoxymethyl-5-methyl-7H-pyrrolo[2,3-d]pyrimidine (Compound 3-63)

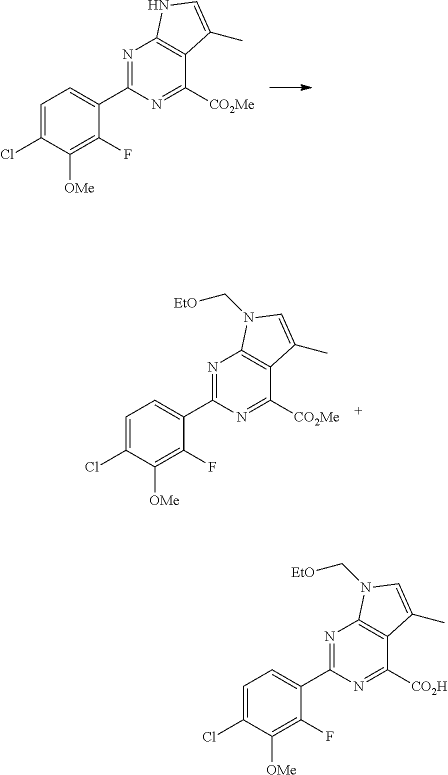

2-(4-Chloro-2-fluoro-3-methoxyphenyl)-4-methoxycarbonyl-5-methyl-7H-pyrrolo[2,3-d]pyrimidine (prepared as described in example 3; 175 mg, 0.5 mmol) was added to a stirred suspension of potassium t-butoxide (112 mg, 1.0 mmol) in tetrahydrofuran (10 ml) at ambient temperature. After stirring for 15mins, chloromethyl ethyl ether (0.09 ml, 1.0 mmol) was added and the reaction mixture stirred for 3 hours, evaporated under reduced pressure and the residue purified using a FractionLynx hplc, to provide 2-(4-chloro-2-fluoro-3-methoxyphenyl)-7-ethoxymethyl-4-methoxycarbonyl-5-methyl-7H-pyrrolo[2,3-d]pyrimidine as an off-white solid (71 mg, 35%).

Characterising data for the compound are as follows:

M.p. 107-108° C.;

¹H NMR (400 MHz, CDCl₃) δ 7.80 (1H, t), 7.30 (1H, m), 7.20 (1H, m), 5.70 (2H, s), 4.10 (3H, s), 4.00 (3H, s), 3.50 (2H, q), 2.50 (3H, s), 1.20 (3H, t) ppm.

Also isolated was 4-carboxy-2-(4-chloro-2-fluoro-3-methoxyphenyl)-7-ethoxymethyl-5-methyl-7H-pyrrolo[2,3-d]pyrimidine as a yellow solid (38 mg, 19%).

Characterising data for the compound are as follows:

M.p. 112-114° C. (dec.);

¹H NMR (500 MHz, d₆-DMSO) δ 7.80 (1H, t), 7.60 (1H, br s), 7.40 (1H, d), 5.60 (2H, s), 3.90 (3H, s), 3.40 (2H, q), 2.30 (3H, s), 1.00 (3H, t) ppm (CO₂H not observed).

Further examples of compounds that were prepared using this method are listed below in Table 37.

TABLE 37
Compounds made according to the method described in Example 24
above. Characteristic data is melting point (° C.) or ¹H NMR (400 MHz,
CDC₃) δ

| Compound | | | |
|---|---|---|---|
| Number | Name | Structure | Characteristic data |
| 6-63 | 7-Benzyl-4-carboxy-2- (4-chloro-2-fluoro-3- methoxyphenyl)-5- methyl-7H-pyrrolo[2,3- d]pyrimidine | 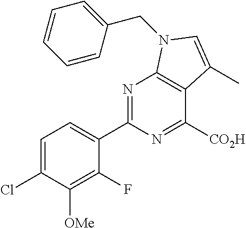 | 7.90 (1H, t), 7.30 (7H, m), 5.50 (2H, s), 4.10 (3H, s), 2.60 (3H, s) ppm (CO₂H not observed) (nmr run in d₆-DMSO) |
| 6-64 | 7-Benzyl-2-(4-chloro-2- (fluoro-3- methoxyphenyl)-4- methoxycarbonyl-5- methyl-7H-pyrrolo[2,3- dlpyrimidine | 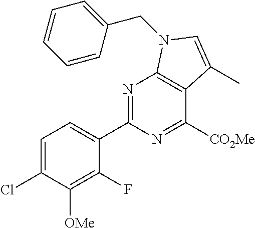 | 7.90 (1H, t), 7.80 1H, s), 7.50 (1H, dd), 7.30 (5H, m), 5.50 (2H, s), 4.10 (3H, s), 4.00 (3H, s), 2.30 (3H, s) ppm |
| 7-63 | 4-Carboxy-2-(4-chloro- 2-fluoro-3- methoxyphenyl)-5- methyl-7-(1-phenyl- ethyl)-7H-pyrrolo[2,3- d]pyrimidine | 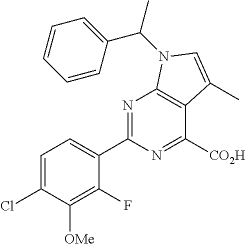 | 175 |
| 7-64 | 2-(4-Chloro-2-fluoro-3- methoxyphenyl)-4- methoxycarbonyl-5- methyl-7-(1-phenyl- ethyl)-7H-pyrrolo[2,3- d]pyrimidine | 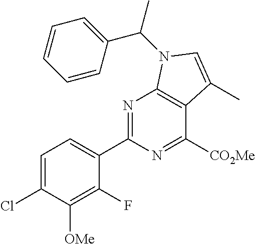 | 7.83 (1H, dd), 7.32 (5H, m), 7.25 (1H, m), 7.16 (1H, s), 6.31 (1H, q), 4.06 (3H, s), 4.03 (3H, s), 2.40 (3H, s), 1.91 (3H, d) ppm |
| 8-63 | 4-Carboxy-2-(4-chloro- 2-fluoro-3- methoxyphenyl)-5- methyl-7-(2- nitrophenyl-methyl)-7H- pyrrolo[2 ,3-d]pyrimidine | 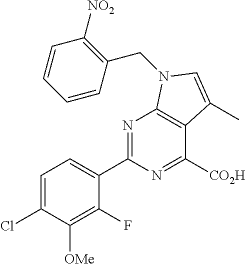 | 165 |
| 8-64 | 2-(4-Chloro-2-fluoro-3- methoxyphenyl)-4- methoxycarbonyl-5- methyl-7-(2- nitrophenyl-methyl)-7H- pyrrolo[2,3-d]pyrimidine | 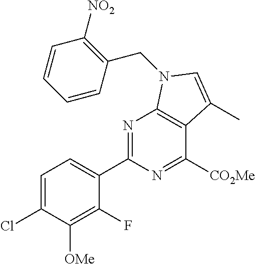 | 8.13 (1H, d), 7.80 (1H, dd), 7.52 (1H, m), 7.47 (1H, m), 7.25 (2H, m), 7.10 (1H, m), 5.88 (2H, s), 4.09 (3H, s), 4.01 (3H, s), 2.44 (3H, s) ppm |
| 9-63 | 4-Carboxy-2-(4-chloro- 2-fluoro-3- methoxyphenyl)-7-(4- fluorophenyl)-methyl)-5- methyl-7H-pyrrolo[2,3- d]pyrimidine | 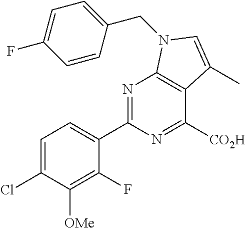 | 187 |
| 10-63 | 4-Carboxy-2-(4-chloro- 2-fluoro-3- methoxyphenyl)-7-(4- methoxyphenyl- methyl)-5-methyl-7H- pyrrolo[2,3-d]pyrimidine | 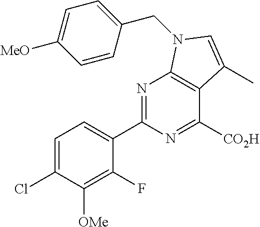 | 120 |
| 12-64 | 2-(4-Chloro-2-fluoro-3- methoxyphenyl)-4- methoxycarbonyl-5- methyl-7-(5- trifluoromethylfuran-2- yl)-methyl)-7H- pyrrolo[2,3-d]pyrimidine | 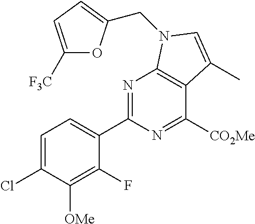 | 100 |
| 16-64 | 7-t-Butylcarbonyl-2-(4- chloro-2-fluoro-3- methoxyphenyl)-4- methoxycarbonyl-5- methyl-7H-pyrrolo[2,3- d]pyrimidine | 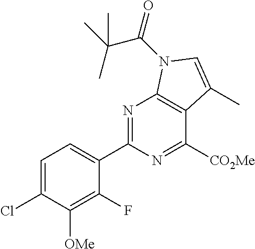 | 147-149 |
| 17-63 | 4-Carboxy-2-(4-chloro- 2-fluoro-3- methoxyphenyl)-7- methoxycarbonyl-5- methyl-7H-pyrrolo[2,3- d]pyrimidine | 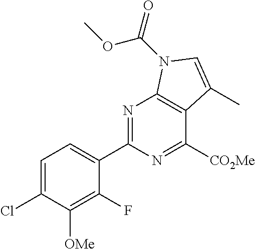 | 155-157 |
| 17-64 | 2-(4-Chloro-2-fluoro-3- methoxyphenyl)-4,7- di(methoxycarbonyl)-5- methyl-7H-pyrrolo[2,3- d]pyrimidine | 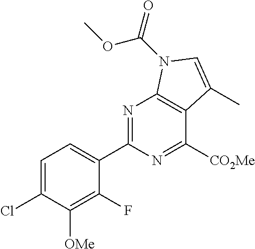 | 204-205 |
| 19-63 | 4-Carboxy-2-(4-chloro- 2-fluoro-3- methoxyphenyl)-5- methyl-7-(4- methylphenyl- sulphonyl)-7H- pyrrolo[2,3-d]pyrimidine | 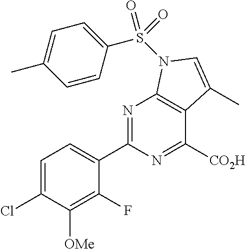 | 8.20 (2H, d), 7.90 (1H, t), 7.70 (1H, s), 7.20 (3H, m), 4.00 (3H, s), 2.50 (3H, s), 2.40 (3H, s) ppm (CO₂H not observed) |
| 36-64 | 6-(4-Chloro-2-fluoro-3- methoxyphenyl)-4- methoxycarbonyl-3- methyl-1- methylcarbonyl-1H- pyrrolo[3,2-c]pyridine | 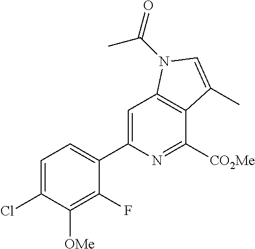 | 8.93 (1H, s), 7.71 (1H, t), 7.35 (1H, d), 7.26 (1H, dd), 4.05 (3H, s), 4.01 (3H, s), 2.66 (3H, s), 2.37 (3H, s) ppm |
| 37-64 | 1-t-Butylcarbonyl-6-(4- chloro-2-fluoro-3- methoxyphenyl)-4- methoxycarbonyl-3- methyl-1H-pyrrolo[3,2- c]pyridine | 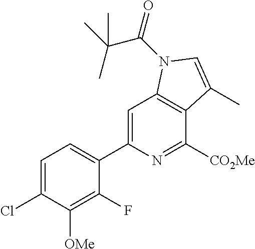 | 9.00 (1H, m), 7.71 (1H, t), 7.66 (1H, s), 7.25 (1H, m), 4.05 (3H, s), 4.01 (3H, s), 2.38 (3H, s), 1.53 (9H, s) ppm |
| 38-64 | 6-(4-Chloro-2-fluoro-3- methoxyphenyl)-1,4- di(methoxycarbonyl)-3- methyl-1H-pyrrolo[3,2- c]pyridine | 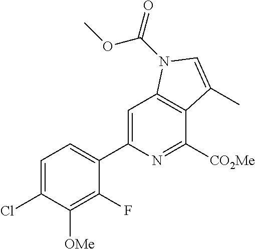 | 8.09 (1H, s), 7.76 (1H, t), 7.54 (1H, s), 7.28 (1H, m), 4.09 (3H, s), 4.06 (3H, s), 4.01 (3H, s), 2.36 (3H, s) ppm |
| 40-64 | 6-(4-Chloro-2-fluoro-3- methoxyphenyl)-4- methoxycarbonyl-3- methyl-1-(4- methylphenyl- sulphonyl)-1H- pyrrolo[3,2-c]pyridine | 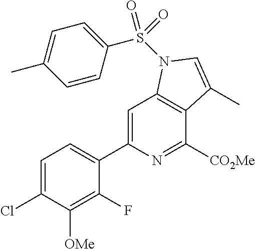 | 8.49 (1H, d), 7.89 (2H, d), 7.74 (2H, m), 7.37 (3H, m), 4.05 (3H, s), 4.00 (3H, s), 2.37 (3H, s), 2.32 (3H, s) ppm (nmr run in CD₃OD) |

### Example 26

### Synthesis of 2-(4-chloro-2-fluoro-3-methoxyphenyl)-9-(2,4-dimethoxyphenylmethyl)-6-(n-propoxycarbonyl)-8-methyl-9H-Purine (Compound 131-30)

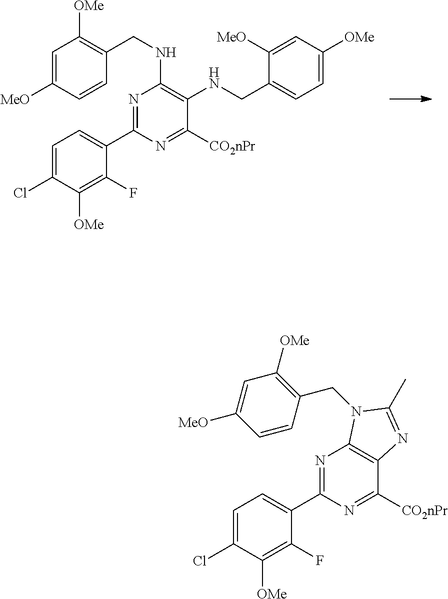

A solution of 2-(4-chloro-2-fluoro-3-methoxyphenyl)-4,5-bis(2,4-dimethoxyphenylmethylamino)-6-(n-propoxycarbonyl)-pyrimidine (prepared as described in example 23; 654 mg, 1.24 mmol) and trifluoroacetic acid (5 ml) in dichloromethane (10 ml) was stirred at ambient temperature for 5 hours, then evaporated under reduced pressure and the residue purified by automated flash chromatography (Presearch Combiflash Rf) on silica, with ethyl acetate in hexane (0% to 100% gradient) as eluent. A solution of the purified material, acetaldehyde (2 ml) and camphor sulphonic acid (35 mg, 0.15 mmol) in dioxane (3 ml) was heated at 100° C. for 30 minutes, then allowed to cool and evaporated under reduced pressure. The residue was purified using a FractionLynx hplc, to provide 2-(4-chloro-2-fluoro-3-methoxyphenyl)-9-(2,4-dimethoxyphenylmethyl)-6-(n-propoxycarbonyl)-8-methyl-9H-purine as an off-white solid (2 mg, 1%).

Characterising data for the compound are as follows:

¹H NMR (400 MHz, CDCl₃) δ 7.90 (1H, t), 7.30 (1H, m), 7.20 (1H, d), 6.40 (2H, m), 5.40 (2H, s), 4.50 (2H, t), 4.00 (3H, s), 3.80 (3H, s), 3.70 (3H, s), 2.70 (3H, s), 1.90 (2H, m), 1.00 (3H, t) ppm.

### Example 27

### Synthesis of 9-benzyl-6-carboxy-2-(4-chloro-2-fluoro-3-methoxyphenyl)-9H-purine (Compound 129-25)

### 27.1 Preparation of 9-benzyl-2,6-dichloro-9H-purine

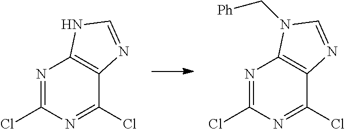

Potassium carbonate (2.07 g, 15 mmol) was added to a solution of 2,6-dichloro-9H-purine (945 mg, 5.0 mmol) in dimethylformamide (20 ml) and the mixture stirred at ambient temperature for 30 minutes. Benzyl bromide (1.2 ml, 10 mmol) was added and the mixture stirred overnight. Water was added and the resulting mixture extracted with ethyl acetate. The combined organic extracts were washed with water and brine, dried over magnesium sulphate, filtered and evaporated under reduced pressure. The residue was purified by automated flash chromatography (Presearch Combiflash Rf) on silica, with ethyl acetate in hexane (10% to 80% gradient) as eluent, to provide 9-benzyl-2,6-dichloro-9H-purine as a white solid (876 mg, 94%).

Characterising data for the compound are as follows:

¹H NMR (400 MHz, CDCl₃) δ 8.10 (1H, s), 7.40 (5H, m), 5.40 (2H, s) ppm.

### 27.2 Preparation of 9-benzyl-2-chloro-6-trimethylstannyl-9H-purine

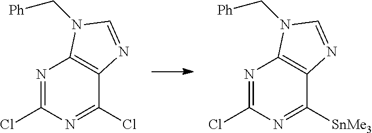

Palladium acetate (112 mg, 0.5 mmol) was added to a mixture of 9-benzyl-2,6-dichloro-9H-purine (1,4 g, 5.0 mmol), hexamethylditin (1.64 g, 5.0 mmol), 1,4-bis(diphenylphosphino)-butane (215 mg, 0.5 mmol) and dioxane (50 ml) and the resulting mixture heated at 110° C. for 4 hours. The reaction mixture was allowed to cool to ambient temperature, evaporated under reduced pressure and ethyl acetate added. The solution was washed with water and brine, dried over magnesium sulphate, filtered and evaporated under reduced pressure. The residue was purified by automated flash chromatography (Presearch Combiflash Rf) on silica, with ethyl acetate in hexane (0% to 100% gradient) to provide 9-benzyl-2-chloro-6-trimethylstannyl-9H-purine as a white solid (907 mg, 44%).

Characterising data for the compound are as follows:

¹H NMR (400 MHz, CDCl₃) δ 7.90 (1H, s), 7.30 (5H, m), 5.40 (2H, s), 0.50 (9H, t) ppm.

### 27.3 Preparation of 9-benzyl-2-chloro-6-(2-furanyl)-9H-purine

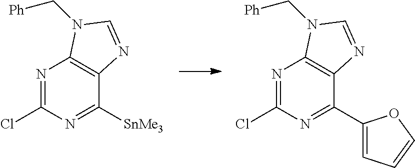

A mixture of 9-benzyl-2-chloro-6-trimethylstannyl-9H-purine (247 mg, 0.60 mmol), 5-bromofuran (105 mg, 0.72 mmol), [1,1′-bis(diphenylphosphino)-ferrocene] dichloropalladium (II) complex with dichloromethane (1:1) (49 mg, 0.06 mmol) and caesium fluoride (181 mg, 1.2 mmol), dimethoxyethane (3 ml) and water (3 ml) was heated in a microwave reactor at 140° C. for 20 minutes, allowed to cool to ambient temperature and extracted with ethyl acetate. The organic extract was washed with water and brine, dried over magnesium sulphate, filtered and evaporated under reduced pressure. The residue was purified by automated flash chromatography (Presearch Combiflash Rf) on silica, with ethyl acetate in hexane (0% to 100% gradient) to provide 9-benzyl-2-chloro-6-(2-furanyl)-9H-purine as a brown oil (79 mg, 42%).

Characterising data for the compound are as follows:

¹H NMR (400 MHz, CDCl₃) δ 8.00 (1H, s), 7.90 (1H, d), 7.80 (1H, d), 7.40 (5H, m), 6.70 (1H, dd), 5.40 (2H, s) ppm.

### 27.4 Preparation of 9-benzyl-2-(4-chloro-2-fluoro-3-methoxyphenyl)-6-(2-furanyl)-9H-purine

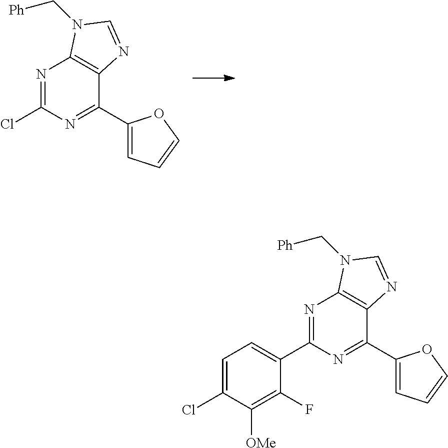

A mixture of 9-benzyl-2-chloro-6-(2-furanyl)-9H-purine (79 mg, 0.25 mmol), 4-chloro-2-fluoro-3-methoxyphenylboronic acid 1,3-propanediol ester (69 mg, 0.28 mmol), [1,1′-bis(diphenylphosphino)-ferrocene]dichloropalladium (II) complex with dichloromethane (1:1) (20 mg, 0.025 mmol), caesium fluoride (76 mg, 0.5 mmol), dimethoxyethane (1.5 ml) and water (1.5 ml) was heated in a microwave reactor at 140° C. for 20 minutes, allowed to cool to ambient temperature and extracted with dichloromethane. The organic extract was washed with brine, dried over magnesium sulphate, filtered and evaporated under reduced pressure. The residue was purified by automated flash chromatography (Presearch Combiflash Rf) on silica, with ethyl acetate in hexane (0% to 100% gradient) to provide 9-benzyl-2-(4-chloro-2-fluoro-3-methoxyphenyl)-6-(2-furanyl)-9H-purine as a yellow solid (80 mg, 72%).

Characterising data for the compound are as follows:

¹H NMR (400 MHz, CDCl₃) δ 8.10 (1H, s), 7.90 (1H, m), 7.85 (1H, d), 7.80 (1H, m), 7.40 (5H, m), 7.30 (1H, dd), 6.70 (1H, m), 5.50 (2H, s), 4.10 (3H, s) ppm.

### 27.5 Preparation of 9-benzyl-6-carboxy-2-(4-chloro-2-fluoro-3-methoxyphenyl)-9H-purine (Compound 129-25)

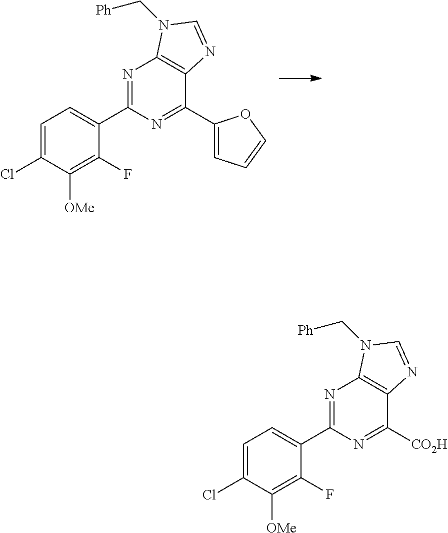

Ozone was bubbled through a solution of 9-benzyl-2-(4-chloro-2-fluoro-3-methoxyphenyl)-6-(2-furanyl)-9H-purine (80 mg, 0.19 mmol) in dichloromethane (40 ml) at −78° C. until a blue colour persisted in the reaction vessel. Oxygen was then bubbled through the reaction mixture until the blue colour disappeared, dimethyl sulphide (4 ml) was added and the mixture was allowed to warm to room temperature and stirred for 4 hours. The solution was evaporated under reduced pressure to provide 9-benzyl-6-carboxy-2-(4-chloro-2-fluoro-3-methoxyphenyl)-9H-purine (78 mg, 100%).

Characterising data for the compound are as follows:

[M−H]⁻ 411, 413.

### Example 28

### Synthesis of 9-benzyl-2-(4-chloro-2-fluoro-3-methoxyphenyl)-6-methoxycarbonyl-9H-purine (Compound 129-26)

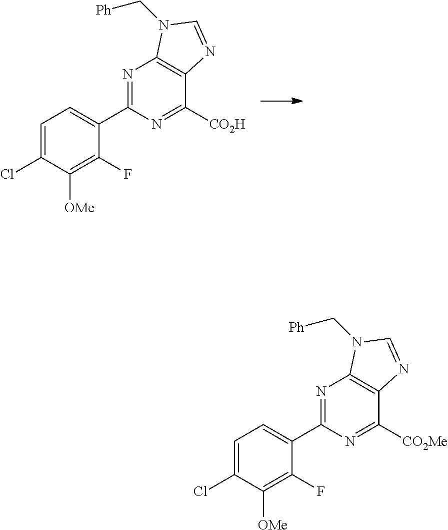

Trimethylsilyldiazomethane (2M in hexane; 0.11 ml, 0.23 mmol) was added to a stirred solution of 9-benzyl-6-carboxy-2-(4-chloro-2-fluoro-3-methoxyphenyl)-9H-purine (prepared as described in example 27; 78 mg, 0.19 mmol) in methanol (1 ml) and dichloromethane (4 ml) and the reaction mixture stirred at ambient temperature for 30 minutes. Glacial acetic acid (0.1 ml) was added, the mxture evaporated under reduced pressure and the residue dissolved in ethyl acetate. The solution was washed with water and brine, dried over magnesium sulphate, filtered and evaporated under reduced pressure. The residue was purified by automated flash chromatography (Presearch Combiflash Rf) on silica, with ethyl acetate in hexane (0% to 100% gradient) to provide 9-benzyl-2-(4-chloro-2-fluoro-3-methoxyphenyl)-6-methoxycarbonyl-9H-purine as a white solid (31 mg, 38%).

Characterising data for the compound are as follows:

¹H NMR (400 MHz, CDCl₃) δ 8.28 (1H, s), 7.91 (1H, t), 7.39 (5H, m), 7.29 (1H, d), 5.52 (2H, s), 4.14 (3H, s), 4:05 (3H, s) ppm.

### Example 29

### Pre-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retroflexus (AMARE) and Ipomoea hederaceae (IPOHE) were sown in standard soil in pots. After cultivation for one day under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 1000 g/ha of test compound.

The test plants were then grown under controlled conditions in the glasshouse (at 24/16° C., day/night; 14 hours light; 65% humidity) and watered twice daily. After 13 days the test was evaluated (100=total damage to plant; 0=no damage to plant). Results are shown below in Table 38.

TABLE 38
Percentage damage caused to weed species by compounds of the invention when applied pre-emergence.

| | | Species | | | | | |
|---|---|---|---|---|---|---|---|
| Compound Number | Rate (g/ha) | SOLNI | AMARE | SETFA | ALOMY | ECHCG | IPOHE |
| 1-2 | 1,000 | 50 | 30 | 0 | 0 | 0 | 20 |
| 1-3 | 1,000 | 20 | 50 | 10 | 0 | 0 | 100 |
| 1-4 | 1,000 | 90 | 90 | 30 | 10 | 30 | 60 |
| 1-6 | 1,000 | 0 | 0 | 0 | 0 | 0 | 0 |
| 1-20 | 1,000 | 0 | 50 | 0 | 0 | 0 | 0 |
| 1-22 | 1,000 | 0 | 0 | 0 | 0 | 0 | 0 |
| 1-24 | 1,000 | 0 | 0 | 0 | 0 | 0 | 0 |
| 1-61 | 1,000 | — | — | 0 | 0 | 0 | 0 |
| 1-63 | 1,000 | 90 | 70 | 10 | 30 | 20 | 80 |
| 1-64 | 1,000 | 70 | 100 | 10 | 20 | 30 | 100 |
| 1-65 | 1,000 | 0 | 0 | 0 | 0 | 0 | 0 |
| 1-66 | 1,000 | 100 | 100 | 70 | 20 | 40 | 90 |
| 1-68 | 1,000 | 0 | 0 | 0 | 0 | 0 | 0 |
| 1-75 | 1,000 | 0 | 0 | 0 | 0 | 0 | 0 |
| 1-77 | 1,000 | 10 | 0 | 10 | 0 | 0 | 0 |
| 1-78 | 1,000 | 40 | 50 | 0 | 0 | 0 | 20 |
| 1-79 | 1,000 | 0 | 0 | 0 | 0 | 0 | 0 |
| 1-80 | 1,000 | 0 | 0 | 0 | 0 | 0 | 0 |
| 1-83 | 1,000 | 30 | 40 | 0 | 0 | 0 | 20 |
| 1-84 | 1,000 | 40 | 20 | 10 | 10 | 10 | 70 |
| 1-85 | 1,000 | 70 | 100 | 0 | 20 | 0 | 10 |
| 1-86 | 1,000 | 10 | — | 0 | 0 | 0 | 0 |
| 1-87 | 1,000 | 20 | 10 | 0 | 0 | 0 | 10 |
| 1-88 | 1,000 | 20 | 10 | 0 | 0 | 10 | 10 |
| 1-108 | 1,000 | 0 | 0 | 0 | 0 | 0 | 0 |
| 1-116 | 1,000 | 0 | 0 | 0 | 0 | 0 | 0 |
| 1-118 | 1,000 | 70 | 40 | 20 | 0 | 10 | 40 |
| 1-120 | 1,000 | 0 | 0 | 0 | 0 | 0 | 0 |
| 1-121 | 1,000 | 0 | 0 | 0 | 0 | 0 | 0 |
| 1-126 | 1,000 | 0 | 0 | 0 | 0 | 0 | 0 |
| 2-64 | 1,000 | 40 | 30 | 0 | 0 | 0 | 50 |
| 3-63 | 1,000 | 50 | 100 | 0 | 0 | 0 | 20 |
| 3-64 | 1,000 | 0 | 0 | 0 | 0 | 0 | 0 |
| 5-18 | 1,000 | 0 | 0 | 0 | 0 | 0 | 0 |
| 5-21 | 1,000 | 40 | 100 | 10 | 10 | 0 | 30 |
| 5-65 | 1,000 | 0 | 0 | 0 | 0 | 0 | 0 |
| 5-66 | 1,000 | 0 | 0 | 0 | 0 | 0 | 0 |
| 6-17 | 1,000 | 0 | 0 | 0 | 0 | 0 | 0 |
| 6-18 | 1,000 | 0 | 0 | 0 | 0 | 0 | 0 |
| 6-21 | 250 | 0 | 0 | 0 | 0 | 0 | 0 |
| 6-63 | 1,000 | 0 | 0 | 0 | 0 | 0 | 0 |
| 6-64 | 1,000 | 50 | 50 | 0 | 0 | 0 | 30 |
| 6-65 | 1,000 | 0 | 0 | 0 | 0 | 0 | 0 |
| 6-66 | 1,000 | 20 | 10 | 0 | 0 | 0 | 0 |
| 7-63 | 1,000 | 0 | 0 | 0 | 0 | 0 | 0 |
| 8-1 | 1,000 | 0 | 0 | 0 | 0 | 0 | 0 |
| 8-2 | 1,000 | 0 | 0 | 0 | 0 | 0 | 0 |
| 8-5 | 1,000 | 0 | 0 | 0 | 0 | 0 | 0 |
| 8-6 | 1,000 | 0 | 0 | 0 | 0 | 0 | 0 |
| 8-17 | 1,000 | 0 | 0 | 0 | 0 | 0 | 0 |
| 8-18 | 1,000 | 0 | 0 | 0 | 0 | 0 | 0 |
| 8-21 | 1,000 | 0 | — | 20 | 0 | 0 | 0 |
| 8-63 | 1,000 | 40 | 10 | 0 | 0 | 0 | 30 |
| 8-65 | 1,000 | 0 | 0 | 0 | 0 | 0 | 0 |
| 8-66 | 1,000 | 10 | 10 | 0 | 0 | 0 | 0 |
| 9-63 | 1,000 | 10 | 20 | 0 | 0 | 0 | 0 |
| 10-63 | 1,000 | 0 | 0 | 0 | 0 | 0 | 0 |
| 11-18 | 1,000 | 0 | 0 | 0 | 0 | 0 | 0 |
| 11-21 | 1,000 | 0 | 0 | 0 | 0 | 0 | 0 |
| 11-66 | 1,000 | 0 | 0 | 0 | 0 | 0 | 0 |
| 11-126 | 1,000 | 0 | 0 | 0 | 0 | 0 | 0 |
| 12-63 | 1,000 | 0 | 60 | 20 | 0 | 0 | 10 |
| 12-64 | 1,000 | 0 | 0 | 0 | 0 | 0 | 0 |
| 14-17 | 1,000 | 0 | 0 | 0 | 0 | 0 | 0 |
| 16-64 | 1,000 | 0 | 0 | 0 | 0 | 0 | 0 |
| 17-63 | 1,000 | 90 | 100 | 20 | 20 | 20 | 100 |
| 17-64 | 1,000 | 0 | 0 | 0 | 0 | 0 | 0 |
| 18-64 | 1,000 | 0 | 0 | 0 | 0 | 0 | 0 |
| 19-63 | 1,000 | 0 | 0 | 0 | 0 | 0 | 0 |
| 21-64 | 1,000 | 0 | 0 | 0 | 0 | 0 | 0 |
| 22-20 | 1,000 | 60 | 100 | 0 | 20 | 20 | 80 |
| 22-62 | 1,000 | 20 | 20 | 0 | 0 | 0 | 50 |
| 22-63 | 1,000 | 60 | 20 | 0 | 0 | 0 | 70 |
| 22-64 | 1,000 | 80 | 90 | 20 | 10 | 70 | 60 |
| 22-66 | 1,000 | 0 | 0 | 0 | 0 | 0 | 0 |
| 22-75 | 1,000 | 80 | 70 | 0 | 10 | 10 | 90 |
| 22-77 | 1,000 | 80 | 20 | 20 | 10 | 0 | 90 |
| 22-78 | 1,000 | 10 | 20 | 0 | 0 | 0 | 0 |
| 22-79 | 1,000 | 50 | 10 | 20 | 10 | 10 | 30 |
| 22-80 | 1,000 | 60 | 40 | 20 | 20 | 20 | 50 |
| 22-82 | 1,000 | 90 | 70 | 0 | 10 | 20 | 90 |
| 22-83 | 1,000 | 80 | 0 | 10 | 20 | 20 | 80 |
| 22-84 | 1,000 | 70 | 40 | 10 | 20 | 20 | 80 |
| 22-85 | 1,000 | 100 | 100 | 10 | 10 | 10 | 80 |
| 22-86 | 1,000 | 10 | 0 | 0 | 0 | 0 | 0 |
| 22-87 | 1,000 | 70 | 20 | 10 | 10 | 10 | 80 |
| 22-88 | 1,000 | 100 | 100 | 10 | 10 | 0 | 50 |
| 22-122 | 1,000 | 0 | 0 | 0 | 0 | 0 | 0 |
| 22-124 | 1,000 | 100 | 100 | 30 | 70 | 20 | 100 |
| 22-132 | 1,000 | 10 | 0 | 0 | 0 | 0 | 40 |
| 32-62 | 1,000 | 80 | 80 | 20 | 30 | 30 | 20 |
| 36-64 | 1,000 | 50 | — | 0 | 0 | 0 | 20 |
| 37-64 | 1,000 | 0 | 0 | 0 | 0 | 0 | 0 |
| 38-64 | 1,000 | 0 | 0 | 0 | 0 | 0 | 0 |
| 40-64 | 1,000 | 40 | 0 | 10 | 0 | 10 | 0 |
| 71-180 | 500 | 0 | 0 | 0 | 0 | 0 | 0 |
| 81-46 | 1,000 | 0 | 0 | 0 | 0 | 0 | 0 |
| 83-48 | 1,000 | 0 | 0 | 0 | 0 | 0 | 0 |
| 91-44 | 1,000 | 30 | 70 | 0 | 30 | 10 | 20 |
| 91-184 | 1,000 | 0 | 0 | 0 | 0 | 0 | 0 |
| 91-203 | 1,000 | 20 | 0 | 0 | 0 | 0 | 10 |
| 91-204 | 1,000 | 10 | 20 | 0 | 0 | 0 | 0 |
| 91-207 | 1,000 | 10 | 20 | 10 | 0 | 0 | 0 |
| 92-163 | 1,000 | 0 | 0 | 0 | 0 | 0 | 0 |
| 92-164 | 1,000 | 0 | 0 | 0 | 0 | 0 | 0 |
| 93-165 | 1,000 | 0 | 0 | 0 | 0 | 0 | 10 |
| 93-166 | 1,000 | 0 | 0 | 0 | 0 | 0 | 0 |
| 95-160 | 250 | 0 | 0 | 0 | 0 | 0 | 0 |
| 95-162 | 1,000 | 0 | 0 | 0 | 10 | 0 | 40 |
| 95-168 | 1,000 | 0 | 0 | 0 | 0 | 0 | 0 |
| 106-74 | 1,000 | 0 | 0 | 0 | 0 | 0 | 0 |
| 119-28 | 1,000 | 0 | 0 | 0 | 0 | 0 | 0 |
| 119-168 | 1,000 | 0 | 0 | 0 | 0 | 0 | 0 |
| 123-50 | 1,000 | 0 | 0 | 0 | 0 | 0 | 0 |
| 125-76 | 1,000 | 70 | 80 | 20 | 10 | 20 | 60 |

### Example 30

### Post-Emergence Biological Efficacy

Seeds of Alopecurus myosuroides (ALOMY), Setaria faberi (SETFA), Echinochloa crusgaffi (ECHCG), Solanum nigrum (SOLNI), Amaranthus retroflexus (AMARE) and Ipomoea hederaceae (IPOHE) were sown in standard soil in pots. After cultivation for 8 days under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 500 or 1000 g/ha of test compound.

The test plants were then grown on under controlled conditions in a glasshouse (at 24/16° C., day/night; 14 hours light; 65% humidity) and watered twice daily. After 13 days the test was evaluated (100=total damage to plant; 0=no damage to plant). Results are shown below in Table 39.

TABLE 39
Percentage damage caused to weed species by compounds of the invention when applied post-emergence

| | | Species | | | | | |
|---|---|---|---|---|---|---|---|
| Compound Number | Rate (g/ha) | SOLNI | AMARE | SETFA | ALOMY | ECHCG | IPOHE |
| 1-2 | 1,000 | 90 | 90 | 90 | 10 | 70 | 100 |
| 1-3 | 1,000 | 80 | 100 | 0 | 0 | 0 | 50 |
| 1-4 | 1,000 | 90 | 100 | 80 | 10 | 70 | 70 |
| 1-6 | 1,000 | 0 | 0 | 0 | 0 | 0 | 0 |
| 1-20 | 1,000 | 70 | 70 | 0 | 0 | 0 | 60 |
| 1-22 | 1,000 | 30 | 20 | 10 | 10 | 0 | 40 |
| 1-24 | 1,000 | 50 | 10 | 0 | 0 | 0 | 20 |
| 1-61 | 1,000 | 70 | 100 | 40 | 10 | 20 | 70 |
| 1-63 | 1,000 | 100 | 100 | 60 | 40 | 60 | 70 |
| 1-64 | 1,000 | 80 | 100 | 70 | 20 | 70 | 70 |
| 1-65 | 1,000 | 60 | 40 | 10 | 0 | 40 | 60 |
| 1-66 | 1,000 | 100 | 100 | 80 | 80 | 80 | 80 |
| 1-68 | 1,000 | 30 | 10 | 0 | 0 | 10 | 60 |
| 1-75 | 1,000 | 70 | 40 | 0 | 0 | 0 | 70 |
| 1-77 | 1,000 | 70 | 10 | 10 | 0 | 0 | 40 |
| 1-78 | 1,000 | 60 | 0 | 0 | 0 | 0 | 70 |
| 1-79 | 1,000 | 10 | 10 | 0 | 0 | 0 | 20 |
| 1-80 | 1,000 | 80 | 60 | 0 | 0 | 10 | 40 |
| 1-83 | 1,000 | 70 | 70 | 10 | 0 | 10 | 40 |
| 1-84 | 1,000 | 70 | 60 | 10 | 10 | 0 | 40 |
| 1-85 | 1,000 | 90 | 100 | 40 | 10 | 60 | 40 |
| 1-86 | 1,000 | 60 | 40 | 10 | 0 | 0 | 30 |
| 1-87 | 1,000 | 90 | 100 | 10 | 0 | 0 | 50 |
| 1-88 | 1,000 | 70 | 50 | 40 | 10 | 10 | 50 |
| 1-108 | 1,000 | 30 | 0 | 0 | 0 | 0 | 50 |
| 1-116 | 1,000 | 40 | 50 | 20 | 0 | 0 | 50 |
| 1-118 | 1,000 | 80 | 100 | 70 | 10 | 50 | 50 |
| 1-120 | 1,000 | 20 | 10 | 0 | 0 | 0 | 20 |
| 1-121 | 1,000 | 10 | 0 | 0 | 0 | 0 | 0 |
| 1-126 | 1,000 | 0 | 0 | 0 | 0 | 0 | 0 |
| 2-64 | 1,000 | 60 | 30 | 0 | 0 | 0 | 40 |
| 3-63 | 1,000 | 80 | 80 | 10 | 0 | 10 | 60 |
| 3-64 | 1,000 | 40 | 10 | 0 | 0 | 0 | 70 |
| 5-18 | 1,000 | 20 | 0 | 0 | 0 | 0 | 0 |
| 5-21 | 1,000 | 50 | 70 | 10 | 10 | 10 | 30 |
| 5-65 | 1,000 | 20 | 10 | 20 | 0 | 0 | 10 |
| 5-66 | 1,000 | 20 | 0 | 20 | 10 | 10 | 0 |
| 6-17 | 1,000 | 40 | 20 | 0 | 0 | 0 | 0 |
| 6-18 | 1,000 | 40 | 0 | 0 | 0 | 0 | 20 |
| 6-21 | 250 | 10 | 0 | 0 | 10 | 0 | 10 |
| 6-63 | 1,000 | 70 | 80 | 50 | 10 | 40 | 60 |
| 6-64 | 1,000 | 70 | 20 | 0 | 0 | 0 | 50 |
| 6-65 | 1,000 | 80 | 20 | 0 | 0 | 0 | 40 |
| 6-66 | 1,000 | 80 | 40 | 10 | 0 | 0 | 40 |
| 7-63 | 1,000 | 60 | 20 | 10 | 0 | 0 | 10 |
| 8-1 | 1,000 | 30 | 0 | 0 | 0 | 10 | 40 |
| 8-2 | 1,000 | 20 | 0 | 0 | 0 | 0 | 20 |
| 8-5 | 1,000 | 0 | 0 | 0 | 0 | 0 | 0 |
| 8-6 | 1,000 | 20 | 20 | 0 | 0 | 0 | 50 |
| 8-17 | 1,000 | 50 | 20 | 0 | 0 | 0 | 50 |
| 8-18 | 1,000 | 30 | 0 | 0 | 0 | 0 | 20 |
| 8-21 | 1,000 | 20 | 0 | 0 | 10 | 0 | 10 |
| 8-63 | 1,000 | 80 | 70 | 20 | 10 | 50 | 60 |
| 8-65 | 1,000 | 10 | 0 | 20 | 0 | 0 | 10 |
| 8-66 | 1,000 | 30 | 0 | 0 | 0 | 0 | 10 |
| 9-63 | 1,000 | 60 | 20 | 0 | 0 | 0 | 40 |
| 10-63 | 1,000 | 20 | 10 | 0 | 0 | 0 | 20 |
| 11-18 | 1,000 | 50 | 0 | 0 | 0 | 0 | 20 |
| 11-21 | 1,000 | 50 | 0 | 0 | 0 | 0 | 20 |
| 11-66 | 1,000 | 50 | 20 | 10 | 10 | 20 | 30 |
| 11-126 | 1,000 | 20 | 0 | 0 | 0 | 0 | 0 |
| 12-63 | 1,000 | 70 | 30 | 0 | 0 | 0 | 50 |
| 12-64 | 1,000 | 30 | 20 | 30 | 10 | 10 | 50 |
| 14-17 | 1,000 | 40 | 0 | 0 | 0 | 0 | 10 |
| 16-64 | 1,000 | 60 | 10 | 0 | 0 | 0 | 70 |
| 17-63 | 1,000 | 90 | 90 | 60 | 30 | 60 | 70 |
| 17-64 | 1,000 | 20 | 0 | 0 | 0 | 0 | 0 |
| 18-64 | 1,000 | 40 | 20 | 30 | 10 | 10 | 40 |
| 19-63 | 1,000 | 70 | 50 | 0 | 0 | 0 | 60 |
| 21-64 | 1,000 | 70 | 80 | 0 | 0 | 10 | 40 |
| 22-20 | 1,000 | 80 | 100 | 60 | 50 | 70 | 70 |
| 22-62 | 1,000 | 70 | 80 | 20 | 0 | 50 | 60 |
| 22-63 | 1,000 | 80 | 70 | 90 | 40 | 70 | 70 |
| 22-64 | 1,000 | 90 | 100 | 70 | 50 | 80 | 70 |
| 22-66 | 1,000 | 40 | 20 | 0 | 0 | 0 | 40 |
| 22-75 | 1,000 | 90 | 80 | 50 | 10 | 20 | 60 |
| 22-77 | 1,000 | 90 | 70 | 0 | 10 | 0 | 30 |
| 22-78 | 1,000 | 60 | 0 | 0 | 0 | 0 | 70 |
| 22-79 | 1,000 | 70 | 40 | 10 | 10 | 0 | 40 |
| 22-80 | 1,000 | 90 | 70 | 20 | 10 | 10 | 50 |
| 22-82 | 1,000 | 90 | 80 | 40 | 20 | 20 | 40 |
| 22-83 | 1,000 | 90 | 90 | 40 | 20 | 20 | 60 |
| 22-84 | 1,000 | 90 | 80 | 30 | 20 | 20 | 40 |
| 22-85 | 1,000 | 90 | 90 | 20 | 30 | 20 | 40 |
| 22-86 | 1,000 | 70 | 40 | 0 | 10 | 10 | 40 |
| 22-87 | 1,000 | 90 | 80 | 40 | 20 | 10 | 70 |
| 22-88 | 1,000 | 90 | 80 | 40 | 30 | 20 | 60 |
| 22-122 | 1,000 | 20 | 60 | 0 | 0 | 0 | 10 |
| 22-124 | 1,000 | 90 | 80 | 40 | 30 | 20 | 80 |
| 22-132 | 1,000 | 60 | 50 | 0 | 0 | 0 | 50 |
| 32-62 | 1,000 | 80 | 80 | 40 | 10 | 50 | 70 |
| 36-64 | 1,000 | 80 | 70 | 10 | 0 | 0 | 70 |
| 37-64 | 1,000 | 50 | 0 | 0 | 0 | 0 | 40 |
| 38-64 | 1,000 | 10 | 0 | 10 | 10 | 0 | 20 |
| 40-64 | 1,000 | 60 | 0 | 0 | 0 | 0 | 50 |
| 71-180 | 500 | 20 | 0 | 0 | 0 | 0 | 40 |
| 81-46 | 1,000 | 10 | 0 | 10 | 0 | 0 | 0 |
| 83-48 | 1,000 | 20 | 0 | 20 | 20 | 20 | 10 |
| 91-44 | 1,000 | 90 | 70 | 50 | 20 | 50 | 60 |
| 91-184 | 1,000 | 50 | 20 | 0 | 0 | 30 | 70 |
| 91-203 | 1,000 | 40 | 10 | 10 | 0 | 0 | 10 |
| 91-204 | 1,000 | 80 | 50 | 0 | 0 | 0 | 40 |
| 91-207 | 1,000 | 70 | 20 | 0 | 0 | 0 | 60 |
| 92-163 | 1,000 | 10 | 0 | 10 | 10 | 10 | 10 |
| 92-164 | 1,000 | 10 | 0 | 0 | 0 | 0 | 0 |
| 93-165 | 1,000 | 30 | 20 | 30 | 10 | 20 | 20 |
| 93-166 | 1,000 | 10 | 0 | 30 | 20 | 10 | 10 |
| 95-160 | 250 | 30 | 20 | 40 | 10 | 10 | 20 |
| 95-162 | 1,000 | 40 | 70 | 0 | 10 | 0 | 30 |
| 95-168 | 1,000 | 10 | 0 | 0 | 0 | 0 | 10 |
| 106-74 | 1,000 | 0 | 0 | 0 | 0 | 0 | 0 |
| 119-28 | 1,000 | 20 | 20 | 0 | 0 | 0 | 10 |
| 119-168 | 1,000 | 30 | 10 | 0 | 0 | 0 | 40 |
| 123-50 | 1,000 | 20 | 0 | 10 | 0 | 10 | 10 |
| 125-76 | 1,000 | 90 | 100 | 80 | 10 | 70 | 80 |

1. A compound having the formula (I): 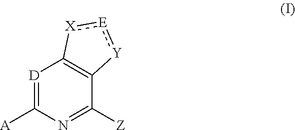
or a salt or N-oxide thereof,
wherein:
A is halogen, C2-C6 alkenyl optionally substituted by 1 to 3 groups R¹, C3-C8 cycloalkyl optionally substituted by 1 to 3 groups R¹, C1-06 alkylthio optionally substituted by 1 to 3 groups R¹, C6-C10 aryl optionally substituted by 1 to 3 groups R² or a mono- or bicyclic heteroaryl group having 5 to 10 ring atoms and at least one ring atom which is nitrogen, oxygen or sulfur optionally substituted by 1 to 3 groups R²;
D is N or CR³;
X is O, S, N or NR⁴;
Y is CR⁵, CR⁵R⁶, N, NR⁵, O or S;
E is —(CR⁷R⁸)ₙ—;
n is 1, 2 or 3; [symbol]
is a bond that is optionally single or double
Z is C(O)R⁹, C(S)R¹⁰, or C(═NR¹¹)R¹²;
each R¹ is independently halogen, hydroxyl, nitro, amino, C1-03 alkylamino, di (C1-C3) alkylamino, cyano, C1-C3 alkyl, C1-C3 haloalkyl, C2-C3 alkenyl, C1-C3 alkoxy, C1-C3 haloalkoxy, C1-C3 alkylthio, C1-C3 alkylsulphonyl, C2-C6 carboxyalkyl, carboxyl, C2-C6 alkoxycarbonyl, C2-C7 alkylcarbonyloxy or C6-C10 aryl optionally substituted by 1 to 3 groups R²;
each R² is independently halogen, hydroxyl, nitro, amino, cyano, C1-C3 alkyl, C1-C3 haloalkyl, C1-C3 alkoxy, C1-C3 haloalkoxy, C1-C3 alkylthio, C1-C3 haloalkylthio, C1-C3 alkylsulpnoyl, C1-C3 alkylsulphonyloxy, C2-C6 carboxyalkyl, C2-C6 alkoxycarbonyl, C2-C7 alkylcarbonyloxy, C1-C3 alkylamino, or di(C1-C3 alkyl)amino;
R³ is hydrogen, halogen, C1-C3 alkyl , C1-C3 haloalkyl, C2-C4 alkoxyalkyl, C2-C4 alkenyl, C2-C4 haloalkenyl or cyclopropyl optionally substituted by 1 to 3 groups R¹;
R⁴ is hydrogen, C1-C6 alkyl optionally substituted by 1 to 3 groups R¹³, C2-C6 alkenyl optionally substituted by 1 to 3 groups R¹³, C2-C6 alkynyl optionally substituted by 1 to 3 groups R¹³, C3-C8 cycloalkyl optionally substituted by 1 to 3 groups R¹³, C1-C6 acyl optionally substituted by 1 to 3 groups R¹, C1-06 alkoxycarbonyl optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R², C1-06 alkylsulphonyl optionally substituted by 1 to 3 groups R¹ or C6-C10 arylsulphonyl optionally substituted by 1 to 3 groups R²;
each of R⁵ and R⁶ is independently hydrogen, halogen, C1-06 alkyl optionally substituted by 1 to 3 groups R¹, C1-C6 alkoxy, C6-C10 aryl optionally substituted by 1 to 3 groups R², carboxyl, C1-C7 acyl, C2-C7 alkoxycarbonyl, or, taken together with the carbon atom to which they are attached, R⁵ and R⁶ form a C1-C6 alkenyl group optionally substituted by 1 to 3 groups R¹, a carbonyl group, or a C3-C6 cycloalkyl group optionally substituted by 1 to 3 groups R¹;
each of R⁷ and R⁸ is independently hydrogen, halogen, C1-C6 alkyl optionally substituted by 1 to 3 groups R¹, C1-C6 alkoxy, C6-C10 aryl optionally substituted by 1 to 3 groups R², carboxyl, C1-C7 acyl, C2-C7 alkoxycarbonyl, or R⁷ represents an additional bond between the carbon atom to which it is attached and the adjacent ring atom or, taken together with the carbon atom to which they are attached, R⁷ and R⁸ form a C1-C6 alkenyl group optionally substituted by 1 to 3 groups R¹, a carbonyl group, or a C3-C6 cycloalkyl group optionally substituted by 1 to 3 groups R¹ or, when n is 2 or 3, taken together with the carbon atoms to which they are attached, any two R⁷ and R⁸ form a 5- or 6-membered saturated, unsaturated or aromatic ring, the ring optionally including 1 to 3 ring atoms which are independently selected from nitrogen, oxygen or sulphur and optionally substituted by 1 to 3 groups R¹ ;
R⁹ is hydrogen, hydroxyl, C1-C10 alkoxy optionally substituted by C1-C6 alkoxy, C1-C6alkoxy-C1-C6alkoxy, phenyl, C5-C10 heteroaryl or C3-C10 heterocyclyl, C2-C10 alkenyloxy, C3-C8 cycloalkoxy optionally substituted by C1-C6 alkoxy or phenyl, C1-C6 alkylthio, amino, C1-C6 alkylamino, or di(C1-C6 alkyl)amino;
R¹⁰ is C1-C10 alkoxy optionally substituted by C1-C6 alkoxy or phenyl, C2-C10 alkenyloxy, C3-C8 cycloalkoxy optionally substituted by C1-C6 alkoxy or phenyl, C1-C6 alkylthio, amino, C1-C6 alkylamino, or di(C1-C6 alkyl)amino;
R¹¹ is hydrogen, C1-C6 alkyl, C1-C6 alkoxy, C3-C8 cycloalkoxy, amino, C1-C6 alkylamino, or di(C1-C6 alkyl)amino;
R¹² is hydrogen, C1-C6 alkoxy, C3-C8 cycloalkoxy, C1-C6 alkylthio, amino, C1-C6 alkylamino, or di(C1-C6 alkyl)amino;
each R¹³ is independently cyano, hydroxyl, carboxyl, C3-C6 cycloalkyl, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R², C1-C4 alkoxy; C1-C4 alkoxy(C1-C4)alkoxy; C1-C4 alkoxycarbonyl; or tri(C1-C4)alkylsilyl
provided that
(i) when Y is NR^{S}, X is N, Z is C(O)R⁹, D is N, E is —(CR⁷R⁸)ₙ—, R⁵ is alkyl or haloalkyl, R⁷ represents an additional bond to X, and R⁹ is alkoxy, then R⁸ is other than H;
(ii) when XEY is —N(R⁴)C(O)NH—, Z is not C(O)NH₂, C(O)NHCH₃ or C(O)N(CH₃)₂;
(iii) the compound of formula (I) is not:
9-benzyl-9H-purine-2,6-dicarboxamide;
9-(2-hydroxyethyl)-2-(prop-1-enyl)-9H-purine-6-carboxamide;
9-(2-hydroxyethyl)-2-phenyl-9H-purine-6-carboxamide;
9-phenyl-2-(pyridin-3-yl)-9H-purine-6-carboxamide;
2-(3-hydroxyphenyl)-9-(2-methoxyphenyl)-9H-purine-6-carboxamide;
2-(2-hydroxyphenyl)-9-(2-methoxyphenyl)-purine-6-carboxamide;
6-oxo-8-phenyl-2-(pyridin-3-yl)-5,6,7,8-tetrahydropteridine-4-carboxamide;
6-oxo-8-phenyl-2-(pyridin-4-yl)-5,6,7,8-tetrahydropteridine-4-carboxamide;
2-(3-hydroxyphenyl)-8-(2-methoxyphenyl)-6-oxo-5,6,7,8-tetrahydropteridine-4-carboxamide;
2-chloro-9-phenyl-9H-purine-6-carboxylic acid;
2-chloro-9-methyl-9H-purine-6-carboxylic acid;
2-chloro-9-methyl-9H-purine-6-carboxylic acid ethyl ester;
2-chloro-9-ethoxycarbonylmethyl-9H-purine-6-carboxylic acid ethyl ester.

2. A compound according to claim 1, wherein A is halogen, C2-C6 alkenyl, C3-C8 cycloalkyl optionally substituted by 1 to 3 groups R¹, C6-C10 aryl optionally substituted by 1 to 3 groups R² or a mono or bicyclic heteroaryl group having 5 to 10 ring atoms and at least one ring atom which is nitrogen, oxygen or sulphur optionally substituted by 1 to 3 groups R².

3. A compound according to claim 1 wherein A is halogen, a phenyl ring optionally substituted by 1 to 3 groups R², or cyclopropyl optionally substituted by 1-2 groups R¹, and R¹ and R² are as defined in claim 1.

4. A compound according to claim 1 wherein D is N, CH, CF, CCl or CMe.

5. A compound according to claim 4 wherein D is N or CH.

6. A compound according to claim 1, wherein X is NR⁴ and R⁴ is as defined in claim 1.

7. A compound according to claim 1 wherein Y is CR⁵ or CR⁵R⁶ and R⁵ and R⁶ are as defined in claim 1.

8. A compound according to claim 1 wherein Z is C(O)R⁹ and R9 is as defined in claim 1.

9. A compound according to claim 1, wherein n is 1, R⁷ represents an additional bond to Y, and R⁹ is selected from H and C1-C6 alkyl.

10. A compound according to claim 1 having the formula 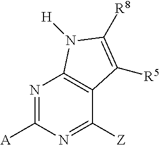
wherein:
A is phenyl optionally substituted by 1 to 3 groups R² or cyclopropyl optionally substituted by 1 to 3 groups R¹;
Z is C(O)R⁹, wherein R⁹ is selected from hydroxyl and C1-C6 alkoxy;
R⁵ is selected from H and C1-C6 alkyl; and
R⁹ is selected from H and C1-C6 alkyl.

11. A compound according to claim 1 having the formula 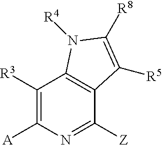
wherein:
A is phenyl optionally substituted by 1 to 3 groups R² or halogen.
Z is C(O)R⁹, wherein R⁹ is selected from hydroxyl and C1-C6 alkoxy;
R³ is H, fluoro or chloro,
R⁴ is H;
R⁵ is selected from H and C1-C6 alkyl and
R⁸ is selected from H and C1-C6 alkyl.

12. A compound according to claim 1 which is one of

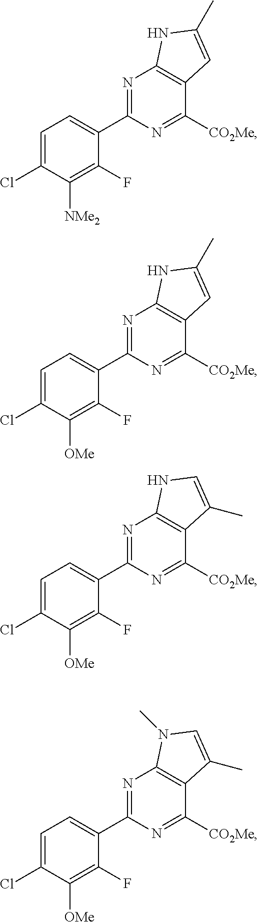

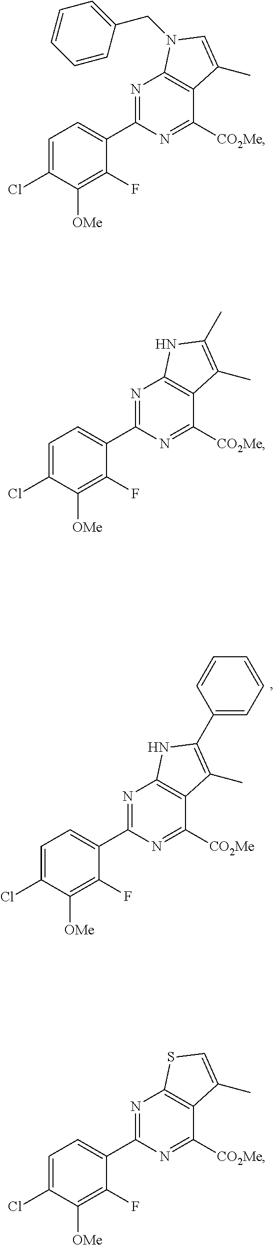

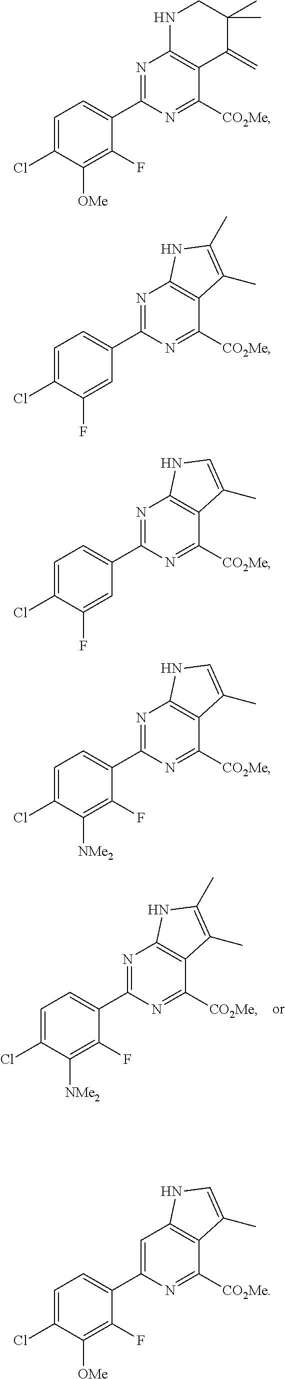

13. A herbicidal composition comprising a compound of formula I wherein
A is
(i) halogen, C1-C6 alkyl optionally substituted by 1 to 3 groups R¹, C1-6 haloalkyl optionally substituted by 1 to 3 groups R¹, C2-C6 alkenyl optionally substituted by 1 to 3 groups R¹, C3-C8 cycloalkyl optionally substituted by 1 to 3 groups R¹, C1-C6 alkylthio optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R², or
(ii) as defined in claim 1, and D, X, E, Y and Z are as defined in claim 1, without the provisos (i), (ii) and (iii) of claim 1, together with at least one agriculturally acceptable adjuvant or diluent.

14. A composition according to claim 13 which comprises a further herbicide in addition to the compound of formula (I).

15. A composition according to claim 13 which comprises a safener.

16. (canceled)

17. A method of controlling weeds in crops of useful plants, comprising applying to said weeds or to the locus of said weeds, or to said useful crop plants, a compound of formula I wherein
A is
(i) halogen, C1-C6 alkyl optionally substituted by 1 to 3 groups R¹, C1-6 haloalkyl optionally substituted by 1 to 3 groups R¹, C2-C6 alkenyl optionally substituted by 1 to 3 groups R¹, C3-C8 cycloalkyl optionally substituted by 1 to 3 groups R¹, C1-C6 alkylthio optionally substituted by 1 to 3 groups R¹, C6-C10 aryl optionally substituted by 1 to 3 groups R², a mono- or bicyclic heteroaryl group having 5 to 10 ring atoms and at least one ring atom which is nitrogen, oxygen or sulfur optionally substituted by 1 to 3 groups R², or
(ii) as defined in claim 1, and D, X, E, Y and Z are as defined in claim 1, without the provisos (i), (ii) and (iii) of claim 1, or a composition as claimed in claims 13.
